(19) United States
(12) Patent Application Publication
(10) Pub. No.: US 2022/0320442 A1
(43) Pub. Date: Oct. 6, 2022

(54) HETEROCYCLIC COMPOUND, ORGANIC LIGHT-EMITTING DIODE COMPRISING SAME, COMPOSITION FOR ORGANIC LAYER OF ORGANIC LIGHT-EMITTING DIODE, AND METHOD FOR MANUFACTURING ORGANIC LIGHT-EMITTING DIODE

(71) Applicant: LT MATERIALS CO., LTD., Yongin-City, Gyeonggi-do (KR)

(72) Inventors: Su-Yeon KIM, Yongin-si (KR); Seung-Gyu YANG, Yongin-si (KR); Young-Seok NO, Yongin-si (KR); Geon-Yu PARK, Yongin-si (KR); Dong-Jun KIM, Yongin-si (KR)

(73) Assignee: LT MATERIALS CO., LTD., Yongin-City, Gyeonggi-do (KR)

(21) Appl. No.: 17/608,799

(22) PCT Filed: Oct. 7, 2020
(86) PCT No.: PCT/KR2020/013665

§ 371 Date: Nov. 4, 2021

(30) Foreign Application Priority Data
Oct. 8, 2019 (KR) 10-2019-0124521

(51) Int. Cl. H01L 51/00; H01L 51/56; C07D 405/14; C07D 409/14; C07D 405/04; C07D 209/86

(52) U.S. Cl. CPC H01L 51/0067; H01L 51/56; C07D 405/14; C07D 409/14; H01L 51/0073; H01L 51/0074; H01L 51/0072; C07D 405/04; C07D 209/86; H01L 51/5012

(57) ABSTRACT

The present specification provides a heterocyclic compound represented by Chemical Formula 1, an organic light emitting device comprising the same, a composition for an organic material layer of an organic light emitting device, and a method for manufacturing an organic light emitting device.

### TECHNICAL FIELD

This application claims priority to and the benefits of Korean Patent Application No. 10-2019-0124521, filed with the Korean Intellectual Property Office on Oct. 8, 2019, the entire contents of which are incorporated herein by reference.

The present specification relates to a heterocyclic compound, an organic light emitting device comprising the same, a composition for an organic material layer of an organic light emitting device, and a method for manufacturing an organic light emitting device.

### BACKGROUND ART

An organic electroluminescent device is one type of self-emissive display devices, and has an advantage of having a wide viewing angle, and a high response speed as well as having an excellent contrast.

An organic light emitting device has a structure disposing an organic thin film between two electrodes. When a voltage is applied to an organic light emitting device having such a structure, electrons and holes injected from the two electrodes bind and pair in the organic thin film, and light emits as these annihilate. The organic thin film may be formed in a single layer or a multilayer as necessary.

A material of the organic thin film may have a light emitting function as necessary. For example, as a material of the organic thin film, compounds capable of forming a light emitting layer themselves alone may be used, or compounds capable of performing a role of a host or a dopant of a host-dopant-based light emitting layer may also be used. In addition thereto, compounds capable of performing roles of hole injection, hole transfer, electron blocking, hole blocking, electron transfer, electron injection and the like may also be used as a material of the organic thin film.

Development of an organic thin film material has been continuously required for enhancing performance, lifetime or efficiency of an organic light emitting device.

Studies on an organic light emitting device comprising a compound capable of satisfying conditions required for materials usable in an organic light emitting device, for example, satisfying proper energy level, electrochemical stability, thermal stability and the like, and having a chemical structure capable of performing various roles required in an organic light emitting device depending on substituents have been required.

### PRIOR ART DOCUMENTS

### Patent Documents

U.S. Pat. No. 4,356,429

### DISCLOSURE

### Technical Problem

The present application relates to a heterocyclic compound, an organic light emitting device comprising the same, a composition for an organic material layer of an organic light emitting device, and a method for manufacturing an organic light emitting device.

### Technical Solution

One embodiment of the present application provides a heterocyclic compound represented by the following Chemical Formula 1.

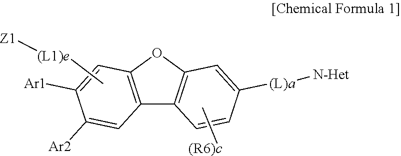

In Chemical Formula 1,

N-Het is a monocyclic or polycyclic C2 to C60 heterocyclic group substituted or unsubstituted and comprising one or more Ns,

L and L1 are the same as or different from each other, and each independently a direct bond; a substituted or unsubstituted C6 to C60 arylene group; or a substituted or unsubstituted C2 to C60 heteroarylene group,

Ar1 and Ar2 are the same as or different from each other, and each independently hydrogen; deuterium; —CN; or a substituted or unsubstituted C1 to C60 alkyl group, and

Z1 is a substituted or unsubstituted C6 to C60 aryl group; or represented by the following Chemical Formula A,

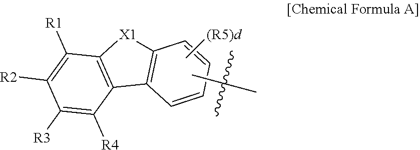

X1 is O; S; CR11R12; or NR13,

R1 to R4 are the same as or different from each other, and each independently hydrogen; a substituted or unsubstituted C1 to C60 alkyl group; a substituted or unsubstituted C6 to C60 aryl group; or a substituted or unsubstituted C2 to C60 heteroaryl group, or two or more groups adjacent to each other bond to each other to form a substituted or unsubstituted C6 to C60 aliphatic or aromatic hydrocarbon ring or a substituted or unsubstituted C2 to C60 heteroring,

R5 and R6 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)RR′; —SiRR′R″ and —NRR′,

R11 to R13, R, R′ and R″ are the same as or different from each other, and each independently a substituted or unsubstituted C1 to C60 alkyl group; a substituted or unsubstituted C6 to C60 aryl group; or a substituted or unsubstituted C2 to C60 heteroaryl group,

c and d are an integer of 0 to 3, and

a and e are an integer of 0 to 5.

In addition, one embodiment of the present application provides an organic light emitting device comprising a first electrode; a second electrode provided opposite to the first electrode; and one or more organic material layers provided between the first electrode and the second electrode, wherein one or more layers of the organic material layers comprise the heterocyclic compound represented by Chemical Formula 1.

In addition, one embodiment of the present application provides a composition for an organic material layer of an organic light emitting device, the composition comprising the heterocyclic compound represented by Chemical Formula 1 and a heterocyclic compound represented by the following Chemical Formula 2.

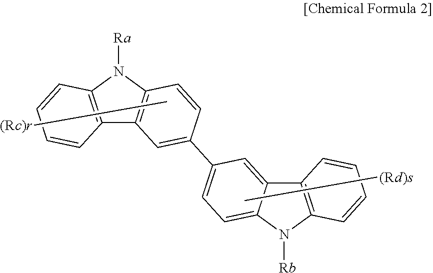

In Chemical Formula 2,

Ra and Rb are the same as or different from each other, and each independently a substituted or unsubstituted C6 to C60 aryl group; or a substituted or unsubstituted C2 to C60 heteroaryl group,

Rc and Rd are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; and a substituted or unsubstituted amine group, and

r and s are an integer of 0 to 7.

Lastly, one embodiment of the present application provides a method for manufacturing an organic light emitting device, the method comprising preparing a substrate; forming a first electrode on the substrate; forming one or more organic material layers on the first electrode; and forming a second electrode on the organic material layer, wherein the forming of organic material layers comprises forming one or more organic material layers using the composition for an organic material layer according to one embodiment of the present application.

### Advantageous Effects

A compound described in the present specification can be used as a material of an organic material layer of an organic light emitting device. In the organic light emitting device, the compound is capable of performing a role of a hole injection material, a hole transfer material, a light emitting material, an electron transfer material, an electron injection material or the like. Particularly, the compound can be used as a light emitting material of an organic light emitting device. For example, the compound can be used alone as a light emitting material, or two of the compounds can be used together as a light emitting material, and can be used as a host material of a light emitting layer.

Particularly, by substituting a No. 3 position of one side benzene ring of the dibenzofuran structure with an N-containing ring and substituting another benzene ring not substituted with the N-containing ring in the dibenzofuran ring with a specific substituent, a compound of Chemical Formula 1 has a more electron-stable structure by delocalizing LUND electrons of the N-containing ring side, and provides proper energy level and thermal stability to a device. By using the compounds of Chemical Formula 1, an organic light emitting device with improved lifetime, driving stability and efficiency can be manufactured.

### DESCRIPTION OF DRAWINGS

FIG. 1 to FIG. 3 are diagrams each schematically illustrating a lamination structure of an organic light emitting device according to one embodiment of the present application.

### REFERENCE NUMERAL

100: Substrate
200: Anode
300: Organic Material Layer
301: Hole Injection Layer
302: Hole Transfer Layer
303: Light Emitting Layer
304: Hole Blocking Layer
305: Electron Transfer Layer
306: Electron Injection Layer
400: Cathode

### MODE FOR DISCLOSURE

Hereinafter, the present application will be described in detail.

In the present specification, a “case of a substituent being not indicated in a chemical formula or compound structure” means that a hydrogen atom bonds to a carbon atom. However, since deuterium (²H) is an isotope of hydrogen, some hydrogen atoms may be deuterium.

In one embodiment of the present application, a “case of a substituent being not indicated in a chemical formula or compound structure” may mean that positions that may come as a substituent may all be hydrogen or deuterium. In other words, since deuterium is an isotope of hydrogen, some hydrogen atoms may be deuterium that is an isotope, and herein, a content of the deuterium may be from 0% to 100%.

In one embodiment of the present application, in a “case of a substituent being not indicated in a chemical formula or compound structure”, hydrogen and deuterium may be mixed in compounds when deuterium is not explicitly excluded such as a deuterium content being 0% or a hydrogen content being 100%. In other words, an expression of “substituent X is hydrogen” does not exclude deuterium such as a hydrogen content being 100% or a deuterium content being 0%, and therefore, may mean a state in which hydrogen and deuterium are mixed.

In one embodiment of the present application, deuterium is one of isotopes of hydrogen, is an element having deuteron formed with one proton and one neutron as a nucleus, and may be expressed as hydrogen-2, and the elemental symbol may also be written as D or 2H.

In one embodiment of the present application, an isotope means an atom with the same atomic number (Z) but with a different mass number (A), and may also be interpreted as an element with the same number of protons but with a different number of neutrons.

In one embodiment of the present application, a meaning of a content T % of a specific substituent may be defined as T2/T1×100=T % when the total number of substituents that a basic compound may have is defined as T1, and the number of specific substituents among these is defined as T2.

In other words, in one example, having a deuterium content of 20% in a phenyl group represented by

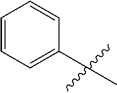 [SMILES: *c1ccccc1]

means that the total number of substituents that the phenyl group may have is 5 (T1 in the formula), and the number of deuterium among these is 1 (T2 in the formula). In other words, having a deuterium content of 20% in a phenyl group may be represented by the following structural formulae.

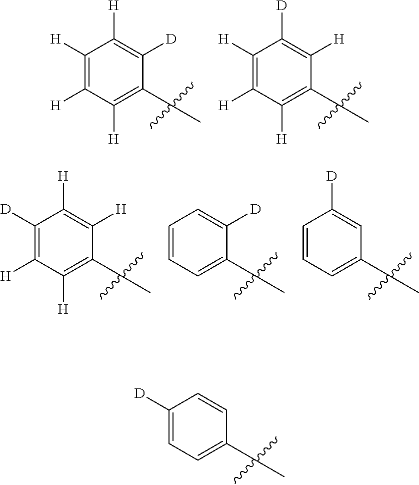 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, in one embodiment of the present application, “a phenyl group having a deuterium content of 0%” may mean a phenyl group that does not comprise a deuterium atom, that is, a phenyl group that has 5 hydrogen atoms.

In the present specification, the halogen may be fluorine, chlorine, bromine or iodine.

In the present specification, the alkyl group comprises linear or branched having 1 to 60 carbon atoms, and may be further substituted with other substituents. The number of carbon atoms of the alkyl group may be from 1 to 60, specifically from 1 to 40 and more specifically from 1 to 20. Specific examples thereof may comprise a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethylbutyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group and the like, but are not limited thereto.

In the present specification, the alkenyl group comprises linear or branched having 2 to 60 carbon atoms, and may be further substituted with other substituents. The number of carbon atoms of the alkenyl group may be from 2 to 60, specifically from 2 to 40 and more specifically from 2 to 20. Specific examples thereof may comprise a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group and the like, but are not limited thereto.

In the present specification, the alkynyl group comprises linear or branched having 2 to 60 carbon atoms, and may be further substituted with other substituents. The number of carbon atoms of the alkynyl group may be from 2 to 60, specifically from 2 to 40 and more specifically from 2 to 20.

In the present specification, the alkoxy group may be linear, branched or cyclic. The number of carbon atoms of the alkoxy group is not particularly limited, but is preferably from 1 to 20. Specific examples thereof may comprise methoxy, ethoxy, n-propoxy, isopropoxy, i-propyloxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy and the like, but are not limited thereto.

In the present specification, the cycloalkyl group comprises monocyclic or polycyclic having 3 to 60 carbon atoms, and may be further substituted with other substituents. Herein, the polycyclic means a group in which the cycloalkyl group is directly linked to or fused with other cyclic groups. Herein, the other cyclic groups may be a cycloalkyl group, but may also be different types of cyclic groups such as a heterocycloalkyl group, an aryl group and a heteroaryl group. The number of carbon groups of the cycloalkyl group may be from 3 to 60, specifically from 3 to 40 and more specifically from 5 to 20. Specific examples thereof may comprise a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group comprises O, S, Se, N or Si as a heteroatom, comprises monocyclic or polycyclic having 2 to 60 carbon atoms, and may be further substituted with other substituents. Herein, the polycyclic means a group in which the heterocycloalkyl group is directly linked to or fused with other cyclic groups. Herein, the other cyclic groups may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group comprises monocyclic or polycyclic having 6 to 60 carbon atoms, and may be further substituted with other substituents. Herein, the polycyclic means a group in which the aryl group is directly linked to or fused with other cyclic groups. Herein, the other cyclic groups may be an aryl group, but may also be different types of cyclic groups such as a cycloalkyl group, a heterocycloalkyl group and a heteroaryl group. The number of carbon atoms of the aryl group may be from 6 to 60, specifically from 6 to 40 and more specifically from 6 to 25. Specific examples of the aryl group may comprise a phenyl group, a biphenyl group, a triphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, an indenyl group, an acenaphthylenyl group, a 2,3-dihydro-1H-indenyl group, a fused ring thereof, and the like, but are not limited thereto.

In the present specification, a fluorenyl group may be substituted, and adjacent substituents may bond to each other to form a ring.

When the fluorenyl group is substituted, the substituted fluorenyl group may be represented by the following structures, but is not limited thereto.

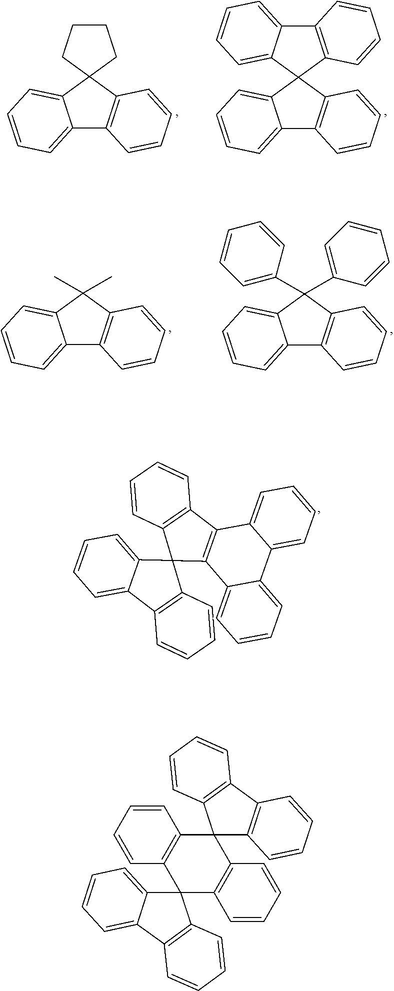

In the present specification, the heteroaryl group comprises S, O, Se, N or Si as a heteroatom, comprises monocyclic or polycyclic having 2 to 60 carbon atoms, and may be further substituted with other substituents. Herein, the polycyclic means a group in which the heteroaryl group is directly linked to or fused with other cyclic groups. Herein, the other cyclic groups may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. Specific examples of the heteroaryl group may comprise a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a qninozolinyl group, a naphthyridyl group, an acridinyl group, a phenanthridinyl group, an imidazopyridinyl group, a diazanaphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi(dibenzosilole), a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepine group, a 9,10-dihydroacridinyl group, a phenanthrazinyl group, a phenothiathiazinyl group, a phthalazinyl group, a naphthylidinyl group, a phenanthrolinyl group, a benzo[c][1,2,5]thiadiazolyl group, a 5,10-dihydrobenzo[b,e][1,4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and although not particularly limited thereto, the number of carbon atoms is preferably from 1 to 30. Specific examples of the amine group may comprise a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the arylene group means the aryl group having two bonding sites, that is, a divalent group. The descriptions on the aryl group provided above may be applied thereto except for those that are each a divalent group. In addition, the heteroarylene group means the heteroaryl group having two bonding sites, that is, a divalent group. The descriptions on the heteroaryl group provided above may be applied thereto except for those that are each a divalent group.

In the present specification, the phosphine oxide group is represented by —P(═O)R101R102, and R101 and R102 are the same as or different from each other and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the phosphine oxide may comprise a diphenylphosphine oxide group, a dinaphthylphosphine oxide group and the like, but are not limited thereto.

In the present specification, the silyl group is a substituent comprising Si, having the Si atom directly linked as a radical, and is represented by —SiR₁₀₄R₁₀₅R₁₀₆. R₁₀₄ to R₁₀₆ are the same as or different from each other, and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the silyl group may comprise a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present specification, the “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting ortho positions in a benzene ring, and two substituents substituting the same carbon in an aliphatic ring may be interpreted as groups “adjacent” to each other.

As the aliphatic or aromatic hydrocarbon ring or heteroring that adjacent groups may form, the structures illustrated as the cycloalkyl group, the cycloheteroalkyl group, the aryl group and the heteroaryl group described above may be used except for those that are not a monovalent group.

In the present specification, the term “substitution” means a hydrogen atom bonding to a carbon atom of a compound being changed to another substituent, and the position of substitution is not limited as long as it is a position at which the hydrogen atom is substituted, that is, a position at which a substituent can substitute, and when two or more substituents substitute, the two or more substituents may be the same as or different from each other.

In the present specification, “substituted or unsubstituted” means being substituted with one or more substituents selected from the group consisting of C1 to C60 linear or branched alkyl; C2 to C60 linear or branched alkenyl; C2 to C60 linear or branched alkynyl; C3 to C60 monocyclic or polycyclic cycloalkyl; C2 to C60 monocyclic or polycyclic heterocycloalkyl; C6 to C60 monocyclic or polycyclic aryl; C2 to C60 monocyclic or polycyclic heteroaryl; —SiRR′R″; —P(═O)RR′; C1 to C20 alkylamine; C6 to C60 monocyclic or polycyclic arylamine; and C2 to C60 monocyclic or polycyclic heteroarylamine, or being unsubstituted, or being substituted with a substituent linking two or more substituents selected from among the substituents illustrated above, or being unsubstituted, and,

R, R′ and R″ are the same as or different from each other, and each independently a substituted or unsubstituted alkyl group; a substituted or unsubstituted aryl group; or a substituted or unsubstituted heteroaryl group.

One embodiment of the present application provides a compound represented by Chemical Formula 1.

In one embodiment of the present application, Chemical Formula 1 may be represented by the following Chemical Formula 1-A or 1-B.

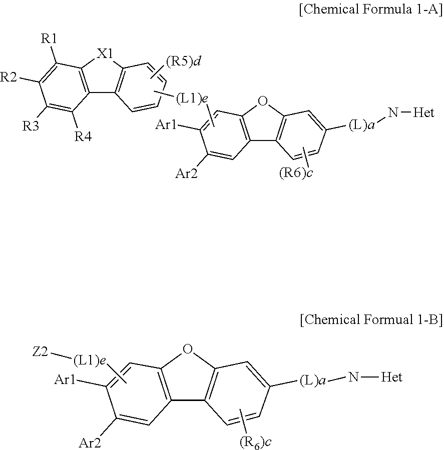

In Chemical Formulae 1-A and 1-B,

R1 to R6, N-Het, L, L1, X1, Ar1, Ar2, a, c, d and e have the same definitions as in Chemical Formula 1, and

Z2 is a substituted or unsubstituted C6 to C60 aryl group.

Particularly, as in Chemical Formula 1-B of one embodiment of the present application, a T1 energy level higher by approximately 2.5 eV or greater is obtained by an aryl group substituting another benzene ring not substituted with the N-containing ring in the dibenzofuran structure, and therefore, energy is readily transferred from a host to a dopant, and superior light emission efficiency is obtained as in arylene group or heteroarylene group-substituted chemical formulae.

In one embodiment of the present application, Chemical Formula 1-A may be represented by the following Chemical Formula 3-A or 4-A.

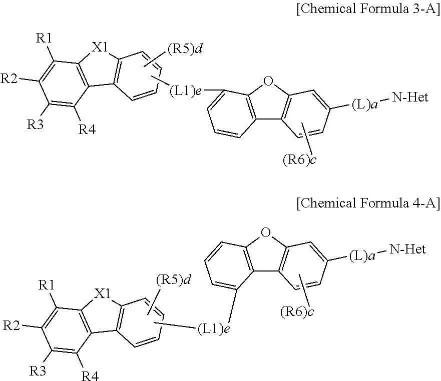

In Chemical Formulae 3-A and 4-A,

N-Het, L, L1, R1 to R6, X1, a, and c to e have the same definitions as in Chemical Formula 1-A.

In one embodiment of the present application, Chemical Formula 1-B may be represented by any one of the following Chemical Formula 3-B or 4-B.

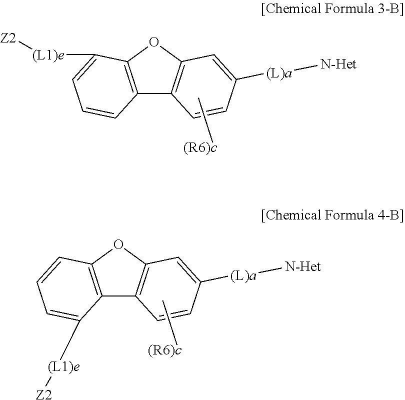

In Chemical Formulae 3-B and 4-B,

N-Het, L, L1, R6, Z2, a, c and e have the same definitions as in Chemical Formula 1-B.

In one embodiment of the present application, when, as in Chemical Formulae 3-A and 3-B, a No. 3 position of one side benzene ring of the dibenzofuran is substituted with N-Het and a No. 4 position of another benzene ring has a specific substituent, a driving voltage is low due to particularly more favorable current density compared to cases of substituting other positions, and triplet energy is also high.

In one embodiment of the present application, when, as in Chemical Formulae 4-A and 4-B, a No. 3 position of one side benzene ring of the dibenzofuran is substituted with N-Het and a No. 1 position of another benzene ring has a specific substituent, thermal stability is superior due to particularly lower Td compared to cases of substituting other positions, and lifetime properties of an organic light emitting device are particularly superior.

In other words, by the compound according to one embodiment of the present application having an N-Het substituent on a No. 3 position of the dibenzofuran and having a substituent on a specific position of another benzene ring, each has superior effects.

In one embodiment of the present application, R5 and R6 are the same as or different from each other, and may be each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)RR′; —SiRR′R″ and —NRR′.

In another embodiment, R5 and R6 are the same as or different from each other, and may be each independently selected from the group consisting of hydrogen; halogen; a substituted or unsubstituted C1 to C60 alkyl group; a substituted or unsubstituted C6 to C60 aryl group; a substituted or unsubstituted C2 to C60 heteroaryl group; —P(═O)RR′; —SiRR′R″ and —NRR′.

In another embodiment, R5 and R6 are the same as or different from each other, and may be each independently selected from the group consisting of hydrogen; halogen; a substituted or unsubstituted C1 to C60 alkyl group; a substituted or unsubstituted C6 to C40 aryl group; a substituted or unsubstituted C2 to C40 heteroaryl group; —P(═O)RR′; —SiRR′R″ and —NRR′.

In another embodiment, R5 and R6 may be hydrogen.

In one embodiment of the present application, Ar1 and Ar2 are the same as or different from each other, and each independently hydrogen; deuterium; —CN; or a substituted or unsubstituted C1 to C60 alkyl group.

In another embodiment, Ar1 and Ar2 are the same as or different from each other, and each independently hydrogen; or a substituted or unsubstituted C1 to C60 alkyl group.

In another embodiment, Ar1 and Ar2 are the same as or different from each other, and each independently hydrogen; or a substituted or unsubstituted C1 to C20 alkyl group.

In another embodiment, Ar1 and Ar2 are the same as or different from each other, and each independently hydrogen; or a C1 to C20 alkyl group.

In another embodiment, Ar1 and Ar2 are hydrogen.

In one embodiment of the present application, L may be a direct bond; a substituted or unsubstituted C6 to C60 arylene group; or a substituted or unsubstituted C2 to C60 heteroarylene group.

In another embodiment, L may be a direct bond; a substituted or unsubstituted C6 to C40 arylene group; or a substituted or unsubstituted C2 to C40 heteroarylene group.

In another embodiment, L may be a direct bond; a substituted or unsubstituted C6 to C40 monocyclic or polycyclic arylene group; or a substituted or unsubstituted C2 to C40 heteroarylene group.

In another embodiment, L may be a direct bond; a substituted or unsubstituted C6 to C40 monocyclic arylene group; or a substituted or unsubstituted C10 to C40 polycyclic arylene group.

In another embodiment, L may be a direct bond; a C6 to C40 monocyclic arylene group; or a C10 to C40 polycyclic arylene group.

In another embodiment, L may be a direct bond; a phenylene group; a biphenylene group; or a naphthylene group.

In another embodiment, L may be a direct bond.

In one embodiment of the present application, L1 may be a direct bond; a substituted or unsubstituted C6 to C60 arylene group; or a substituted or unsubstituted C2 to C60 heteroarylene group.

In another embodiment, L1 may be a direct bond; a substituted or unsubstituted C6 to C40 arylene group; or a substituted or unsubstituted C2 to C40 heteroarylene group.

In another embodiment, L1 may be a direct bond; a substituted or unsubstituted C6 to C40 monocyclic or polycyclic arylene group; or a substituted or unsubstituted C2 to C40 heteroarylene group.

In another embodiment, L1 may be a direct bond; a substituted or unsubstituted C6 to C40 monocyclic arylene group; or a substituted or unsubstituted C10 to C40 polycyclic arylene group.

In another embodiment, L1 may be a direct bond; a C6 to C40 monocyclic arylene group; or a C10 to C40 polycyclic arylene group.

In another embodiment, L1 may be a direct bond; a phenylene group; a biphenylene group; or a naphthylene group.

In one embodiment of the present application, R1 to R4 are the same as or different from each other, and each independently hydrogen; a substituted or unsubstituted C1 to C60 alkyl group; a substituted or unsubstituted C6 to C60 aryl group; or a substituted or unsubstituted C2 to C60 heteroaryl group, or two or more groups adjacent to each other may bond to each other to form a substituted or unsubstituted C6 to C60 aromatic hydrocarbon ring.

In another embodiment, R1 to R4 are the same as or different from each other, and each independently hydrogen; a substituted or unsubstituted C1 to C40 alkyl group; a substituted or unsubstituted C6 to C40 aryl group; or a substituted or unsubstituted C2 to C40 heteroaryl group, or two or more groups adjacent to each other may bond to each other to form a substituted or unsubstituted C6 to C40 aromatic hydrocarbon ring.

In another embodiment, R1 to R4 are the same as or different from each other, and each independently hydrogen; a C1 to C40 alkyl group; a C6 to C40 aryl group; or a C2 to C40 heteroaryl group, or two or more groups adjacent to each other may bond to each other to form a C6 to C40 aromatic hydrocarbon ring.

In another embodiment, R1 to R4 are the same as or different from each other, and each independently hydrogen; a C6 to C40 monocyclic aryl group; or a C10 to C40 polycyclic aryl group, or two or more groups adjacent to each other may bond to each other to form a C6 to C40 monocyclic aromatic hydrocarbon ring.

In another embodiment, R1 to R4 are the same as or different from each other, and each independently hydrogen; a phenyl group; a biphenyl group; or a triphenylenyl group, or two or more groups adjacent to each other may bond to each other to form a benzene ring.

In one embodiment of the present application, X1 may be O; S; CR11R12; or NR13.

In one embodiment of the present application, X1 may be O.

In one embodiment of the present application, X1 may be S.

In one embodiment of the present application, X1 may be CR11R12.

In one embodiment of the present application, X1 may be NR13.

Particularly, although the HOMO energy level is localized to one side when X1 has a substituent of NR13, the HOMO energy level is relatively delocalized when X1 has O, S and the like, which leads to a more stable electron-stable structure, and an organic light emitting device with improved lifetime, driving stability and efficiency may be manufactured.

In one embodiment of the present application, R11 to R13 are the same as or different from each other, and may be each independently a substituted or unsubstituted C1 to C60 alkyl group; a substituted or unsubstituted C6 to C60 aryl group; or a substituted or unsubstituted C2 to C60 heteroaryl group.

In another embodiment, R11 to R13 are the same as or different from each other, and may be each independently a C1 to C60 alkyl group; a C6 to C60 aryl group; or a C2 to C60 heteroaryl group.

In another embodiment, R11 to R13 are the same as or different from each other, and may be each independently a C6 to C60 aryl group.

In another embodiment, R11 to R13 are the same as or different from each other, and may be each independently a C6 to C40 monocyclic aryl group.

In another embodiment, R11 to R13 may be a phenyl group.

In one embodiment of the present application, R13 may be a phenyl group.

In one embodiment of the present application, Chemical Formula A of Chemical Formula 1 may be represented by any one of the following Chemical Formulae 1-1 to 1-6.

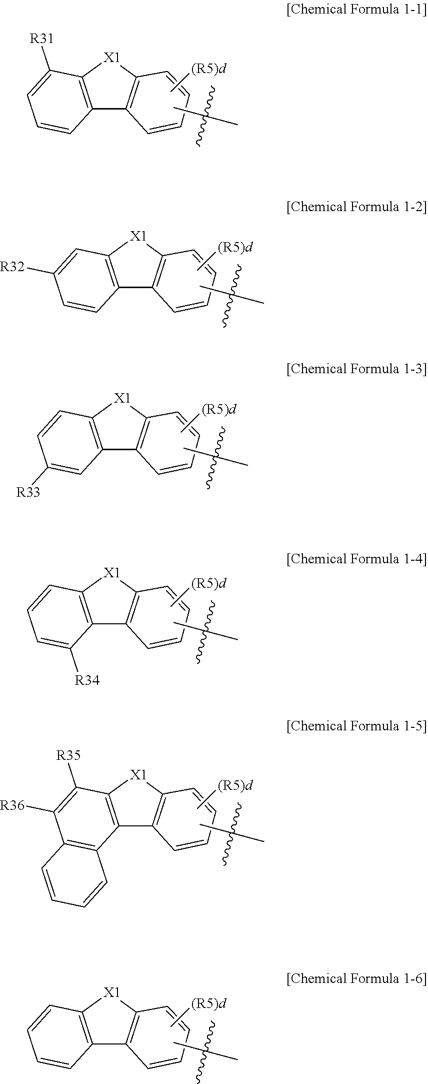

In Chemical Formulae 1-1 to 1-6,

X1, R5 and d have the same definitions as in Chemical Formula 1,

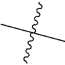

means a position linked to L1 of Chemical Formula 1,

R31 to R34 are the same as or different from each other, and each independently a substituted or unsubstituted C1 to C60 alkyl group; a substituted or unsubstituted C& to C60 aryl group; or a substituted or unsubstituted C2 to C60 heteroaryl group, and

R35 and R36 are the same as or different from each other, and each independently hydrogen; a substituted or unsubstituted C1 to C60 alkyl group; a substituted or unsubstituted C6 to C60 aryl group; or a substituted or unsubstituted C2 to C60 heteroaryl group.

In one embodiment of the present application, Z1 may be a substituted or unsubstituted C6 to C60 aryl group or represented by Chemical Formula A, and specifically, Z1 may be a substituted or unsubstituted C& to C60 aryl group.

In another embodiment, Z1 may be a substituted or unsubstituted C6 to C40 aryl group.

In another embodiment, Z1 may be a substituted or unsubstituted monocyclic or polycyclic C6 to C40 aryl group.

In another embodiment, Z1 may be a substituted or unsubstituted monocyclic C6 to C40 aryl group.

In another embodiment, Z1 may be a substituted or unsubstituted polycyclic C10 to C40 aryl group.

In another embodiment, Z1 may be a monocyclic C6 to C40 aryl group.

In another embodiment, Z1 may be a polycyclic C10 to C40 aryl group.

In another embodiment, Z1 may be a phenyl group; or a triphenylenyl group.

In one embodiment of the present application, Z2 may be a substituted or unsubstituted C6 to C60 aryl group.

In another embodiment, Z2 may be a substituted or unsubstituted C6 to C40 aryl group.

In another embodiment, Z2 may be a substituted or unsubstituted monocyclic or polycyclic C6 to C40 aryl group.

In another embodiment, Z2 may be a substituted or unsubstituted monocyclic C6 to C40 aryl group.

In another embodiment, Z2 may be a substituted or unsubstituted polycyclic C10 to C40 aryl group.

In another embodiment, Z2 may be a monocyclic C6 to C40 aryl group.

In another embodiment, Z2 may be a polycyclic C10 to C40 aryl group.

In another embodiment, Z2 may be a phenyl group; or a triphenylenyl group.

In one embodiment of the present application, R31 to R34 are the same as or different from each other, and may be each independently a substituted or unsubstituted C1 to C60 alkyl group; a substituted or unsubstituted C6 to C60 aryl group; or a substituted or unsubstituted C2 to C60 heteroaryl group.

In another embodiment, R31 to R34 are the same as or different from each other, and may be each independently a substituted or unsubstituted C6 to C60 aryl group.

In another embodiment, R31 to R34 are the same as or different from each other, and may be each independently a substituted or unsubstituted C6 to C40 aryl group.

In another embodiment, R31 to R34 are the same as or different from each other, and may be each independently a substituted or unsubstituted C6 to C40 monocyclic or polycyclic aryl group.

In another embodiment, R31 to R34 are the same as or different from each other, and may be each independently a C6 to C40 monocyclic aryl group; or a C10 to C40 polycyclic aryl group.

In another embodiment, R31 to R34 are the same as or different from each other, and may be each independently a phenyl group; or a triphenylenyl group.

In one embodiment of the present application, R35 and R36 may be hydrogen.

In one embodiment of the present application, N-Het may be a monocyclic or polycyclic C2 to C60 heterocyclic group substituted or unsubstituted and comprising one or more Ns.

In another embodiment, N-Het may be a monocyclic or polycyclic C2 to C60 heterocyclic group substituted or unsubstituted and comprising one or more and three or less Ns.

In another embodiment, N-Het may be a monocyclic C2 to C60 heterocyclic group substituted or unsubstituted and comprising one or more and three or less Ns.

In another embodiment, N-Het may be a monocyclic or polycyclic C2 to C40 heterocyclic group substituted or unsubstituted and comprising one or more and three or less Ns.

In another embodiment, N-Het may be a monocyclic C2 to C40 heterocyclic group substituted or unsubstituted and comprising one or more and three or less Ns.

In another embodiment, N-Het may be a monocyclic C2 to C40 heterocyclic group unsubstituted or substituted with one or more substituents selected from the group consisting of a C1 to C20 alkyl group, a C6 to C40 aryl group, a C2 to C40 heteroaryl group, —P(═)ORR′ and —SiRR′R″ or a substituent linking two or more of the above-described substituents, and comprising one or more and three or less Ns.

In another embodiment, N-Het may be a pyridine group; a pyrimidine group; or a triazine group unsubstituted or substituted with one or more substituents selected from the group consisting of a C1 to C20 alkyl group, a C6 to C40 aryl group, a C2 to C40 heteroaryl group, —P(═)ORR′ and —SiRR′R″ or a substituent linking two or more of the above-described substituents.

In another embodiment, N-Het may be a pyridine group unsubstituted or substituted with a phenyl group; a pyrimidine group unsubstituted or substituted with a phenyl group; or a triazine group unsubstituted or substituted with one or more substituents selected from the group consisting of a triphenylenyl group, a diphenylfluorene group, a phenyl group unsubstituted or substituted with —P(═)ORR′ or —SiRR′R″, a biphenyl group, a dibenzofuran group, a dimethylfluorene group and a dibenzothiophene group.

In one embodiment of the present application, N-Het may be selected from among the following structural formulae.

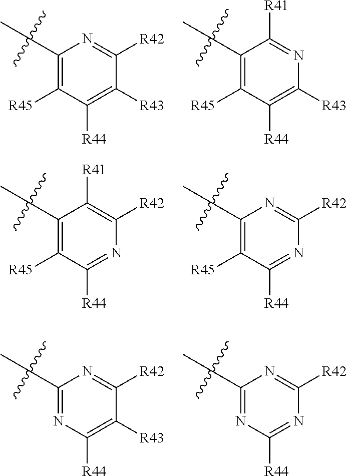

In the structural formulae,

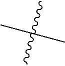

means a position linked to L of Chemical Formula 1, and

R41 to R45 are the same as or different from each other, and each independently hydrogen; a substituted or unsubstituted C1 to C60 alkyl group; a substituted or unsubstituted C6 to C60 aryl group; or a substituted or unsubstituted C2 to C60 heteroaryl group.

In one embodiment of the present application, R41 to R45 are the same as or different from each other, and may be each independently hydrogen; a substituted or unsubstituted C1 to C60 alkyl group; a substituted or unsubstituted C6 to C60 aryl group; or a substituted or unsubstituted C2 to C60 heteroaryl group.

In another embodiment, R41 to R45 are the same as or different from each other, and may be each independently hydrogen; a substituted or unsubstituted C1 to C40 alkyl group; a substituted or unsubstituted C6 to C40 aryl group; or a substituted or unsubstituted C2 to C40 heteroaryl group.

In another embodiment, R41 to R45 are the same as or different from each other, and may be each independently hydrogen; a C6 to C40 aryl group unsubstituted or substituted with one or more substituents selected from the group consisting of a C1 to C20 alkyl group, a C6 to C40 aryl group, a C2 to C40 heteroaryl group, —P(═)ORR′ and —SiRR′R″; or a C2 to C40 heteroaryl group.

In another embodiment, R41 to R45 are the same as or different from each other, and each independently hydrogen; a phenyl group unsubstituted or substituted with one or more substituents selected from the group consisting of a phenyl group, a triphenylenyl group, a diphenylfluorenyl group, —P(═)ORR′ and —SiRR′R″; a biphenyl group; a dibenzofuran group; a dibenzothiophene group; or a dimethylfluorenyl group.

In one embodiment of the present application, R, R′ and R″ are the same as or different from each other, and may be each independently a substituted or unsubstituted C1 to C60 alkyl group; a substituted or unsubstituted C6 to C60 aryl group; or a substituted or unsubstituted C2 to C60 heteroaryl group.

In another embodiment, R, R′ and R″ are the same as or different from each other, and may be each independently a substituted or unsubstituted C6 to C60 aryl group.

In another embodiment, R, R′ and R″ are the same as or different from each other, and may be each independently a substituted or unsubstituted C6 to C60 monocyclic or polycyclic aryl group.

In another embodiment, R, R′ and R″ are the same as or different from each other, and may be each independently a substituted or unsubstituted C6 to C40 monocyclic aryl group.

In another embodiment, R, R′ and R″ are the same as or different from each other, and may be each independently a C6 to C20 monocyclic aryl group.

In another embodiment, R, R′ and R″ may be a phenyl group.

According to one embodiment of the present application, Chemical Formula 1 may be represented by any one of the following compounds, but is not limited thereto.

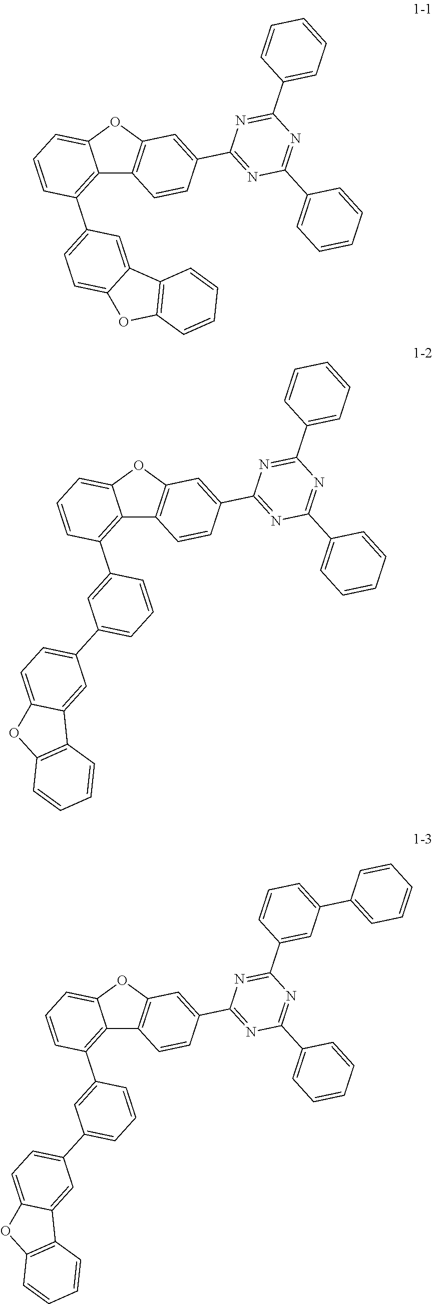 [SMILES: c1ccc(-c2nc(-c3ccccc3)nc(-c3ccc4c(c3)oc3cccc(-c5ccc6oc7ccccc7c6c5)c34)n2)cc1 ; c1ccc(-c2nc(-c3ccccc3)nc(-c3ccc4c(c3)oc3cccc(-c5cccc(-c6ccc7oc8ccccc8c7c6)c5)c34)n2)cc1 ; c1ccc(-c2cccc(-c3nc(-c4ccccc4)nc(-c4ccc5c(c4)oc4cccc(-c6cccc(-c7ccc8oc9ccccc9c8c7)c6)c45)n3)c2)cc1]

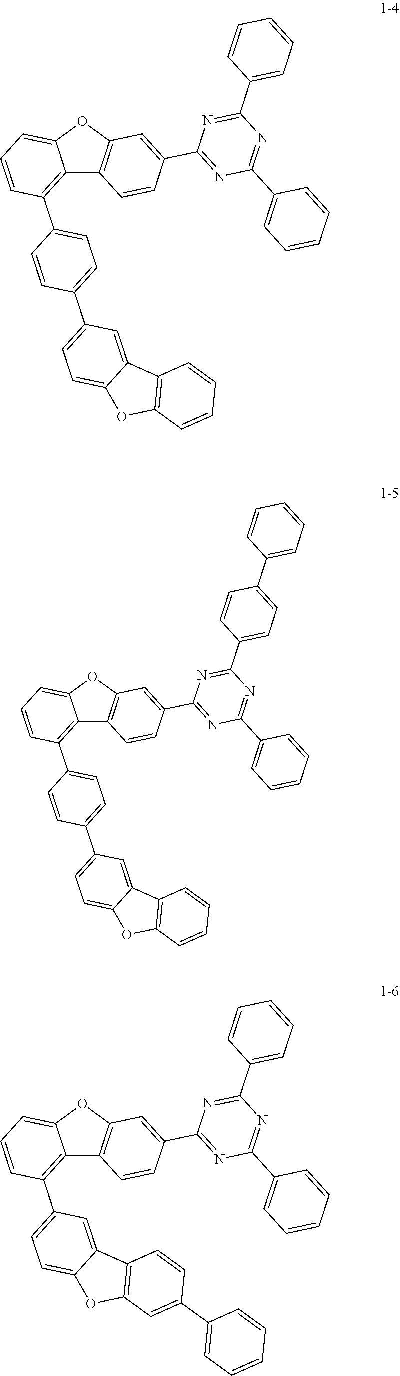 [SMILES: c1ccc(-c2nc(-c3ccccc3)nc(-c3ccc4c(c3)oc3cccc(-c5ccc(-c6ccc7oc8ccccc8c7c6)cc5)c34)n2)cc1 ; c1ccc(-c2ccc(-c3nc(-c4ccccc4)nc(-c4ccc5c(c4)oc4cccc(-c6ccc(-c7ccc8oc9ccccc9c8c7)cc6)c45)n3)cc2)cc1 ; c1ccc(-c2ccc3c(c2)oc2ccc(-c4cccc5oc6cc(-c7nc(-c8ccccc8)nc(-c8ccccc8)n7)ccc6c45)cc23)cc1]

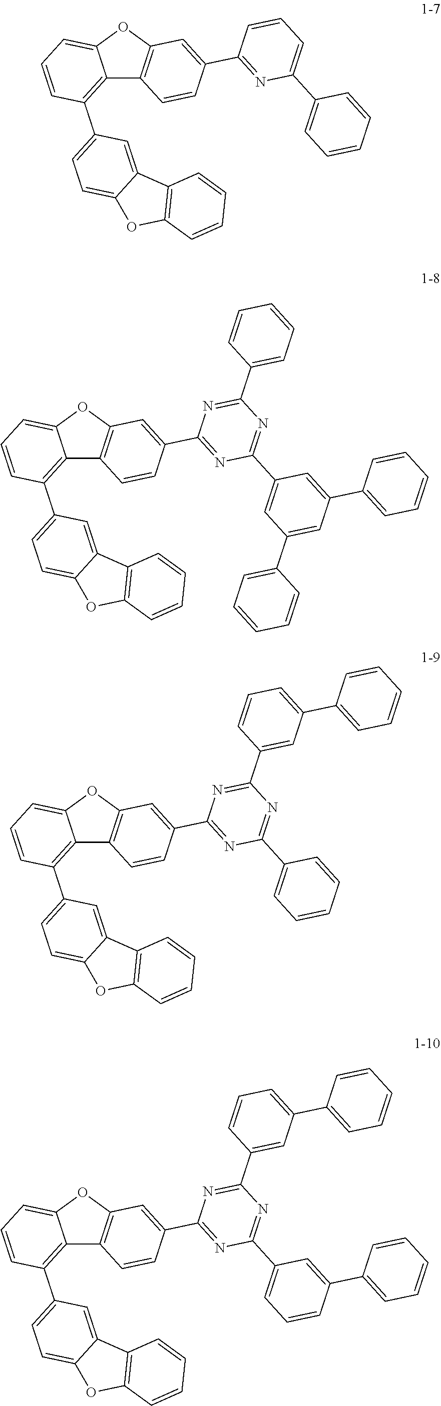 [SMILES: c1ccc(-c2cccc(-c3ccc4c(c3)oc3cccc(-c5ccc6oc7ccccc7c6c5)c34)n2)cc1 ; c1ccc(-c2cc(-c3ccccc3)cc(-c3nc(-c4ccccc4)nc(-c4ccc5c(c4)oc4cccc(-c6ccc7oc8ccccc8c7c6)c45)n3)c2)cc1 ; c1ccc(-c2cccc(-c3nc(-c4ccccc4)nc(-c4ccc5c(c4)oc4cccc(-c6ccc7oc8ccccc8c7c6)c45)n3)c2)cc1 ; c1ccc(-c2cccc(-c3nc(-c4cccc(-c5ccccc5)c4)nc(-c4ccc5c(c4)oc4cccc(-c6ccc7oc8ccccc8c7c6)c45)n3)c2)cc1]

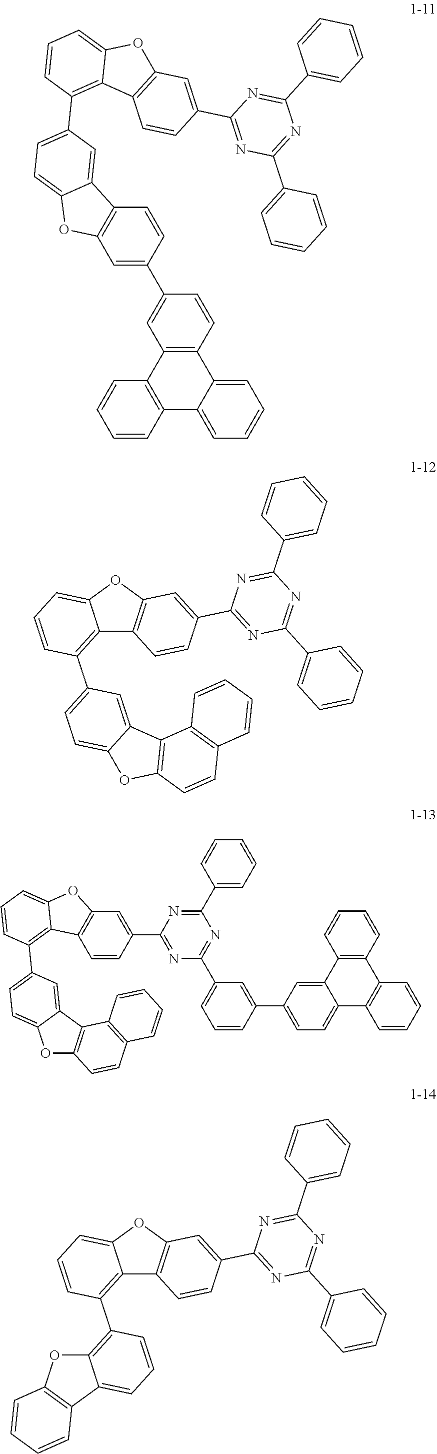 [SMILES: c1ccc(-c2nc(-c3ccccc3)nc(-c3ccc4c(c3)oc3cccc(-c5ccc6oc7cc(-c8ccc9c%10ccccc%10c%10ccccc%10c9c8)ccc7c6c5)c34)n2)cc1 ; c1ccc(-c2nc(-c3ccccc3)nc(-c3ccc4c(c3)oc3cccc(-c5ccc6oc7ccc8ccccc8c7c6c5)c34)n2)cc1 ; c1ccc(-c2nc(-c3cccc(-c4ccc5c6ccccc6c6ccccc6c5c4)c3)nc(-c3ccc4c(c3)oc3cccc(-c5ccc6oc7ccc8ccccc8c7c6c5)c34)n2)cc1 ; c1ccc(-c2nc(-c3ccccc3)nc(-c3ccc4c(c3)oc3cccc(-c5cccc6c5oc5ccccc56)c34)n2)cc1]

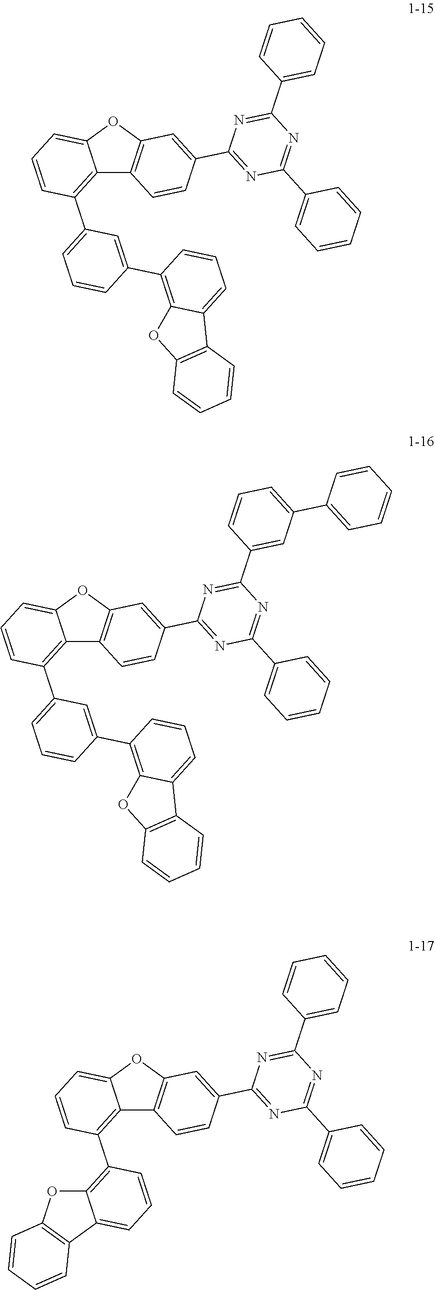 [SMILES: c1ccc(-c2nc(-c3ccccc3)nc(-c3ccc4c(c3)oc3cccc(-c5cccc(-c6cccc7c6oc6ccccc67)c5)c34)n2)cc1 ; c1ccc(-c2cccc(-c3nc(-c4ccccc4)nc(-c4ccc5c(c4)oc4cccc(-c6cccc(-c7cccc8c7oc7ccccc78)c6)c45)n3)c2)cc1 ; c1ccc(-c2nc(-c3ccccc3)nc(-c3ccc4c(c3)oc3cccc(-c5cccc6c5oc5ccccc56)c34)n2)cc1]

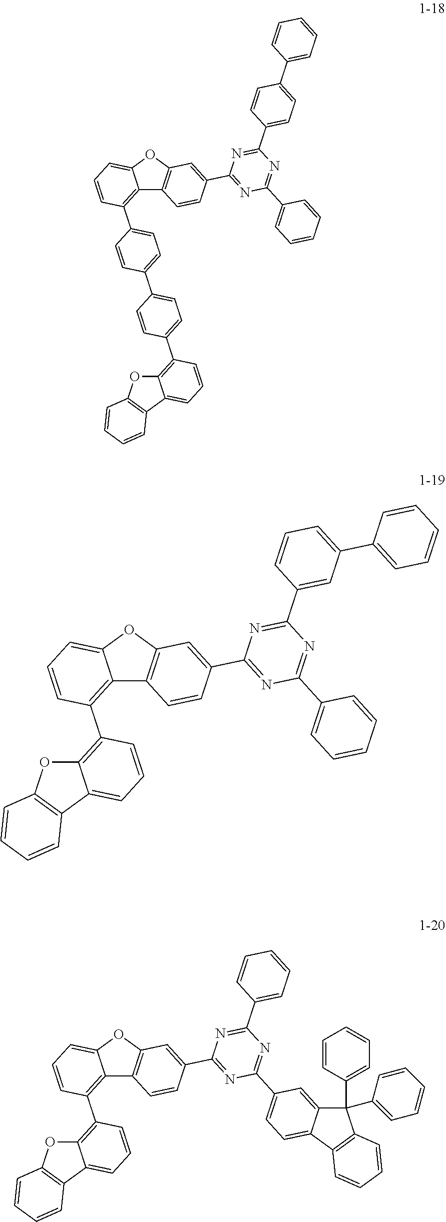 [SMILES: c1ccc(-c2ccc(-c3nc(-c4ccccc4)nc(-c4ccc5c(c4)oc4cccc(-c6ccc(-c7ccc(-c8cccc9c8oc8ccccc89)cc7)cc6)c45)n3)cc2)cc1 ; c1ccc(-c2cccc(-c3nc(-c4ccccc4)nc(-c4ccc5c(c4)oc4cccc(-c6cccc7c6oc6ccccc67)c45)n3)c2)cc1 ; c1ccc(-c2nc(-c3ccc4c(c3)C(c3ccccc3)(c3ccccc3)c3ccccc3-4)nc(-c3ccc4c(c3)oc3cccc(-c5cccc6c5oc5ccccc56)c34)n2)cc1]

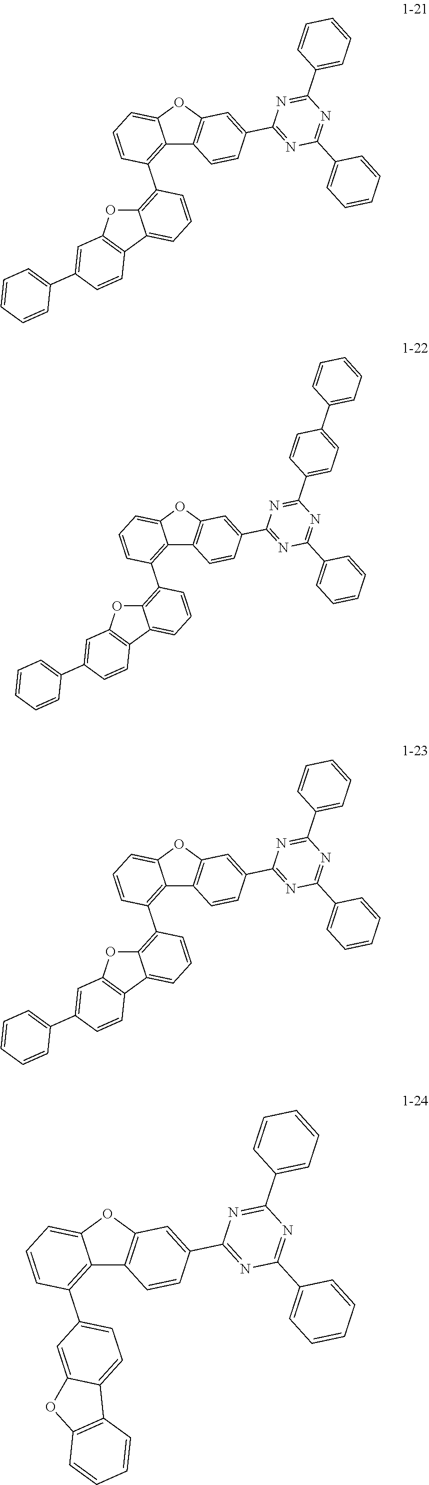 [SMILES: c1ccc(-c2ccc3c(c2)oc2c(-c4cccc5oc6cc(-c7nc(-c8ccccc8)nc(-c8ccccc8)n7)ccc6c45)cccc23)cc1 ; c1ccc(-c2ccc(-c3nc(-c4ccccc4)nc(-c4ccc5c(c4)oc4cccc(-c6cccc7c6oc6cc(-c8ccccc8)ccc67)c45)n3)cc2)cc1 ; c1ccc(-c2ccc3c(c2)oc2c(-c4cccc5oc6cc(-c7nc(-c8ccccc8)nc(-c8ccccc8)n7)ccc6c45)cccc23)cc1 ; c1ccc(-c2nc(-c3ccccc3)nc(-c3ccc4c(c3)oc3cccc(-c5ccc6c(c5)oc5ccccc56)c34)n2)cc1]

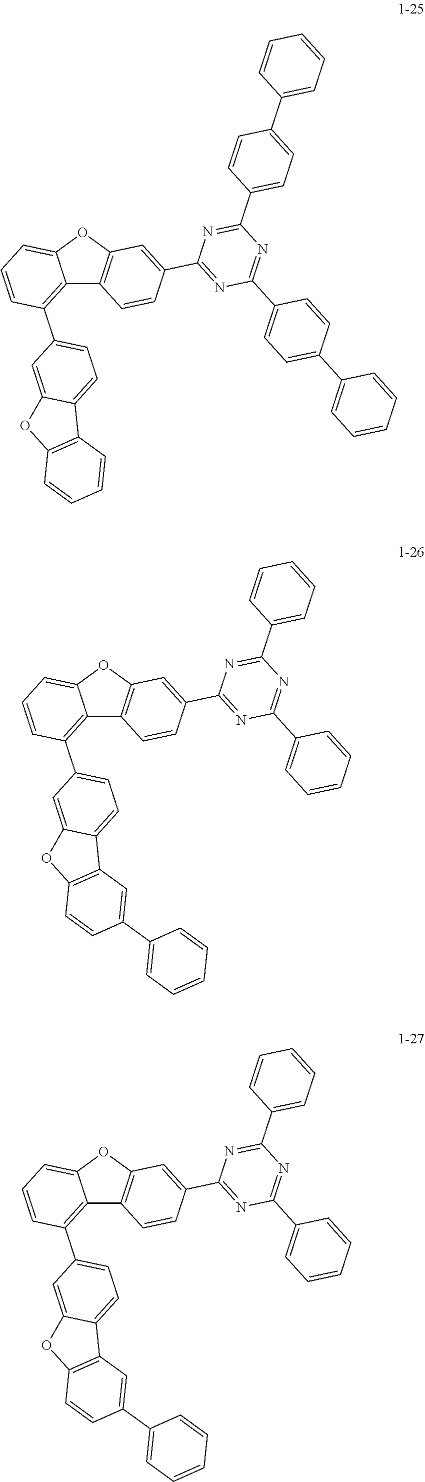 [SMILES: c1ccc(-c2ccc(-c3nc(-c4ccc(-c5ccccc5)cc4)nc(-c4ccc5c(c4)oc4cccc(-c6ccc7c(c6)oc6ccccc67)c45)n3)cc2)cc1 ; c1ccc(-c2ccc3oc4cc(-c5cccc6oc7cc(-c8nc(-c9ccccc9)nc(-c9ccccc9)n8)ccc7c56)ccc4c3c2)cc1 ; c1ccc(-c2ccc3oc4cc(-c5cccc6oc7cc(-c8nc(-c9ccccc9)nc(-c9ccccc9)n8)ccc7c56)ccc4c3c2)cc1]

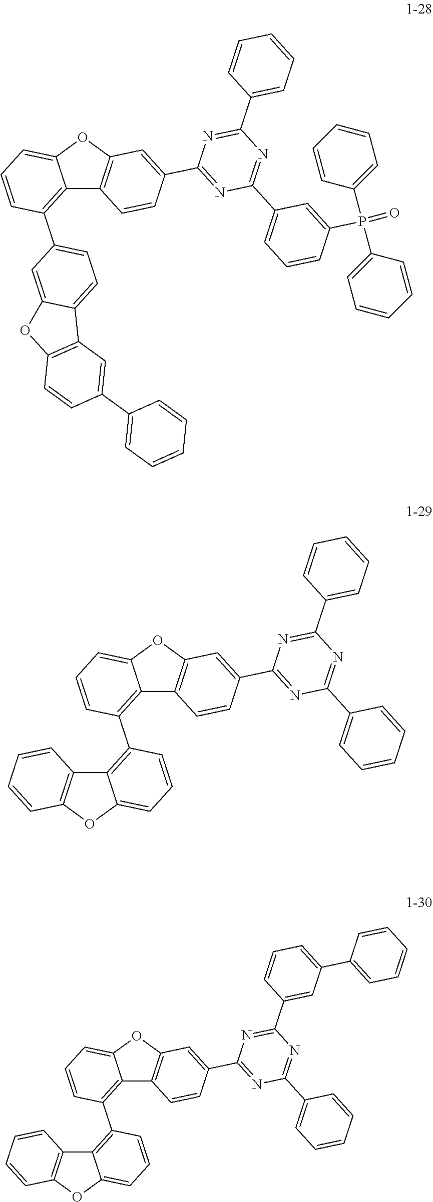 [SMILES: O=P(c1ccccc1)(c1ccccc1)c1cccc(-c2nc(-c3ccccc3)nc(-c3ccc4c(c3)oc3cccc(-c5ccc6c(c5)oc5ccc(-c7ccccc7)cc56)c34)n2)c1 ; c1ccc(-c2nc(-c3ccccc3)nc(-c3ccc4c(c3)oc3cccc(-c5cccc6oc7ccccc7c56)c34)n2)cc1 ; c1ccc(-c2cccc(-c3nc(-c4ccccc4)nc(-c4ccc5c(c4)oc4cccc(-c6cccc7oc8ccccc8c67)c45)n3)c2)cc1]

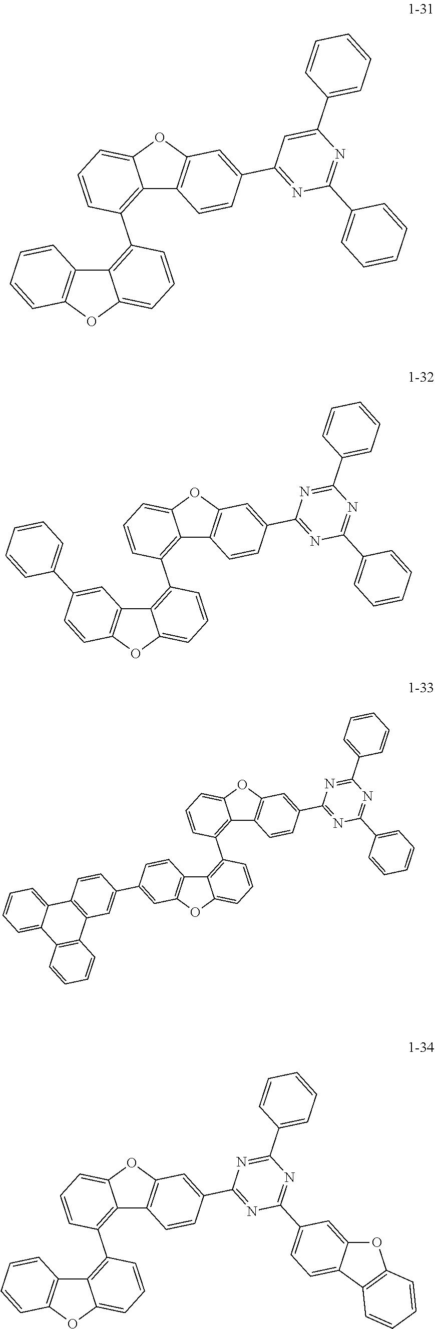 [SMILES: c1ccc(-c2cc(-c3ccc4c(c3)oc3cccc(-c5cccc6oc7ccccc7c56)c34)nc(-c3ccccc3)n2)cc1 ; c1ccc(-c2ccc3oc4cccc(-c5cccc6oc7cc(-c8nc(-c9ccccc9)nc(-c9ccccc9)n8)ccc7c56)c4c3c2)cc1 ; c1ccc(-c2nc(-c3ccccc3)nc(-c3ccc4c(c3)oc3cccc(-c5cccc6oc7cc(-c8ccc9c%10ccccc%10c%10ccccc%10c9c8)ccc7c56)c34)n2)cc1 ; c1ccc(-c2nc(-c3ccc4c(c3)oc3ccccc34)nc(-c3ccc4c(c3)oc3cccc(-c5cccc6oc7ccccc7c56)c34)n2)cc1]

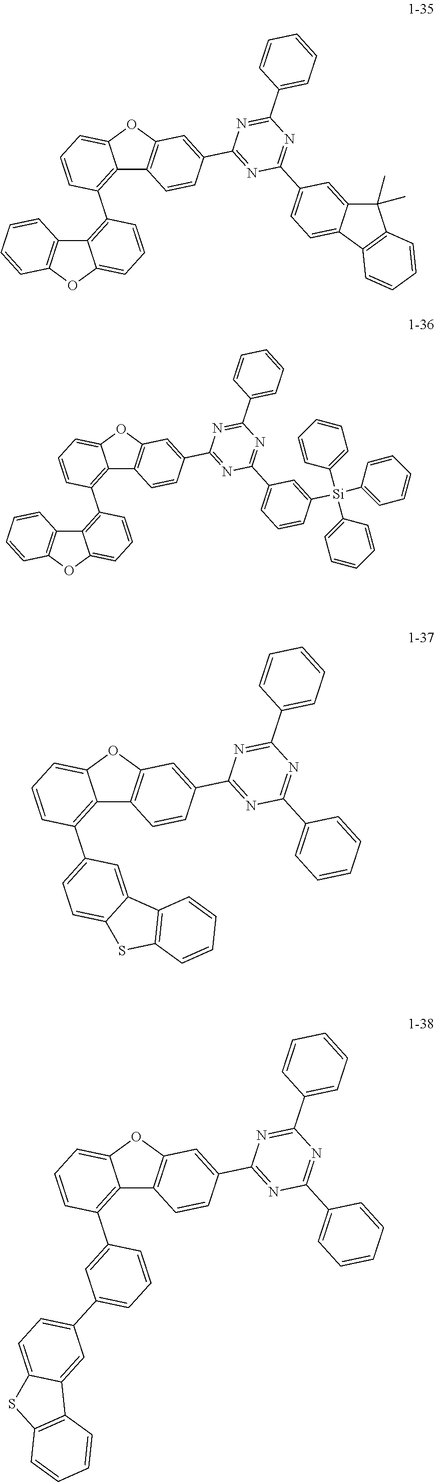 [SMILES: CC1(C)c2ccccc2-c2ccc(-c3nc(-c4ccccc4)nc(-c4ccc5c(c4)oc4cccc(-c6cccc7oc8ccccc8c67)c45)n3)cc21 ; c1ccc(-c2nc(-c3cccc([Si](c4ccccc4)(c4ccccc4)c4ccccc4)c3)nc(-c3ccc4c(c3)oc3cccc(-c5cccc6oc7ccccc7c56)c34)n2)cc1 ; c1ccc(-c2nc(-c3ccccc3)nc(-c3ccc4c(c3)oc3cccc(-c5ccc6sc7ccccc7c6c5)c34)n2)cc1 ; c1ccc(-c2nc(-c3ccccc3)nc(-c3ccc4c(c3)oc3cccc(-c5cccc(-c6ccc7sc8ccccc8c7c6)c5)c34)n2)cc1]

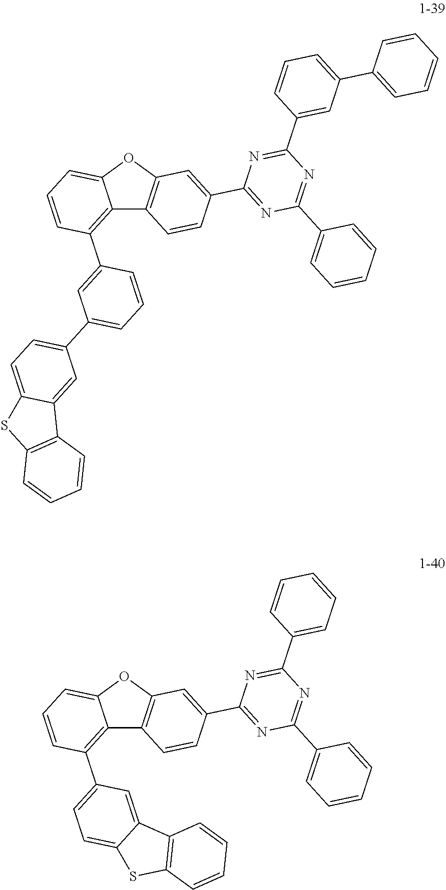 [SMILES: c1ccc(-c2cccc(-c3nc(-c4ccccc4)nc(-c4ccc5c(c4)oc4cccc(-c6cccc(-c7ccc8sc9ccccc9c8c7)c6)c45)n3)c2)cc1 ; c1ccc(-c2nc(-c3ccccc3)nc(-c3ccc4c(c3)oc3cccc(-c5ccc6sc7ccccc7c6c5)c34)n2)cc1]

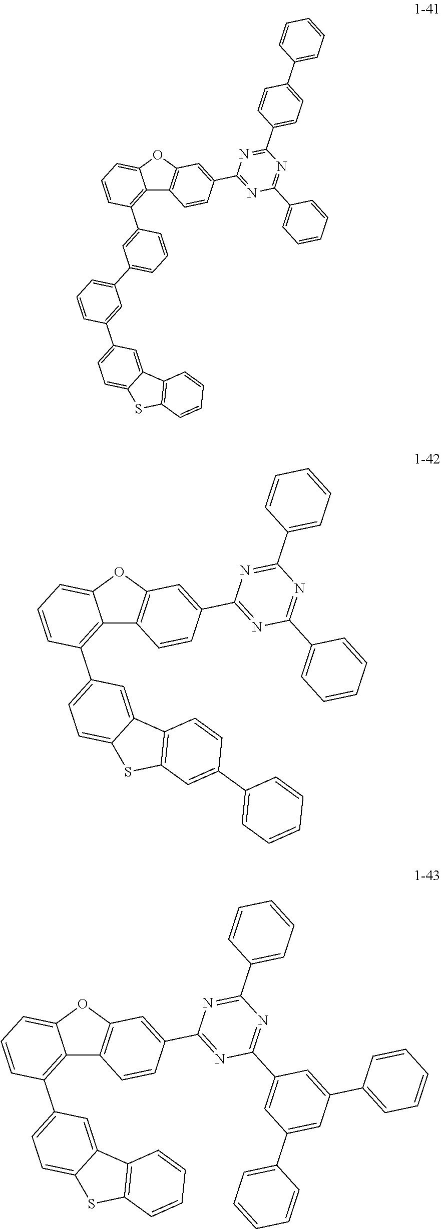 [SMILES: c1ccc(-c2ccc(-c3nc(-c4ccccc4)nc(-c4ccc5c(c4)oc4cccc(-c6cccc(-c7cccc(-c8ccc9sc%10ccccc%10c9c8)c7)c6)c45)n3)cc2)cc1 ; c1ccc(-c2ccc3c(c2)sc2ccc(-c4cccc5oc6cc(-c7nc(-c8ccccc8)nc(-c8ccccc8)n7)ccc6c45)cc23)cc1 ; c1ccc(-c2cc(-c3ccccc3)cc(-c3nc(-c4ccccc4)nc(-c4ccc5c(c4)oc4cccc(-c6ccc7sc8ccccc8c7c6)c45)n3)c2)cc1]

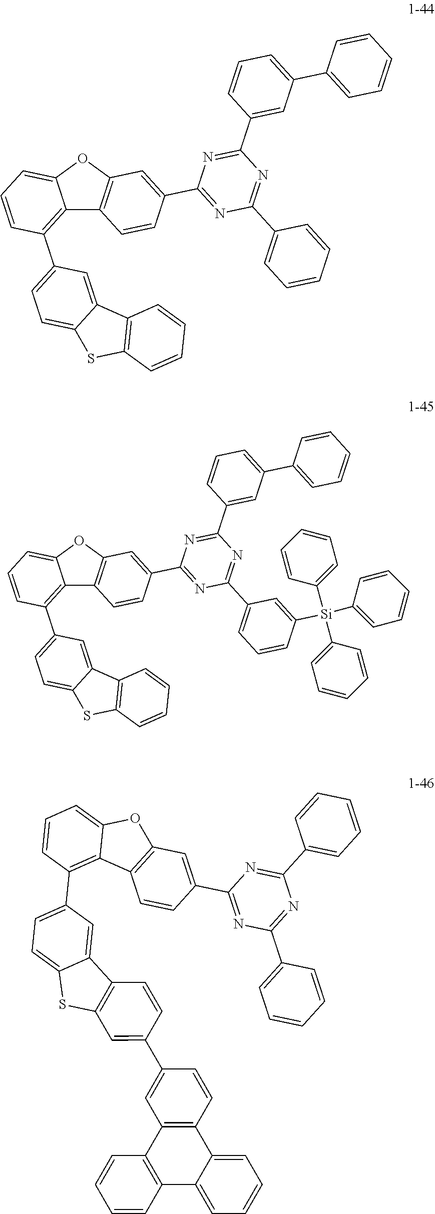 [SMILES: c1ccc(-c2cccc(-c3nc(-c4ccccc4)nc(-c4ccc5c(c4)oc4cccc(-c6ccc7sc8ccccc8c7c6)c45)n3)c2)cc1 ; c1ccc(-c2cccc(-c3nc(-c4cccc([Si](c5ccccc5)(c5ccccc5)c5ccccc5)c4)nc(-c4ccc5c(c4)oc4cccc(-c6ccc7sc8ccccc8c7c6)c45)n3)c2)cc1 ; c1ccc(-c2nc(-c3ccccc3)nc(-c3ccc4c(c3)oc3cccc(-c5ccc6sc7cc(-c8ccc9c%10ccccc%10c%10ccccc%10c9c8)ccc7c6c5)c34)n2)cc1]

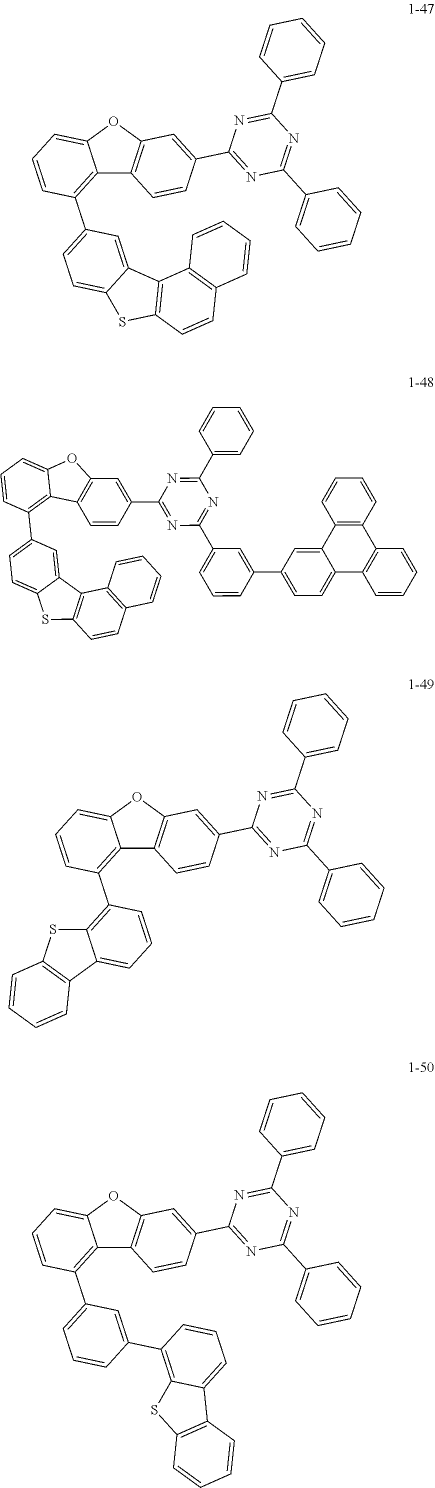 [SMILES: c1ccc(-c2nc(-c3ccccc3)nc(-c3ccc4c(c3)oc3cccc(-c5ccc6sc7ccc8ccccc8c7c6c5)c34)n2)cc1 ; c1ccc(-c2nc(-c3cccc(-c4ccc5c6ccccc6c6ccccc6c5c4)c3)nc(-c3ccc4c(c3)oc3cccc(-c5ccc6sc7ccc8ccccc8c7c6c5)c34)n2)cc1 ; c1ccc(-c2nc(-c3ccccc3)nc(-c3ccc4c(c3)oc3cccc(-c5cccc6c5sc5ccccc56)c34)n2)cc1 ; c1ccc(-c2nc(-c3ccccc3)nc(-c3ccc4c(c3)oc3cccc(-c5cccc(-c6cccc7c6sc6ccccc67)c5)c34)n2)cc1]

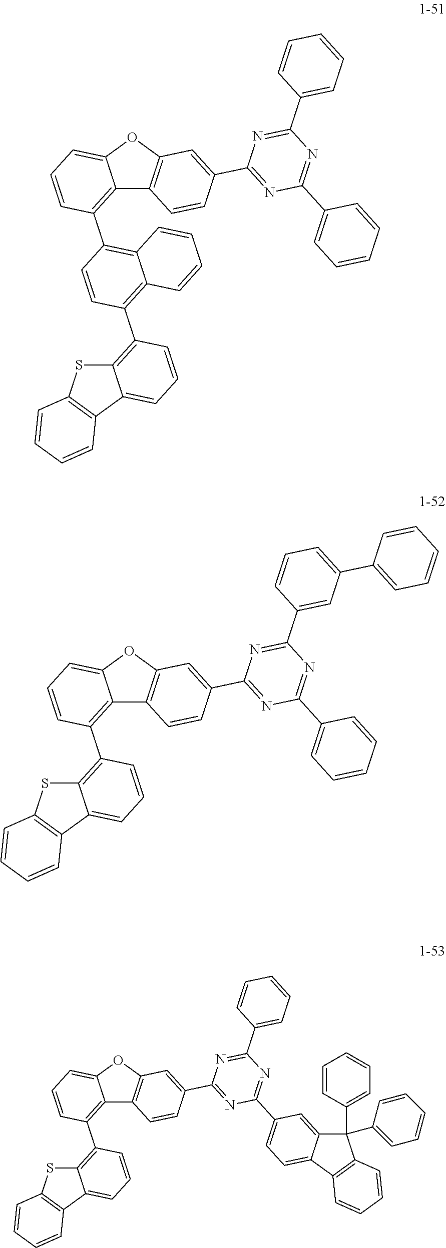 [SMILES: c1ccc(-c2nc(-c3ccccc3)nc(-c3ccc4c(c3)oc3cccc(-c5ccc(-c6cccc7c6sc6ccccc67)c6ccccc56)c34)n2)cc1 ; c1ccc(-c2cccc(-c3nc(-c4ccccc4)nc(-c4ccc5c(c4)oc4cccc(-c6cccc7c6sc6ccccc67)c45)n3)c2)cc1 ; c1ccc(-c2nc(-c3ccc4c(c3)C(c3ccccc3)(c3ccccc3)c3ccccc3-4)nc(-c3ccc4c(c3)oc3cccc(-c5cccc6c5sc5ccccc56)c34)n2)cc1]

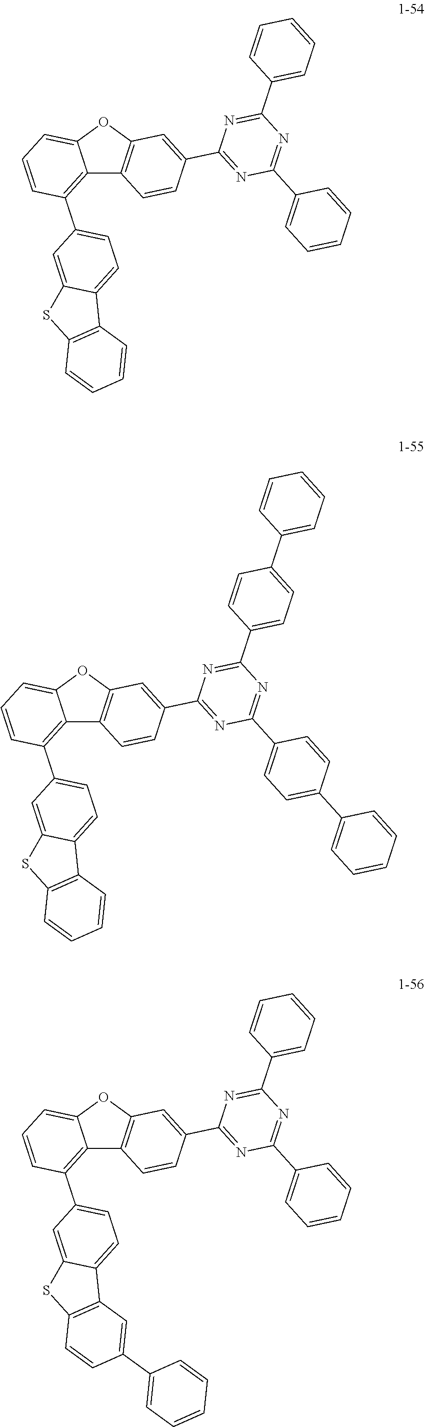 [SMILES: c1ccc(-c2nc(-c3ccccc3)nc(-c3ccc4c(c3)oc3cccc(-c5ccc6c(c5)sc5ccccc56)c34)n2)cc1 ; c1ccc(-c2ccc(-c3nc(-c4ccc(-c5ccccc5)cc4)nc(-c4ccc5c(c4)oc4cccc(-c6ccc7c(c6)sc6ccccc67)c45)n3)cc2)cc1 ; c1ccc(-c2ccc3sc4cc(-c5cccc6oc7cc(-c8nc(-c9ccccc9)nc(-c9ccccc9)n8)ccc7c56)ccc4c3c2)cc1]

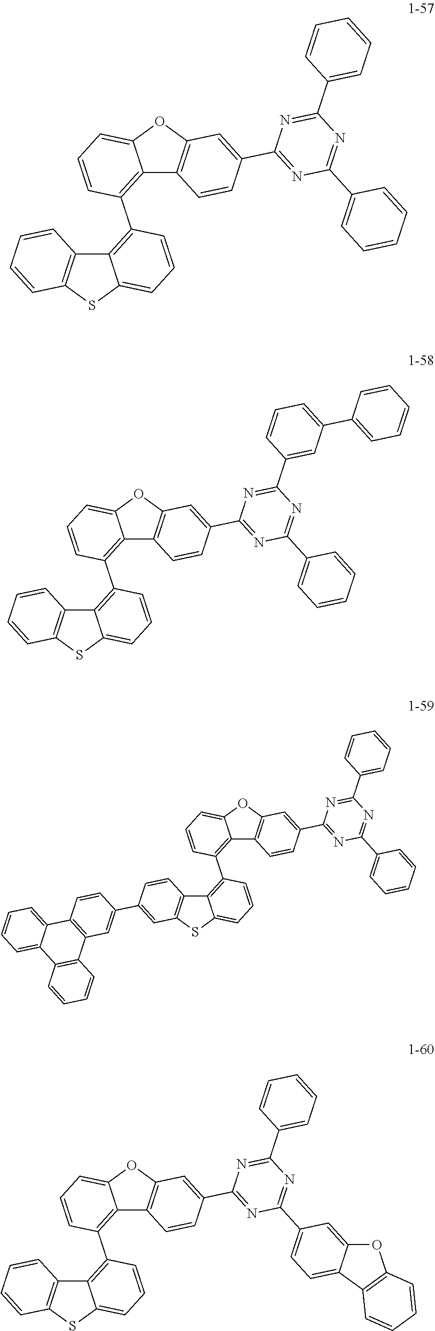 [SMILES: c1ccc(-c2nc(-c3ccccc3)nc(-c3ccc4c(c3)oc3cccc(-c5cccc6sc7ccccc7c56)c34)n2)cc1 ; c1ccc(-c2cccc(-c3nc(-c4ccccc4)nc(-c4ccc5c(c4)oc4cccc(-c6cccc7sc8ccccc8c67)c45)n3)c2)cc1 ; c1ccc(-c2nc(-c3ccccc3)nc(-c3ccc4c(c3)oc3cccc(-c5cccc6sc7cc(-c8ccc9c%10ccccc%10c%10ccccc%10c9c8)ccc7c56)c34)n2)cc1 ; c1ccc(-c2nc(-c3ccc4c(c3)oc3ccccc34)nc(-c3ccc4c(c3)oc3cccc(-c5cccc6sc7ccccc7c56)c34)n2)cc1]

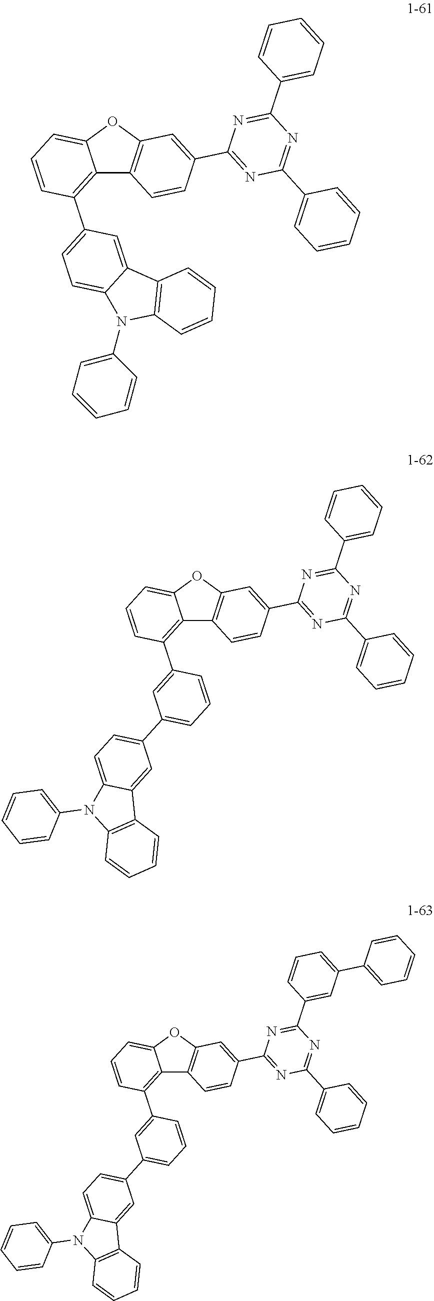 [SMILES: c1ccc(-c2nc(-c3ccccc3)nc(-c3ccc4c(c3)oc3cccc(-c5ccc6c(c5)c5ccccc5n6-c5ccccc5)c34)n2)cc1 ; c1ccc(-c2nc(-c3ccccc3)nc(-c3ccc4c(c3)oc3cccc(-c5cccc(-c6ccc7c(c6)c6ccccc6n7-c6ccccc6)c5)c34)n2)cc1 ; c1ccc(-c2cccc(-c3nc(-c4ccccc4)nc(-c4ccc5c(c4)oc4cccc(-c6cccc(-c7ccc8c(c7)c7ccccc7n8-c7ccccc7)c6)c45)n3)c2)cc1]

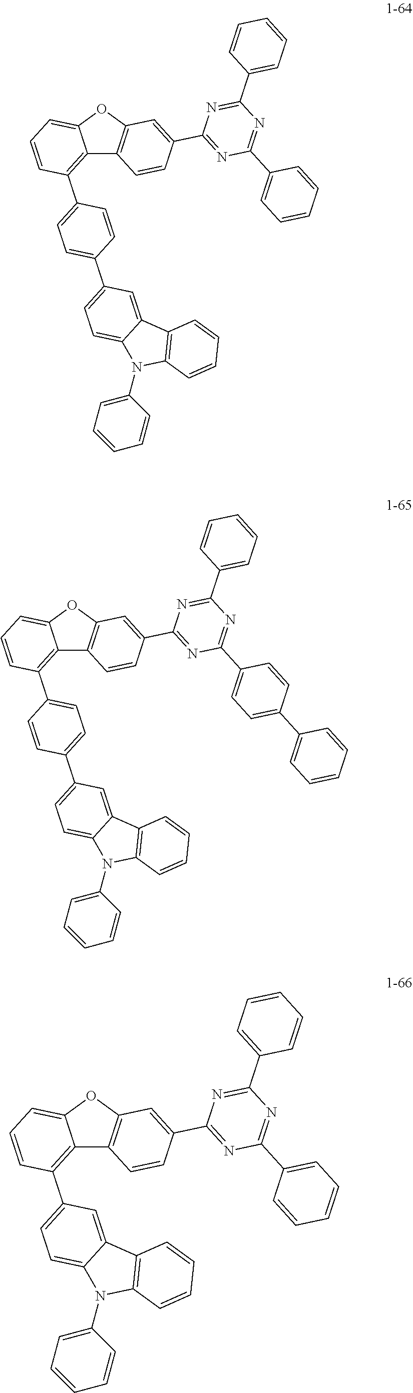 [SMILES: c1ccc(-c2nc(-c3ccccc3)nc(-c3ccc4c(c3)oc3cccc(-c5ccc(-c6ccc7c(c6)c6ccccc6n7-c6ccccc6)cc5)c34)n2)cc1 ; c1ccc(-c2ccc(-c3nc(-c4ccccc4)nc(-c4ccc5c(c4)oc4cccc(-c6ccc(-c7ccc8c(c7)c7ccccc7n8-c7ccccc7)cc6)c45)n3)cc2)cc1 ; c1ccc(-c2nc(-c3ccccc3)nc(-c3ccc4c(c3)oc3cccc(-c5ccc6c(c5)c5ccccc5n6-c5ccccc5)c34)n2)cc1]

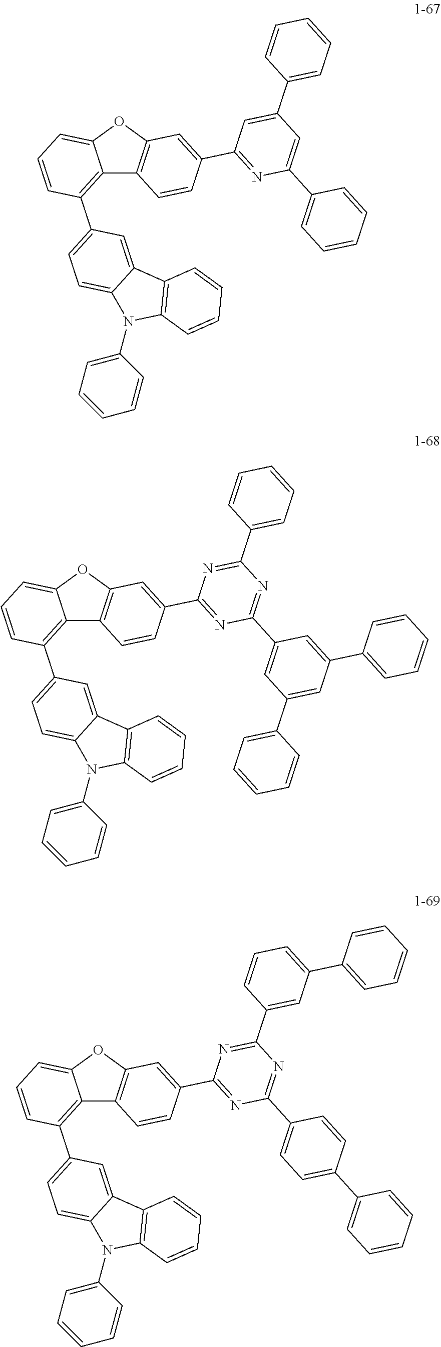 [SMILES: c1ccc(-c2cc(-c3ccccc3)nc(-c3ccc4c(c3)oc3cccc(-c5ccc6c(c5)c5ccccc5n6-c5ccccc5)c34)c2)cc1 ; c1ccc(-c2cc(-c3ccccc3)cc(-c3nc(-c4ccccc4)nc(-c4ccc5c(c4)oc4cccc(-c6ccc7c(c6)c6ccccc6n7-c6ccccc6)c45)n3)c2)cc1 ; c1ccc(-c2ccc(-c3nc(-c4cccc(-c5ccccc5)c4)nc(-c4ccc5c(c4)oc4cccc(-c6ccc7c(c6)c6ccccc6n7-c6ccccc6)c45)n3)cc2)cc1]

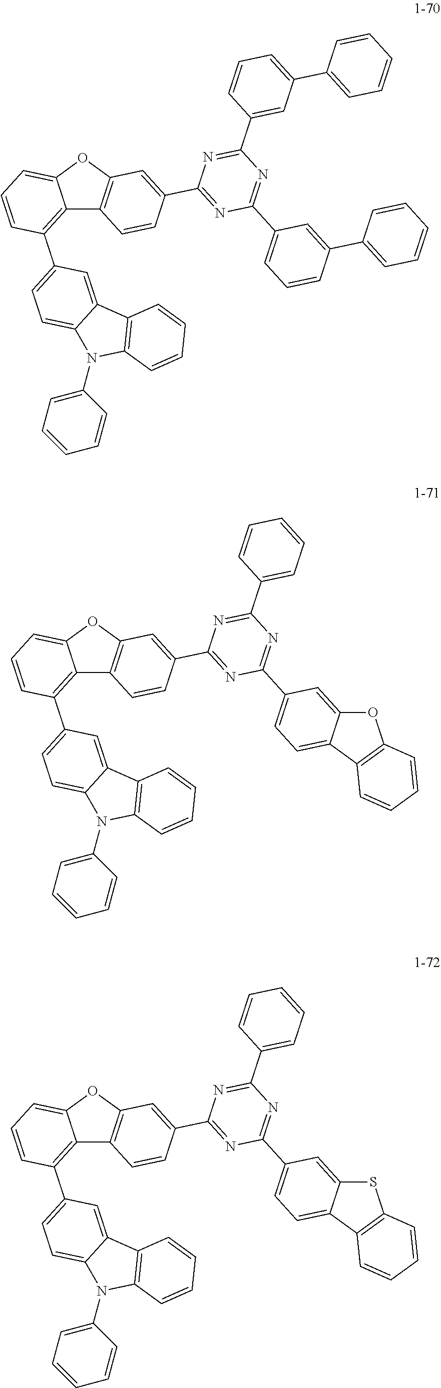 [SMILES: c1ccc(-c2cccc(-c3nc(-c4cccc(-c5ccccc5)c4)nc(-c4ccc5c(c4)oc4cccc(-c6ccc7c(c6)c6ccccc6n7-c6ccccc6)c45)n3)c2)cc1 ; c1ccc(-c2nc(-c3ccc4c(c3)oc3ccccc34)nc(-c3ccc4c(c3)oc3cccc(-c5ccc6c(c5)c5ccccc5n6-c5ccccc5)c34)n2)cc1 ; c1ccc(-c2nc(-c3ccc4c(c3)oc3cccc(-c5ccc6c(c5)c5ccccc5n6-c5ccccc5)c34)nc(-c3ccc4c(c3)sc3ccccc34)n2)cc1]

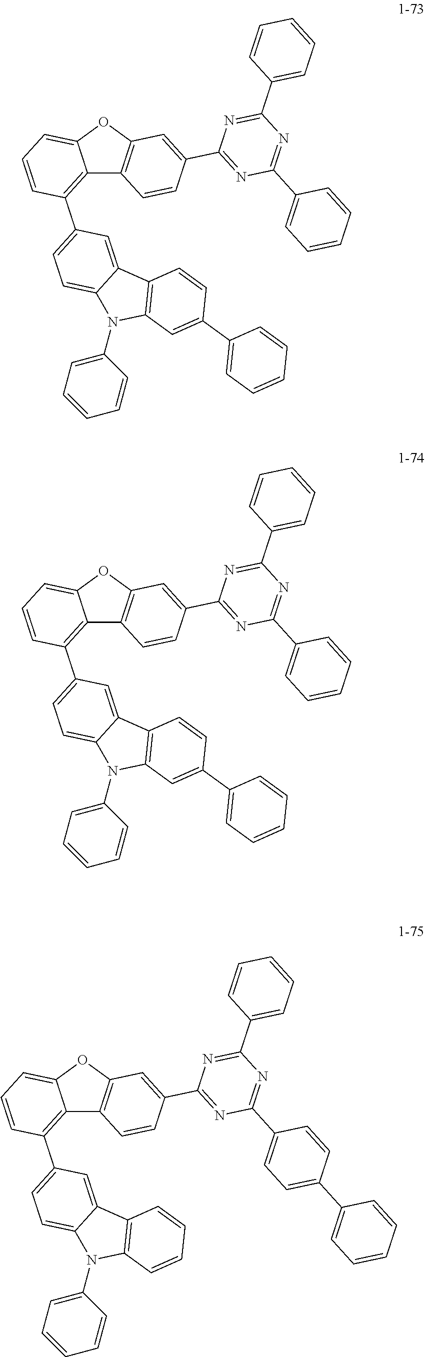 [SMILES: c1ccc(-c2ccc3c4cc(-c5cccc6oc7cc(-c8nc(-c9ccccc9)nc(-c9ccccc9)n8)ccc7c56)ccc4n(-c4ccccc4)c3c2)cc1 ; c1ccc(-c2ccc3c4cc(-c5cccc6oc7cc(-c8nc(-c9ccccc9)nc(-c9ccccc9)n8)ccc7c56)ccc4n(-c4ccccc4)c3c2)cc1 ; c1ccc(-c2ccc(-c3nc(-c4ccccc4)nc(-c4ccc5c(c4)oc4cccc(-c6ccc7c(c6)c6ccccc6n7-c6ccccc6)c45)n3)cc2)cc1]

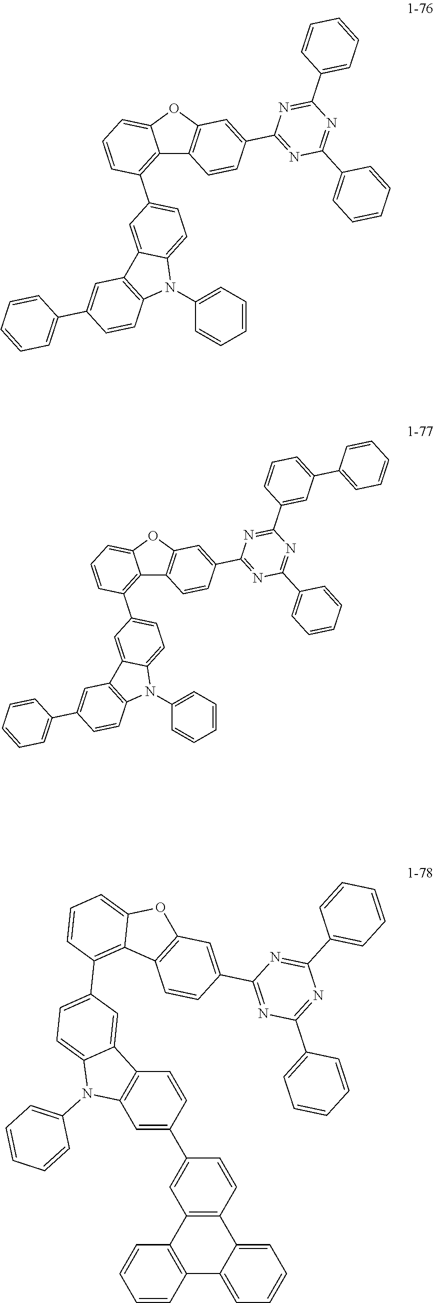 [SMILES: c1ccc(-c2ccc3c(c2)c2cc(-c4cccc5oc6cc(-c7nc(-c8ccccc8)nc(-c8ccccc8)n7)ccc6c45)ccc2n3-c2ccccc2)cc1 ; c1ccc(-c2cccc(-c3nc(-c4ccccc4)nc(-c4ccc5c(c4)oc4cccc(-c6ccc7c(c6)c6cc(-c8ccccc8)ccc6n7-c6ccccc6)c45)n3)c2)cc1 ; c1ccc(-c2nc(-c3ccccc3)nc(-c3ccc4c(c3)oc3cccc(-c5ccc6c(c5)c5ccc(-c7ccc8c9ccccc9c9ccccc9c8c7)cc5n6-c5ccccc5)c34)n2)cc1]

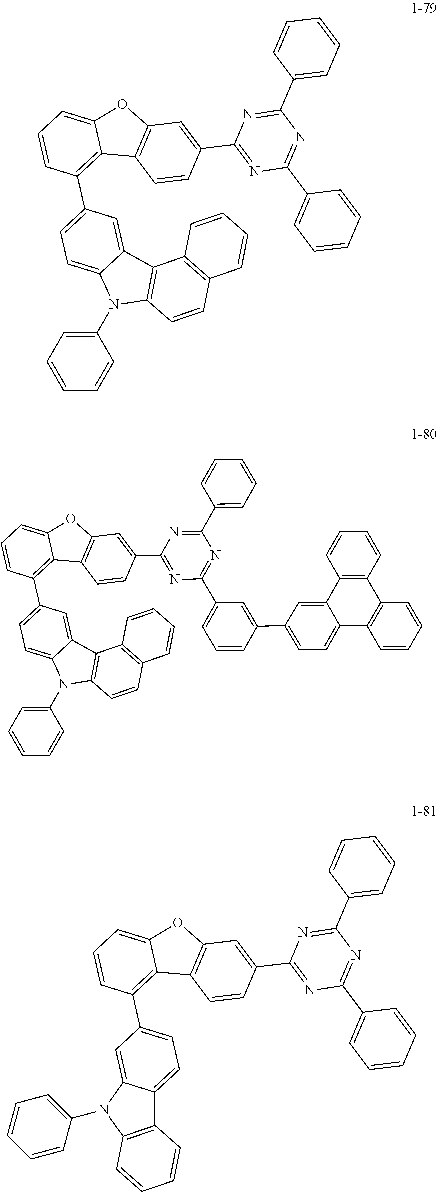 [SMILES: c1ccc(-c2nc(-c3ccccc3)nc(-c3ccc4c(c3)oc3cccc(-c5ccc6c(c5)c5c7ccccc7ccc5n6-c5ccccc5)c34)n2)cc1 ; c1ccc(-c2nc(-c3cccc(-c4ccc5c6ccccc6c6ccccc6c5c4)c3)nc(-c3ccc4c(c3)oc3cccc(-c5ccc6c(c5)c5c7ccccc7ccc5n6-c5ccccc5)c34)n2)cc1 ; c1ccc(-c2nc(-c3ccccc3)nc(-c3ccc4c(c3)oc3cccc(-c5ccc6c7ccccc7n(-c7ccccc7)c6c5)c34)n2)cc1]

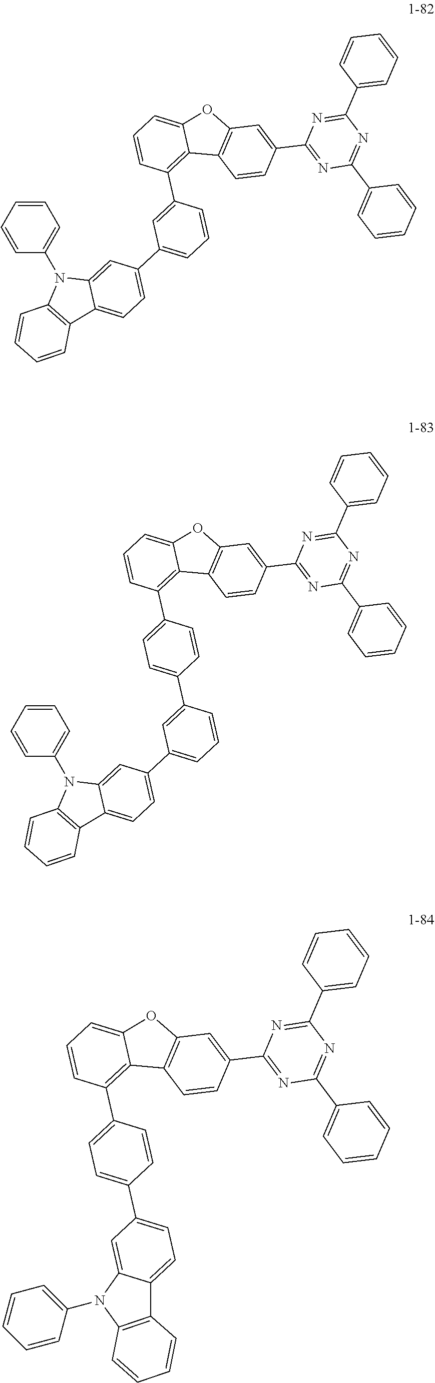 [SMILES: c1ccc(-c2nc(-c3ccccc3)nc(-c3ccc4c(c3)oc3cccc(-c5cccc(-c6ccc7c8ccccc8n(-c8ccccc8)c7c6)c5)c34)n2)cc1 ; c1ccc(-c2nc(-c3ccccc3)nc(-c3ccc4c(c3)oc3cccc(-c5ccc(-c6cccc(-c7ccc8c9ccccc9n(-c9ccccc9)c8c7)c6)cc5)c34)n2)cc1 ; c1ccc(-c2nc(-c3ccccc3)nc(-c3ccc4c(c3)oc3cccc(-c5ccc(-c6ccc7c8ccccc8n(-c8ccccc8)c7c6)cc5)c34)n2)cc1]

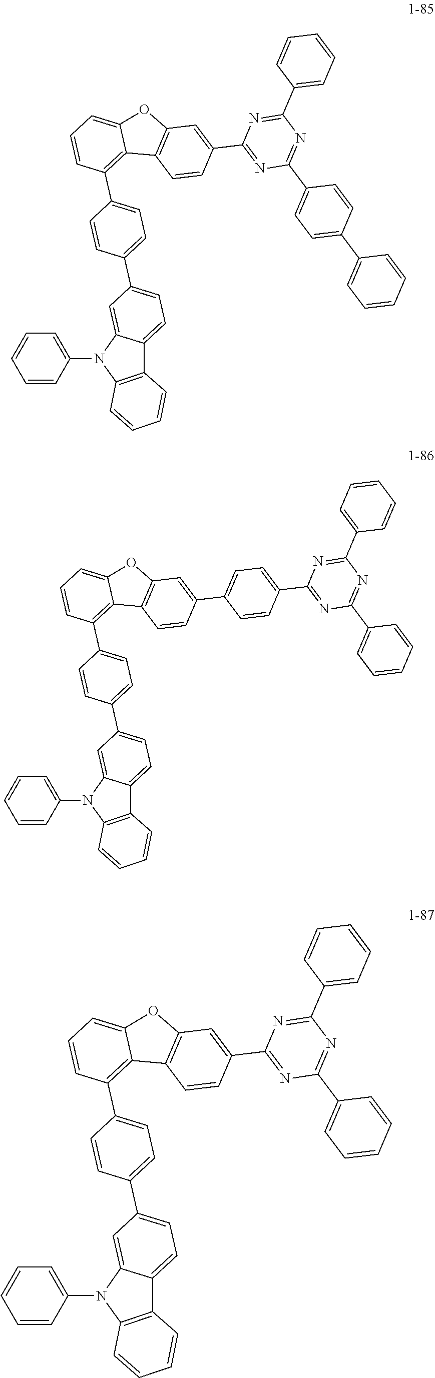 [SMILES: c1ccc(-c2ccc(-c3nc(-c4ccccc4)nc(-c4ccc5c(c4)oc4cccc(-c6ccc(-c7ccc8c9ccccc9n(-c9ccccc9)c8c7)cc6)c45)n3)cc2)cc1 ; c1ccc(-c2nc(-c3ccccc3)nc(-c3ccc(-c4ccc5c(c4)oc4cccc(-c6ccc(-c7ccc8c9ccccc9n(-c9ccccc9)c8c7)cc6)c45)cc3)n2)cc1 ; c1ccc(-c2nc(-c3ccccc3)nc(-c3ccc4c(c3)oc3cccc(-c5ccc(-c6ccc7c8ccccc8n(-c8ccccc8)c7c6)cc5)c34)n2)cc1]

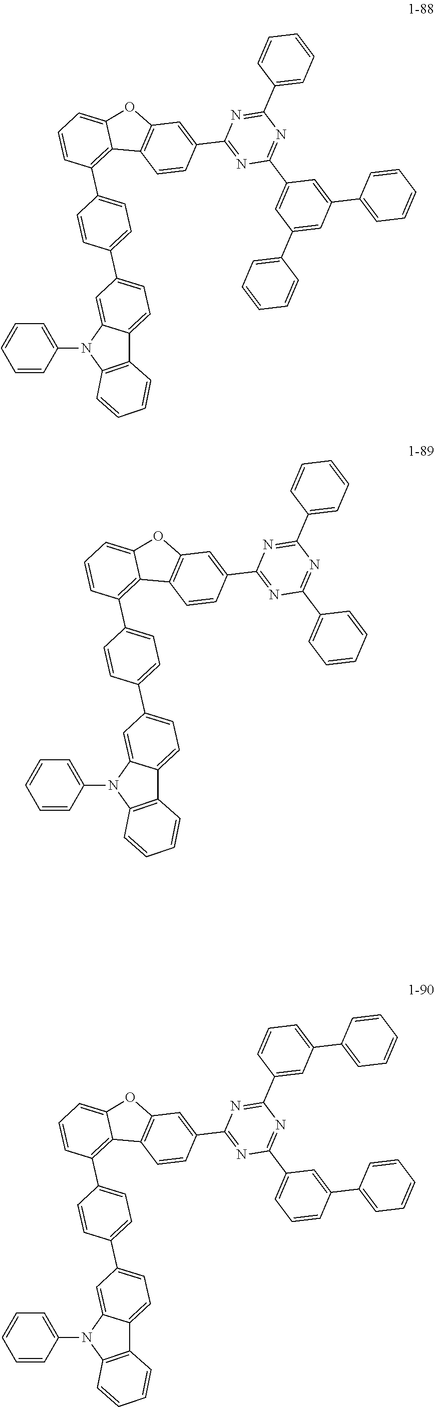 [SMILES: c1ccc(-c2cc(-c3ccccc3)cc(-c3nc(-c4ccccc4)nc(-c4ccc5c(c4)oc4cccc(-c6ccc(-c7ccc8c9ccccc9n(-c9ccccc9)c8c7)cc6)c45)n3)c2)cc1 ; c1ccc(-c2nc(-c3ccccc3)nc(-c3ccc4c(c3)oc3cccc(-c5ccc(-c6ccc7c8ccccc8n(-c8ccccc8)c7c6)cc5)c34)n2)cc1 ; c1ccc(-c2cccc(-c3nc(-c4cccc(-c5ccccc5)c4)nc(-c4ccc5c(c4)oc4cccc(-c6ccc(-c7ccc8c9ccccc9n(-c9ccccc9)c8c7)cc6)c45)n3)c2)cc1]

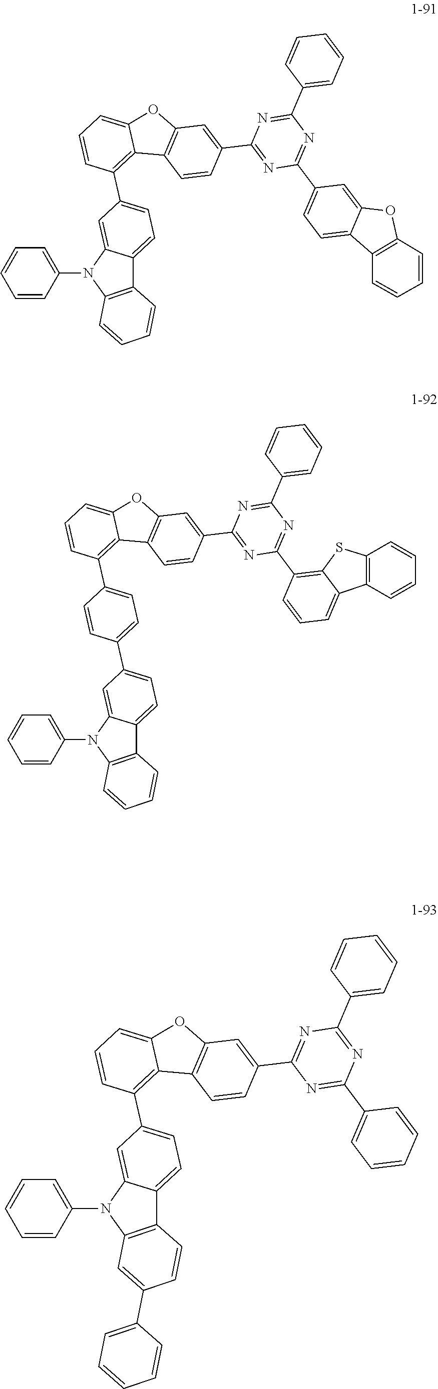 [SMILES: c1ccc(-c2nc(-c3ccc4c(c3)oc3ccccc34)nc(-c3ccc4c(c3)oc3cccc(-c5ccc6c7ccccc7n(-c7ccccc7)c6c5)c34)n2)cc1 ; c1ccc(-c2nc(-c3ccc4c(c3)oc3cccc(-c5ccc(-c6ccc7c8ccccc8n(-c8ccccc8)c7c6)cc5)c34)nc(-c3cccc4c3sc3ccccc34)n2)cc1 ; c1ccc(-c2ccc3c4ccc(-c5cccc6oc7cc(-c8nc(-c9ccccc9)nc(-c9ccccc9)n8)ccc7c56)cc4n(-c4ccccc4)c3c2)cc1]

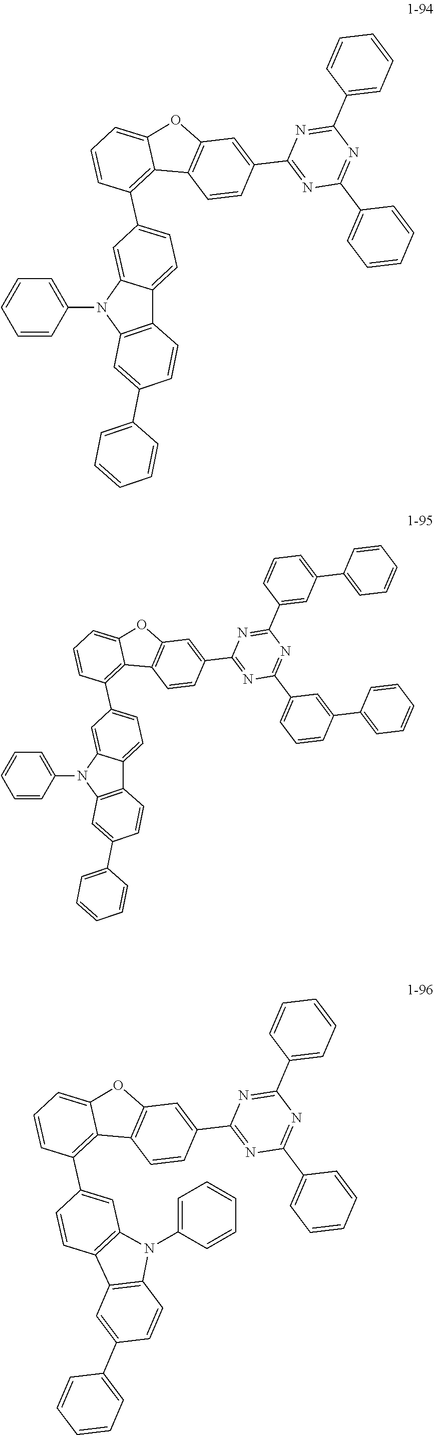 [SMILES: c1ccc(-c2ccc3c4ccc(-c5cccc6oc7cc(-c8nc(-c9ccccc9)nc(-c9ccccc9)n8)ccc7c56)cc4n(-c4ccccc4)c3c2)cc1 ; c1ccc(-c2cccc(-c3nc(-c4cccc(-c5ccccc5)c4)nc(-c4ccc5c(c4)oc4cccc(-c6ccc7c8ccc(-c9ccccc9)cc8n(-c8ccccc8)c7c6)c45)n3)c2)cc1 ; c1ccc(-c2ccc3c(c2)c2ccc(-c4cccc5oc6cc(-c7nc(-c8ccccc8)nc(-c8ccccc8)n7)ccc6c45)cc2n3-c2ccccc2)cc1]

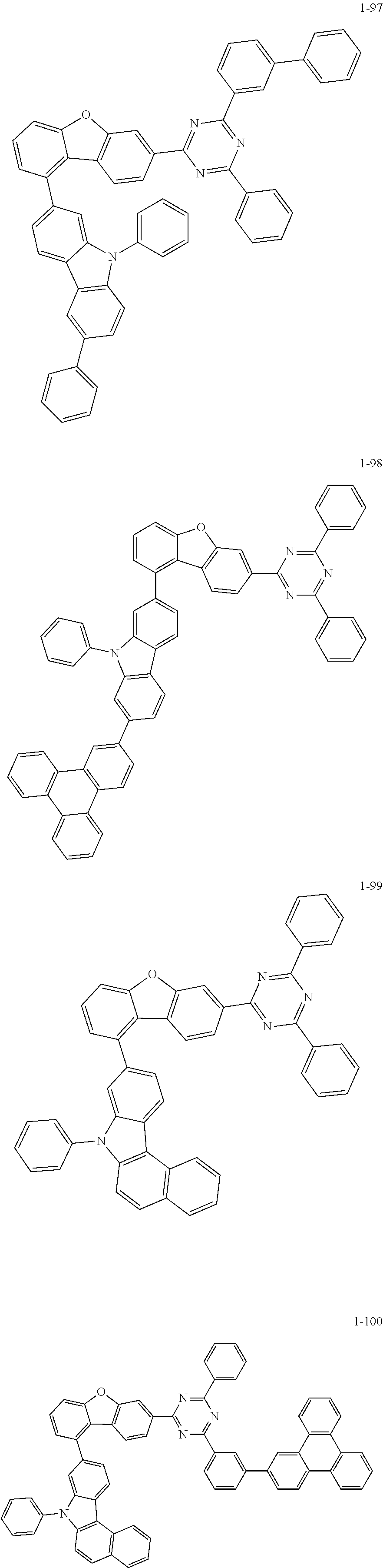 [SMILES: c1ccc(-c2cccc(-c3nc(-c4ccccc4)nc(-c4ccc5c(c4)oc4cccc(-c6ccc7c8cc(-c9ccccc9)ccc8n(-c8ccccc8)c7c6)c45)n3)c2)cc1 ; c1ccc(-c2nc(-c3ccccc3)nc(-c3ccc4c(c3)oc3cccc(-c5ccc6c7ccc(-c8ccc9c%10ccccc%10c%10ccccc%10c9c8)cc7n(-c7ccccc7)c6c5)c34)n2)cc1 ; c1ccc(-c2nc(-c3ccccc3)nc(-c3ccc4c(c3)oc3cccc(-c5ccc6c7c8ccccc8ccc7n(-c7ccccc7)c6c5)c34)n2)cc1 ; c1ccc(-c2nc(-c3cccc(-c4ccc5c6ccccc6c6ccccc6c5c4)c3)nc(-c3ccc4c(c3)oc3cccc(-c5ccc6c7c8ccccc8ccc7n(-c7ccccc7)c6c5)c34)n2)cc1]

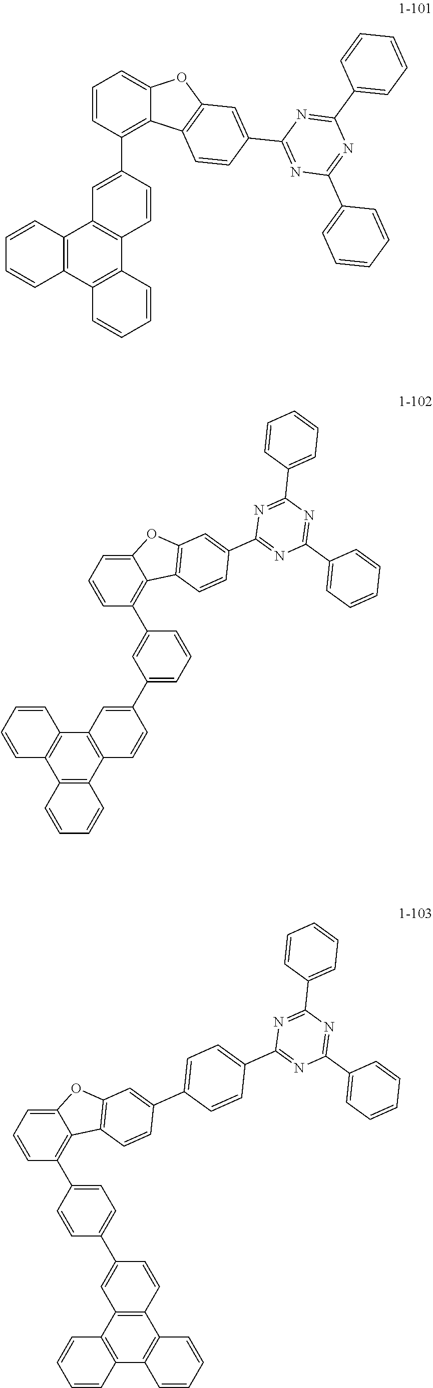 [SMILES: c1ccc(-c2nc(-c3ccccc3)nc(-c3ccc4c(c3)oc3cccc(-c5ccc6c7ccccc7c7ccccc7c6c5)c34)n2)cc1 ; c1ccc(-c2nc(-c3ccccc3)nc(-c3ccc4c(c3)oc3cccc(-c5cccc(-c6ccc7c8ccccc8c8ccccc8c7c6)c5)c34)n2)cc1 ; c1ccc(-c2nc(-c3ccccc3)nc(-c3ccc(-c4ccc5c(c4)oc4cccc(-c6ccc(-c7ccc8c9ccccc9c9ccccc9c8c7)cc6)c45)cc3)n2)cc1]

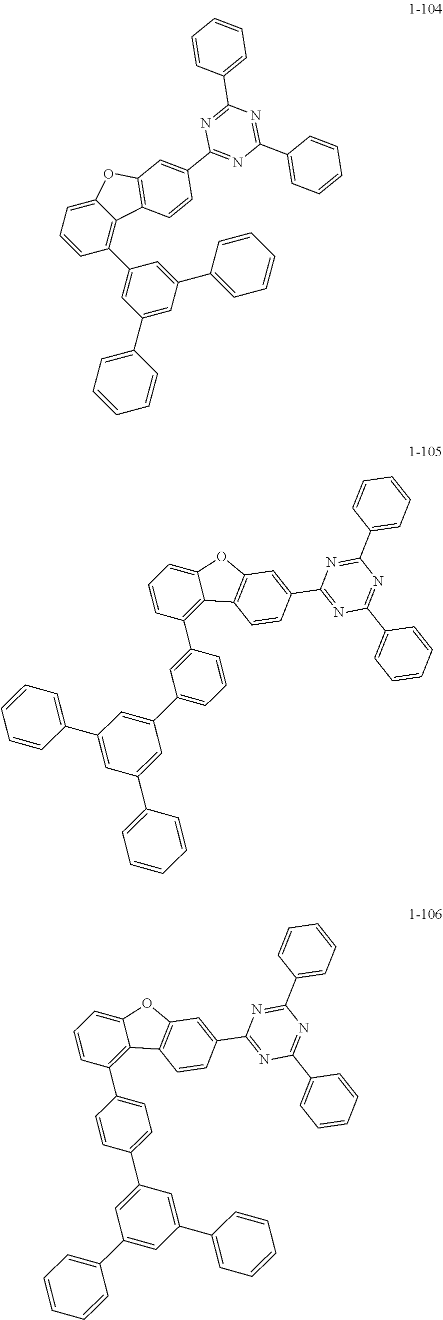 [SMILES: c1ccc(-c2cc(-c3ccccc3)cc(-c3cccc4oc5cc(-c6nc(-c7ccccc7)nc(-c7ccccc7)n6)ccc5c34)c2)cc1 ; c1ccc(-c2cc(-c3ccccc3)cc(-c3cccc(-c4cccc5oc6cc(-c7nc(-c8ccccc8)nc(-c8ccccc8)n7)ccc6c45)c3)c2)cc1 ; c1ccc(-c2cc(-c3ccccc3)cc(-c3ccc(-c4cccc5oc6cc(-c7nc(-c8ccccc8)nc(-c8ccccc8)n7)ccc6c45)cc3)c2)cc1]

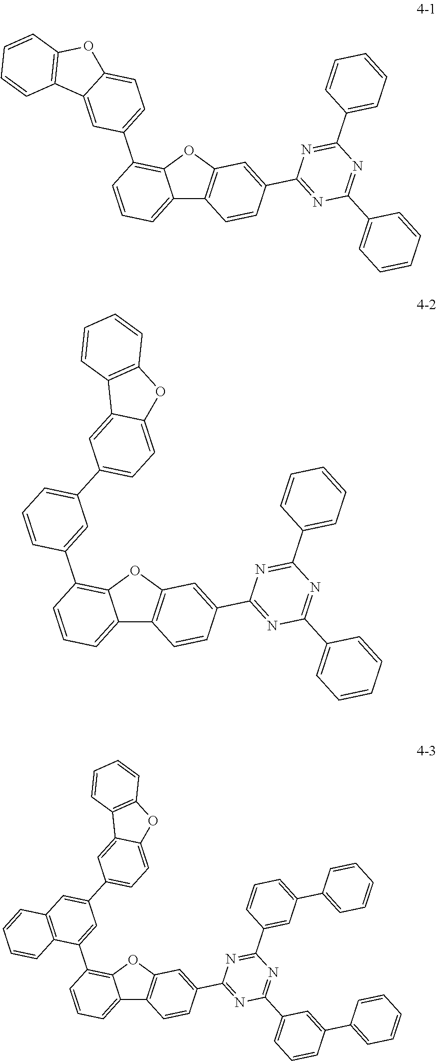 [SMILES: c1ccc(-c2nc(-c3ccccc3)nc(-c3ccc4c(c3)oc3c(-c5ccc6oc7ccccc7c6c5)cccc34)n2)cc1 ; c1ccc(-c2nc(-c3ccccc3)nc(-c3ccc4c(c3)oc3c(-c5cccc(-c6ccc7oc8ccccc8c7c6)c5)cccc34)n2)cc1 ; c1ccc(-c2cccc(-c3nc(-c4cccc(-c5ccccc5)c4)nc(-c4ccc5c(c4)oc4c(-c6cc(-c7ccc8oc9ccccc9c8c7)cc7ccccc67)cccc45)n3)c2)cc1]

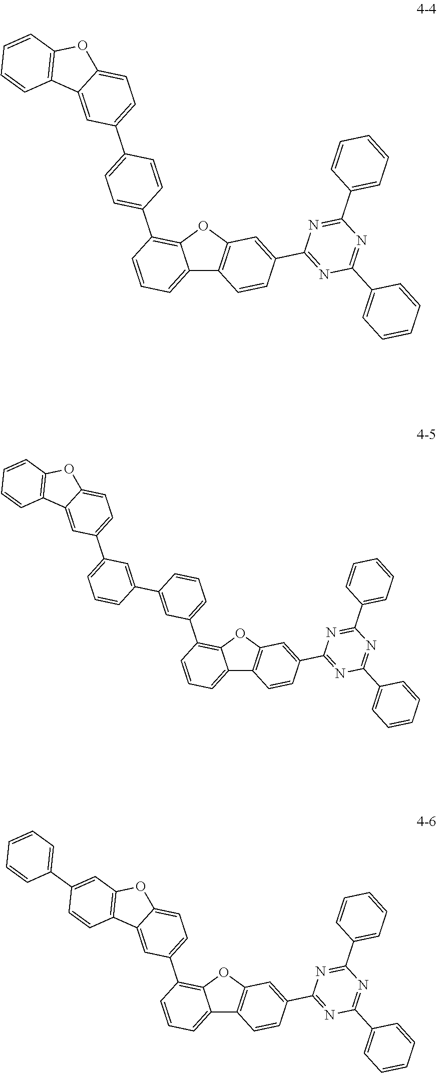 [SMILES: c1ccc(-c2nc(-c3ccccc3)nc(-c3ccc4c(c3)oc3c(-c5ccc(-c6ccc7oc8ccccc8c7c6)cc5)cccc34)n2)cc1 ; c1ccc(-c2nc(-c3ccccc3)nc(-c3ccc4c(c3)oc3c(-c5cccc(-c6cccc(-c7ccc8oc9ccccc9c8c7)c6)c5)cccc34)n2)cc1 ; c1ccc(-c2ccc3c(c2)oc2ccc(-c4cccc5c4oc4cc(-c6nc(-c7ccccc7)nc(-c7ccccc7)n6)ccc45)cc23)cc1]

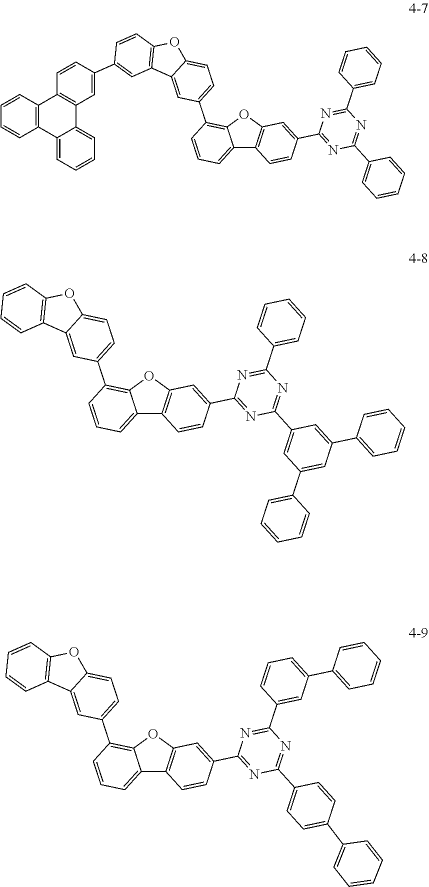 [SMILES: c1ccc(-c2nc(-c3ccccc3)nc(-c3ccc4c(c3)oc3c(-c5ccc6oc7ccc(-c8ccc9c%10ccccc%10c%10ccccc%10c9c8)cc7c6c5)cccc34)n2)cc1 ; c1ccc(-c2cc(-c3ccccc3)cc(-c3nc(-c4ccccc4)nc(-c4ccc5c(c4)oc4c(-c6ccc7oc8ccccc8c7c6)cccc45)n3)c2)cc1 ; c1ccc(-c2ccc(-c3nc(-c4cccc(-c5ccccc5)c4)nc(-c4ccc5c(c4)oc4c(-c6ccc7oc8ccccc8c7c6)cccc45)n3)cc2)cc1]

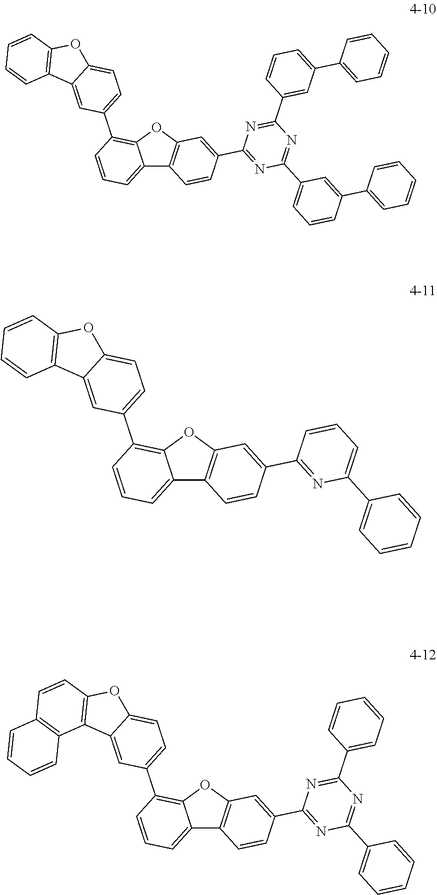 [SMILES: c1ccc(-c2cccc(-c3nc(-c4cccc(-c5ccccc5)c4)nc(-c4ccc5c(c4)oc4c(-c6ccc7oc8ccccc8c7c6)cccc45)n3)c2)cc1 ; c1ccc(-c2cccc(-c3ccc4c(c3)oc3c(-c5ccc6oc7ccccc7c6c5)cccc34)n2)cc1 ; c1ccc(-c2nc(-c3ccccc3)nc(-c3ccc4c(c3)oc3c(-c5ccc6oc7ccc8ccccc8c7c6c5)cccc34)n2)cc1]

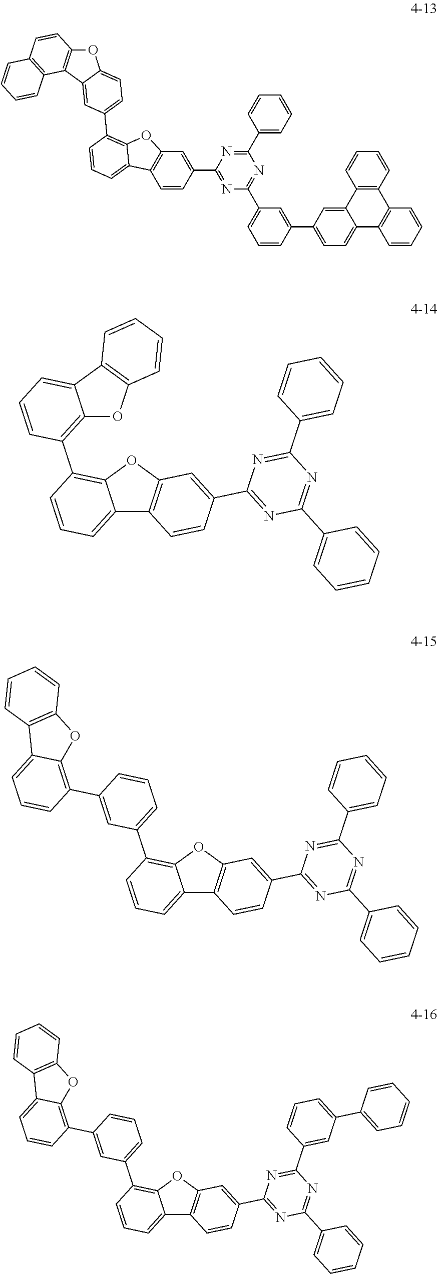 [SMILES: c1ccc(-c2nc(-c3cccc(-c4ccc5c6ccccc6c6ccccc6c5c4)c3)nc(-c3ccc4c(c3)oc3c(-c5ccc6oc7ccc8ccccc8c7c6c5)cccc34)n2)cc1 ; c1ccc(-c2nc(-c3ccccc3)nc(-c3ccc4c(c3)oc3c(-c5cccc6c5oc5ccccc56)cccc34)n2)cc1 ; c1ccc(-c2nc(-c3ccccc3)nc(-c3ccc4c(c3)oc3c(-c5cccc(-c6cccc7c6oc6ccccc67)c5)cccc34)n2)cc1 ; c1ccc(-c2cccc(-c3nc(-c4ccccc4)nc(-c4ccc5c(c4)oc4c(-c6cccc(-c7cccc8c7oc7ccccc78)c6)cccc45)n3)c2)cc1]

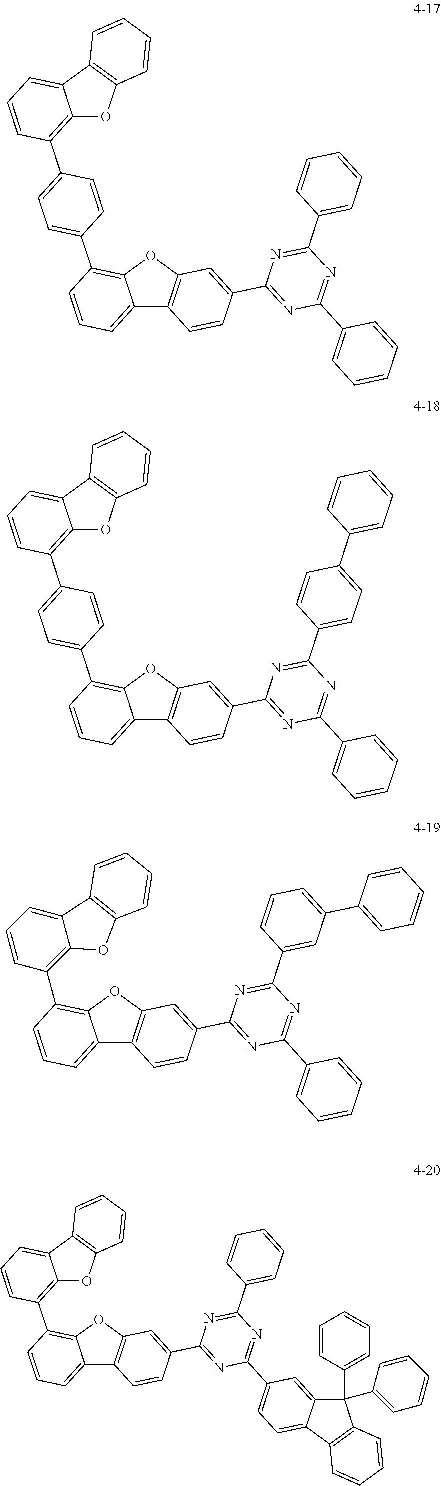 [SMILES: c1ccc(-c2nc(-c3ccccc3)nc(-c3ccc4c(c3)oc3c(-c5ccc(-c6cccc7c6oc6ccccc67)cc5)cccc34)n2)cc1 ; c1ccc(-c2ccc(-c3nc(-c4ccccc4)nc(-c4ccc5c(c4)oc4c(-c6ccc(-c7cccc8c7oc7ccccc78)cc6)cccc45)n3)cc2)cc1 ; c1ccc(-c2cccc(-c3nc(-c4ccccc4)nc(-c4ccc5c(c4)oc4c(-c6cccc7c6oc6ccccc67)cccc45)n3)c2)cc1 ; c1ccc(-c2nc(-c3ccc4c(c3)C(c3ccccc3)(c3ccccc3)c3ccccc3-4)nc(-c3ccc4c(c3)oc3c(-c5cccc6c5oc5ccccc56)cccc34)n2)cc1]

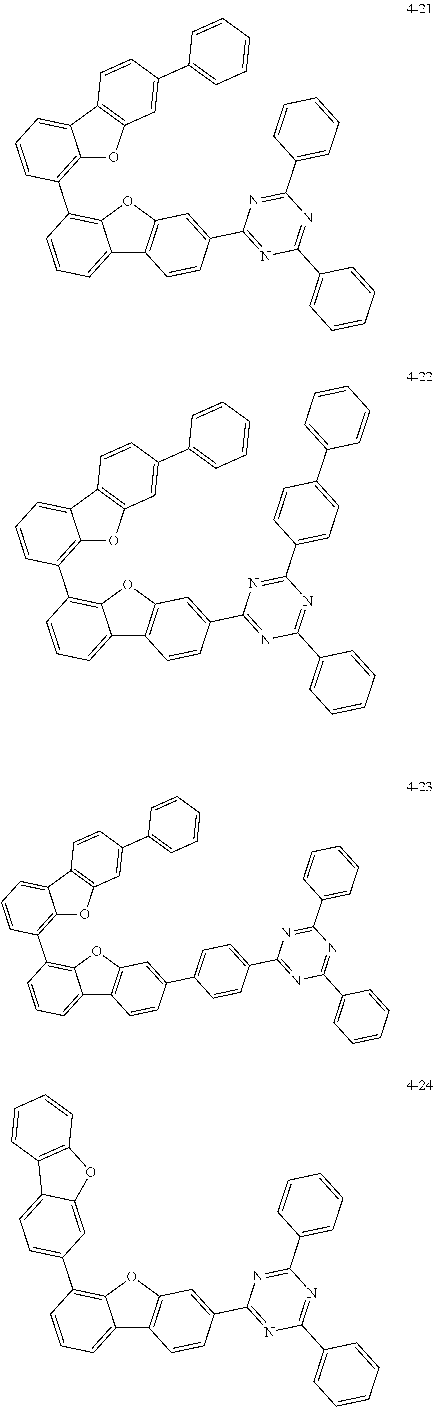 [SMILES: c1ccc(-c2ccc3c(c2)oc2c(-c4cccc5c4oc4cc(-c6nc(-c7ccccc7)nc(-c7ccccc7)n6)ccc45)cccc23)cc1 ; c1ccc(-c2ccc(-c3nc(-c4ccccc4)nc(-c4ccc5c(c4)oc4c(-c6cccc7c6oc6cc(-c8ccccc8)ccc67)cccc45)n3)cc2)cc1 ; c1ccc(-c2ccc3c(c2)oc2c(-c4cccc5c4oc4cc(-c6ccc(-c7nc(-c8ccccc8)nc(-c8ccccc8)n7)cc6)ccc45)cccc23)cc1 ; c1ccc(-c2nc(-c3ccccc3)nc(-c3ccc4c(c3)oc3c(-c5ccc6c(c5)oc5ccccc56)cccc34)n2)cc1]

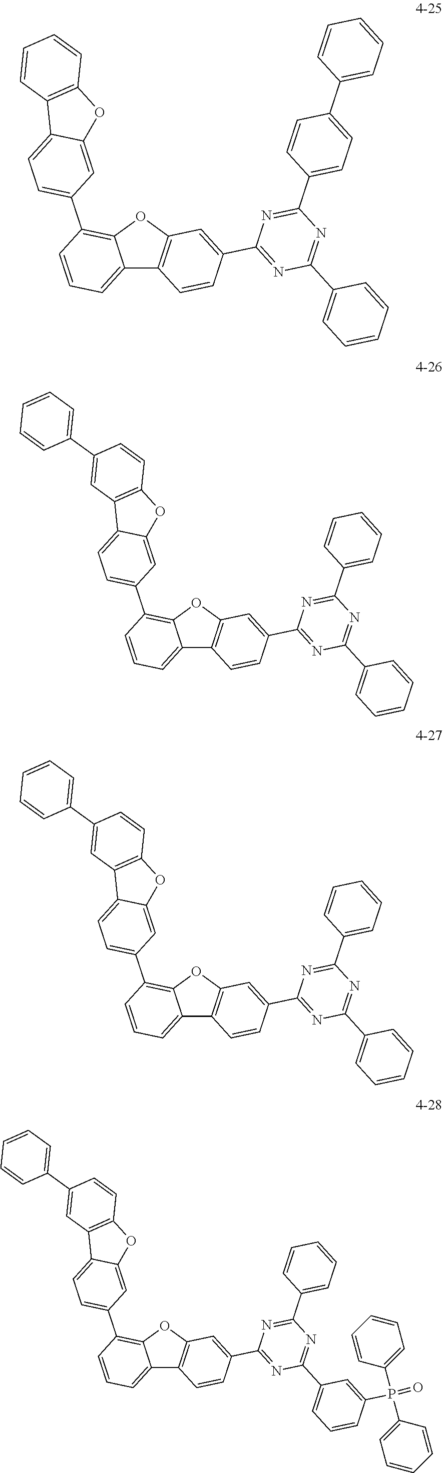 [SMILES: c1ccc(-c2ccc(-c3nc(-c4ccccc4)nc(-c4ccc5c(c4)oc4c(-c6ccc7c(c6)oc6ccccc67)cccc45)n3)cc2)cc1 ; c1ccc(-c2ccc3oc4cc(-c5cccc6c5oc5cc(-c7nc(-c8ccccc8)nc(-c8ccccc8)n7)ccc56)ccc4c3c2)cc1 ; c1ccc(-c2ccc3oc4cc(-c5cccc6c5oc5cc(-c7nc(-c8ccccc8)nc(-c8ccccc8)n7)ccc56)ccc4c3c2)cc1 ; O=P(c1ccccc1)(c1ccccc1)c1cccc(-c2nc(-c3ccccc3)nc(-c3ccc4c(c3)oc3c(-c5ccc6c(c5)oc5ccc(-c7ccccc7)cc56)cccc34)n2)c1]

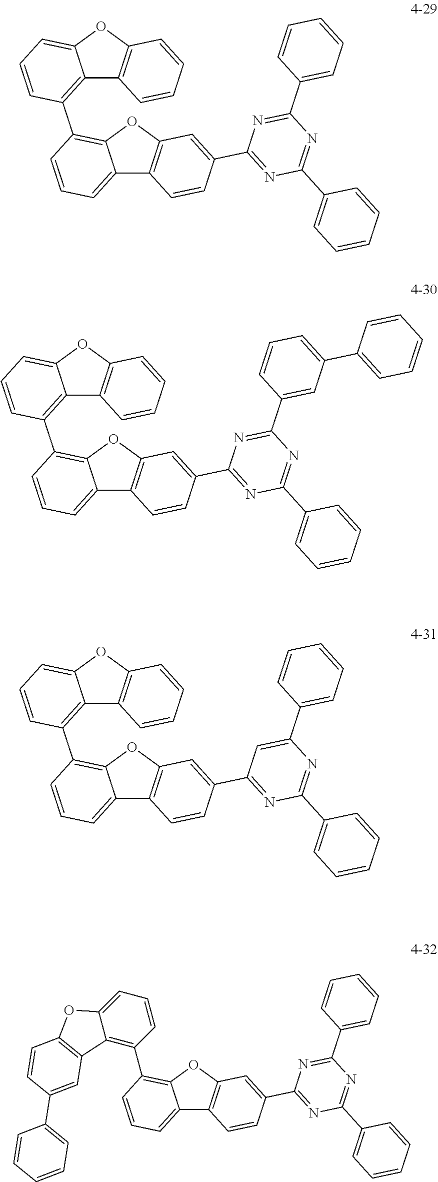 [SMILES: c1ccc(-c2nc(-c3ccccc3)nc(-c3ccc4c(c3)oc3c(-c5cccc6oc7ccccc7c56)cccc34)n2)cc1 ; c1ccc(-c2cccc(-c3nc(-c4ccccc4)nc(-c4ccc5c(c4)oc4c(-c6cccc7oc8ccccc8c67)cccc45)n3)c2)cc1 ; c1ccc(-c2cc(-c3ccc4c(c3)oc3c(-c5cccc6oc7ccccc7c56)cccc34)nc(-c3ccccc3)n2)cc1 ; c1ccc(-c2ccc3oc4cccc(-c5cccc6c5oc5cc(-c7nc(-c8ccccc8)nc(-c8ccccc8)n7)ccc56)c4c3c2)cc1]

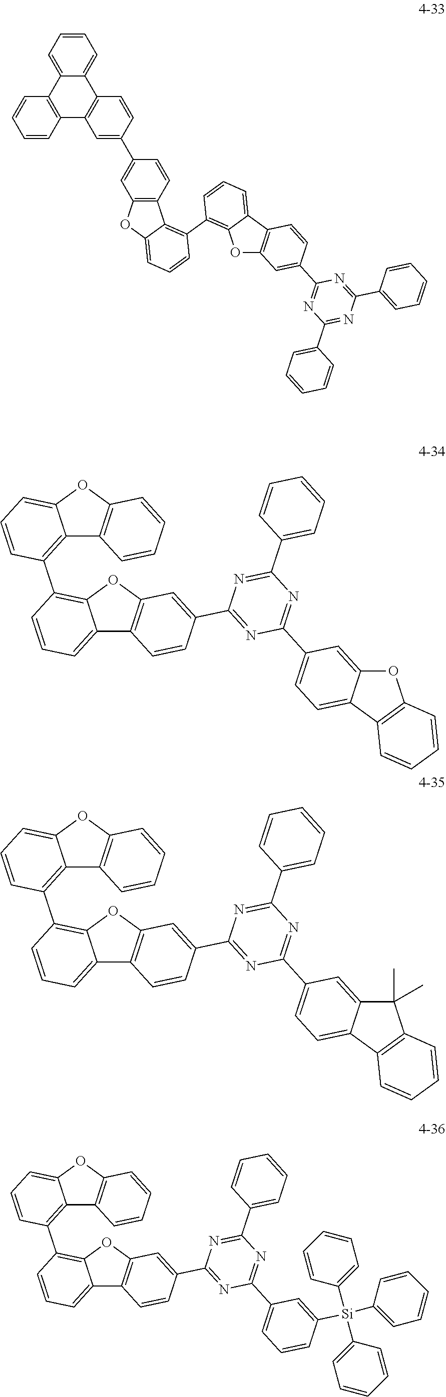 [SMILES: c1ccc(-c2nc(-c3ccccc3)nc(-c3ccc4c(c3)oc3c(-c5cccc6oc7cc(-c8ccc9c%10ccccc%10c%10ccccc%10c9c8)ccc7c56)cccc34)n2)cc1 ; c1ccc(-c2nc(-c3ccc4c(c3)oc3ccccc34)nc(-c3ccc4c(c3)oc3c(-c5cccc6oc7ccccc7c56)cccc34)n2)cc1 ; CC1(C)c2ccccc2-c2ccc(-c3nc(-c4ccccc4)nc(-c4ccc5c(c4)oc4c(-c6cccc7oc8ccccc8c67)cccc45)n3)cc21 ; c1ccc(-c2nc(-c3cccc([Si](c4ccccc4)(c4ccccc4)c4ccccc4)c3)nc(-c3ccc4c(c3)oc3c(-c5cccc6oc7ccccc7c56)cccc34)n2)cc1]

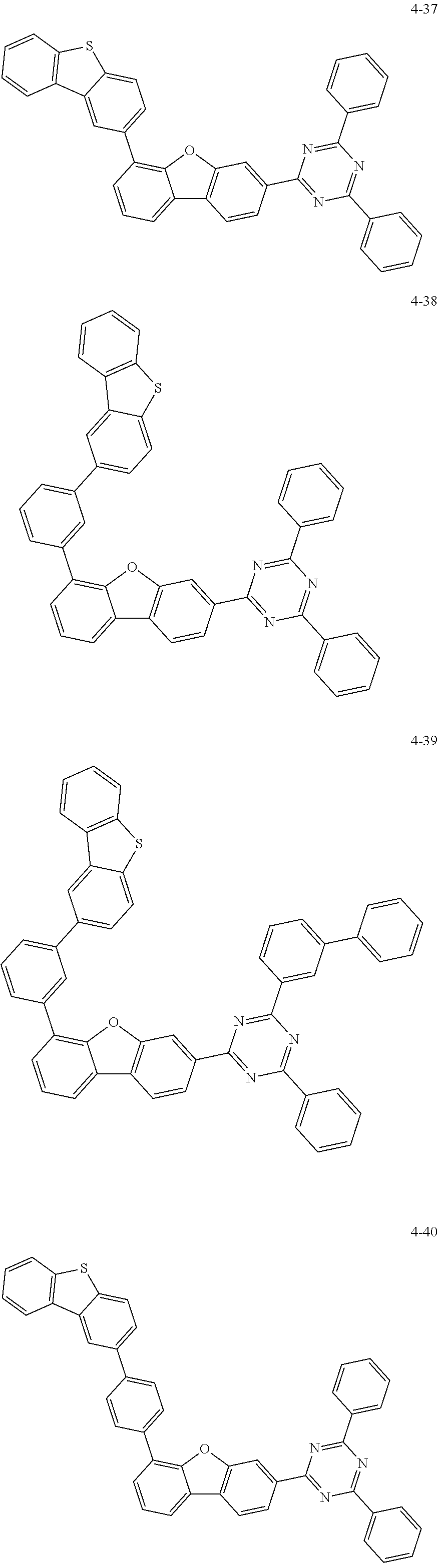 [SMILES: c1ccc(-c2nc(-c3ccccc3)nc(-c3ccc4c(c3)oc3c(-c5ccc6sc7ccccc7c6c5)cccc34)n2)cc1 ; c1ccc(-c2nc(-c3ccccc3)nc(-c3ccc4c(c3)oc3c(-c5cccc(-c6ccc7sc8ccccc8c7c6)c5)cccc34)n2)cc1 ; c1ccc(-c2cccc(-c3nc(-c4ccccc4)nc(-c4ccc5c(c4)oc4c(-c6cccc(-c7ccc8sc9ccccc9c8c7)c6)cccc45)n3)c2)cc1 ; c1ccc(-c2nc(-c3ccccc3)nc(-c3ccc4c(c3)oc3c(-c5ccc(-c6ccc7sc8ccccc8c7c6)cc5)cccc34)n2)cc1]

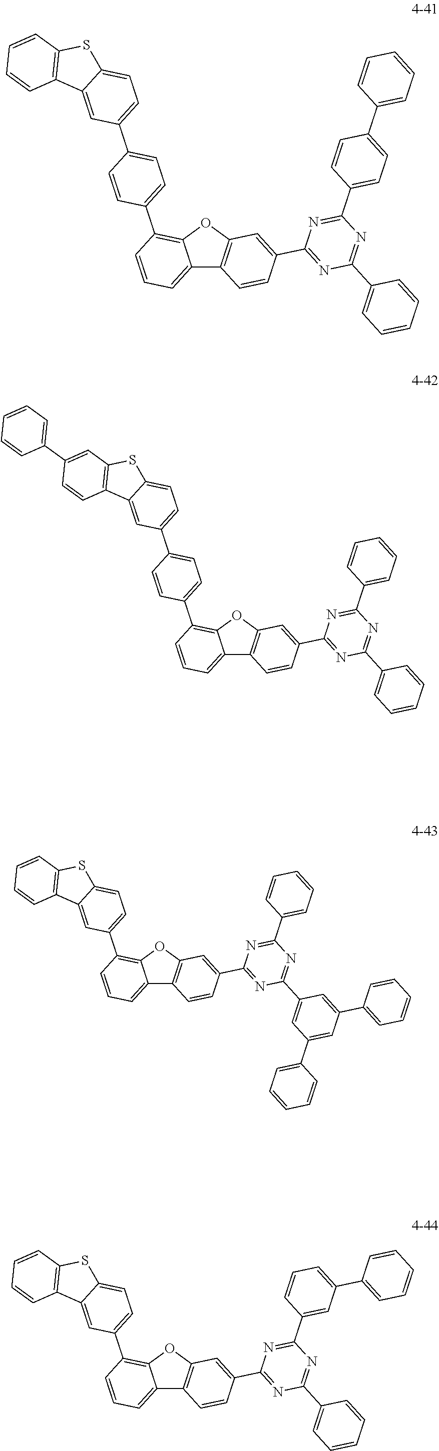 [SMILES: c1ccc(-c2ccc(-c3nc(-c4ccccc4)nc(-c4ccc5c(c4)oc4c(-c6ccc(-c7ccc8sc9ccccc9c8c7)cc6)cccc45)n3)cc2)cc1 ; c1ccc(-c2ccc3c(c2)sc2ccc(-c4ccc(-c5cccc6c5oc5cc(-c7nc(-c8ccccc8)nc(-c8ccccc8)n7)ccc56)cc4)cc23)cc1 ; c1ccc(-c2cc(-c3ccccc3)cc(-c3nc(-c4ccccc4)nc(-c4ccc5c(c4)oc4c(-c6ccc7sc8ccccc8c7c6)cccc45)n3)c2)cc1 ; c1ccc(-c2cccc(-c3nc(-c4ccccc4)nc(-c4ccc5c(c4)oc4c(-c6ccc7sc8ccccc8c7c6)cccc45)n3)c2)cc1]

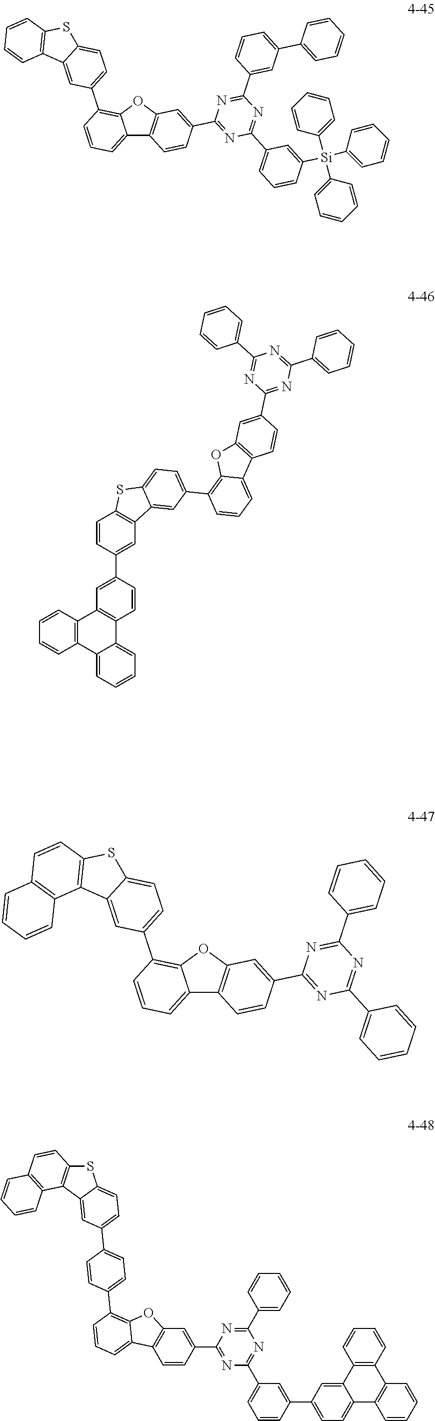 [SMILES: c1ccc(-c2cccc(-c3nc(-c4cccc([Si](c5ccccc5)(c5ccccc5)c5ccccc5)c4)nc(-c4ccc5c(c4)oc4c(-c6ccc7sc8ccccc8c7c6)cccc45)n3)c2)cc1 ; c1ccc(-c2nc(-c3ccccc3)nc(-c3ccc4c(c3)oc3c(-c5ccc6sc7ccc(-c8ccc9c%10ccccc%10c%10ccccc%10c9c8)cc7c6c5)cccc34)n2)cc1 ; c1ccc(-c2nc(-c3ccccc3)nc(-c3ccc4c(c3)oc3c(-c5ccc6sc7ccc8ccccc8c7c6c5)cccc34)n2)cc1 ; c1ccc(-c2nc(-c3cccc(-c4ccc5c6ccccc6c6ccccc6c5c4)c3)nc(-c3ccc4c(c3)oc3c(-c5ccc(-c6ccc7sc8ccc9ccccc9c8c7c6)cc5)cccc34)n2)cc1]

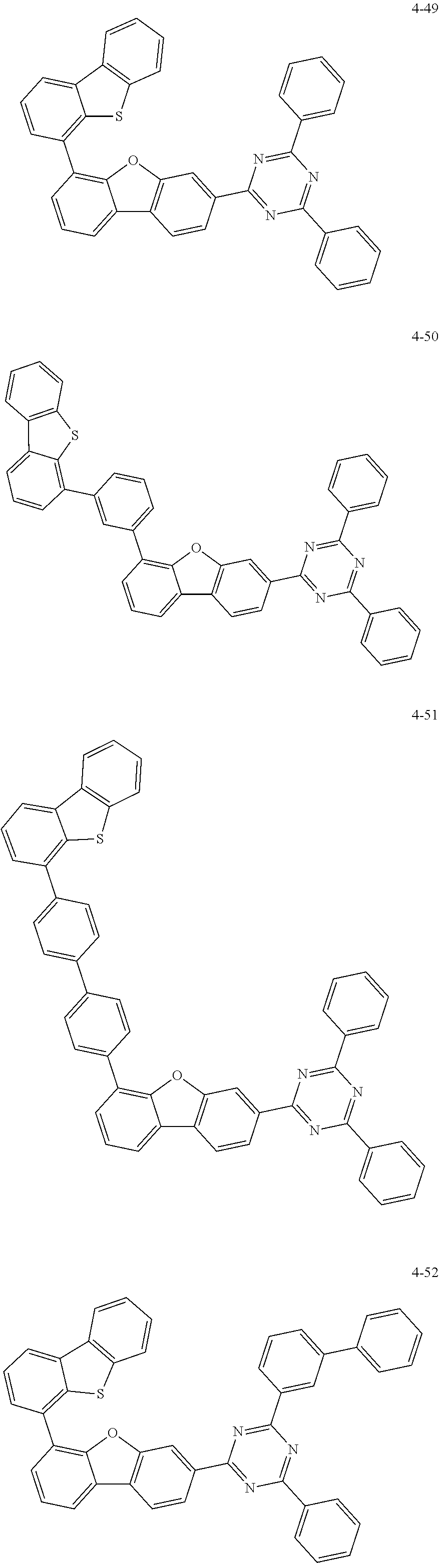 [SMILES: c1ccc(-c2nc(-c3ccccc3)nc(-c3ccc4c(c3)oc3c(-c5cccc6c5sc5ccccc56)cccc34)n2)cc1 ; c1ccc(-c2nc(-c3ccccc3)nc(-c3ccc4c(c3)oc3c(-c5cccc(-c6cccc7c6sc6ccccc67)c5)cccc34)n2)cc1 ; c1ccc(-c2nc(-c3ccccc3)nc(-c3ccc4c(c3)oc3c(-c5ccc(-c6ccc(-c7cccc8c7sc7ccccc78)cc6)cc5)cccc34)n2)cc1 ; c1ccc(-c2cccc(-c3nc(-c4ccccc4)nc(-c4ccc5c(c4)oc4c(-c6cccc7c6sc6ccccc67)cccc45)n3)c2)cc1]

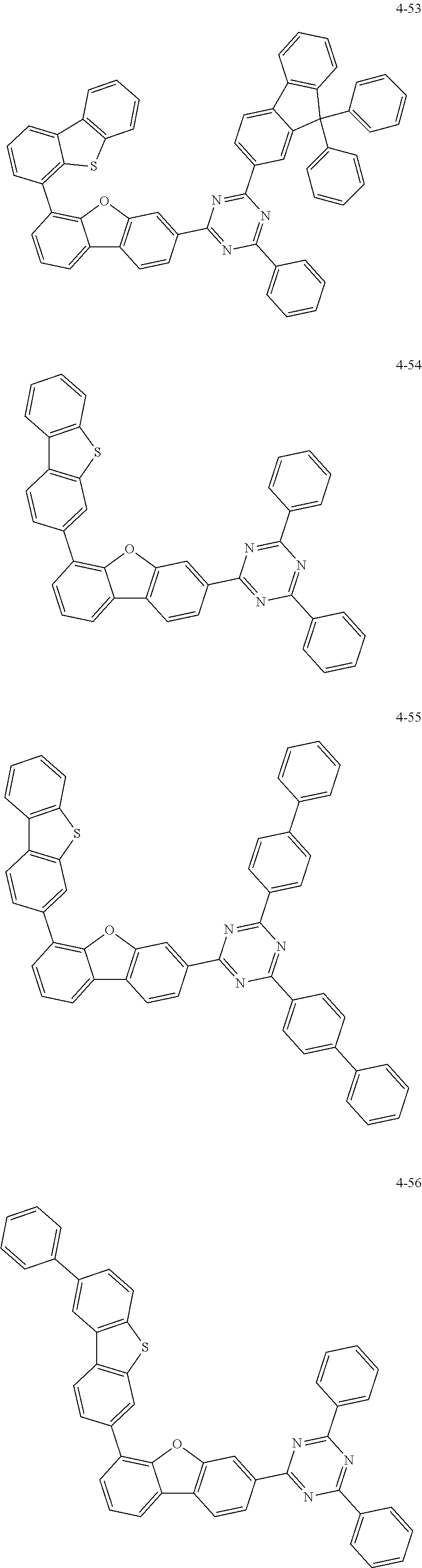 [SMILES: c1ccc(-c2nc(-c3ccc4c(c3)C(c3ccccc3)(c3ccccc3)c3ccccc3-4)nc(-c3ccc4c(c3)oc3c(-c5cccc6c5sc5ccccc56)cccc34)n2)cc1 ; c1ccc(-c2nc(-c3ccccc3)nc(-c3ccc4c(c3)oc3c(-c5ccc6c(c5)sc5ccccc56)cccc34)n2)cc1 ; c1ccc(-c2ccc(-c3nc(-c4ccc(-c5ccccc5)cc4)nc(-c4ccc5c(c4)oc4c(-c6ccc7c(c6)sc6ccccc67)cccc45)n3)cc2)cc1 ; c1ccc(-c2ccc3sc4cc(-c5cccc6c5oc5cc(-c7nc(-c8ccccc8)nc(-c8ccccc8)n7)ccc56)ccc4c3c2)cc1]

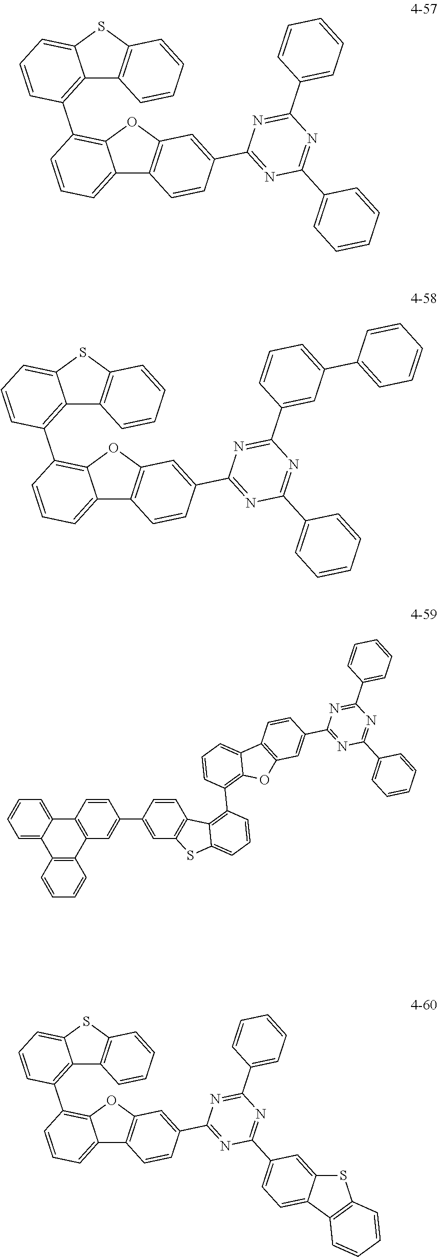 [SMILES: c1ccc(-c2nc(-c3ccccc3)nc(-c3ccc4c(c3)oc3c(-c5cccc6sc7ccccc7c56)cccc34)n2)cc1 ; c1ccc(-c2cccc(-c3nc(-c4ccccc4)nc(-c4ccc5c(c4)oc4c(-c6cccc7sc8ccccc8c67)cccc45)n3)c2)cc1 ; c1ccc(-c2nc(-c3ccccc3)nc(-c3ccc4c(c3)oc3c(-c5cccc6sc7cc(-c8ccc9c%10ccccc%10c%10ccccc%10c9c8)ccc7c56)cccc34)n2)cc1 ; c1ccc(-c2nc(-c3ccc4c(c3)oc3c(-c5cccc6sc7ccccc7c56)cccc34)nc(-c3ccc4c(c3)sc3ccccc34)n2)cc1]

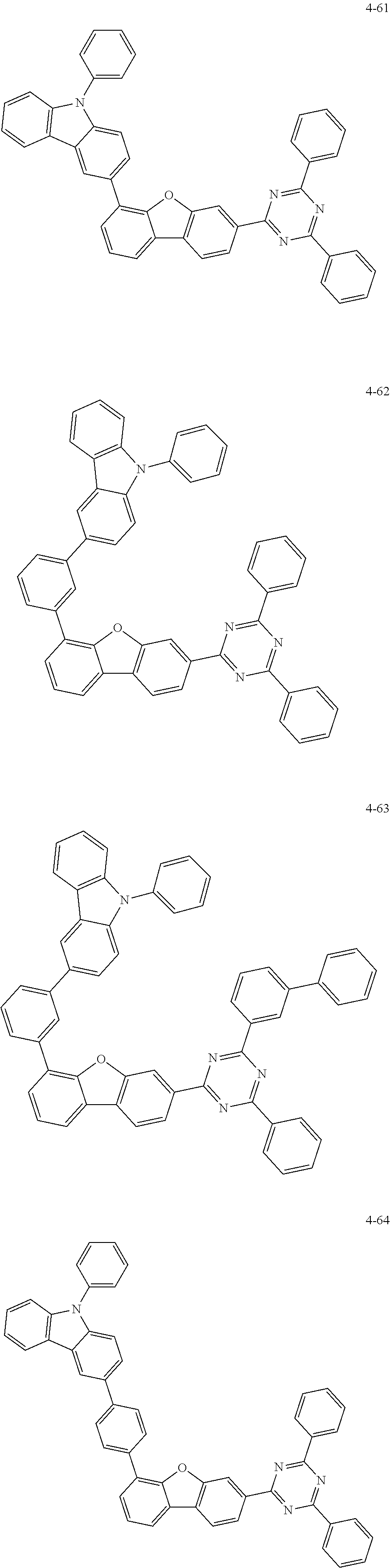 [SMILES: c1ccc(-c2nc(-c3ccccc3)nc(-c3ccc4c(c3)oc3c(-c5ccc6c(c5)c5ccccc5n6-c5ccccc5)cccc34)n2)cc1 ; c1ccc(-c2nc(-c3ccccc3)nc(-c3ccc4c(c3)oc3c(-c5cccc(-c6ccc7c(c6)c6ccccc6n7-c6ccccc6)c5)cccc34)n2)cc1 ; c1ccc(-c2cccc(-c3nc(-c4ccccc4)nc(-c4ccc5c(c4)oc4c(-c6cccc(-c7ccc8c(c7)c7ccccc7n8-c7ccccc7)c6)cccc45)n3)c2)cc1 ; c1ccc(-c2nc(-c3ccccc3)nc(-c3ccc4c(c3)oc3c(-c5ccc(-c6ccc7c(c6)c6ccccc6n7-c6ccccc6)cc5)cccc34)n2)cc1]

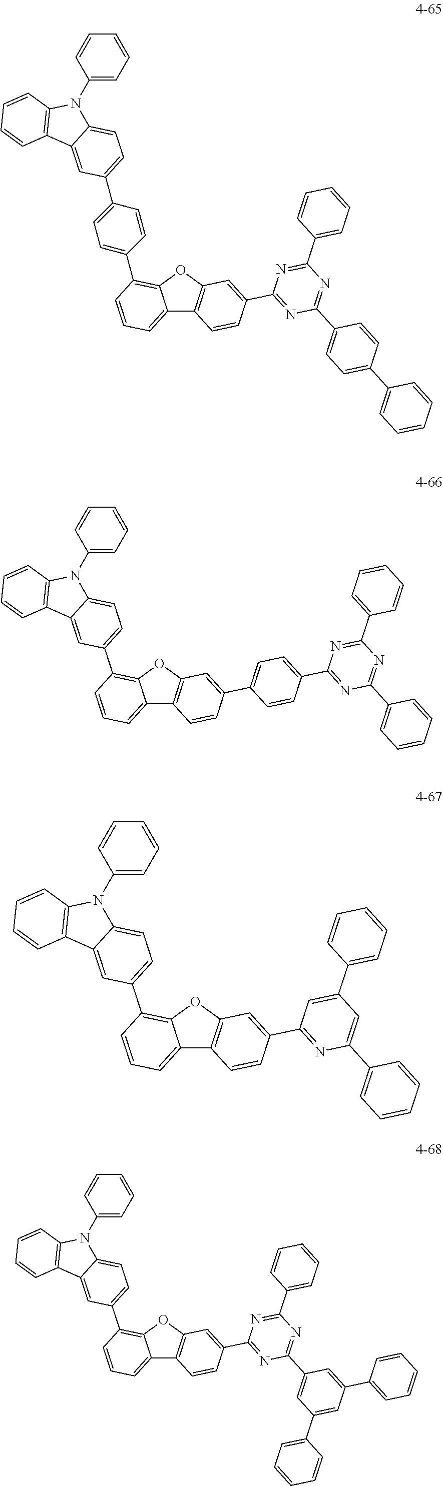 [SMILES: c1ccc(-c2ccc(-c3nc(-c4ccccc4)nc(-c4ccc5c(c4)oc4c(-c6ccc(-c7ccc8c(c7)c7ccccc7n8-c7ccccc7)cc6)cccc45)n3)cc2)cc1 ; c1ccc(-c2nc(-c3ccccc3)nc(-c3ccc(-c4ccc5c(c4)oc4c(-c6ccc7c(c6)c6ccccc6n7-c6ccccc6)cccc45)cc3)n2)cc1 ; c1ccc(-c2cc(-c3ccccc3)nc(-c3ccc4c(c3)oc3c(-c5ccc6c(c5)c5ccccc5n6-c5ccccc5)cccc34)c2)cc1 ; c1ccc(-c2cc(-c3ccccc3)cc(-c3nc(-c4ccccc4)nc(-c4ccc5c(c4)oc4c(-c6ccc7c(c6)c6ccccc6n7-c6ccccc6)cccc45)n3)c2)cc1]

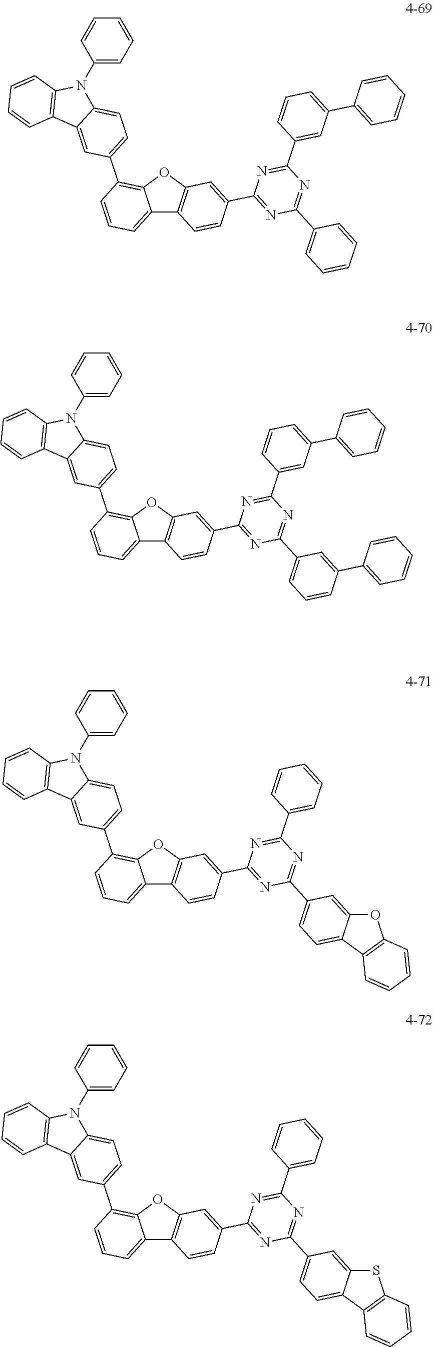 [SMILES: c1ccc(-c2cccc(-c3nc(-c4ccccc4)nc(-c4ccc5c(c4)oc4c(-c6ccc7c(c6)c6ccccc6n7-c6ccccc6)cccc45)n3)c2)cc1 ; c1ccc(-c2cccc(-c3nc(-c4cccc(-c5ccccc5)c4)nc(-c4ccc5c(c4)oc4c(-c6ccc7c(c6)c6ccccc6n7-c6ccccc6)cccc45)n3)c2)cc1 ; c1ccc(-c2nc(-c3ccc4c(c3)oc3ccccc34)nc(-c3ccc4c(c3)oc3c(-c5ccc6c(c5)c5ccccc5n6-c5ccccc5)cccc34)n2)cc1 ; c1ccc(-c2nc(-c3ccc4c(c3)oc3c(-c5ccc6c(c5)c5ccccc5n6-c5ccccc5)cccc34)nc(-c3ccc4c(c3)sc3ccccc34)n2)cc1]

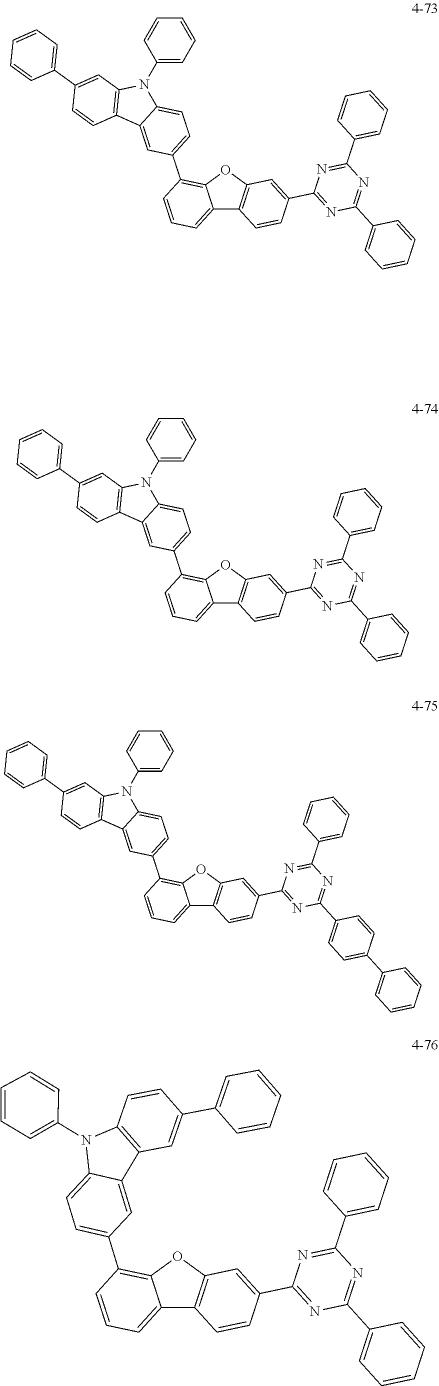 [SMILES: c1ccc(-c2ccc3c4cc(-c5cccc6c5oc5cc(-c7nc(-c8ccccc8)nc(-c8ccccc8)n7)ccc56)ccc4n(-c4ccccc4)c3c2)cc1 ; c1ccc(-c2ccc3c4cc(-c5cccc6c5oc5cc(-c7nc(-c8ccccc8)nc(-c8ccccc8)n7)ccc56)ccc4n(-c4ccccc4)c3c2)cc1 ; c1ccc(-c2ccc(-c3nc(-c4ccccc4)nc(-c4ccc5c(c4)oc4c(-c6ccc7c(c6)c6ccc(-c8ccccc8)cc6n7-c6ccccc6)cccc45)n3)cc2)cc1 ; c1ccc(-c2ccc3c(c2)c2cc(-c4cccc5c4oc4cc(-c6nc(-c7ccccc7)nc(-c7ccccc7)n6)ccc45)ccc2n3-c2ccccc2)cc1]

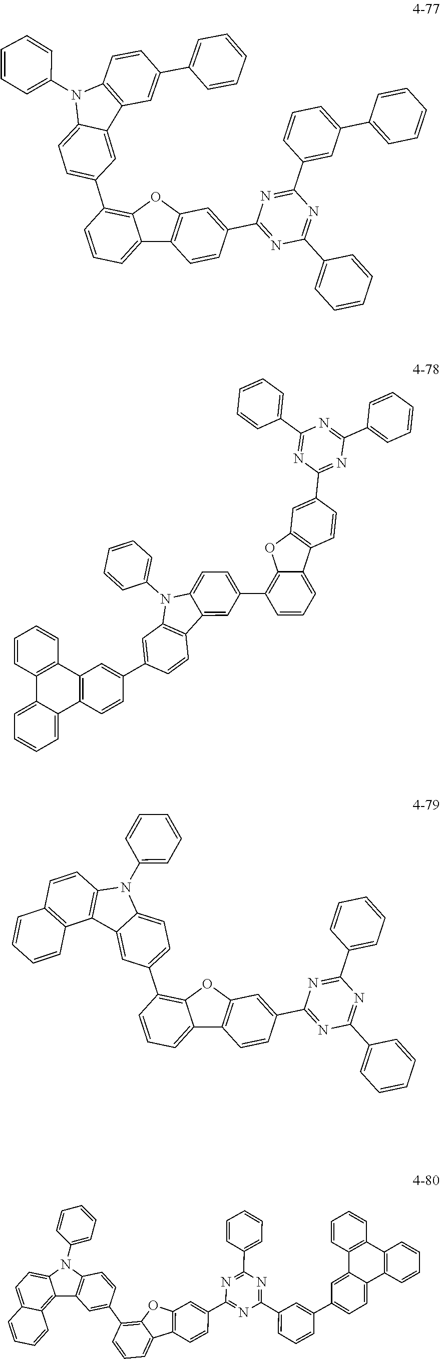 [SMILES: c1ccc(-c2cccc(-c3nc(-c4ccccc4)nc(-c4ccc5c(c4)oc4c(-c6ccc7c(c6)c6cc(-c8ccccc8)ccc6n7-c6ccccc6)cccc45)n3)c2)cc1 ; c1ccc(-c2nc(-c3ccccc3)nc(-c3ccc4c(c3)oc3c(-c5ccc6c(c5)c5ccc(-c7ccc8c9ccccc9c9ccccc9c8c7)cc5n6-c5ccccc5)cccc34)n2)cc1 ; c1ccc(-c2nc(-c3ccccc3)nc(-c3ccc4c(c3)oc3c(-c5ccc6c(c5)c5c7ccccc7ccc5n6-c5ccccc5)cccc34)n2)cc1 ; c1ccc(-c2nc(-c3cccc(-c4ccc5c6ccccc6c6ccccc6c5c4)c3)nc(-c3ccc4c(c3)oc3c(-c5ccc6c(c5)c5c7ccccc7ccc5n6-c5ccccc5)cccc34)n2)cc1]

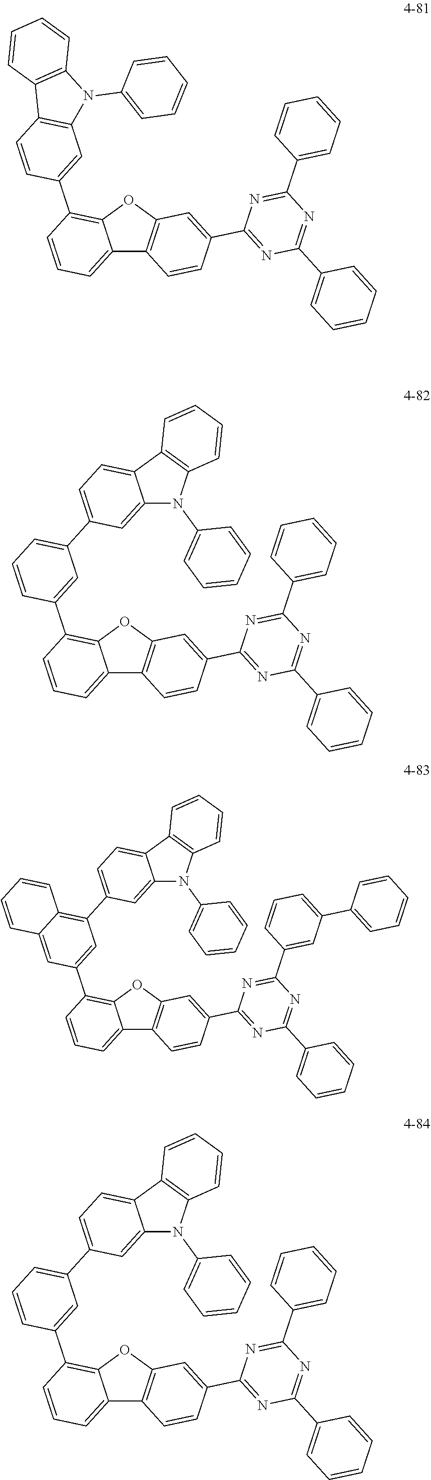 [SMILES: c1ccc(-c2nc(-c3ccccc3)nc(-c3ccc4c(c3)oc3c(-c5ccc6c7ccccc7n(-c7ccccc7)c6c5)cccc34)n2)cc1 ; c1ccc(-c2nc(-c3ccccc3)nc(-c3ccc4c(c3)oc3c(-c5cccc(-c6ccc7c8ccccc8n(-c8ccccc8)c7c6)c5)cccc34)n2)cc1 ; c1ccc(-c2cccc(-c3nc(-c4ccccc4)nc(-c4ccc5c(c4)oc4c(-c6cc(-c7ccc8c9ccccc9n(-c9ccccc9)c8c7)c7ccccc7c6)cccc45)n3)c2)cc1 ; c1ccc(-c2nc(-c3ccccc3)nc(-c3ccc4c(c3)oc3c(-c5cccc(-c6ccc7c8ccccc8n(-c8ccccc8)c7c6)c5)cccc34)n2)cc1]

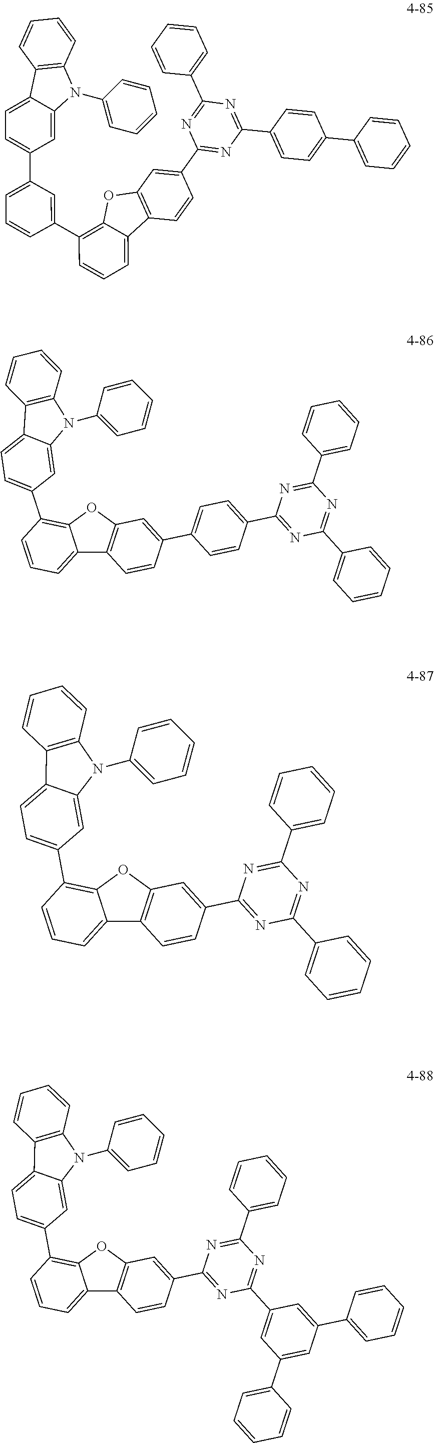 [SMILES: c1ccc(-c2ccc(-c3nc(-c4ccccc4)nc(-c4ccc5c(c4)oc4c(-c6cccc(-c7ccc8c9ccccc9n(-c9ccccc9)c8c7)c6)cccc45)n3)cc2)cc1 ; c1ccc(-c2nc(-c3ccccc3)nc(-c3ccc(-c4ccc5c(c4)oc4c(-c6ccc7c8ccccc8n(-c8ccccc8)c7c6)cccc45)cc3)n2)cc1 ; c1ccc(-c2nc(-c3ccccc3)nc(-c3ccc4c(c3)oc3c(-c5ccc6c7ccccc7n(-c7ccccc7)c6c5)cccc34)n2)cc1 ; c1ccc(-c2cc(-c3ccccc3)cc(-c3nc(-c4ccccc4)nc(-c4ccc5c(c4)oc4c(-c6ccc7c8ccccc8n(-c8ccccc8)c7c6)cccc45)n3)c2)cc1]

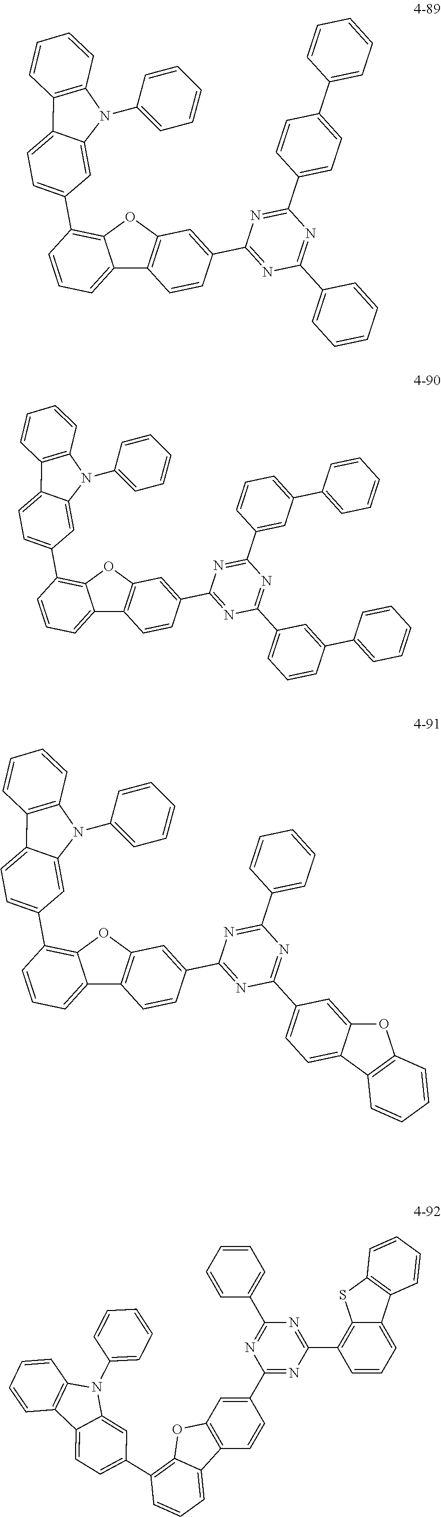 [SMILES: c1ccc(-c2ccc(-c3nc(-c4ccccc4)nc(-c4ccc5c(c4)oc4c(-c6ccc7c8ccccc8n(-c8ccccc8)c7c6)cccc45)n3)cc2)cc1 ; c1ccc(-c2cccc(-c3nc(-c4cccc(-c5ccccc5)c4)nc(-c4ccc5c(c4)oc4c(-c6ccc7c8ccccc8n(-c8ccccc8)c7c6)cccc45)n3)c2)cc1 ; c1ccc(-c2nc(-c3ccc4c(c3)oc3ccccc34)nc(-c3ccc4c(c3)oc3c(-c5ccc6c7ccccc7n(-c7ccccc7)c6c5)cccc34)n2)cc1 ; c1ccc(-c2nc(-c3ccc4c(c3)oc3c(-c5ccc6c7ccccc7n(-c7ccccc7)c6c5)cccc34)nc(-c3cccc4c3sc3ccccc34)n2)cc1]

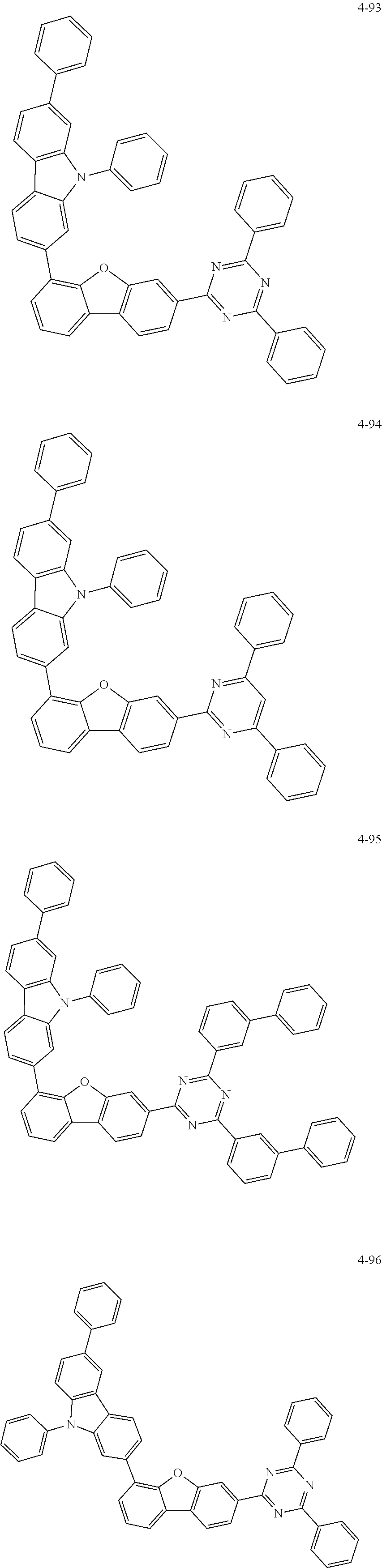 [SMILES: c1ccc(-c2ccc3c4ccc(-c5cccc6c5oc5cc(-c7nc(-c8ccccc8)nc(-c8ccccc8)n7)ccc56)cc4n(-c4ccccc4)c3c2)cc1 ; c1ccc(-c2ccc3c4ccc(-c5cccc6c5oc5cc(-c7nc(-c8ccccc8)cc(-c8ccccc8)n7)ccc56)cc4n(-c4ccccc4)c3c2)cc1 ; c1ccc(-c2cccc(-c3nc(-c4cccc(-c5ccccc5)c4)nc(-c4ccc5c(c4)oc4c(-c6ccc7c8ccc(-c9ccccc9)cc8n(-c8ccccc8)c7c6)cccc45)n3)c2)cc1 ; c1ccc(-c2ccc3c(c2)c2ccc(-c4cccc5c4oc4cc(-c6nc(-c7ccccc7)nc(-c7ccccc7)n6)ccc45)cc2n3-c2ccccc2)cc1]

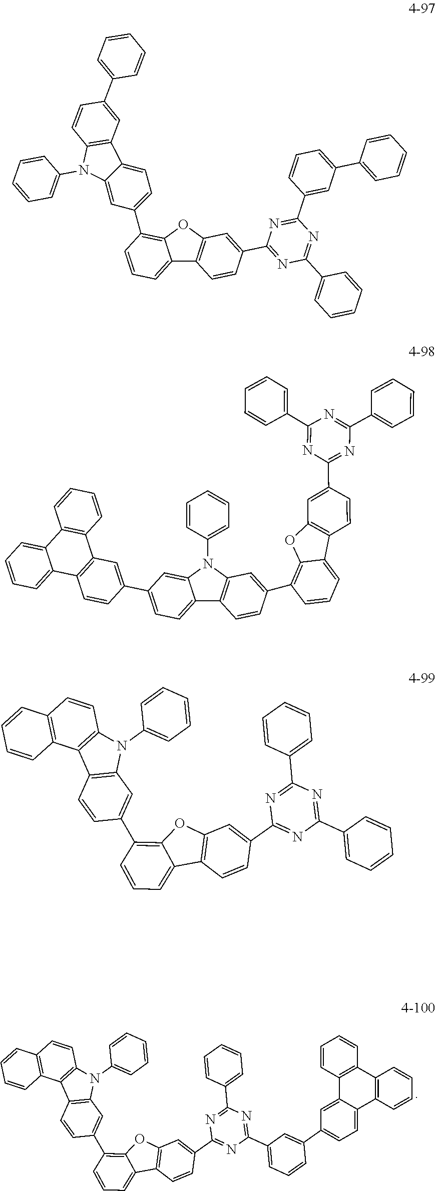 [SMILES: c1ccc(-c2cccc(-c3nc(-c4ccccc4)nc(-c4ccc5c(c4)oc4c(-c6ccc7c8cc(-c9ccccc9)ccc8n(-c8ccccc8)c7c6)cccc45)n3)c2)cc1 ; c1ccc(-c2nc(-c3ccccc3)nc(-c3ccc4c(c3)oc3c(-c5ccc6c7ccc(-c8ccc9c%10ccccc%10c%10ccccc%10c9c8)cc7n(-c7ccccc7)c6c5)cccc34)n2)cc1 ; c1ccc(-c2nc(-c3ccccc3)nc(-c3ccc4c(c3)oc3c(-c5ccc6c7c8ccccc8ccc7n(-c7ccccc7)c6c5)cccc34)n2)cc1 ; c1ccc(-c2nc(-c3cccc(-c4ccc5c6ccccc6c6ccccc6c5c4)c3)nc(-c3ccc4c(c3)oc3c(-c5ccc6c7c8ccccc8ccc7n(-c7ccccc7)c6c5)cccc34)n2)cc1]

In addition, by introducing various substituents to the structure of Chemical Formula 1, compounds having unique properties of the introduced substituents may be synthesized. For example, by introducing substituents normally used as hole injection layer materials, hole transfer layer materials, light emitting layer materials, electron transfer layer materials and charge generation layer materials used for manufacturing an organic light emitting device to the core structure, materials satisfying conditions required for each organic material layer may be synthesized.

In addition, by introducing various substituents to the structure of Chemical Formula 1, the energy band gap may be finely controlled, and meanwhile, properties at interfaces between organic materials are enhanced, and material applications may become diverse.

In addition, one embodiment of the present application provides an organic light emitting device comprising a first electrode; a second electrode provided opposite to the first electrode; and one or more organic material layers provided between the first electrode and the second electrode, wherein one or more layers of the organic material layers comprise the heterocyclic compound according to Chemical Formula 1.

Another embodiment of the present application provides an organic light emitting device comprising a first electrode; a second electrode provided opposite to the first electrode; and one or more organic material layers provided between the first electrode and the second electrode, wherein one or more layers of the organic material layers comprise one heterocyclic compound according to Chemical Formula 1.

Specific descriptions on the heterocyclic compound represented by Chemical Formula 1 are the same as the descriptions provided above.

In one embodiment of the present application, the first electrode may be an anode, and the second electrode may be a cathode.

In another embodiment, the first electrode may be a cathode, and the second electrode may be an anode.

In one embodiment of the present application, the organic light emitting device may be a blue organic light emitting device, and the heterocyclic compound according to Chemical Formula 1 may be used as a material of the blue organic light emitting device. For example, the heterocyclic compound according to Chemical Formula 1 may be included in a host material of a blue light emitting layer of the blue organic light emitting device.

In one embodiment of the present application, the organic light emitting device may be a green organic light emitting device, and the heterocyclic compound according to Chemical Formula 1 may be used as a material of the green organic light emitting device. For example, the heterocyclic compound according to Chemical Formula 1 may be included in a host material of a green light emitting layer of the green organic light emitting device.

In one embodiment of the present application, the organic light emitting device may be a red organic light emitting device, and the heterocyclic compound according to Chemical Formula 1 may be used as a material of the red organic light emitting device. For example, the heterocyclic compound according to Chemical Formula 1 may be included in a host material of a red light emitting layer of the red organic light emitting device.

The organic light emitting device of the present disclosure may be manufactured using common organic light emitting device manufacturing methods and materials except that one or more of the organic material layers are formed using the heterocyclic compound described above.

The heterocyclic compound may be formed into an organic material layer through a solution coating method as well as a vacuum deposition method when manufacturing the organic light emitting device. Herein, the solution coating method means spin coating, dip coating, inkjet printing, screen printing, a spray method, roll coating and the like, but is not limited thereto.

The organic material layer of the organic light emitting device of the present disclosure may be formed in a single layer structure, but may be formed in a multilayer structure in which two or more organic material layers are laminated. For example, the organic light emitting device of the present disclosure may have a structure comprising a hole injection layer, a hole transfer layer, a light emitting layer, an electron transfer layer, an electron injection layer and the like as the organic material layer. However, the structure of the organic light emitting device is not limited thereto, and may comprise a smaller number of organic material layers.

In the organic light emitting device of the present disclosure, the organic material layer may comprise a light emitting layer, and the light emitting layer may comprise the heterocyclic compound.

In another organic light emitting device, the organic material layer comprises a light emitting layer, the light emitting layer comprises a host material, and the host material may comprise the heterocyclic compound.

As another example, the organic material layer comprising the heterocyclic compound comprises the heterocyclic compound represented by Chemical Formula 1 as a host, and an iridium-based dopant may be used therewith.

In the organic light emitting device of the present disclosure, the organic material layer comprises an electron injection layer or an electron transfer layer, and the electron transfer layer or the electron injection layer may comprise the heterocyclic compound.

In another organic light emitting device, the organic material layer comprises an electron blocking layer or a hole blocking layer, and the electron blocking layer or the hole blocking layer may comprise the heterocyclic compound.

The organic light emitting device of the present disclosure may further comprise one, two or more layers selected from the group consisting of a light emitting layer, a hole injection layer, a hole transfer layer, an electron injection layer, an electron transfer layer, an electron blocking layer and a hole blocking layer.

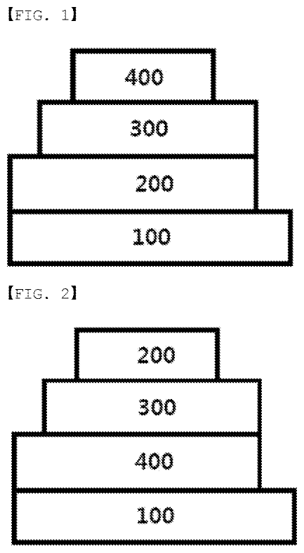

FIG. 1 to FIG. 3 illustrate a lamination order of electrodes and organic material layers of an organic light emitting device according to one embodiment of the present application. However, the scope of the present application is not limited to these diagrams, and structures of organic light emitting devices known in the art may also be used in the present application.

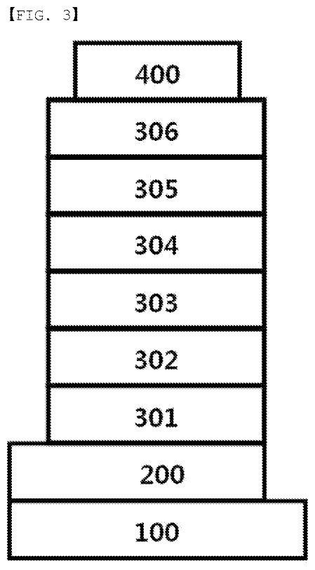

FIG. 1 illustrates an organic light emitting device in which an anode (200), an organic material layer (300) and a cathode (400) are consecutively laminated on a substrate (100). However, the structure is not limited to such a structure, and as illustrated in FIG. 2, an organic light emitting device in which a cathode, an organic material layer and an anode are consecutively laminated on a substrate may also be obtained.

FIG. 3 illustrates a case of the organic material layer being a multilayer. The organic light emitting device according to FIG. 3 comprises a hole injection layer (301), a hole transfer layer (302), a light emitting layer (303), a hole blocking layer (304), an electron transfer layer (305) and an electron injection layer (306). However, the scope of the present application is not limited to such a lamination structure, and as necessary, layers other than the light emitting layer may not be included, and other necessary functional layers may be further added.

The organic material layer comprising the compound of Chemical Formula 1 may further comprise other materials as necessary.

In the organic light emitting device according to one embodiment of the present application, the organic material layer may further comprise a heterocyclic compound of the following Chemical Formula 2.

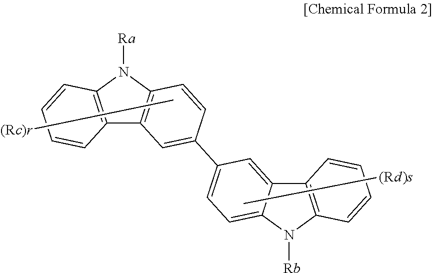

In Chemical Formula 2,

Ra and Rb are the same as or different from each other, and each independently a substituted or unsubstituted C6 to C60 aryl group; or a substituted or unsubstituted C2 to C60 heteroaryl group,

Rc and Rd are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; and a substituted or unsubstituted amine group, and

r and s are an integer of 0 to 7.

Effects of more superior efficiency and lifetime are obtained when comprising the compound of Chemical Formula 1 and the compound of Chemical Formula 2 at the same time in the organic material layer of the organic light emitting device. Such results may lead to a forecast that an exciplex phenomenon occurs when comprising the two compounds at the same time.

The exciplex phenomenon is a phenomenon of releasing energy having sizes of a donor (p-host) HOMO level and an acceptor (n-host) LUND level due to electron exchanges between two molecules. When the exciplex phenomenon occurs between two molecules, reverse intersystem crossing (RISC) occurs, and as a result, internal quantum efficiency of fluorescence may increase up to 100%. When a donor (p-host) having a favorable hole transfer ability and an acceptor (n-host) having a favorable electron transfer ability are used as a host of a light emitting layer, holes are injected to the p-host and electrons are injected to the n-host, and therefore, a driving voltage may be lowered, which resultantly helps with enhancement in the lifetime.

In one embodiment of the present application, Rc and Rd may be hydrogen.

In one embodiment of the present application, Ra and Rb of Chemical Formula 2 are the same as or different from each other, and may be each independently a substituted or unsubstituted C6 to C60 aryl group.

In another embodiment, Ra and Rb of Chemical Formula 2 are the same as or different from each other, and may be each independently a substituted or unsubstituted C6 to C40 aryl group.

In another embodiment, Ra and Rb of Chemical Formula 2 are the same as or different from each other, and may be each independently a C6 to C40 aryl group unsubstituted or substituted with one or more substituents selected from the group consisting of a C1 to C40 alkyl group, a C6 to C40 aryl group, —CN and —SiR201R202R203.

In another embodiment, Ra and Rb of Chemical Formula 2 are the same as or different from each other, and may be each independently a phenyl group unsubstituted or substituted with a phenyl group, —CN or —SiR201R202R203; a biphenyl group unsubstituted or substituted with a phenyl group; a naphthyl group; a fluorene group unsubstituted or substituted with a methyl group or a phenyl group; a spirobifluorene group; or a triphenylene group.

In one embodiment of the present application, R201, R202 and R203 of Chemical Formula 2 may be a C6 to C60 aryl group.

In another embodiment, R201, R202 and R203 of Chemical Formula 2 may be a C6 to C40 aryl group.

In one embodiment of the present application, R201, R202 and R203 of Chemical Formula 2 may be a phenyl group.

In one embodiment of the present application, Chemical Formula 2 may be represented by any one of the following compounds, but is not limited thereto.

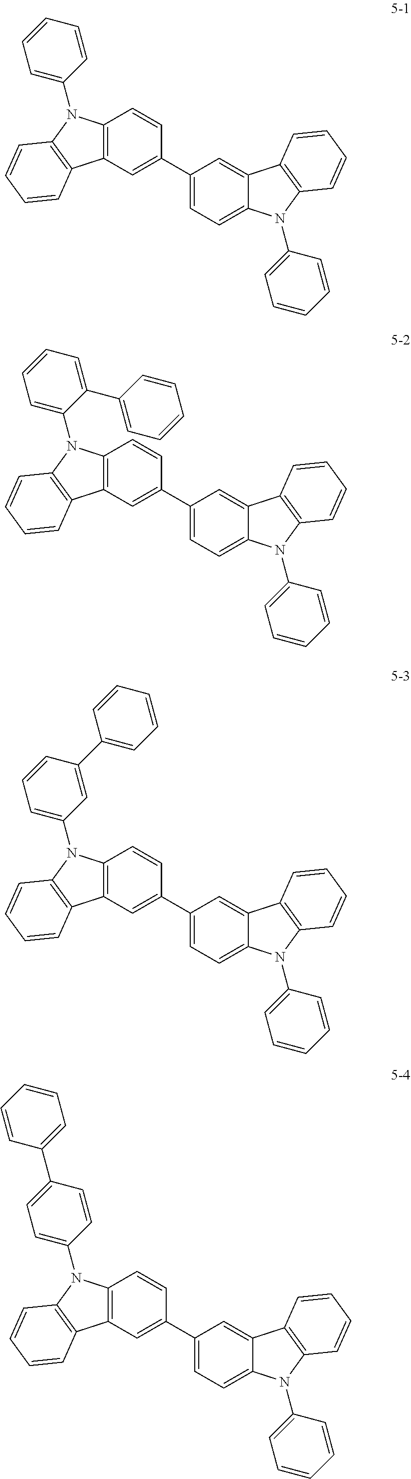 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1]

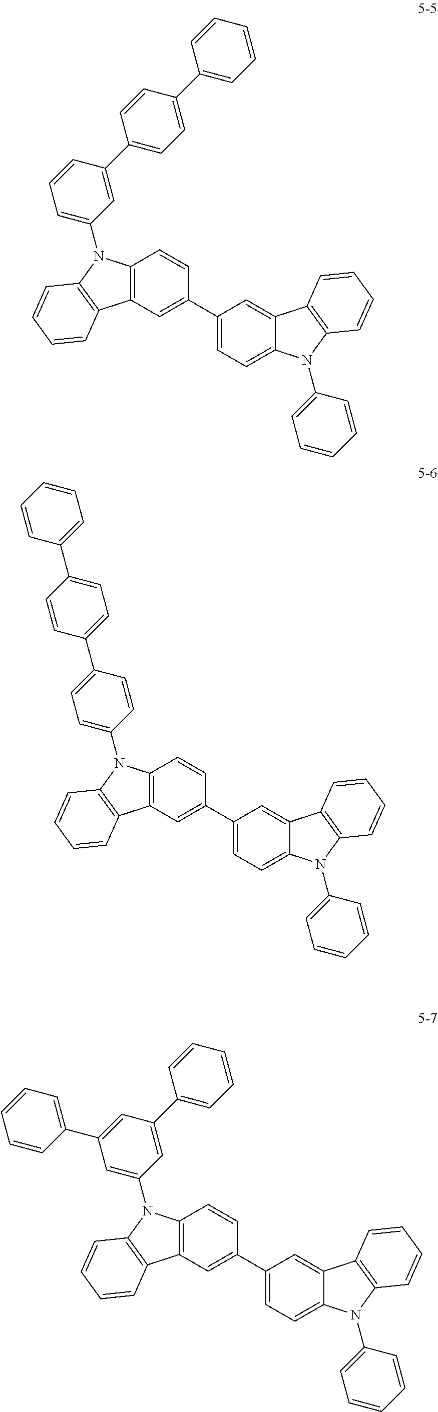 [SMILES: c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1 ; c1ccc(-c2cc(-c3ccccc3)cc(-n3c4ccccc4c4cc(-c5ccc6c(c5)c5ccccc5n6-c5ccccc5)ccc43)c2)cc1]

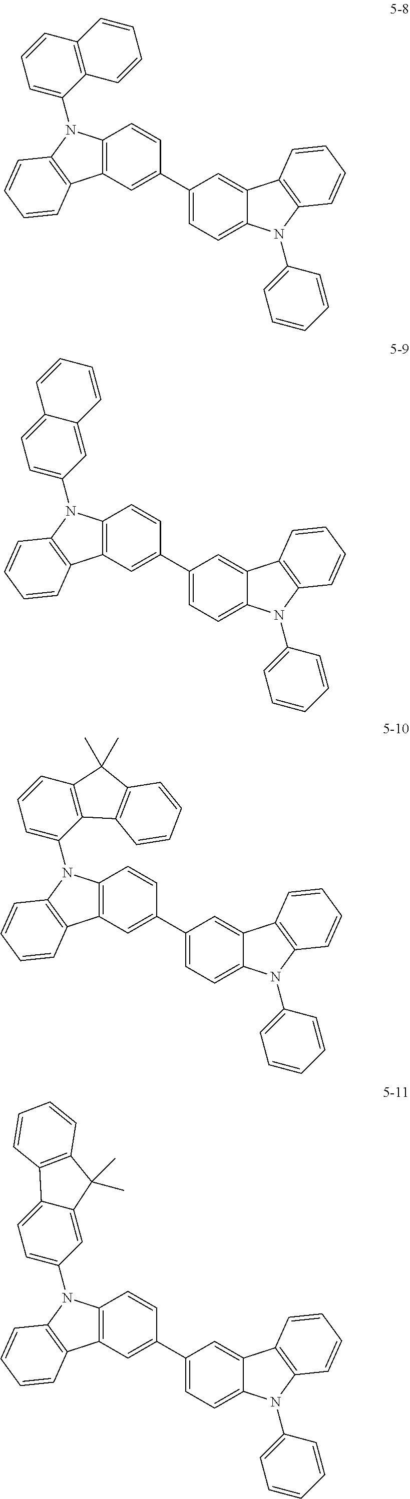 [SMILES: c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5ccccc5c4)ccc32)cc1 ; CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2ccc(-n3c4ccccc4c4cc(-c5ccc6c(c5)c5ccccc5n6-c5ccccc5)ccc43)cc21]

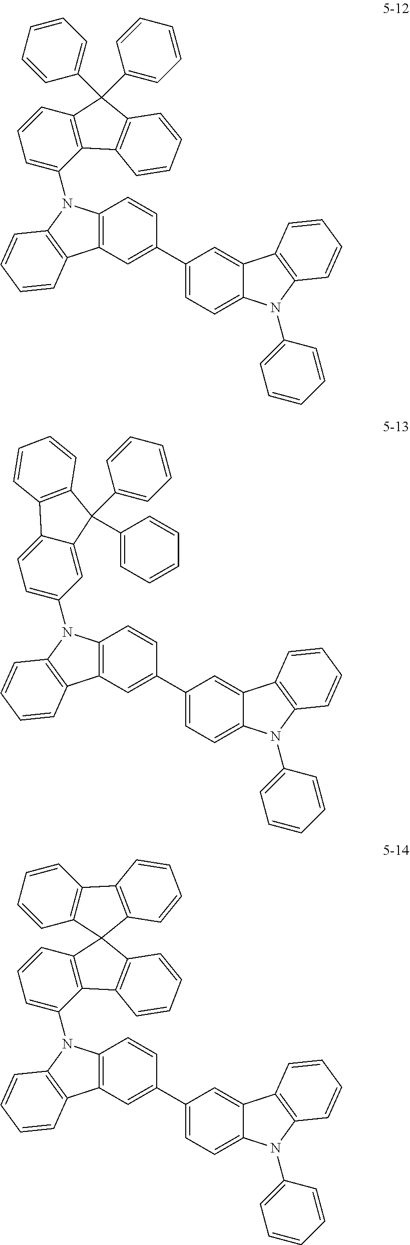 [SMILES: c1ccc(-n2c3ccccc3c3cc(-c4ccc5c(c4)c4ccccc4n5-c4cccc5c4-c4ccccc4C5(c4ccccc4)c4ccccc4)ccc32)cc1 ; c1ccc(-n2c3ccccc3c3cc(-c4ccc5c(c4)c4ccccc4n5-c4ccc5c(c4)C(c4ccccc4)(c4ccccc4)c4ccccc4-5)ccc32)cc1 ; c1ccc(-n2c3ccccc3c3cc(-c4ccc5c(c4)c4ccccc4n5-c4cccc5c4-c4ccccc4C54c5ccccc5-c5ccccc54)ccc32)cc1]

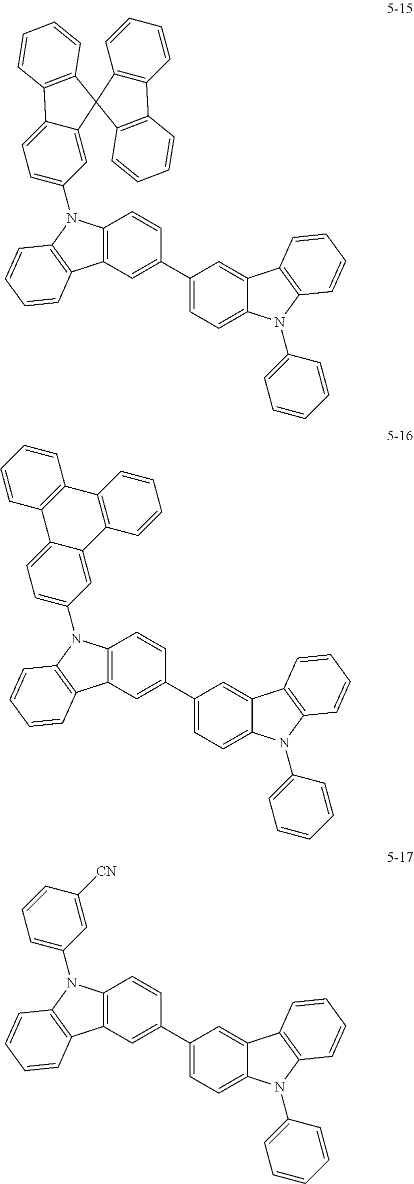 [SMILES: c1ccc(-n2c3ccccc3c3cc(-c4ccc5c(c4)c4ccccc4n5-c4ccc5c(c4)C4(c6ccccc6-c6ccccc64)c4ccccc4-5)ccc32)cc1 ; c1ccc(-n2c3ccccc3c3cc(-c4ccc5c(c4)c4ccccc4n5-c4ccc5c6ccccc6c6ccccc6c5c4)ccc32)cc1 ; N#Cc1cccc(-n2c3ccccc3c3cc(-c4ccc5c(c4)c4ccccc4n5-c4ccccc4)ccc32)c1]

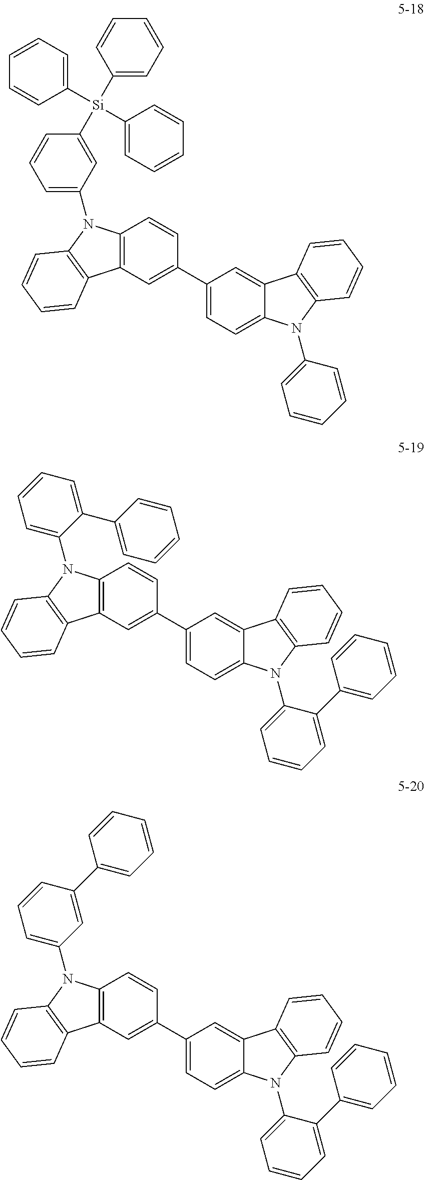 [SMILES: c1ccc(-n2c3ccccc3c3cc(-c4ccc5c(c4)c4ccccc4n5-c4cccc([Si](c5ccccc5)(c5ccccc5)c5ccccc5)c4)ccc32)cc1 ; c1ccc(-c2ccccc2-n2c3ccccc3c3cc(-c4ccc5c(c4)c4ccccc4n5-c4ccccc4-c4ccccc4)ccc32)cc1 ; c1ccc(-c2cccc(-n3c4ccccc4c4cc(-c5ccc6c(c5)c5ccccc5n6-c5ccccc5-c5ccccc5)ccc43)c2)cc1]

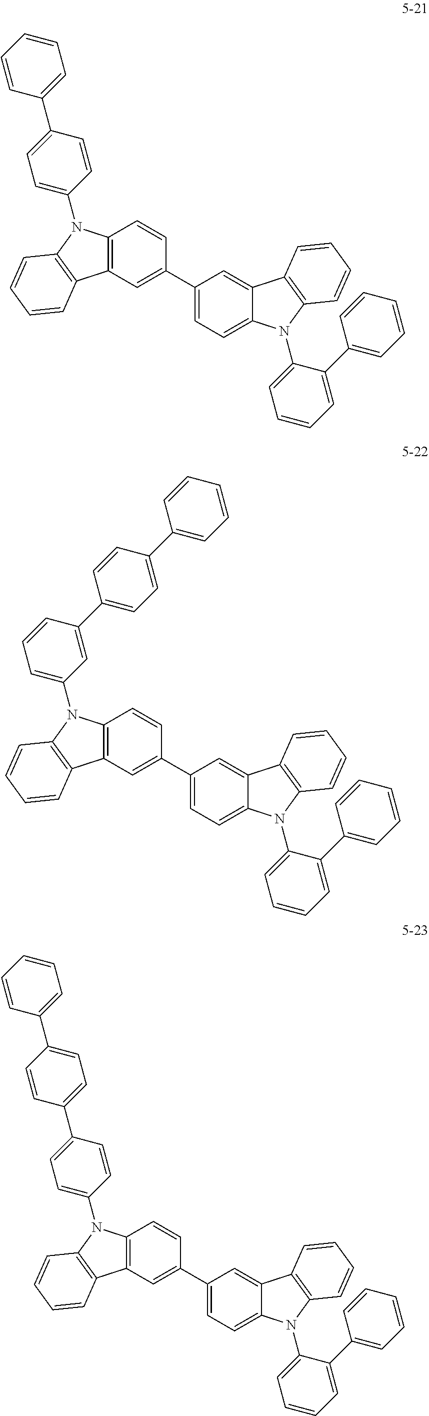 [SMILES: c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2ccc(-c3cccc(-n4c5ccccc5c5cc(-c6ccc7c(c6)c6ccccc6n7-c6ccccc6-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6-c6ccccc6)ccc54)cc3)cc2)cc1]

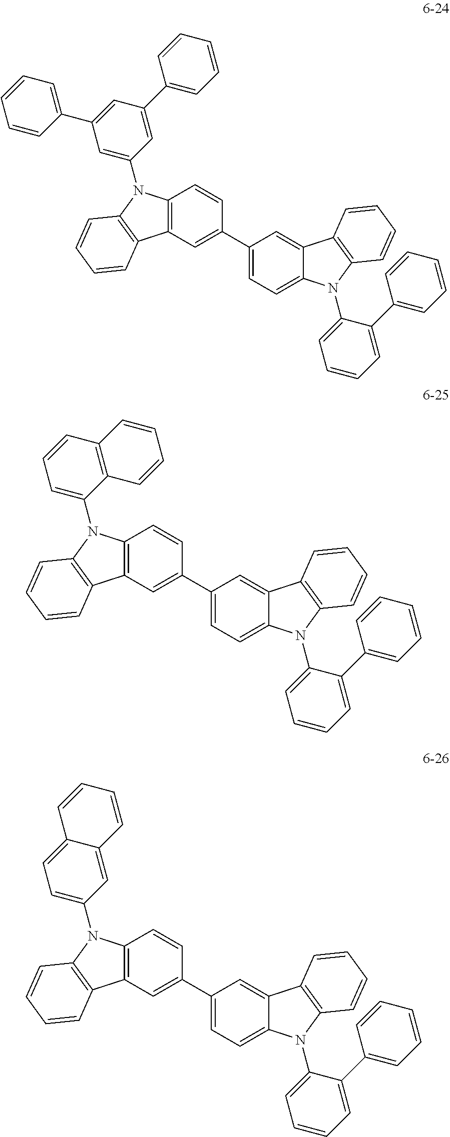 [SMILES: c1ccc(-c2cc(-c3ccccc3)cc(-n3c4ccccc4c4cc(-c5ccc6c(c5)c5ccccc5n6-c5ccccc5-c5ccccc5)ccc43)c2)cc1 ; c1ccc(-c2ccccc2-n2c3ccccc3c3cc(-c4ccc5c(c4)c4ccccc4n5-c4cccc5ccccc45)ccc32)cc1 ; c1ccc(-c2ccccc2-n2c3ccccc3c3cc(-c4ccc5c(c4)c4ccccc4n5-c4ccc5ccccc5c4)ccc32)cc1]

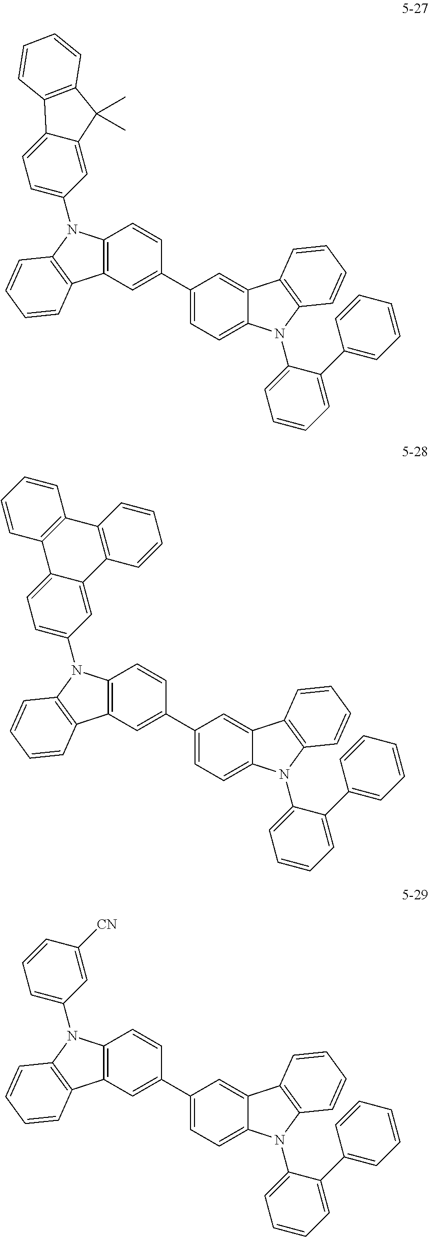 [SMILES: CC1(C)c2ccccc2-c2ccc(-n3c4ccccc4c4cc(-c5ccc6c(c5)c5ccccc5n6-c5ccccc5-c5ccccc5)ccc43)cc21 ; c1ccc(-c2ccccc2-n2c3ccccc3c3cc(-c4ccc5c(c4)c4ccccc4n5-c4ccc5c6ccccc6c6ccccc6c5c4)ccc32)cc1 ; N#Cc1cccc(-n2c3ccccc3c3cc(-c4ccc5c(c4)c4ccccc4n5-c4ccccc4-c4ccccc4)ccc32)c1]

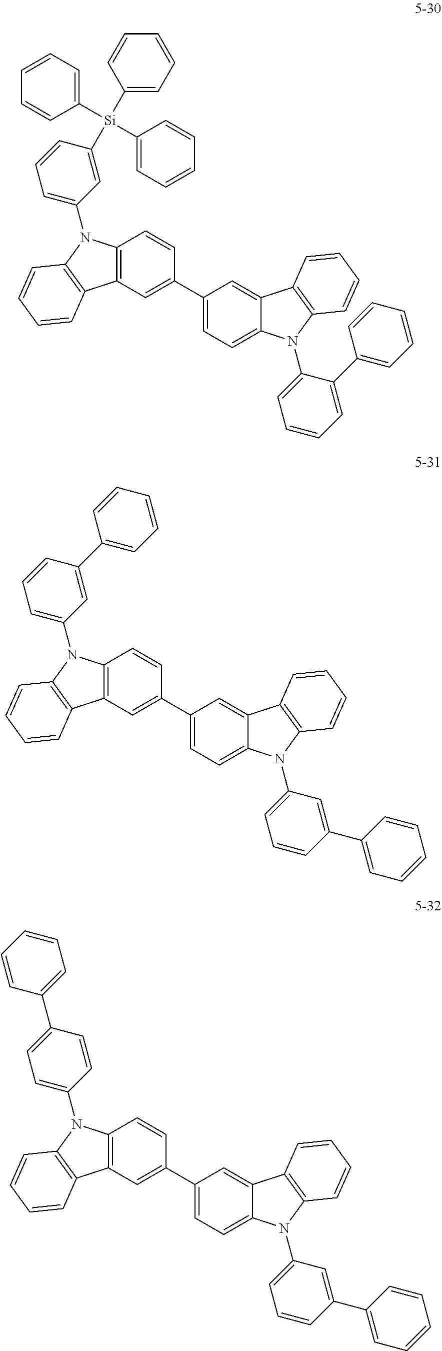 [SMILES: c1ccc(-c2ccccc2-n2c3ccccc3c3cc(-c4ccc5c(c4)c4ccccc4n5-c4cccc([Si](c5ccccc5)(c5ccccc5)c5ccccc5)c4)ccc32)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

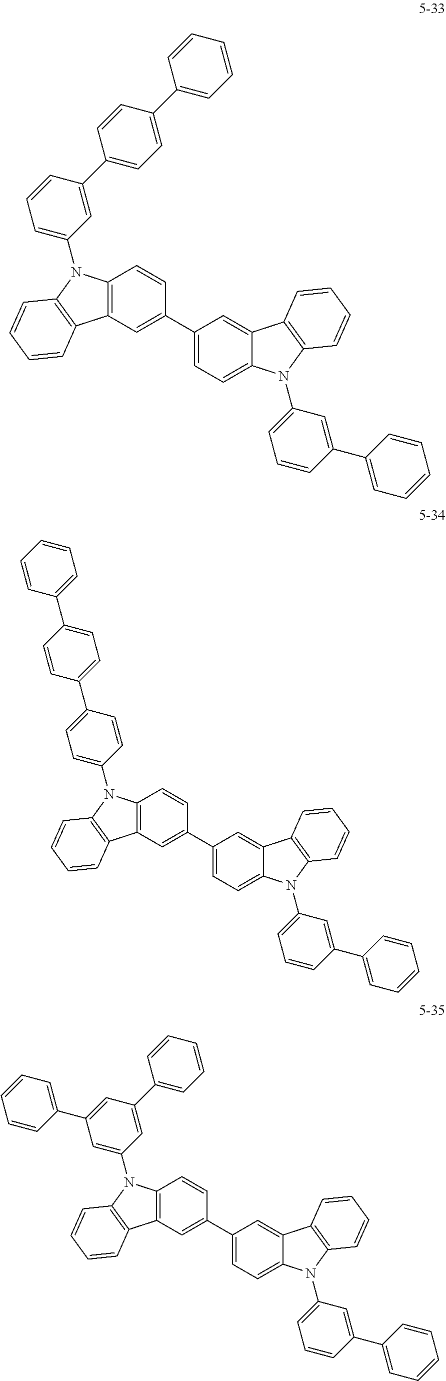 [SMILES: c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1 ; c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1]

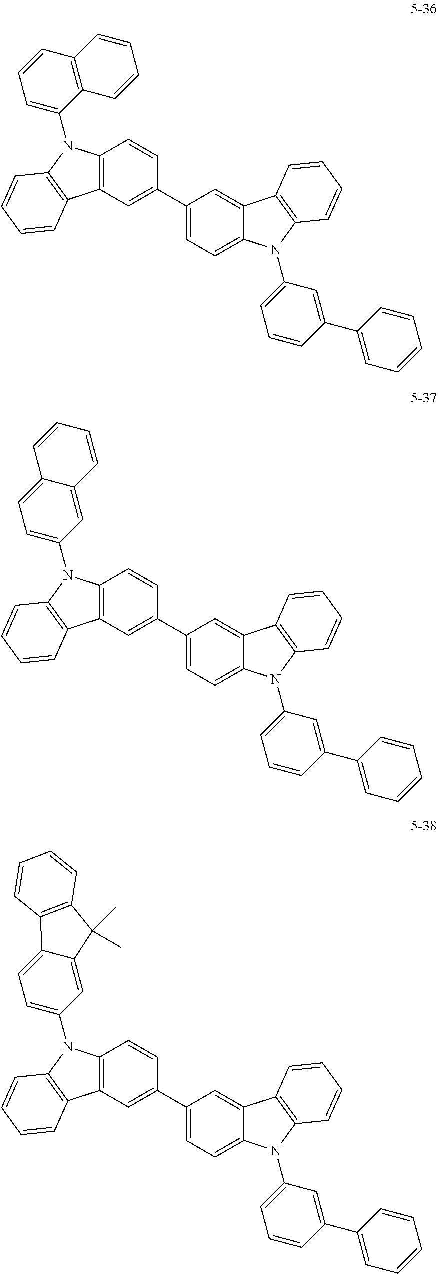 [SMILES: c1ccc(-c2cccc(-n3c4ccccc4c4cc(-c5ccc6c(c5)c5ccccc5n6-c5cccc6ccccc56)ccc43)c2)cc1 ; c1ccc(-c2cccc(-n3c4ccccc4c4cc(-c5ccc6c(c5)c5ccccc5n6-c5ccc6ccccc6c5)ccc43)c2)cc1 ; CC1(C)c2ccccc2-c2ccc(-n3c4ccccc4c4cc(-c5ccc6c(c5)c5ccccc5n6-c5cccc(-c6ccccc6)c5)ccc43)cc21]

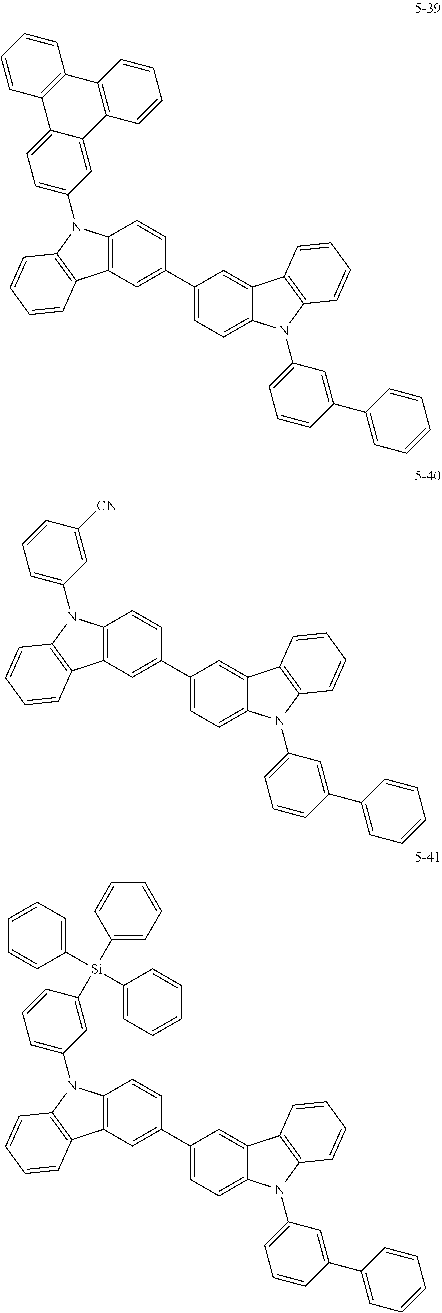 [SMILES: c1ccc(-c2cccc(-n3c4ccccc4c4cc(-c5ccc6c(c5)c5ccccc5n6-c5ccc6c7ccccc7c7ccccc7c6c5)ccc43)c2)cc1 ; N#Cc1cccc(-n2c3ccccc3c3cc(-c4ccc5c(c4)c4ccccc4n5-c4cccc(-c5ccccc5)c4)ccc32)c1 ; c1ccc(-c2cccc(-n3c4ccccc4c4cc(-c5ccc6c(c5)c5ccccc5n6-c5cccc([Si](c6ccccc6)(c6ccccc6)c6ccccc6)c5)ccc43)c2)cc1]

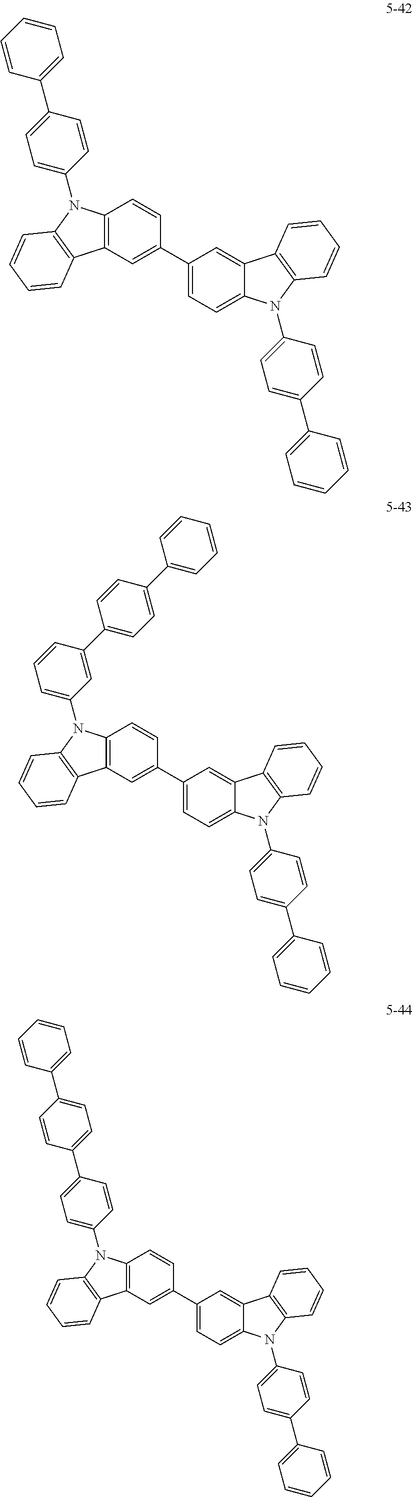 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1 ; c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1]

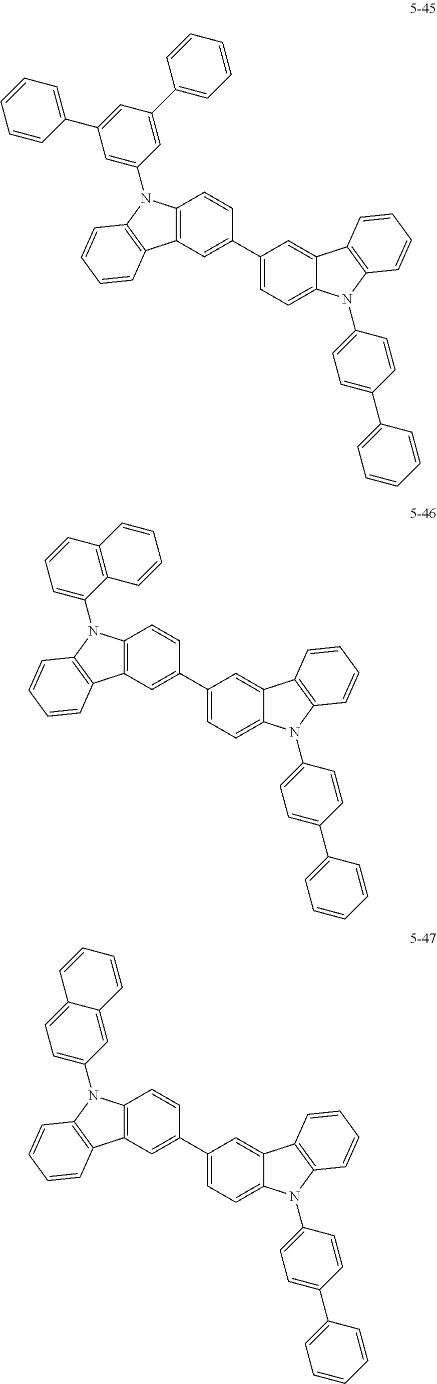 [SMILES: c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2ccc(-n3c4ccccc4c4cc(-c5ccc6c(c5)c5ccccc5n6-c5cccc6ccccc56)ccc43)cc2)cc1 ; c1ccc(-c2ccc(-n3c4ccccc4c4cc(-c5ccc6c(c5)c5ccccc5n6-c5ccc6ccccc6c5)ccc43)cc2)cc1]

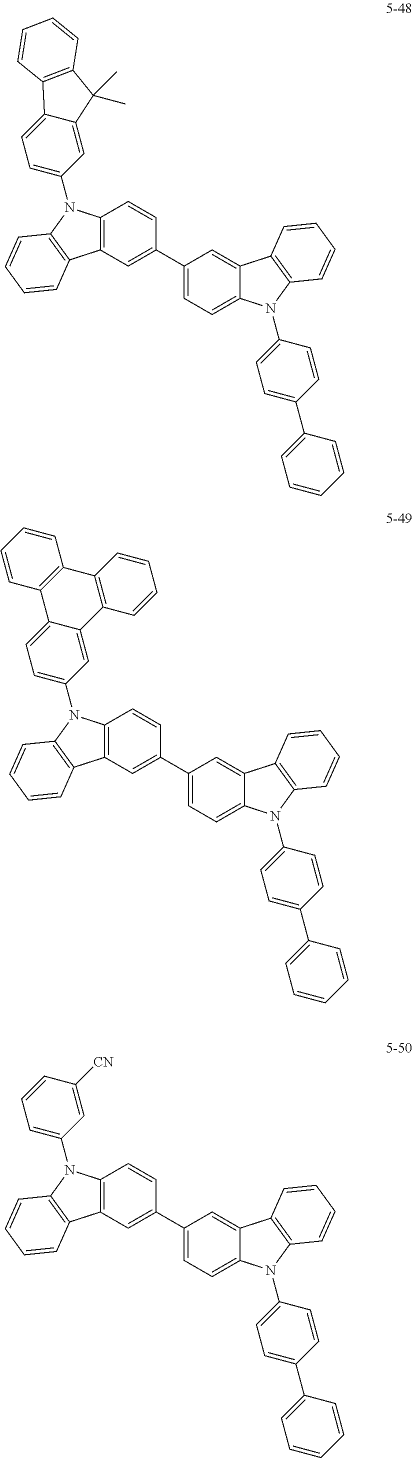 [SMILES: CC1(C)c2ccccc2-c2ccc(-n3c4ccccc4c4cc(-c5ccc6c(c5)c5ccccc5n6-c5ccc(-c6ccccc6)cc5)ccc43)cc21 ; c1ccc(-c2ccc(-n3c4ccccc4c4cc(-c5ccc6c(c5)c5ccccc5n6-c5ccc6c7ccccc7c7ccccc7c6c5)ccc43)cc2)cc1 ; N#Cc1cccc(-n2c3ccccc3c3cc(-c4ccc5c(c4)c4ccccc4n5-c4ccc(-c5ccccc5)cc4)ccc32)c1]

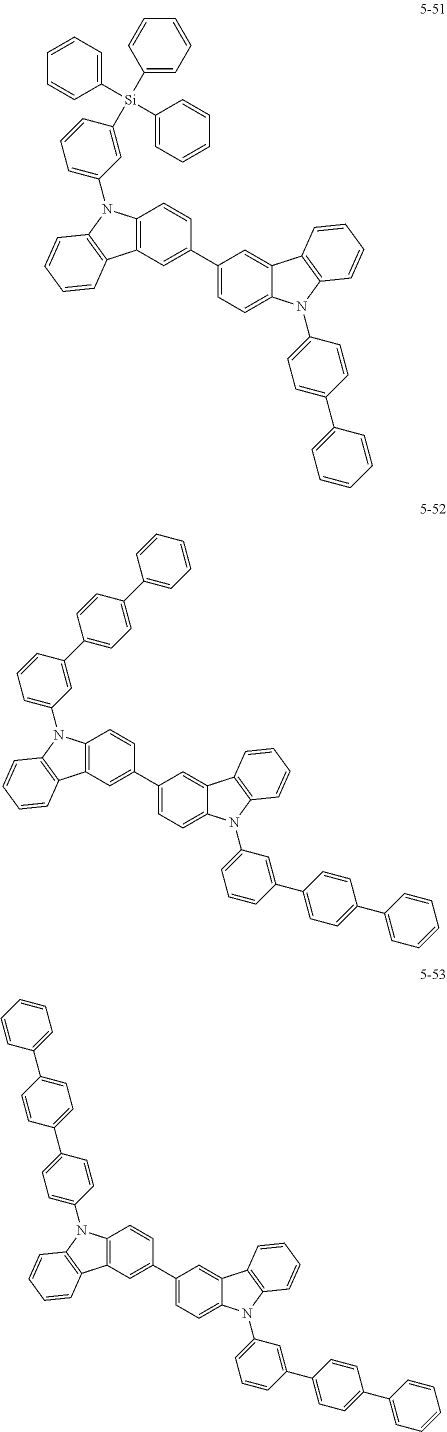 [SMILES: c1ccc(-c2ccc(-n3c4ccccc4c4cc(-c5ccc6c(c5)c5ccccc5n6-c5cccc([Si](c6ccccc6)(c6ccccc6)c6ccccc6)c5)ccc43)cc2)cc1 ; c1ccc(-c2ccc(-c3cccc(-n4c5ccccc5c5cc(-c6ccc7c(c6)c6ccccc6n7-c6cccc(-c7ccc(-c8ccccc8)cc7)c6)ccc54)c3)cc2)cc1 ; c1ccc(-c2ccc(-c3ccc(-n4c5ccccc5c5cc(-c6ccc7c(c6)c6ccccc6n7-c6cccc(-c7ccc(-c8ccccc8)cc7)c6)ccc54)cc3)cc2)cc1]

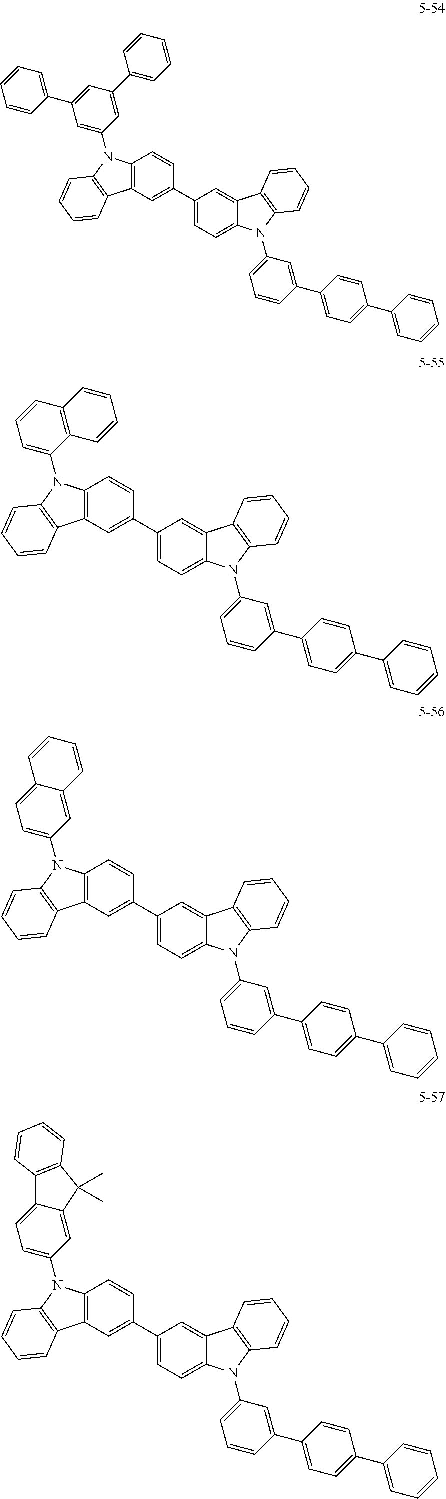 [SMILES: c1ccc(-c2ccc(-c3cccc(-n4c5ccccc5c5cc(-c6ccc7c(c6)c6ccccc6n7-c6cc(-c7ccccc7)cc(-c7ccccc7)c6)ccc54)c3)cc2)cc1 ; c1ccc(-c2ccc(-c3cccc(-n4c5ccccc5c5cc(-c6ccc7c(c6)c6ccccc6n7-c6cccc7ccccc67)ccc54)c3)cc2)cc1 ; c1ccc(-c2ccc(-c3cccc(-n4c5ccccc5c5cc(-c6ccc7c(c6)c6ccccc6n7-c6ccc7ccccc7c6)ccc54)c3)cc2)cc1 ; CC1(C)c2ccccc2-c2ccc(-n3c4ccccc4c4cc(-c5ccc6c(c5)c5ccccc5n6-c5cccc(-c6ccc(-c7ccccc7)cc6)c5)ccc43)cc21]

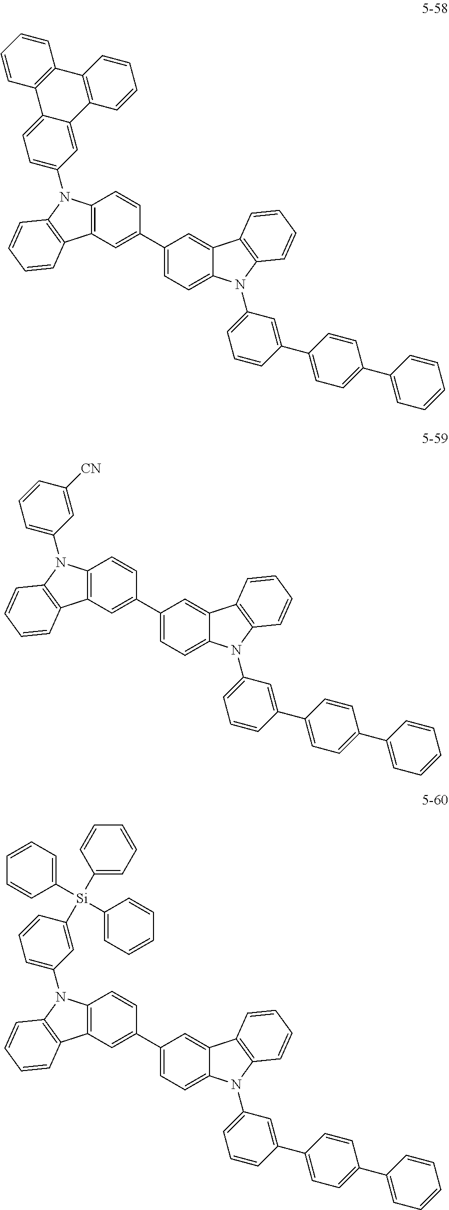 [SMILES: c1ccc(-c2ccc(-c3cccc(-n4c5ccccc5c5cc(-c6ccc7c(c6)c6ccccc6n7-c6ccc7c8ccccc8c8ccccc8c7c6)ccc54)c3)cc2)cc1 ; N#Cc1cccc(-n2c3ccccc3c3cc(-c4ccc5c(c4)c4ccccc4n5-c4cccc(-c5ccc(-c6ccccc6)cc5)c4)ccc32)c1 ; c1ccc(-c2ccc(-c3cccc(-n4c5ccccc5c5cc(-c6ccc7c(c6)c6ccccc6n7-c6cccc([Si](c7ccccc7)(c7ccccc7)c7ccccc7)c6)ccc54)c3)cc2)cc1]

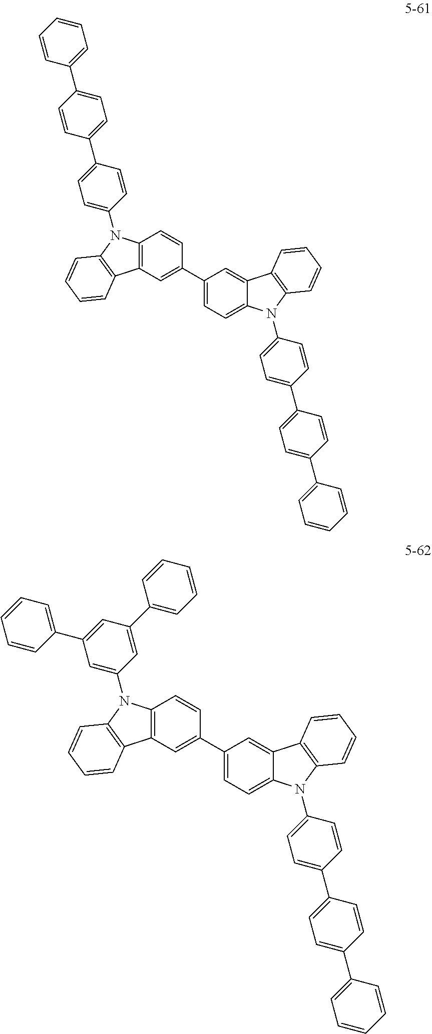 [SMILES: c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1 ; c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1]

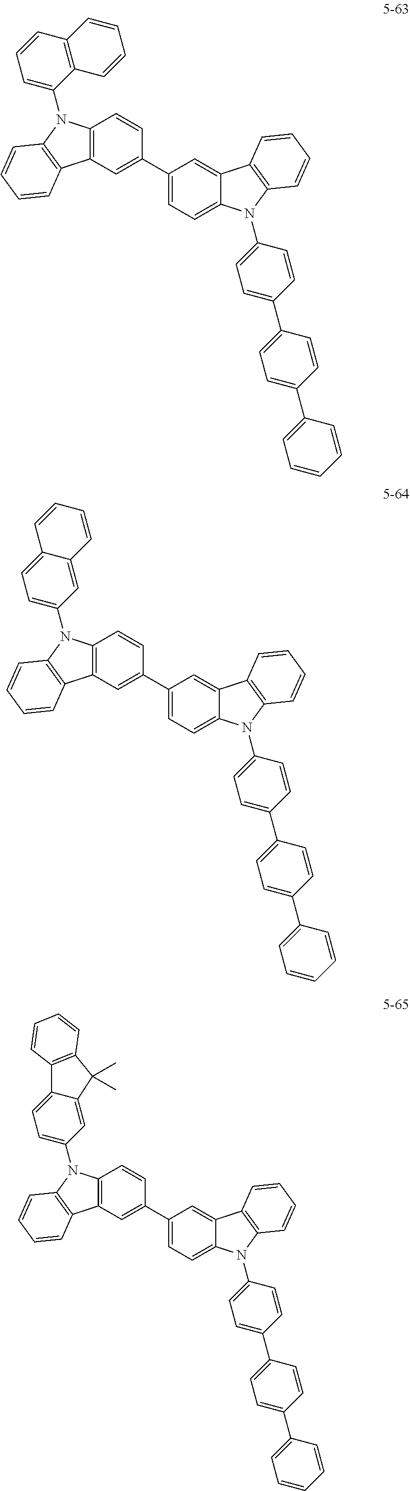 [SMILES: c1ccc(-c2ccc(-c3ccc(-n4c5ccccc5c5cc(-c6ccc7c(c6)c6ccccc6n7-c6cccc7ccccc67)ccc54)cc3)cc2)cc1 ; c1ccc(-c2ccc(-c3ccc(-n4c5ccccc5c5cc(-c6ccc7c(c6)c6ccccc6n7-c6ccc7ccccc7c6)ccc54)cc3)cc2)cc1 ; CC1(C)c2ccccc2-c2ccc(-n3c4ccccc4c4cc(-c5ccc6c(c5)c5ccccc5n6-c5ccc(-c6ccc(-c7ccccc7)cc6)cc5)ccc43)cc21]

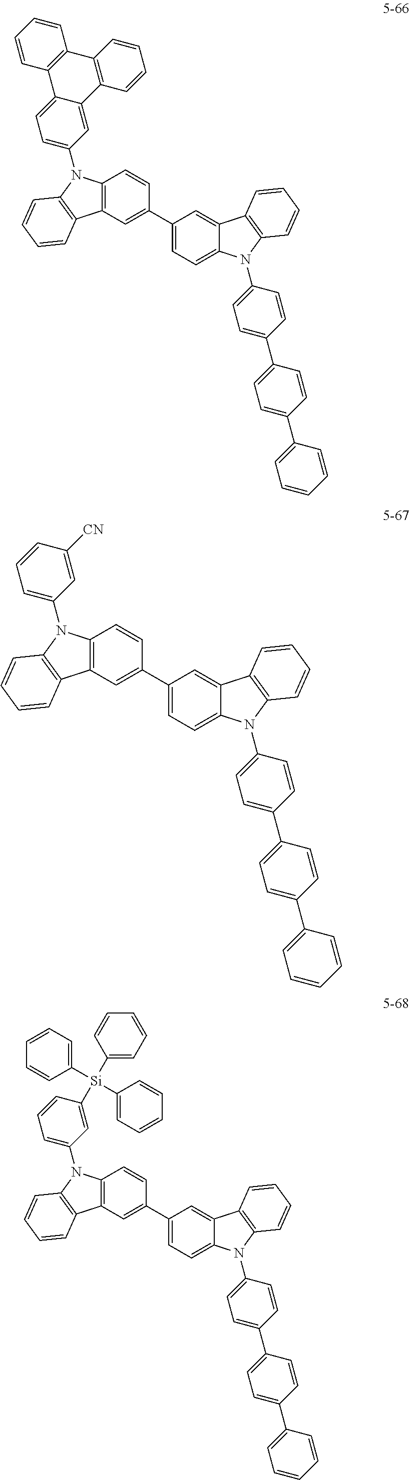 [SMILES: c1ccc(-c2ccc(-c3ccc(-n4c5ccccc5c5cc(-c6ccc7c(c6)c6ccccc6n7-c6ccc7c8ccccc8c8ccccc8c7c6)ccc54)cc3)cc2)cc1 ; N#Cc1cccc(-n2c3ccccc3c3cc(-c4ccc5c(c4)c4ccccc4n5-c4ccc(-c5ccc(-c6ccccc6)cc5)cc4)ccc32)c1 ; c1ccc(-c2ccc(-c3ccc(-n4c5ccccc5c5cc(-c6ccc7c(c6)c6ccccc6n7-c6cccc([Si](c7ccccc7)(c7ccccc7)c7ccccc7)c6)ccc54)cc3)cc2)cc1]

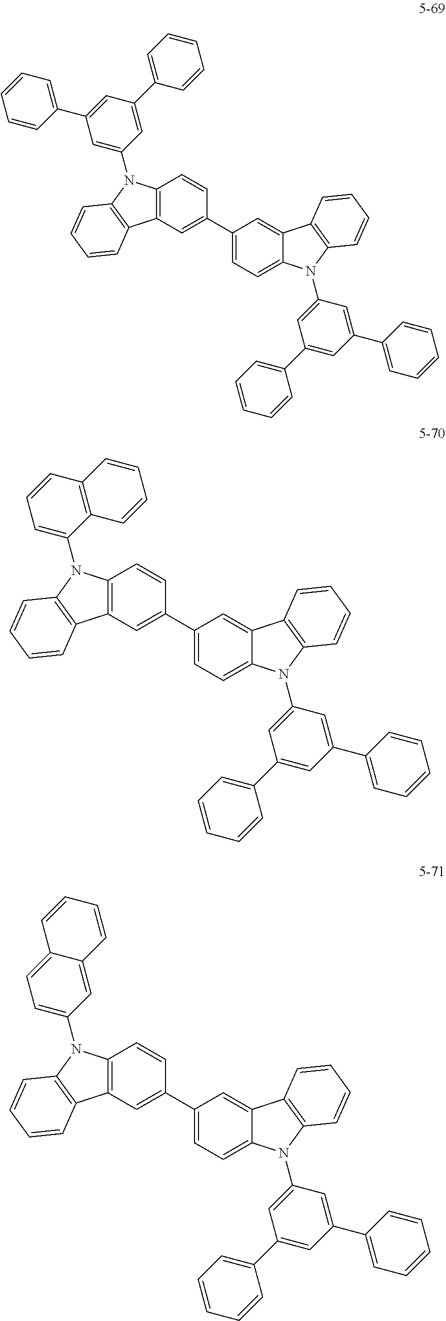 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cc(-c3ccccc3)cc(-n3c4ccccc4c4cc(-c5ccc6c(c5)c5ccccc5n6-c5cccc6ccccc56)ccc43)c2)cc1 ; c1ccc(-c2cc(-c3ccccc3)cc(-n3c4ccccc4c4cc(-c5ccc6c(c5)c5ccccc5n6-c5ccc6ccccc6c5)ccc43)c2)cc1]

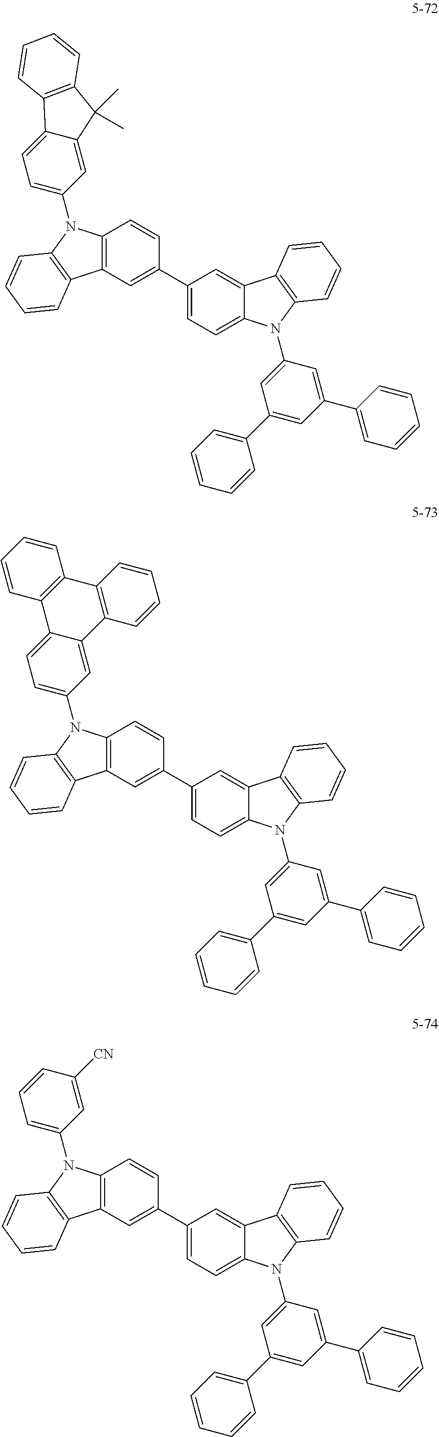 [SMILES: CC1(C)c2ccccc2-c2ccc(-n3c4ccccc4c4cc(-c5ccc6c(c5)c5ccccc5n6-c5cc(-c6ccccc6)cc(-c6ccccc6)c5)ccc43)cc21 ; c1ccc(-c2cc(-c3ccccc3)cc(-n3c4ccccc4c4cc(-c5ccc6c(c5)c5ccccc5n6-c5ccc6c7ccccc7c7ccccc7c6c5)ccc43)c2)cc1 ; N#Cc1cccc(-n2c3ccccc3c3cc(-c4ccc5c(c4)c4ccccc4n5-c4cc(-c5ccccc5)cc(-c5ccccc5)c4)ccc32)c1]

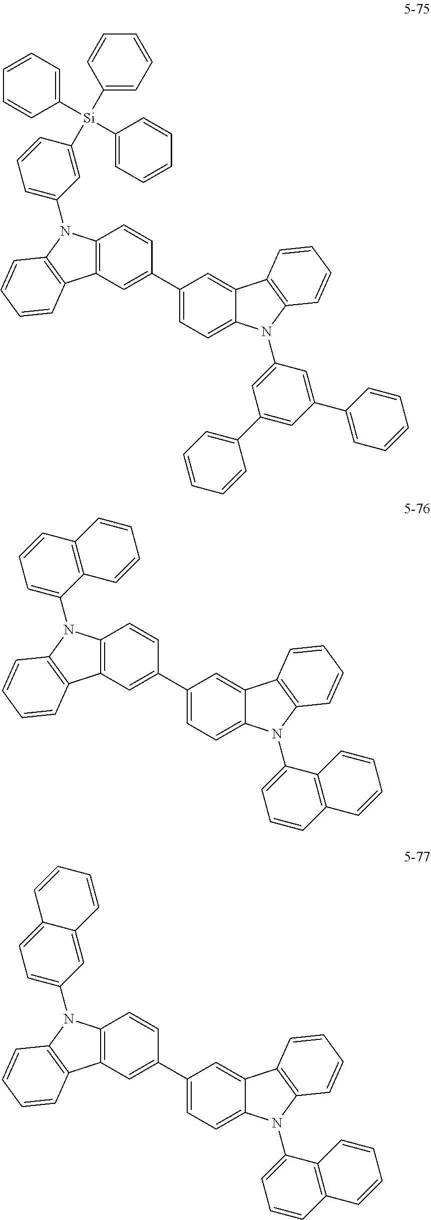 [SMILES: c1ccc(-c2cc(-c3ccccc3)cc(-n3c4ccccc4c4cc(-c5ccc6c(c5)c5ccccc5n6-c5cccc([Si](c6ccccc6)(c6ccccc6)c6ccccc6)c5)ccc43)c2)cc1 ; c1ccc2c(-n3c4ccccc4c4cc(-c5ccc6c(c5)c5ccccc5n6-c5cccc6ccccc56)ccc43)cccc2c1 ; c1ccc2cc(-n3c4ccccc4c4cc(-c5ccc6c(c5)c5ccccc5n6-c5cccc6ccccc56)ccc43)ccc2c1]

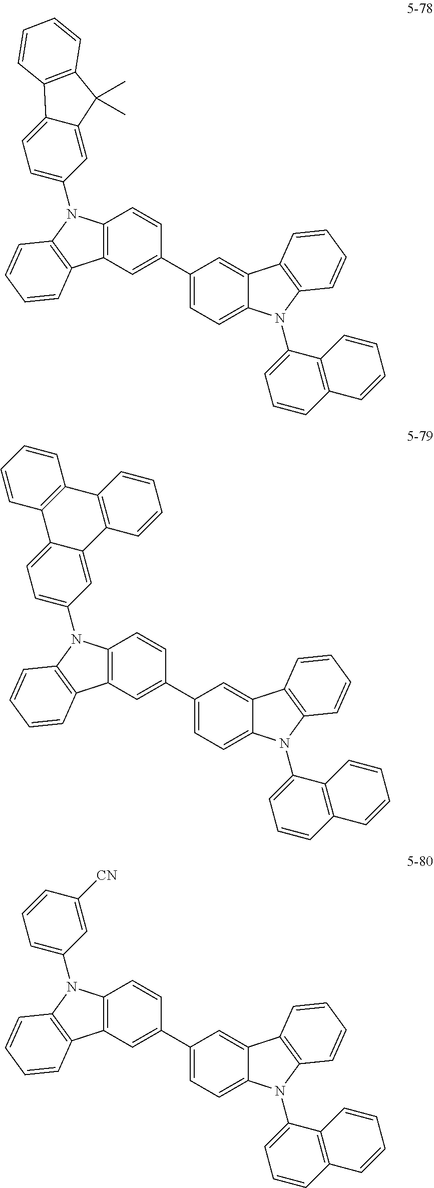 [SMILES: CC1(C)c2ccccc2-c2ccc(-n3c4ccccc4c4cc(-c5ccc6c(c5)c5ccccc5n6-c5cccc6ccccc56)ccc43)cc21 ; c1ccc2c(-n3c4ccccc4c4cc(-c5ccc6c(c5)c5ccccc5n6-c5ccc6c7ccccc7c7ccccc7c6c5)ccc43)cccc2c1 ; N#Cc1cccc(-n2c3ccccc3c3cc(-c4ccc5c(c4)c4ccccc4n5-c4cccc5ccccc45)ccc32)c1]

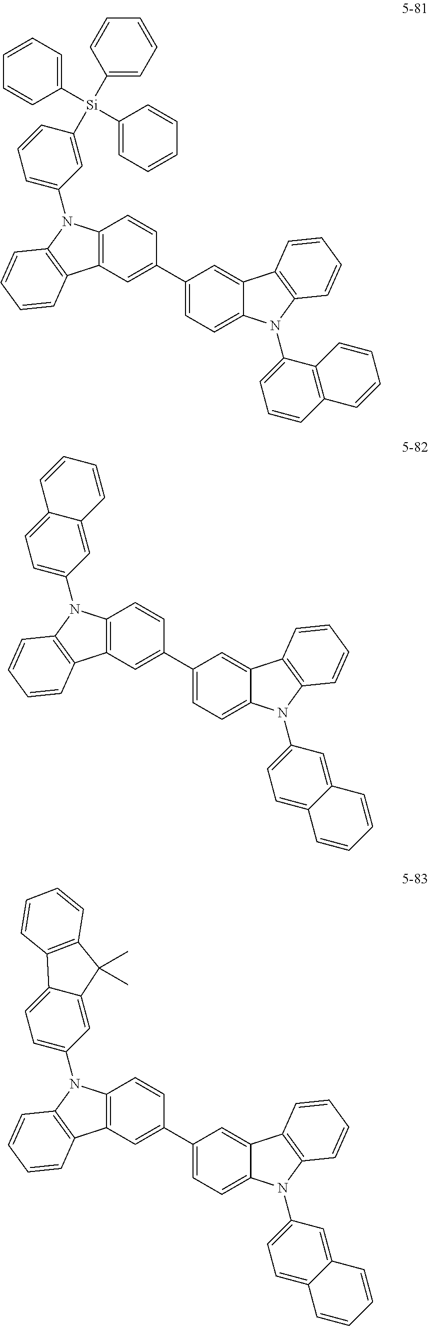 [SMILES: c1ccc([Si](c2ccccc2)(c2ccccc2)c2cccc(-n3c4ccccc4c4cc(-c5ccc6c(c5)c5ccccc5n6-c5cccc6ccccc56)ccc43)c2)cc1 ; c1ccc2cc(-n3c4ccccc4c4cc(-c5ccc6c(c5)c5ccccc5n6-c5ccc6ccccc6c5)ccc43)ccc2c1 ; CC1(C)c2ccccc2-c2ccc(-n3c4ccccc4c4cc(-c5ccc6c(c5)c5ccccc5n6-c5ccc6ccccc6c5)ccc43)cc21]

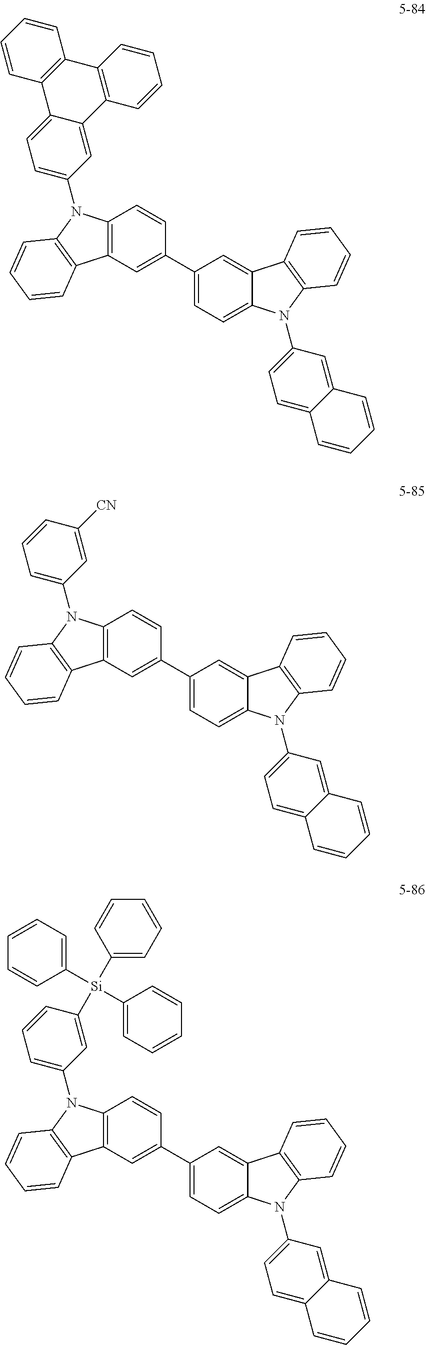 [SMILES: c1ccc2cc(-n3c4ccccc4c4cc(-c5ccc6c(c5)c5ccccc5n6-c5ccc6c7ccccc7c7ccccc7c6c5)ccc43)ccc2c1 ; N#Cc1cccc(-n2c3ccccc3c3cc(-c4ccc5c(c4)c4ccccc4n5-c4ccc5ccccc5c4)ccc32)c1 ; c1ccc([Si](c2ccccc2)(c2ccccc2)c2cccc(-n3c4ccccc4c4cc(-c5ccc6c(c5)c5ccccc5n6-c5ccc6ccccc6c5)ccc43)c2)cc1]

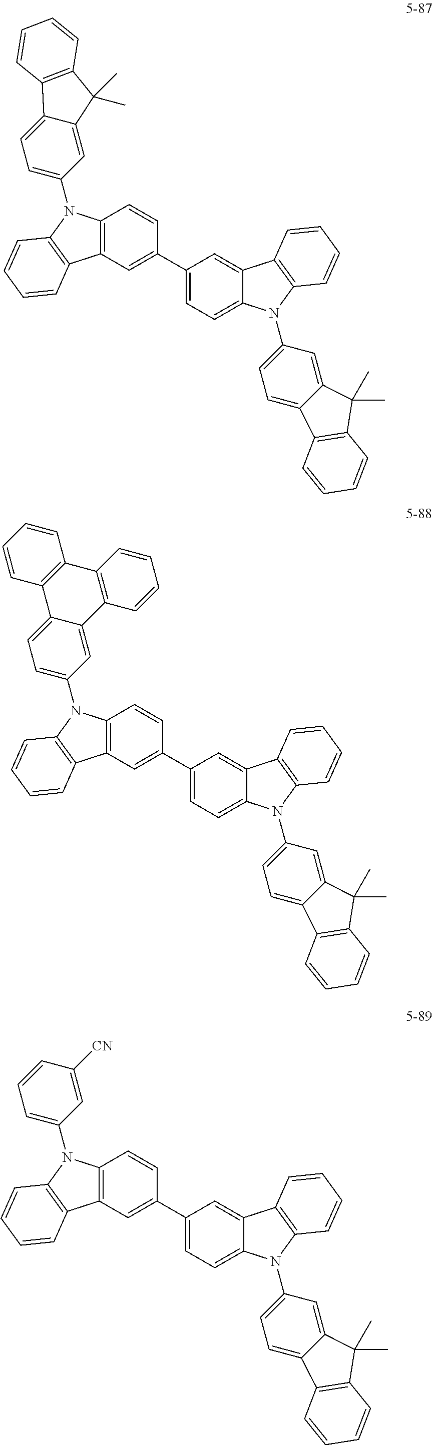 [SMILES: CC1(C)c2ccccc2-c2ccc(-n3c4ccccc4c4cc(-c5ccc6c(c5)c5ccccc5n6-c5ccc6c(c5)C(C)(C)c5ccccc5-6)ccc43)cc21 ; CC1(C)c2ccccc2-c2ccc(-n3c4ccccc4c4cc(-c5ccc6c(c5)c5ccccc5n6-c5ccc6c7ccccc7c7ccccc7c6c5)ccc43)cc21 ; CC1(C)c2ccccc2-c2ccc(-n3c4ccccc4c4cc(-c5ccc6c(c5)c5ccccc5n6-c5cccc(C#N)c5)ccc43)cc21]

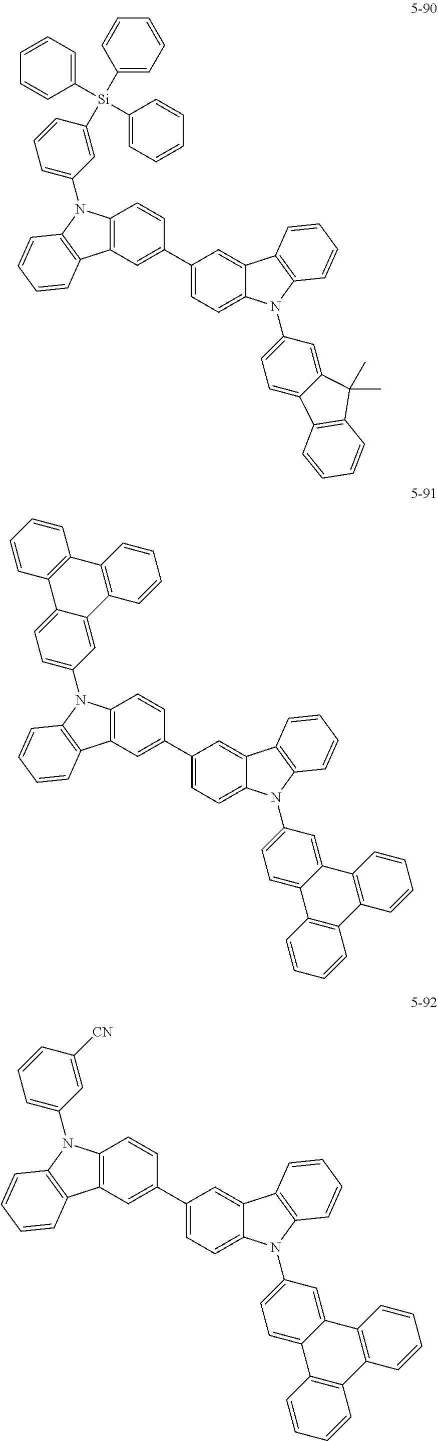 [SMILES: CC1(C)c2ccccc2-c2ccc(-n3c4ccccc4c4cc(-c5ccc6c(c5)c5ccccc5n6-c5cccc([Si](c6ccccc6)(c6ccccc6)c6ccccc6)c5)ccc43)cc21 ; c1ccc2c(c1)c1ccccc1c1cc(-n3c4ccccc4c4cc(-c5ccc6c(c5)c5ccccc5n6-c5ccc6c7ccccc7c7ccccc7c6c5)ccc43)ccc21 ; N#Cc1cccc(-n2c3ccccc3c3cc(-c4ccc5c(c4)c4ccccc4n5-c4ccc5c6ccccc6c6ccccc6c5c4)ccc32)c1]

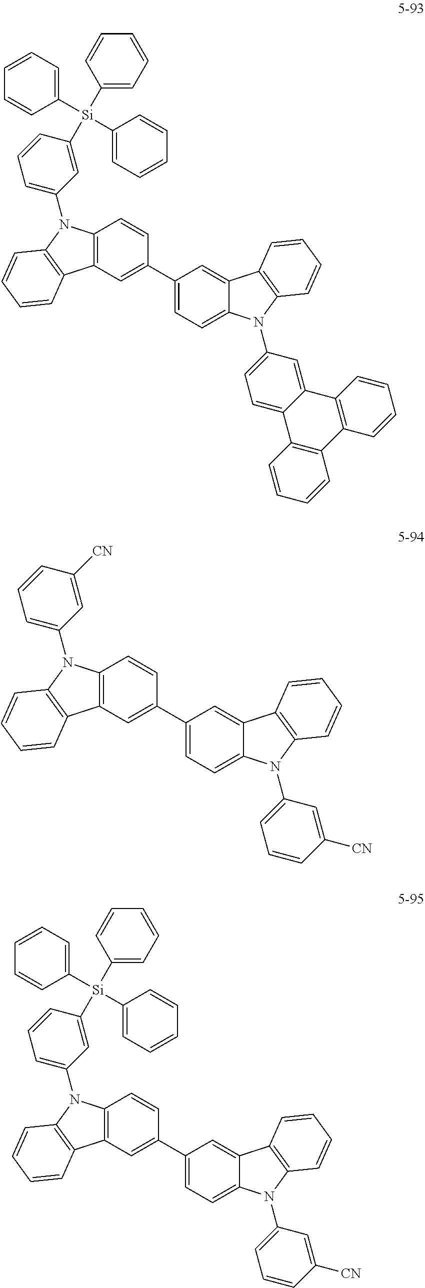 [SMILES: c1ccc([Si](c2ccccc2)(c2ccccc2)c2cccc(-n3c4ccccc4c4cc(-c5ccc6c(c5)c5ccccc5n6-c5ccc6c7ccccc7c7ccccc7c6c5)ccc43)c2)cc1 ; N#Cc1cccc(-n2c3ccccc3c3cc(-c4ccc5c(c4)c4ccccc4n5-c4cccc(C#N)c4)ccc32)c1 ; N#Cc1cccc(-n2c3ccccc3c3cc(-c4ccc5c(c4)c4ccccc4n5-c4cccc([Si](c5ccccc5)(c5ccccc5)c5ccccc5)c4)ccc32)c1]

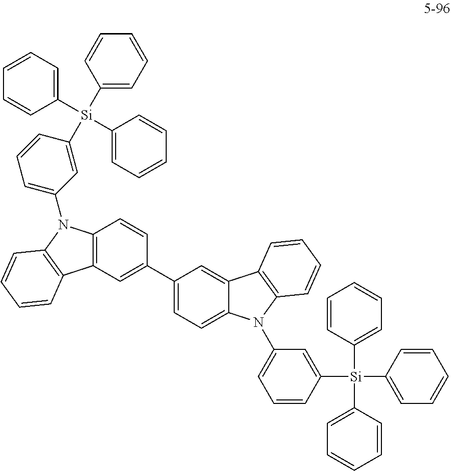 [SMILES: c1ccc([Si](c2ccccc2)(c2ccccc2)c2cccc(-n3c4ccccc4c4cc(-c5ccc6c(c5)c5ccccc5n6-c5cccc([Si](c6ccccc6)(c6ccccc6)c6ccccc6)c5)ccc43)c2)cc1]

In the organic light emitting device according to one embodiment of the present application, the compound of Chemical Formula 2 may be included in a light emitting layer of the organic material layer.

In the organic light emitting device according to one embodiment of the present application, the compound of Chemical Formula 2 may be included in a light emitting layer of the organic material layer, and may be specifically used as a host material of the light emitting layer.

In one embodiment of the present application, the host material of the light emitting layer of the organic light emitting device may comprise the heterocyclic compound of Chemical Formula 1 and the heterocyclic compound of Chemical Formula 2 at the same time.

One embodiment of the present application provides a composition for an organic material layer of an organic light emitting device, the composition comprising the heterocyclic compound represented by Chemical Formula 1 and the heterocyclic compound represented by Chemical Formula 2.

In the composition, the heterocyclic compound represented by Chemical Formula 1: the heterocyclic compound represented by Chemical Formula 2 may have a weight ratio of 1:10 to 10:1, and the weight ratio may be from 1:8 to 8:1, 1:5 to 5:1 or 1:2 to 2:1, but is not limited thereto.

One embodiment of the present application provides a method for manufacturing an organic light emitting device, the method comprising preparing a substrate; forming a first electrode on the substrate; forming one or more organic material layers on the first electrode; and forming a second electrode on the organic material layer, wherein the forming of organic material layers comprises forming one or more organic material layers using the composition for an organic material layer according to one embodiment of the present application.

In the method for manufacturing an organic light emitting device provided in one embodiment of the present application, the forming of organic material layers is forming the heterocyclic compound represented by Chemical Formula 1 using a thermal vacuum deposition method.

In the method for manufacturing an organic light emitting device provided in one embodiment of the present application, the forming of organic material layers is forming two types of the heterocyclic compound represented by Chemical Formula 1 and the heterocyclic compound represented by Chemical Formula 2 using a thermal vacuum deposition method after pre-mixing.

The pre-mixing means first mixing two types of the heterocyclic compound represented by Chemical Formula 1 and the heterocyclic compound represented by Chemical Formula 2 in one source of supply before depositing on the organic material layer.

The premixed material may be referred to as the composition for an organic material layer according to one embodiment of the present application.

In the organic light emitting device according to one embodiment of the present application, materials other than the compound of Chemical Formula 1 are illustrated below, however, these are for illustrative purposes only and not for limiting the scope of the present application, and may be replaced by materials known in the art.

As the anode material, materials having relatively large work function may be used, and transparent conductive oxides, metals, conductive polymers or the like may be used. Specific examples of the anode material comprise metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene] (PEDOT), polypyrrole and polyaniline, and the like, but are not limited thereto.

As the cathode material, materials having relatively small work function may be used, and metals, metal oxides, conductive polymers or the like may be used. Specific examples of the cathode material comprise metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection material, known hole injection materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tri[phenyl(m-tolyl)amino]triphenylamine (m-MTDATA) or 1,3,5-tris[4-(3-methylphenylphenylamino)phenyl]benzene (m-MTDAPB) described in the literature [Advanced Material, 6, p. 677 (1994)], polyaniline/dodecylbenzene sulfonic acid, poly(3,4-ethylenedioxythiophene)/poly(4-styrenesulfonate), polyaniline/camphor sulfonic acid or polyaniline/poly(4-styrene-sulfonate) that are conductive polymers having solubility, and the like, may be used.

As the hole transfer material, pyrazoline derivatives, arylamine-based derivatives, stilbene derivatives, triphenyldiamine derivatives and the like may be used, and low molecular or high molecular materials may also be used.

As the electron transfer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials may also be used as well as low molecular materials.

As examples of the electron injection material, LiF is typically used in the art, however, the present application is not limited thereto.

As the light emitting material, red, green or blue light emitting materials may be used, and as necessary, two or more light emitting materials may be mixed and used. Herein, two or more light emitting materials may be used by being deposited as individual sources of supply or by being premixed and deposited as one source of supply. In addition, fluorescent materials may also be used as the light emitting material, however, phosphorescent materials may also be used. As the light emitting material, materials emitting light by bonding electrons and holes injected from an anode and a cathode, respectively, may be used alone, however, materials having a host material and a dopant material involving in light emission together may also be used.

When mixing light emitting material hosts, same series hosts may be mixed, or different series hosts may be mixed. For example, any two or more types of materials among n-type host materials or p-type host materials may be selected and used as a host material of a light emitting layer.

The organic light emitting device according to one embodiment of the present application may be a top-emission type, a bottom-emission type or a dual-emission type depending on the materials used.

The heterocyclic compound according to one embodiment of the present application may also be used in an organic electronic device comprising an organic solar cell, an organic photo conductor, an organic transistor and the like under a similar principle used in the organic light emitting device.

Hereinafter, the present specification will be described in more detail with reference to examples, however, these are for illustrative purposes only, and the scope of the present application is not limited thereto.

### <Preparation Example 1> Preparation of Compound 1-1

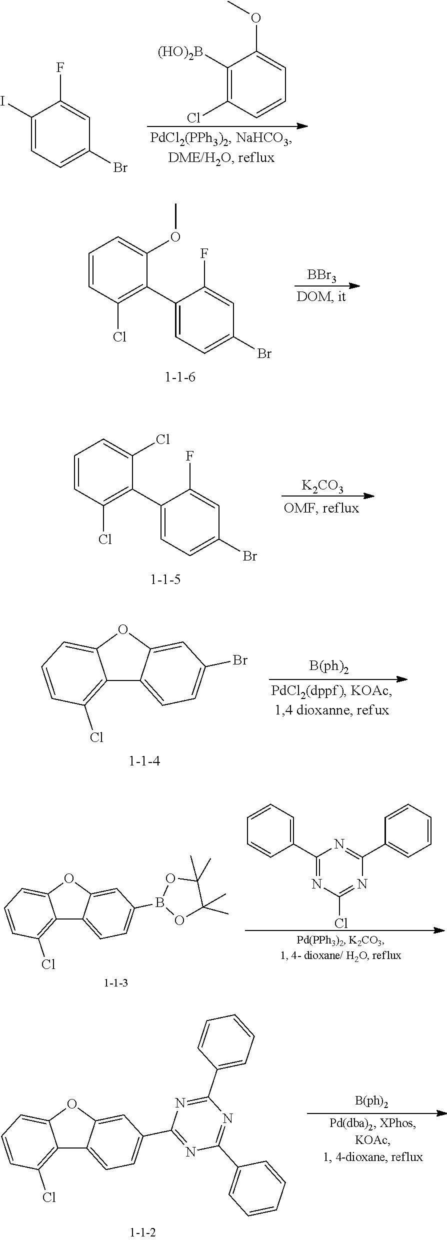

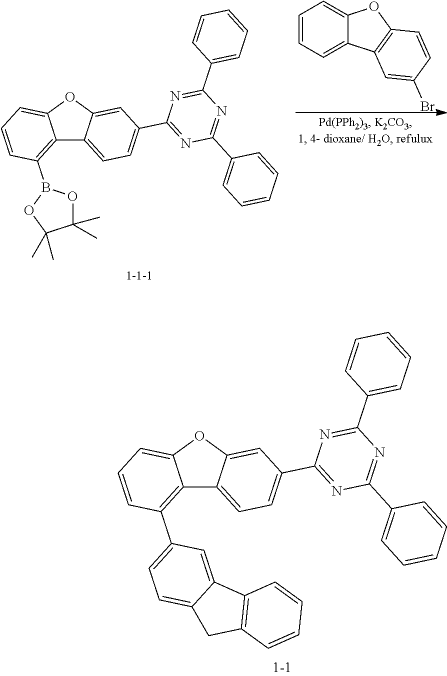 [SMILES: CC1(C)OB(c2cccc3oc4cc(-c5nc(-c6ccccc6)nc(-c6ccccc6)n5)ccc4c23)OC1(C)C ; Brc1ccc2oc3ccccc3c2c1 ; c1ccc(-c2nc(-c3ccccc3)nc(-c3ccc4c(c3)oc3cccc(-c5ccc6c(c5)-c5ccccc5C6)c34)n2)cc1]

1) Preparation of Compound 1-1-6

After dissolving 4-bromo-2-fluoro-1-iodobenzene (200.0 g, 664.7 mM), (2-chloro-6-methoxyphenyl)boronic acid (148.7 g, 794.6 mM), Pd(PPh)₄ (38.4 g, 33.2 mM) and K₂CO₃ (183.7 g, 1329.4 mM) in 1,4-dioxane/H₂O (1 L/200 mL), the result was refluxed for 24 hours. After the reaction was completed, the result was extracted by introducing distilled water and dichloromethane (DCM) thereto at room temperature. The organic layer was dried with MgSO₄, and then the solvent was removed using a rotary evaporator. The reaction material was purified by column chromatography (DCM:Hex=1:3) to obtain target Compound 1-1-6 (178 g, 85%).

2) Preparation of Compound 1-1-5

After dissolving Compound 1-1-6 (178 g, 564.1 mM) and BBr₃ (107 mL, 1128.2 mM) in DCM (800 mL), the result was refluxed for 1 hour. After the reaction was completed, the result was extracted by introducing distilled water and DCM thereto at room temperature. The organic layer was dried with MgSO₄, and then the solvent was removed using a rotary evaporator. The reaction material was purified by column chromatography (DCM:Hex=1:1) to obtain target Compound 1-1-5 (153.1 g, 90%).

3) Preparation of Compound 1-1-4

After dissolving Compound 1-1-5 (153 g, 507.4 mM) and K₂CO₃ (140.3 g, 1014.8 mM) in dimethylformamide (DMF) (800 mL), the result was refluxed for 4 hours. After the reaction was completed, the result was extracted by introducing distilled water and DCM thereto at room temperature. The organic layer was dried with MgSO₄, and then the solvent was removed using a rotary evaporator. The reaction material was purified by column chromatography (DCM:Hex=1:9), and recrystallized with methanol to obtain target Compound 1-1-4 (88.5 g, 62%).

4) Preparation of Compound 1-1-3

After dissolving Compound 1-1-4 (88.5 g, 314.4 mM), bis(pinacolato)diboron (159.7 g, 628.8 mM), PdCl₂(dppf) (23.0 g, 31.4 mM) and KOAc (92.6 g, 943.2 mM) in 1,4-dioxane (500 mL), the result was refluxed for 24 hours. After the reaction was completed, the result was extracted by introducing distilled water and DCM thereto at room temperature. The organic layer was dried with MgSO₄, and then the solvent was removed using a rotary evaporator. The reaction material was purified by column chromatography (DCM:Hex=1:5) to obtain target Compound 1-1-3 (85.7 g, 831).

5) Preparation of Compound 1-1-2

After dissolving Compound 1-1-3 (85.0 g, 258.7 mM), 2-chloro-4,6-diphenyl-1,3,5-triazine (69.3 g, 258.7 mM), Pd(PPh)₄ (14.9 g, 12.9 mM) and K₂CO₃ (71.5 g, 517.4 mM) in 1,4-dioxane/H₂O (1000/200 mL), the result was refluxed for 24 hours. After the reaction was completed, the result was extracted by introducing distilled water and DCM thereto at room temperature. The organic layer was dried with MgSO₄, and then the solvent was removed using a rotary evaporator. The reaction material was purified by column chromatography (DCM:Hex=1:4), and recrystallized with methanol to obtain target Compound 1-1-2 (79.7 g, 71%).

6) Preparation of Compound 1-1-1

After dissolving Compound 1-1-2 (79.0 g, 182.1 mM), bis(pinacolato)diboron (92.5 g, 364.2 mM), Pd(dba)₂ (10.5 g, 18.2 mM), XPhos (17.4 g, 36.4 mM) and KOAc (53.6 g, 546.3 mM) in 1,4-dioxane (800 mL), the result was refluxed for 24 hours. After the reaction was completed, the result was extracted by introducing distilled water and DCM thereto at room temperature. The organic layer was dried with MgSO₄, and then the solvent was removed using a rotary evaporator. The reaction material was purified by column chromatography (DCM:Hex=1:5) to obtain target Compound 1-1-1 (84.2 g, 88%).

7) Preparation of Compound 1-1

After dissolving Compound 1-1-1 (15.0 g, 28.5 mM), 2-bromodibenzo[b,d]furan (7.8 g, 31.4 WM), Pd(PPh)₄ (1.6 g, 1.4 mM) and K₂CO₃ (7.9 g, 57.0 mM) in 1,4-dioxane/H₂O (200/40 mL), the result was refluxed for 24 hours. After the reaction was completed, the result was extracted by introducing distilled water and DCM thereto at room temperature. The organic layer was dried with MgSO₄, and then the solvent was removed using a rotary evaporator. The reaction material was purified by column chromatography (DCM:Hex=1:3), and recrystallized with methanol to obtain target Compound 1-1 (13.2 g, 82%).

Target Compound A was synthesized in the same manner as in Preparation Example 1 except that Intermediate A of the following Table 1 was used instead of 2-chloro-4,6-diphenyl-1,3,5-triazine, and Intermediate B of the following Table 1 was used instead of 2-bromodibenzo[b,d]furan.

TABLE 1

| Com- | | | | |
|---|---|---|---|---|
| pound | | | | |
| No. | Intermediate A | Intermediate B | Target Compound A | Yield |
| 1-2 | 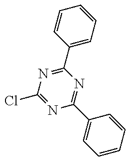 [SMILES: Clc1nc(-c2ccccc2)nc(-c2ccccc2)n1] | 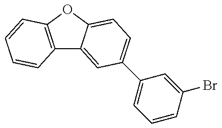 [SMILES: Brc1cccc(-c2ccc3oc4ccccc4c3c2)c1] | 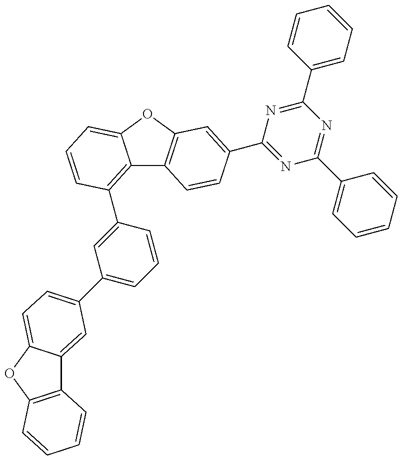 [SMILES: c1ccc(-c2nc(-c3ccccc3)nc(-c3ccc4c(c3)oc3cccc(-c5cccc(-c6ccc7oc8ccccc8c7c6)c5)c34)n2)cc1] | 20% |
| 1-14 | | 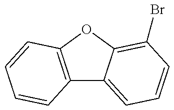 [SMILES: Brc1cccc2c1oc1ccccc12] | 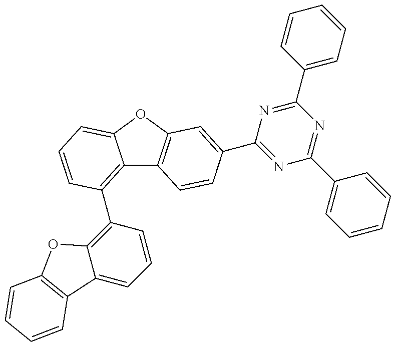 [SMILES: c1ccc(-c2nc(-c3ccccc3)nc(-c3ccc4c(c3)oc3cccc(-c5cccc6c5oc5ccccc56)c34)n2)cc1] | 26% |
| 1-18 | | 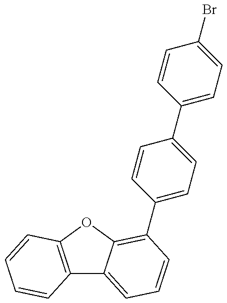 [SMILES: Brc1ccc(-c2ccc(-c3cccc4c3oc3ccccc34)cc2)cc1] | 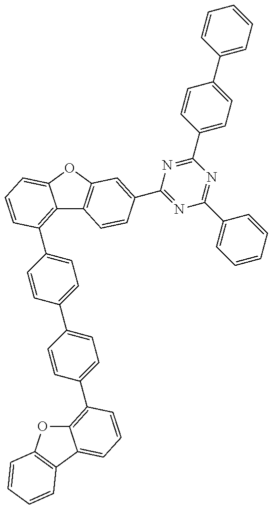 [SMILES: c1ccc(-c2ccc(-c3nc(-c4ccccc4)nc(-c4ccc5c(c4)oc4cccc(-c6ccc(-c7ccc(-c8cccc9c8oc8ccccc89)cc7)cc6)c45)n3)cc2)cc1] | 22% |
| 1-37 | | 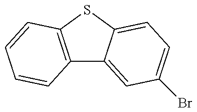 [SMILES: Brc1ccc2sc3ccccc3c2c1] | 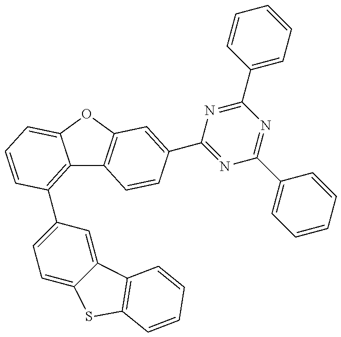 [SMILES: c1ccc(-c2nc(-c3ccccc3)nc(-c3ccc4c(c3)oc3cccc(-c5ccc6sc7ccccc7c6c5)c34)n2)cc1] | 23% |
| 1-38 | | 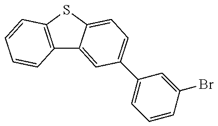 [SMILES: Brc1cccc(-c2ccc3sc4ccccc4c3c2)c1] | 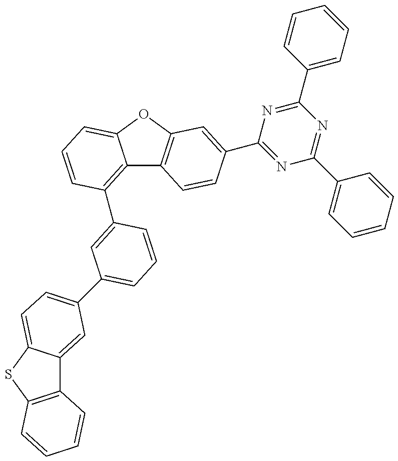 [SMILES: c1ccc(-c2nc(-c3ccccc3)nc(-c3ccc4c(c3)oc3cccc(-c5cccc(-c6ccc7sc8ccccc8c7c6)c5)c34)n2)cc1] | 25% |
| 1-49 | | 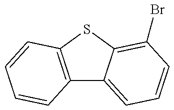 [SMILES: Brc1cccc2c1sc1ccccc12] | 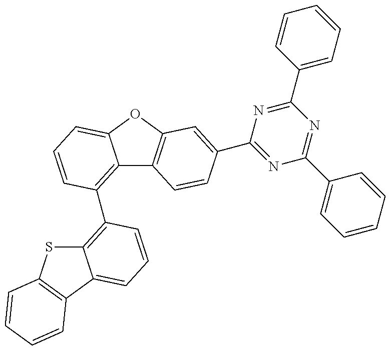 [SMILES: c1ccc(-c2nc(-c3ccccc3)nc(-c3ccc4c(c3)oc3cccc(-c5cccc6c5sc5ccccc56)c34)n2)cc1] | 24% |
| 1-50 | | 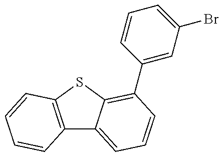 [SMILES: Brc1cccc(-c2cccc3c2sc2ccccc23)c1] | 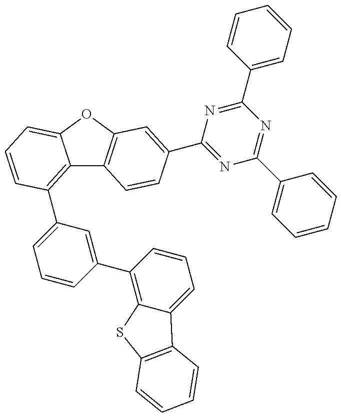 [SMILES: c1ccc(-c2nc(-c3ccccc3)nc(-c3ccc4c(c3)oc3cccc(-c5cccc(-c6cccc7c6sc6ccccc67)c5)c34)n2)cc1] | 22% |
| 1-61 | | 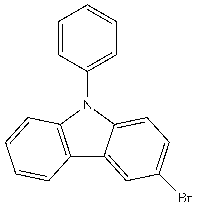 [SMILES: Brc1ccc2c(c1)c1ccccc1n2-c1ccccc1] | 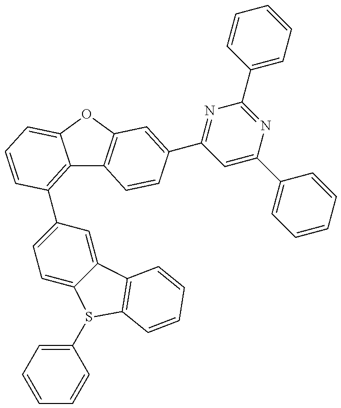 | 26% |
| 1-81 | | 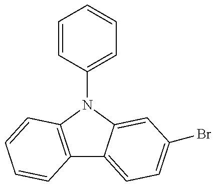 [SMILES: Brc1ccc2c3ccccc3n(-c3ccccc3)c2c1] | 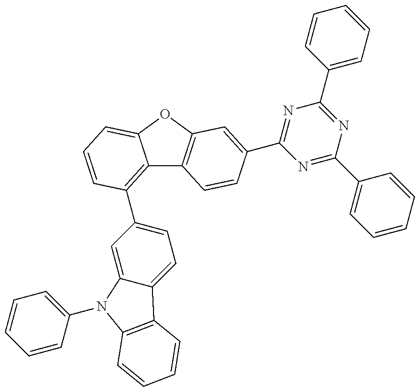 [SMILES: c1ccc(-c2nc(-c3ccccc3)nc(-c3ccc4c(c3)oc3cccc(-c5ccc6c7ccccc7n(-c7ccccc7)c6c5)c34)n2)cc1] | 23% |
| 1-102 | | 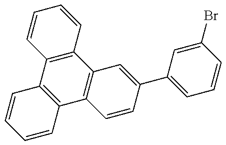 [SMILES: Brc1cccc(-c2ccc3c4ccccc4c4ccccc4c3c2)c1] | 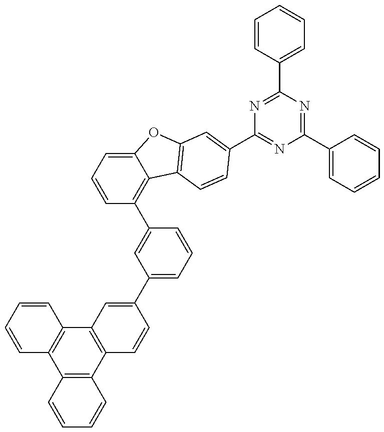 [SMILES: c1ccc(-c2nc(-c3ccccc3)nc(-c3ccc4c(c3)oc3cccc(-c5cccc(-c6ccc7c8ccccc8c8ccccc8c7c6)c5)c34)n2)cc1] | 26% |
| 1-106 | | 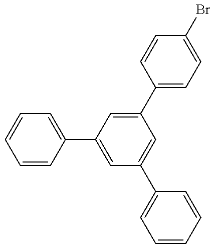 [SMILES: Brc1ccc(-c2cc(-c3ccccc3)cc(-c3ccccc3)c2)cc1] | 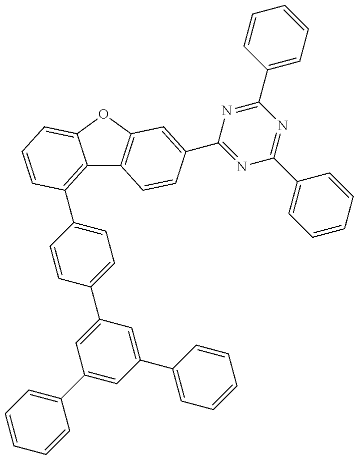 [SMILES: c1ccc(-c2cc(-c3ccccc3)cc(-c3ccc(-c4cccc5oc6cc(-c7nc(-c8ccccc8)nc(-c8ccccc8)n7)ccc6c45)cc3)c2)cc1] | 25% |

### <Preparation Example 2> Preparation of Compound 4-1

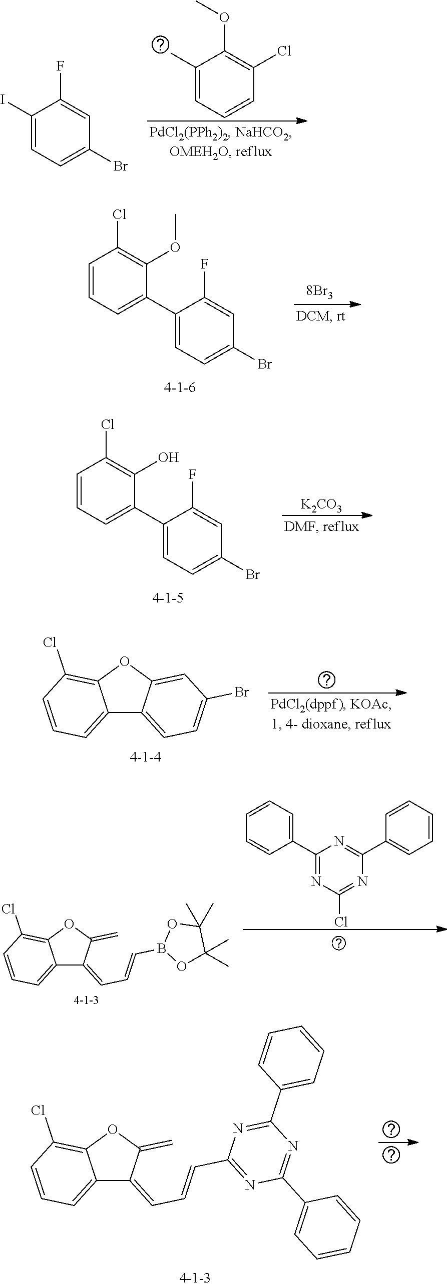

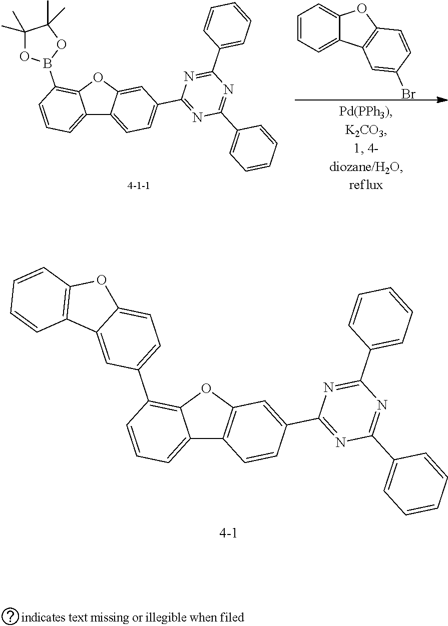 [SMILES: CC1(C)OB(c2cccc3c2oc2cc(-c4nc(-c5ccccc5)nc(-c5ccccc5)n4)ccc23)OC1(C)C ; Brc1ccc2oc3ccccc3c2c1 ; c1ccc(-c2nc(-c3ccccc3)nc(-c3ccc4c(c3)oc3c(-c5ccc6oc7ccccc7c6c5)cccc34)n2)cc1]

1) Preparation of Compound 4-1-6

After dissolving 4-bromo-2-fluoro-1-iodobenzene (200.0 g, 664.7 mM), (3-chloro-2-methoxyphenyl)boronic acid (143.7 g, 794.6 mM), Pd(PPh)₄ (38.4 g, 33.2 mM) and K₂CO₃ (183.7 g, 1329.4 mM) in 1,4-dioxane/H₂O (1 L/200 mL), the result was refluxed for 24 hours. After the reaction was completed, the result was extracted by introducing distilled water and DCM thereto at room temperature. The organic layer was dried with MgSO₄, and then the solvent was removed using a rotary evaporator. The reaction material was purified by column chromatography (DCM:Hex=1:4) to obtain target Compound 4-1-6 (169 g, 81%).

2) Preparation of Compound 4-1-5

After dissolving Compound 4-1-6 (169 g, 535.5 mM) and BBr₃ (103 mL, 1071.0 mM) in dichloromethane (DCM) (800 mL), the result was refluxed for 1 hour. After the reaction was completed, the result was extracted by introducing distilled water and DCM thereto at room temperature. The organic layer was dried with MgSO₄, and then the solvent was removed using a rotary evaporator. The reaction material was purified by column chromatography (DCM:Hex=1:1) to obtain target Compound 4-1-5 (145.3 g, 90%).

3) Preparation of Compound 4-1-4

After dissolving Compound 4-1-5 (145.3 g, 481.9 mM) and K₂CO₃ (133.2 g, 963.9 mM) in IF (800 mL), the result was refluxed for 4 hours. After the reaction was completed, the result was extracted by introducing distilled water and DCM thereto at room temperature. The organic layer was dried with MgSO₄, and then the solvent was removed using a rotary evaporator. The reaction material was purified by column chromatography (DCM:Hex=1:8), and recrystallized with methanol to obtain target Compound 4-1-4 (74.6 g, 57%).

4) Preparation of Compound 4-1-3

After dissolving Compound 4-1-4 (74.6 g, 265.1 mM), bis(pinacolato)diboron (134.6 g, 530.2 mM), PdCl₂(dppf) (19.4 g, 26.5 mM) and KOAc (78.1 g, 795.3 mM) in 1,4-dioxane (500 mL), the result was refluxed for 24 hours. After the reaction was completed, the result was extracted by introducing distilled water and DCM thereto at room temperature. The organic layer was dried with MgSO₄, and then the solvent was removed using a rotary evaporator. The reaction material was purified by column chromatography (DCM:Hex=1:5) to obtain target Compound 4-1-3 (76.7 g, 88%).

5) Preparation of Compound 4-1-2

After dissolving Compound 4-1-3 (76.7 g, 233.28 mM), 2-chloro-4,6-diphenyl-1,3,5-triazine (67.7 g, 233.28 mM), Pd(PPh)₄ (13.5 g, 11.7 mM) and K₂CO₃ (64.5 g, 466.6 mM) in 1,4-dioxane/H₂O (800/160 mL), the result was refluxed for 24 hours. After the reaction was completed, the result was extracted by introducing distilled water and DCM thereto at room temperature. The organic layer was dried with MgSO₄, and then the solvent was removed using a rotary evaporator. The reaction material was purified by column chromatography (DCM:Hex=1:5), and recrystallized with methanol to obtain target Compound 4-1-2 (70.9 g, 701).

6) Preparation of Compound 4-1-1

After dissolving Compound 4-1-2 (74.0 g, 163.3 mM), bis(pinacolato)diboron, 326.6 mM), Pd(dba)₂ (9.4 g, 16.3 mM), XPhos (15.6 g, 32.7 mM) and KOAc (48.1 g, 489.9 mM) in 1,4-dioxane (740 mL), the result was refluxed for 24 hours. After the reaction was completed, the result was extracted by introducing distilled water and DCM thereto at room temperature. The organic layer was dried with MgSO₄, and then the solvent was removed using a rotary evaporator. The reaction material was purified by column chromatography (DCM:Hex=1:4) to obtain target Compound 4-1-1 (72.9 g, 85%).

7) Preparation of Compound 4-1

After dissolving Compound 4-1-1 (15.0 g, 28.5 mM), 2-bromodibenzo[b,d]furan (7.8 g, 31.4 mM), Pd(PPh)₄ (1.6 g, 1.4 mM) and K₂CO₃ (7.9 g, 57.0 mM) in 1,4-dioxane/H₂O (200/40 mL), the result was refluxed for 24 hours. After the reaction was completed, the result was extracted by introducing distilled water and DCM thereto at room temperature. The organic layer was dried with MgSO₄, and then the solvent was removed using a rotary evaporator. The reaction material was purified by column chromatography (DCM:Hex=1:3), and recrystallized with methanol to obtain target Compound 4-1 (12.9 g, 80%).

Target Compound A was synthesized in the same manner as in Preparation Example 2 except that Intermediate A of the following Table 2 was used instead of 2-chloro-4,6-diphenyl-1,3,5-triazine, and Intermediate B of the following Table 2 was used instead of 2-bromodibenzo[b,d]furan.

TABLE 2

| Com- | | | | |
|---|---|---|---|---|
| pound | | | | |
| No. | Intermediate A | Intermediate B | Target Compound A | Yield |
| 4-2 | 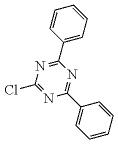 [SMILES: Clc1nc(-c2ccccc2)nc(-c2ccccc2)n1] | 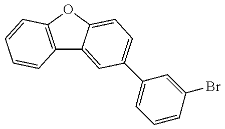 [SMILES: Brc1cccc(-c2ccc3oc4ccccc4c3c2)c1] | 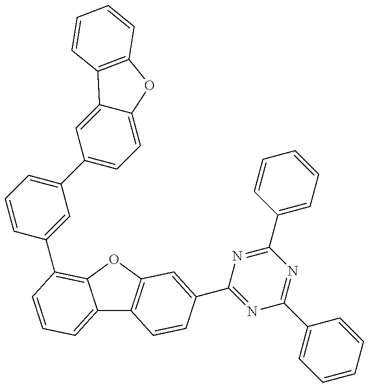 [SMILES: c1ccc(-c2nc(-c3ccccc3)nc(-c3ccc4c(c3)oc3c(-c5cccc(-c6ccc7oc8ccccc8c7c6)c5)cccc34)n2)cc1] | 22% |
| 4-4 | | 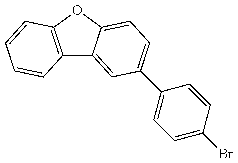 [SMILES: Brc1ccc(-c2ccc3oc4ccccc4c3c2)cc1] | 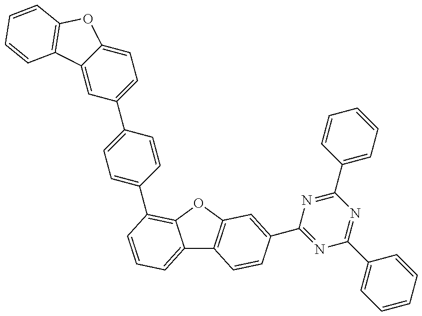 [SMILES: c1ccc(-c2nc(-c3ccccc3)nc(-c3ccc4c(c3)oc3c(-c5ccc(-c6ccc7oc8ccccc8c7c6)cc5)cccc34)n2)cc1] | 23% |
| 4-14 | | 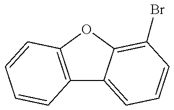 [SMILES: Brc1cccc2c1oc1ccccc12] | 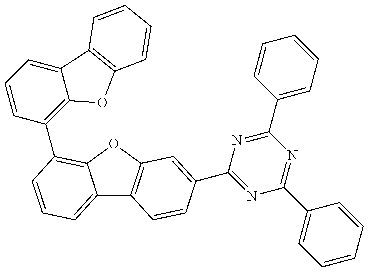 [SMILES: c1ccc(-c2nc(-c3ccccc3)nc(-c3ccc4c(c3)oc3c(-c5cccc6c5oc5ccccc56)cccc34)n2)cc1] | 27% |
| 4-37 | | 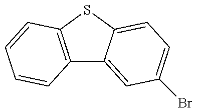 [SMILES: Brc1ccc2sc3ccccc3c2c1] | 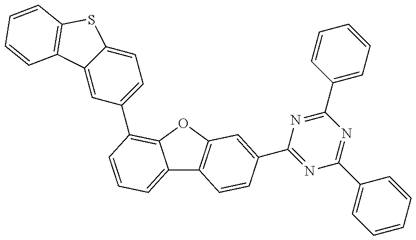 [SMILES: c1ccc(-c2nc(-c3ccccc3)nc(-c3ccc4c(c3)oc3c(-c5ccc6sc7ccccc7c6c5)cccc34)n2)cc1] | 24% |
| 4-40 | | 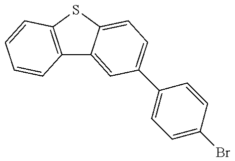 [SMILES: Brc1ccc(-c2ccc3sc4ccccc4c3c2)cc1] | 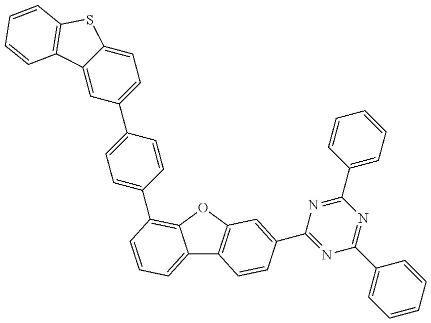 [SMILES: c1ccc(-c2nc(-c3ccccc3)nc(-c3ccc4c(c3)oc3c(-c5ccc(-c6ccc7sc8ccccc8c7c6)cc5)cccc34)n2)cc1] | 20% |
| 4-49 | | 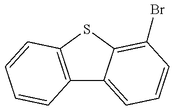 [SMILES: Brc1cccc2c1sc1ccccc12] | 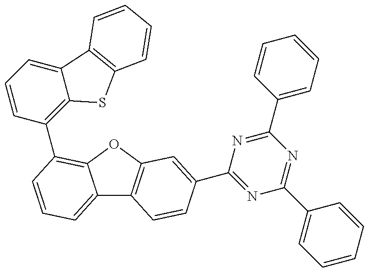 [SMILES: c1ccc(-c2nc(-c3ccccc3)nc(-c3ccc4c(c3)oc3c(-c5cccc6c5sc5ccccc56)cccc34)n2)cc1] | 25% |
| 4-51 | | 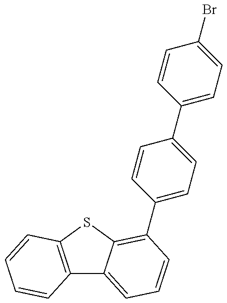 [SMILES: Brc1ccc(-c2ccc(-c3cccc4c3sc3ccccc34)cc2)cc1] | 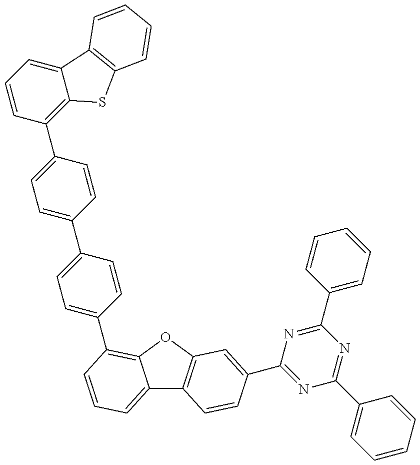 [SMILES: c1ccc(-c2nc(-c3ccccc3)nc(-c3ccc4c(c3)oc3c(-c5ccc(-c6ccc(-c7cccc8c7sc7ccccc78)cc6)cc5)cccc34)n2)cc1] | 22% |
| 4-61 | | 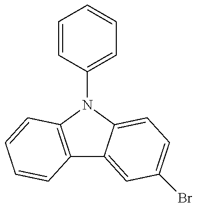 [SMILES: Brc1ccc2c(c1)c1ccccc1n2-c1ccccc1] | 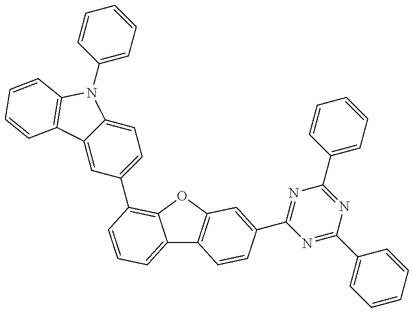 [SMILES: c1ccc(-c2nc(-c3ccccc3)nc(-c3ccc4c(c3)oc3c(-c5ccc6c(c5)c5ccccc5n6-c5ccccc5)cccc34)n2)cc1] | 26% |
| 4-81 | | 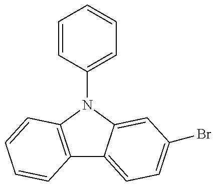 [SMILES: Brc1ccc2c3ccccc3n(-c3ccccc3)c2c1] | 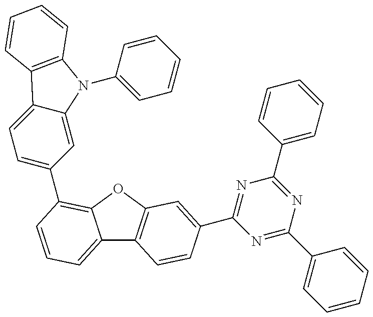 [SMILES: c1ccc(-c2nc(-c3ccccc3)nc(-c3ccc4c(c3)oc3c(-c5ccc6c7ccccc7n(-c7ccccc7)c6c5)cccc34)n2)cc1] | 23% |

### <Preparation Example 3> Synthesis of Compound 5-3

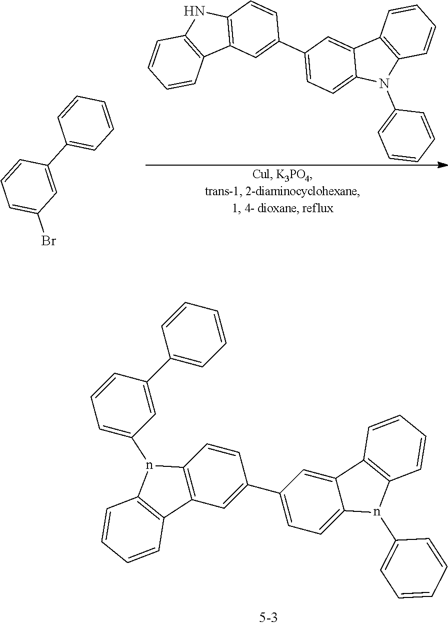

1) Preparation of Compound 5-3

After dissolving 3-bromo-1,1′-biphenyl (3.7 g, 15.8 mM), 9-phenyl-9H,9′H-3,3′-bicarbazole (6.5 g, 15.8 mM), CuI (3.0 g, 15.3 mM), trans-1,2-diaminocyclohexane (1.9 mL, 15.8 mM) and K₃PO₄ (3.3 g, 31.6 mM) in 1,4-dioxane (100 mL), the result was refluxed for 24 hours. After the reaction was completed, the result was extracted by introducing distilled water and DCM thereto at room temperature. The organic layer was dried with MgSO₄, and then the solvent was removed using a rotary evaporator. The reaction material was purified by column chromatography (DCM:Hex=1:3), and recrystallized with methanol to obtain target Compound 5-3 (7.5 g, 85%).

Target Compound A was synthesized in the same manner as in Preparation Example 3 except that Intermediate A of the following Table 3 was used instead of 3-bromo-1,1′-biphenyl, and Intermediate B of the following Table 3 was used instead of 9-phenyl-9H,9′H-3,3′-bicarbazole.

TABLE 3

| Com- | | | | Total |
|---|---|---|---|---|
| pound | | | | |
| No. | Intermediate A | Intermediate B | Target Compound A | Yield |
| 5-4 | 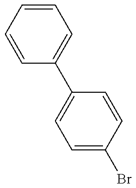 [SMILES: Brc1ccc(-c2ccccc2)cc1] | 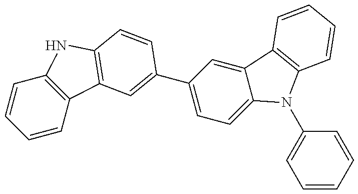 [SMILES: c1ccc(-n2c3ccccc3c3cc(-c4ccc5[nH]c6ccccc6c5c4)ccc32)cc1] | 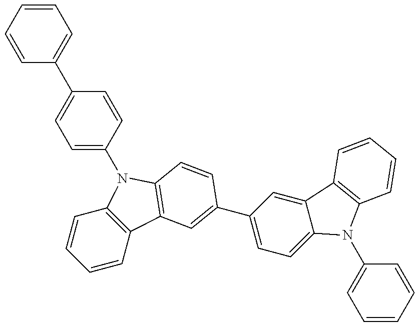 [SMILES: c1ccc(-c2ccc(-n3c4ccccc4c4cc(-c5ccc6c(c5)c5ccccc5n6-c5ccccc5)ccc43)cc2)cc1] | 83% |
| 5-7 | 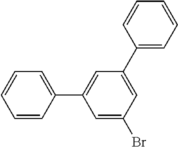 [SMILES: Brc1cc(-c2ccccc2)cc(-c2ccccc2)c1] | | 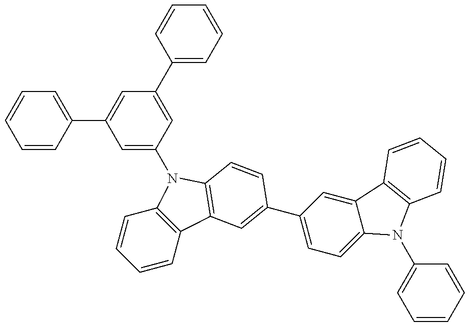 [SMILES: c1ccc(-c2cc(-c3ccccc3)cc(-n3c4ccccc4c4cc(-c5ccc6c(c5)c5ccccc5n6-c5ccccc5)ccc43)c2)cc1] | 84% |
| 5-31 | 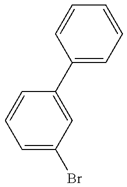 [SMILES: Brc1cccc(-c2ccccc2)c1] | 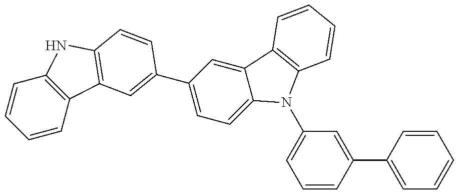 [SMILES: c1ccc(-c2cccc(-n3c4ccccc4c4cc(-c5ccc6[nH]c7ccccc7c6c5)ccc43)c2)cc1] | 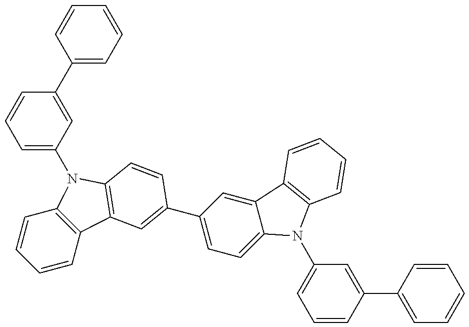 [SMILES: c1ccc(-c2cccc(-n3c4ccccc4c4cc(-c5ccc6c(c5)c5ccccc5n6-c5cccc(-c6ccccc6)c5)ccc43)c2)cc1] | 81% |
| 5-32 | 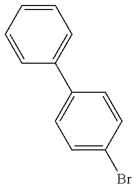 [SMILES: Brc1ccc(-c2ccccc2)cc1] | | 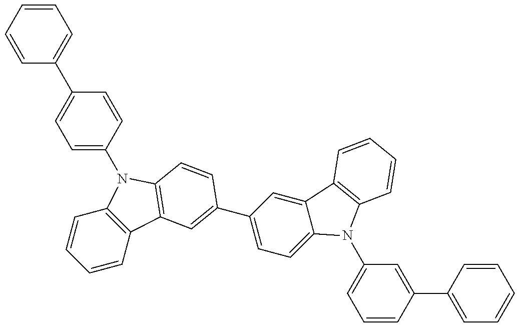 [SMILES: c1ccc(-c2ccc(-n3c4ccccc4c4cc(-c5ccc6c(c5)c5ccccc5n6-c5cccc(-c6ccccc6)c5)ccc43)cc2)cc1] | 80% |
| 5-42 | 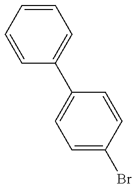 [SMILES: Brc1ccc(-c2ccccc2)cc1] | 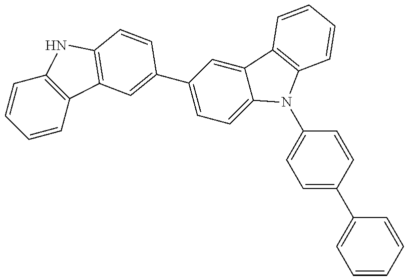 [SMILES: c1ccc(-c2ccc(-n3c4ccccc4c4cc(-c5ccc6[nH]c7ccccc7c6c5)ccc43)cc2)cc1] | 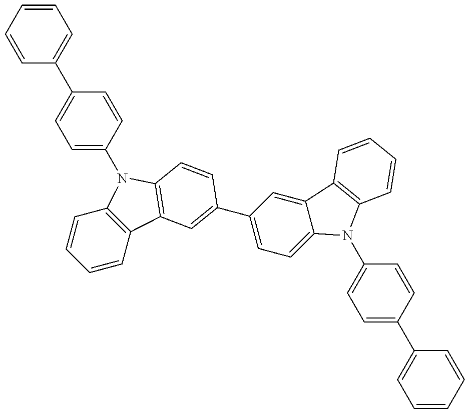 [SMILES: c1ccc(-c2ccc(-n3c4ccccc4c4cc(-c5ccc6c(c5)c5ccccc5n6-c5ccc(-c6ccccc6)cc5)ccc43)cc2)cc1] | 82% |

Heterocyclic compounds corresponding to Chemical Formula 1 and Chemical Formula 2 other than the compounds described in Preparation Examples 1 to 3 and Tables 1 to 3 were also prepared in the same manner as in the methods described in the preparation examples described above.

Synthesis identification data of the compounds prepared above are as described in the following [Table 4] and [Table 5].

TABLE 4

| Compound | |
|---|---|
| No. | ¹H NMR (CDCl₃, 200 Mz) |
| 1-1 | δ = 8.28(4H, d), 7.95(1H, d), 7.89(1H, d), 7.62-7.81(8H, m), |
| | 7.32-7.51(9H, m) |
| 1-2 | δ = 8.28(4H, d), 7.95(1H, d), 7.89(1H, d), 7.32- |
| | 7.81(21H, m) |
| 1-14 | δ = 8.28(4H, d), 7.95(1H, d), 7.89(1H, d), 7.85(1H, d), |
| | 7.81(1H, d), 7.75(2H, d), 7.66(1H, d), 7.64(1H, s), |
| | 7.62 (1H, d), 7.32-7.51(10H, m) |
| 1-18 | δ = 8.28(2H, d), 7.81-7.89(6H, m) 7.66(2H, d), 7.51- |
| | 7.60(8H, m), 7.25-7.41(17H, m) |
| 1-37 | δ = 8.45(1H, d), 8.28(4H, d), 7.95-8.00(4H, m), 7.86(1H, d), |
| | 7.75(2H, d), 7.64(1H, s), 7.62(1H, d), 7.41-7.52(9H, m) |
| 1-38 | δ = 8.45(1H, d), 8.28(4H, d), 7.95-8.00(4H, m), 7.86(1H, d), |
| | 7.75(2H, d), 7.70(1H, s), 7.41-7.64(14H, m) |
| 1-49 | δ = 8.45(1H, d), 8.41(1H, d), 8.28(4H, d), 8.20(1H, d), |
| | 7.98(1H, d), 7.95(1H, d), 7.75(2H, d), 7.41-7.64(12H, m) |
| 1-50 | δ = 8.45(1H, d), 8.41(1H, d), 8.28(4H, d), 8.20(1H, d), |
| | 7.98(1H, d), 7.95(1H, d), 7.75(2H, d), 7.70(1H, s), |
| | 7.41-7.62(15H, m) |
| 1-61 | δ = 8.28(4H, d), 8.18(1H, d), 8.12(1H, d), 8.00(1H, d), |
| | 7.95(1H, d), 7.77(1H, s), 7.75(2H, d), 7.41-7.77(16H, m), |
| | 7.29(1H, t) |
| 1-81 | δ = 8.49(1H, d), 8.28(4H, d), 8.12(1H, d), 8.10(1H, d), |
| | 7.95(1H, d), 7.75(2H, d), 7.41-7.64(17H, m), 7.29(1H, t) |
| 1-102 | δ = 9.15(1H, s), 8.93(2H, d), 8.28(4H, d), 8.04-8.18(4H, m), |
| | 7.41-7.95(20H, m) |
| 1-106 | δ = 8.28(4H, d), 7.95(1H, d), 7.75(2H, d), 7.62-7.66(5H, m), |
| | 7.41-7.52(17H, m), 7.25(4H, s) |
| 4-1 | δ = 8.28(4H, d), 7.64-7.95(10H, m), 7.32-7.51(9H, m) |
| 4-2 | δ = 8.36(4H, m), 8.08-7.73(11H, m), 7.61(2H, d), 7.54- |
| | 7.50(8H, m), 7.39(1H, t), 7.31(1H, t) |
| 4-4 | δ = 8.36(4H, m), 8.08-7.94(4H, m), 7.88(1H, d), 7.83- |
| | 7.76(4H m), 7.54-7.50(8H, m), 7.39-7.25(6H, m), |
| 4-14 | δ = 8.36(4H, m), 8.08(2H, d), 8.03-7.98(4H, m), 7.82(1H, d), |
| | 7.76(1H, s), 7.54-7.50(9H, m), 7.39(1H, t), 7.31(1H, t) |
| 4-37 | δ = 8.45(1H, d), 8.36(4H, m), 8.12-7.99(6H, m), 7.93(1H, d), |
| | 7.82(1H, d), 7.76(1H, s), 7.56-7.49(9H, m), |
| 4-40 | δ = 8.45(1H, d), 8.36(4H, m), 8.12-7.99(8H, m), 7.93(1H, d), |
| | 7.82(1H, d), 7.76(1H, d), 7.59-7.46(9H, m), 7.25(4H, s) |
| 4-49 | δ = 8.55(1H, d), 8.45(1H, d), 8.36(4H, m), 8.08(1H, d), |
| | 8.03-8.02(2H, m), 7.93(1H, d), 7.82(1H, d) 7.76(1H, s), |
| | 7.70(1H, t), 7.56-7.49(10H, m) |
| 4-51 | δ = 8.55(1H, d), 8.45(1H, d), 8.36-8.32(5H, m), 8.08(1H, |
| | d), 8.03-8.02(2H, m), 7.93(1H, d), 7.82(1H, d), |
| | 7.76(1H, s), 7.70(1H, t), 7.56-7.49(9H, m), 7.24(8H, s) |
| 4-61 | δ = 8.36-8.30(5H, m), 8.19(1H, d), 8.13-8.03(4H, m), |
| | 7.89(1H, s), 7.82(1H, d), 7.76(1H, s), 7.62-7.48(14H, m), |
| | 7.20(1H, t) |
| 4-81 | δ = 8.62(1H, d), 8.36(4H, m), 8.22(2H, t), 8.08-8.02(3H, m), |
| | 7.82(1H, d), 7.76(1H, s) 7.74(1H, s), 7.62-7.48(14H, m), |
| | 7.20(1H, t) |
| 5-3 | δ = 8.55(1H, d), 8.30(1H, d), 8.21-8.13(3H, m), 7.99- |
| | 7.89(3H, m), 7.77-7.35(17H, m), 7.20-7.16(2H, m) |
| 5-4 | δ = 8.55(1H, d), 8.30(1H, d), 8.19-8.13(2H, m), 7.99- |
| | 7.89(8H, m), 7.77-7.75(3H, m), 7.62-7.35(11H, m), |
| | 7.20-7.16(2H, m) |
| 5-7 | δ = 8.55(1H, d), 8.31-8.30(3H, d), 8.19-8.13(2H, m), |
| | 7.99-7.89(5H, m), 7.77-7.75(5H, m), 7.62-7.35(14H, m), |
| | 7.20-7.16(2H, m) |
| 5-31 | δ = 8.55(1H, d), 8.30(1H, d), 8.21-8.13(4H, m), 7.99- |
| | 7.89(4H, m), 7.77-7.35(20H, m), 7.20-7.16(2H, m) |
| 5-32 | δ = 8.55(1H, d), 8.30(1H, d), 8.21-8.13(3H, m), 7.99- |
| | 7.89(8H, m), 7.77-7.35(17H, m), 7.20-7.16(2H, m) |
| 5-42 | δ = 8.55(1H, d), 8.30(1H, d), 8.19(1H, d), 8.13(1H, d), |
| | 7.99-7.89(12H, m), 7.77-7.75(5H, m), 7.58(1H, d), |
| | 7.49-7.35(8H, m), 7.20-7.16(2H, m) |

TABLE 5

| Compound | FD-MS |
|---|---|
| 1-1 | m/z = 565.18 (C₃₉H₂₃N₃O₂ = 565.62) |
| 1-2 | m/z = 641.21 (C₄₅H₂₇N₃O₂ = 641.71) |
| 1-14 | m/z = 565.18 (C₃₉H₂₃N₃O₂ = 565.62) |
| 1-18 | m/z = 793.27 (C₅₇H₃₅N₃O₂ = 793.91) |
| 1-37 | m/z = 581.16 (C₃₉H₂₃N₃OS = 581.68) |
| 1-38 | m/z = 657.19 (C₄₅H₂₇N₃OS = 657.78) |
| 1-49 | m/z = 581.16 (C₃₉H₂₃N₃OS = 581.68) |
| 1-50 | m/z = 657.19 (C₄₅H₂₇N₃OS = 657.78) |
| 1-61 | m/z = 640.23 (C₄₅H₂₈N₄O = 640.73) |
| 1-81 | m/z = 640.23 (C₄₅H₂₈N₄O = 640.73) |
| 1-102 | m/z = 701.25 (C₅₁H₃₁N₃O₀ = 701.81) |
| 1-106 | m/z = 641.21 (C₅₁H₃₃N₃O = 641.71) |
| 4-1 | m/z = 565.13 (C₃₉H₂₃N₃O₂ = 565.62) |
| 4-2 | m/z = 641.21 (C₄₅H₂₇N₃O₂ = 641.71) |
| 4-4 | m/z = 641.21 (C₄₅H₂₇N₃O₂ = 641.71) |
| 4-14 | m/z = 565.13 (C₃₉H₂₃N₃O₂ = 565.62) |
| 4-37 | m/z = 581.16 (C₃₉H₂₃N₃OS = 581.68) |
| 4-40 | m/z = 657.19 (C₄₅H₂₇N₃OS = 657.78) |
| 4-49 | m/z = 581.16 (C₃₉H₂₃N₃OS = 581.68) |
| 4-51 | m/z = 733.22 (C₅₁H₃₁N₃OS = 733.88) |
| 4-61 | m/z = 640.23 (C₄₅H₂₈N₄O = 640.73) |
| 4-81 | m/z = 640.23 (C₄₅H₂₈N₄O = 640.73) |
| 5-3 | m/z = 560.23 (C₄₂H₂₈N₂ = 560.70) |
| 5-4 | m/z = 560.23 (C₄₂H₂₈N₂ = 560.70) |
| 5-7 | m/z = 636.26 (C₄₈H₃₂N₂ = 636.80) |
| 5-31 | m/z = 636.26 (C₄₈H₃₂N₂ = 636.80) |
| 5-32 | m/z = 636.26 (C₄₈H₃₂N₂ = 636.80) |
| 5-42 | m/z = 636.26 (C₄₈H₃₂N₂ = 636.78) |

### <Experimental Example 1>—Manufacture of Organic Light Emitting Device

A glass substrate on which indium tin oxide (ITO) was coated as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and ultraviolet ozone (UVO) treatment was conducted for 5 minutes using UV in a UV cleaner. After that, the substrate was transferred to a plasma cleaner (PT), and after conducting plasma treatment under vacuum for ITO work function and residual film removal, the substrate was transferred to a thermal deposition apparatus for organic deposition.

On the transparent ITO electrode (anode), a hole injection layer 2-TNATA (4,4′,4″-tris[2-naphthyl(phenyl)amino]triphenylamine) and a hole transfer layer NPB (N,N′-di(1-naphthyl)-N,N′-diphenyl-(1,1′-biphenyl)-4,4′-diamine), which are common layers, were formed.

A light emitting layer was thermal vacuum deposited thereon as follows. As the light emitting layer, a compound of the following Table 6 was deposited to 400 Å as a host, and as a green phosphorescent dopant, Ir(ppy)₃ was doped and deposited by 7% with respect to the deposited thickness of the light emitting layer. After that, BCP was deposited to 60 Å as a hole blocking layer, and Alq₃ was deposited to 200 Å thereon as an electron transfer layer. Lastly, an electron injection layer was formed on the electron transfer layer by depositing lithium fluoride (LiF) to a thickness of 10 Å, and then a cathode was formed on the electron injection layer by depositing an aluminum (Al) cathode to a thickness of 1,200 Å, and as a result, an organic electroluminescent device was manufactured.

Meanwhile, all the organic compounds required to manufacture the OLED were vacuum sublimation purified under 10⁻⁸ torr to 10⁻⁶ torr for each material to be used in the OLED manufacture.

For each of the organic electroluminescent devices manufactured as above, electroluminescent (EL) properties were measured using M7000 manufactured by McScience Inc., and with the measurement results, T₉₀ was measured when standard luminance was 6,000 cd/m² through a lifetime measurement system (M6000) manufactured by McScience Inc.

Results of measuring driving voltage, light emission efficiency, color coordinate (CIE) and lifetime of the manufactured organic light emitting devices are as shown in the following Table 6.

TABLE 6

| | Light | | | | |
|---|---|---|---|---|---|
| | Emitting | Driving | | Color | |
| | Layer | Voltage | Efficiency | Coordinate | Lifetime |
| | Compound | (V) | (cd/A) | (x, y) | (T₉₀) |
| Comparative | 5-3 | 2.63 | 29.2 | (0.233, | 45 |
| Example 1 | | | | 0.671) | |
| Comparative | 5-4 | 2.59 | 31.3 | (0.231, | 48 |
| Example 2 | | | | 0.673) | |
| Comparative | 5-7 | 2.61 | 30.4 | (0.237, | 44 |
| Example 3 | | | | 0.677) | |
| Comparative | 5-31 | 2.65 | 30.8 | (0.234, | 43 |
| Example 4 | | | | 0.674) | |
| Comparative | 5-32 | 2.58 | 29.8 | (0.231, | 44 |
| Example 5 | | | | 0.681) | |
| Comparative | 5-42 | 2.59 | 30.1 | (0.233, | 43 |
| Example 6 | | | | 0.682) | |
| Comparative | Ref. 1 | 6.14 | 38.9 | (0.236, | 133 |
| Example 7 | | | | 0.687) | |
| Comparative | Ref. 2 | 6.54 | 35.3 | (0.236, | 69 |
| Example 8 | | | | 0.666) | |
| Comparative | Ref. 3 | 4.85 | 43.4 | (0.237, | 85 |
| Example 9 | | | | 0.677) | |
| Comparative | Ref. 4 | 2.91 | 29.7 | (0.233, | 50 |
| Example 10 | | | | 0.683) | |
| Comparative | Ref. 5 | 6.66 | 35.8 | (0.233, | 64 |
| Example 11 | | | | 0.681) | |
| Comparative | Ref. 6 | 6.15 | 34.9 | (0.231, | 77 |
| Example 12 | | | | 0.671) | |
| Comparative | Ref. 7 | 6.30 | 30.5 | (0.233, | 64 |
| Example 13 | | | | 0.678) | |
| Comparative | Ref. 8 | 5.54 | 43.9 | (0.241, | 175 |
| Example 14 | | | | 0.682) | |
| Comparative | Ref. 9 | 5.84 | 49.4 | (0.241, | 185 |
| Example 15 | | | | 0.673) | |
| Comparative | Ref. 10 | 5.31 | 45.9 | (0.240, | 173 |
| Example 16 | | | | 0.681) | |
| Example 1 | 1-1 | 4.31 | 53.2 | (0.247, | 227 |
| | | | | 0.667) | |
| Example 2 | 1-2 | 4.30 | 55.8 | (0.241, | 224 |
| | | | | 0.671) | |
| Example 3 | 1-14 | 4.45 | 52.7 | (0.251, | 225 |
| | | | | 0.674) | |
| Example 4 | 1-18 | 4.38 | 54.0 | (0.240, | 228 |
| | | | | 0.672) | |
| Example 5 | 1-37 | 4.34 | 54.1 | (0.242, | 232 |
| | | | | 0.673) | |
| Example 6 | 1-38 | 4.31 | 55.2 | (0.231, | 238 |
| | | | | 0.681) | |
| Example 7 | 1-49 | 4.41 | 53.7 | (0.241, | 233 |
| | | | | 0.683) | |
| Example 8 | 1-50 | 4.39 | 55.0 | (0.231, | 241 |
| | | | | 0.674) | |
| Example 9 | 1-61 | 4.15 | 50.4 | (0.231, | 195 |
| | | | | 0.684) | |
| Example 10 | 1-81 | 4.12 | 50.8 | (0.246, | 190 |
| | | | | 0.677) | |
| Example 11 | 1-102 | 4.42 | 55.7 | (0.239, | 222 |
| | | | | 0.682) | |
| Example 12 | 1-106 | 4.27 | 54.1 | (0.243, | 220 |
| | | | | 0.671) | |
| Example 13 | 4-1 | 4.20 | 55.8 | (0.247, | 259 |
| | | | | 0.685) | |
| Example 14 | 4-2 | 4.21 | 57.9 | (0.241, | 261 |
| | | | | 0.668) | |
| Example 15 | 4-4 | 4.29 | 55.3 | (0.251, | 262 |
| | | | | 0.672) | |
| Example 16 | 4-14 | 4.28 | 56.0 | (0.240, | 260 |
| | | | | 0.674) | |
| Example 17 | 4-37 | 4.19 | 57.3 | (0.242, | 264 |
| | | | | 0.683) | |
| Example 18 | 4-40 | 4.21 | 56.5 | (0.231, | 270 |
| | | | | 0.667) | |
| Example 19 | 4-49 | 4.31 | 55.6 | (0.241, | 261 |
| | | | | 0.671) | |
| Example 20 | 4-51 | 4.27 | 55.7 | (0.231, | 273 |
| | | | | 0.682) | |
| Example 21 | 4-61 | 3.99 | 51.1 | (0.231, | 211 |
| | | | | 0.677) | |
| Example 22 | 4-81 | 4.01 | 50.3 | (0.246, | 206 |
| | | | | 0.673) | |

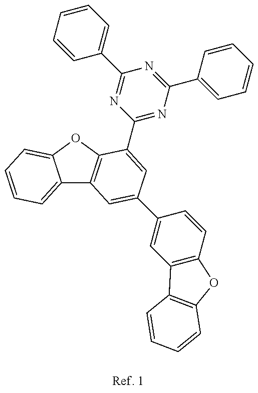 [SMILES: c1ccc(-c2nc(-c3ccccc3)nc(-c3cc(-c4ccc5oc6ccccc6c5c4)cc4c3oc3ccccc34)n2)cc1]

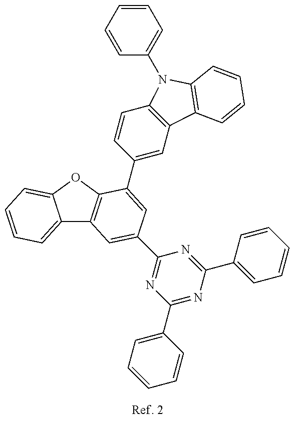 [SMILES: c1ccc(-c2nc(-c3ccccc3)nc(-c3cc(-c4ccc5c(c4)c4ccccc4n5-c4ccccc4)c4oc5ccccc5c4c3)n2)cc1]

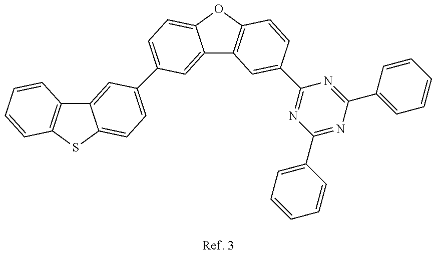 [SMILES: c1ccc(-c2nc(-c3ccccc3)nc(-c3ccc4oc5ccc(-c6ccc7sc8ccccc8c7c6)cc5c4c3)n2)cc1]

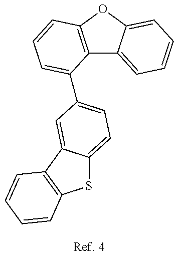 [SMILES: c1ccc2c(c1)oc1cccc(-c3ccc4sc5ccccc5c4c3)c12]

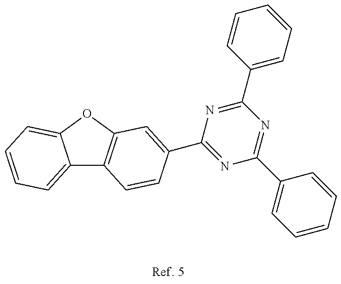 [SMILES: c1ccc(-c2nc(-c3ccccc3)nc(-c3ccc4c(c3)oc3ccccc34)n2)cc1]

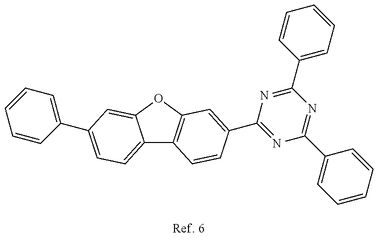 [SMILES: c1ccc(-c2ccc3c(c2)oc2cc(-c4nc(-c5ccccc5)nc(-c5ccccc5)n4)ccc23)cc1]

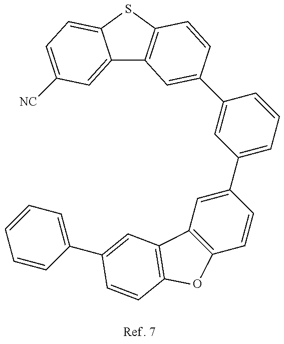 [SMILES: N#Cc1ccc2sc3ccc(-c4cccc(-c5ccc6oc7ccc(-c8ccccc8)cc7c6c5)c4)cc3c2c1]

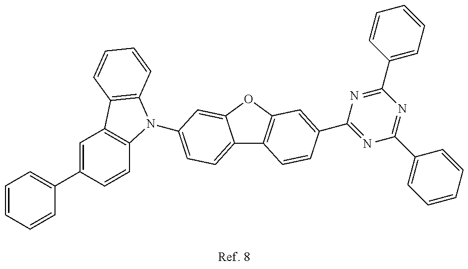 [SMILES: c1ccc(-c2ccc3c(c2)c2ccccc2n3-c2ccc3c(c2)oc2cc(-c4nc(-c5ccccc5)nc(-c5ccccc5)n4)ccc23)cc1]

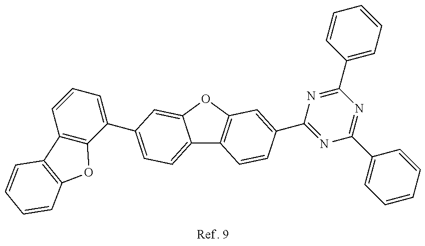 [SMILES: c1ccc(-c2nc(-c3ccccc3)nc(-c3ccc4c(c3)oc3cc(-c5cccc6c5oc5ccccc56)ccc34)n2)cc1]

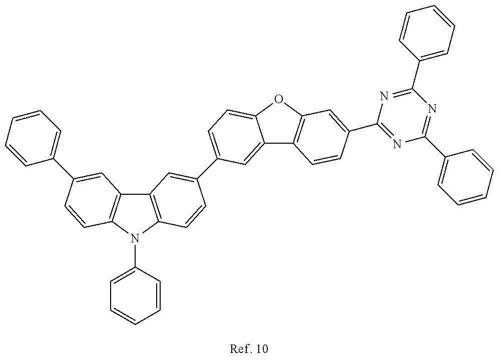 [SMILES: c1ccc(-c2ccc3c(c2)c2cc(-c4ccc5oc6cc(-c7nc(-c8ccccc8)nc(-c8ccccc8)n7)ccc6c5c4)ccc2n3-c2ccccc2)cc1]

### <Experimental Example 2>—Manufacture of Organic Light Emitting Device

A glass substrate on which ITO was coated as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and UVO treatment was conducted for 5 minutes using UV in a UV cleaner. After that, the substrate was transferred to a plasma cleaner (PT), and after conducting plasma treatment under vacuum for ITO work function and residual film removal, the substrate was transferred to a thermal deposition apparatus for organic deposition.

On the transparent ITO electrode (anode), a hole injection layer 2-TNATA (4,4′,4″-tris[2-naphthyl(phenyl)amino]triphenylamine) and a hole transfer layer NPB (N,N′-di(1-naphthyl)-N,N′-diphenyl-(1,1′-biphenyl)-4,4′-diamine), which are common layers, were formed.

A light emitting layer was thermal vacuum deposited thereon as follows. As the light emitting layer, one type of compound described as Chemical Formula 1 and one type of compound described as Chemical Formula 2 as in the following Table 7 were premixed and then deposited in one source of supply to 400 Å as a host, and as a green phosphorescent dopant, Ir(ppy)₃ was doped and deposited by 7% with respect to the deposited thickness of the light emitting layer. After that, BCP was deposited to 60 Å as a hole blocking layer, and Alq₃ was deposited to 200 Å thereon as an electron transfer layer. Lastly, an electron injection layer was formed on the electron transfer layer by depositing lithium fluoride (LiF) to a thickness of 10 Å, and then a cathode was formed on the electron injection layer by depositing an aluminum (Al) cathode to a thickness of 1,200 Å, and as a result, an organic electroluminescent device was manufactured.

Meanwhile, all the organic compounds required to manufacture the OLED were vacuum sublimation purified under 10⁻⁸ torr to 10⁻⁶ torr for each material to be used in the OLED manufacture.

For each of the organic electroluminescent devices manufactured as above, electroluminescent (EL) properties were measured using M7000 manufactured by McScience Inc., and with the measurement results, T₉₀ was measured when standard luminance was 6,000 cd/m² through a lifetime measurement system (M6000) manufactured by McScience Inc.

Results of measuring driving voltage, light emission efficiency, color coordinate (CIE) and lifetime of the organic light emitting devices manufactured according to the present disclosure are as shown in the following Table 7.

| | Light | | | | | |
|---|---|---|---|---|---|---|
| | Emitting | | Driving | | Color | Life- |
| | Layer | | Voltage | Efficiency | Coordinate | time |
| | Compound | Ratio | (V) | (cd/A) | (x, y) | (T₉₀) |
| Example 43 | 1-1.5-3 | 1:8 | 4.77 | 55.2 | (0.233, | 314 |
| | | | | | 0.714) | |
| Example 44 | | 1:5 | 4.55 | 57.2 | (0.243, | 355 |
| | | | | | 0.714) | |
| Example 45 | | 1:2 | 4.13 | 75.3 | (0.241, | 497 |
| | | | | | 0.711) | |
| Example 46 | | 1:1 | 3.81 | 74.9 | (0.231, | 481 |
| | | | | | 0.711) | |
| Example 47 | | 2:1 | 3.88 | 71.2 | (0.251, | 470 |
| | | | | | 0.714) | |
| Example 48 | | 5:1 | 4.33 | 68.3 | (0.241, | 401 |
| | | | | | 0.711) | |
| Example 49 | | 3:1 | 4.64 | 51.0 | (0.247, | 361 |
| | | | | | 0.727) | |
| Example 50 | | 1:3 | 4.51 | 66.2 | (0.243, | 405 |
| | | | | | 0.714) | |
| Example 51 | | 1:2 | 4.20 | 76.3 | (0.241, | 490 |
| | | | | | 0.714) | |
| Example 52 | 1-1:5-4 | 1:1 | 3.91 | 75.1 | (0.233, | 481 |
| | | | | | 0.712) | |
| Example 53 | | 2:1 | 3.96 | 73.4 | (0.251, | 462 |
| | | | | | 0.712) | |
| Example 54 | | 3:1 | 4.23 | 70.9 | (0.247, | 434 |
| | | | | | 0.711) | |
| Example 55 | | 1:2 | 4.18 | 77.2 | (0.250, | 505 |
| | | | | | 0.714) | |
| Example 56 | 1-49:5-7 | 1:1 | 3.82 | 78.4 | (0.231, | 527 |
| | | | | | 0.714) | |
| Example 57 | | 2:1 | 3.90 | 76.8 | (0.251, | 472 |
| | | | | | 0.712) | |
| Example 58 | 1-102:5- | 1:2 | 4.02 | 73.7 | (0.242 | 492 |
| | 31 | | | | 0.722) | |
| Example 59 | | 1:1 | 3.89 | 75.2 | (0.231, | 513 |
| | | | | | 0.711) | |
| Example 60 | | 2:1 | 4.08 | 74.1 | (0.251- | 485 |
| | | | | | 0.724) | |
| Example 61 | 4-1:5-31 | 1:2 | 4.11 | 79.1 | (0.242, | 566 |
| | | | | | 0.724) | |
| Example 62 | | 1:1 | 3.95 | 89.5 | (0.231, | 624 |
| | | | | | 0.711) | |
| Example 63 | | 2:1 | 3.86 | 77.3 | (0.251, | 540 |
| | | | | | 0.711) | |
| Example 64 | | 1:2 | 4.12 | 79.4 | (0.241, | 575 |
| | | | | | 0.712) | |
| Example 65 | 4-1:5-42 | 1:1 | 3.69 | 87.7 | (0.251, | 618 |
| | | | | | 0.714) | |
| Example 66 | | 2:1 | 3.79 | 76.3 | (0.233, | 542 |
| | | | | | 0.714) | |
| Example 67 | | 1:2 | 4.10 | 78.1 | (0.231, | 581 |
| | | | | | 0.712) | |
| Example 68 | 4-14:5-32 | 1:1 | 3.85 | 85.9 | (0.231, | 610 |
| | .2 | | | | 0.711) | |
| Example 69 | | 2:1 | 3.86 | 76.2 | (0.251, | 557 |
| | | | | | 0.712) | |
| Example 70 | | 1:2 | 4.08 | 77.4 | (0.247, | 562 |
| | | | | | 0.710) | |
| Example 71 | 4-14:5- | 1:1 | 3.81 | 81.0 | (0.243, | 603 |
| | 42 | | | | 0.712) | |
| Example 72 | | 2:1 | 3.80 | 78.4 | (0.232, | 542 |
| | | | | | 0.714) | |
| Example 73 | 4-49:5- | 1:2 | 3.99 | 78.1 | (0.241, | 547 |
| | 32 | | | | 0.714) | |
| Example 74 | | 1:1 | 3.75 | 80.2 | (0.251, | 610 |
| | | | | | 0.720) | |
| Example 75 | | 2:1 | 3.77 | 76.1 | (0.251, | 516 |
| | | | | | 0.715) | |
| Example 76 | 4-61:5- | 1:2 | 4.87 | 76.7 | (0.232, | 551 |
| | 42 | | | | 0.714) | |
| Example 77 | | 1:1 | 4.01 | 77.9 | (0.231, | 575 |
| | | | | | 0.714) | |
| Example 78 | 2:1 | | 4.23 | 75.2 | (0.235, | 547 |
| | | | | | 0.711) | |
| Comparative | Ref. 8: | 1:2 | 3.87 | 72.7 | (0.231, | 440 |
| Example 17 | 5-32 | | | | 0.714) | |
| Comparative | | 1:1 | 3.95 | 71.5 | (0.242, | 480 |
| Example 18 | | | | | 0.711) | |
| Comparative | | 2:1 | 3.61 | 77.9 | (0.231, | 498 |
| Example 19 | | | | | 0.712) | |

As seen from the results of Table 6, it was identified that the organic electroluminescent device using the light emitting layer material of the organic electroluminescent device of the present disclosure had lower driving voltage, enhanced light emission efficiency and significantly improved lifetime compared to Comparative Examples 7 to 14.

It was identified that, in Examples 11 and 12 having an aryl group substituting another benzene ring not substituted with the N-containing ring in the dibenzofuran structure, a T1 energy level higher by approximately 2.5 eV or greater was obtained, which readily transferred energy from the host to the dopant, and superior light emission efficiency was obtained as in the arylene group or heteroarylene group-substituted chemical formulae.

Particularly, it was identified that, although the HOMO energy level was localized to one side when X1 has a substituent of NR13, the HOMO energy level was relatively delocalized when X1 has O, S and the like, which leads to a more stable electron-stable structure, and an organic light emitting device with improved lifetime, driving stability and efficiency was manufactured.

It was identified that, when the No. 3 position of one side benzene ring of the dibenzofuran is substituted with N-Het and the No. 4 position of another benzene ring has a specific substituent, a driving voltage was low due to particularly more favorable current density compared to cases of substituting other positions, and triplet energy was also high. It was identified that, when the No. 3 position of one side benzene ring of the dibenzofuran is substituted with N-Het and the No. 1 position of another benzene ring has a specific substituent, thermal stability was superior due to particularly lower Td compared to cases of substituting other positions, and lifetime properties of the organic light emitting device were particularly superior.

In addition, as seen from the results of Tables 6 and 7, effects of more superior efficiency and lifetime were obtained when comprising the compound of Chemical Formula 1 and the compound of Chemical Formula 2 at the same time. Such results may lead to a forecast that an exciplex phenomenon occurred when comprising the two compounds at the same time.

The exciplex phenomenon is a phenomenon of releasing energy having sizes of a donor (p-host) HOMO level and an acceptor (n-host) LUMO level due to electron exchanges between two molecules. When the exciplex phenomenon occurs between two molecules, reverse intersystem crossing (RISC) occurs, and as a result, internal quantum efficiency of fluorescence may increase up to 100%. When a donor (p-host) having a favorable hole transfer ability and an acceptor (n-host) having a favorable electron transfer ability are used as a host of a light emitting layer, holes are injected to the p-host and electrons are injected to the n-host, and therefore, a driving voltage may be lowered, which resultantly helps with enhancement in the lifetime. In the invention of the present application, it was identified that excellent device properties were obtained when, as the light emitting layer host, the compound of Chemical Formula 2 performing a donor role and the compound of Chemical Formula 1 performing an acceptor role were used.

The compounds of Comparative Examples 7, 8 and 9 had a different position of substitution from the compound of the present disclosure, and in the compounds of Comparative Examples 10 and 11, one of the two substituents having the dibenzofuran structure of Chemical Formula 1 of the present application was not present, and it was identified that this broke a balance between holes and electrons in the light emitting layer leading to a decrease in the lifetime. In addition, it was identified that, when the N portion of carbazole bonds to the dibenzofuran as in the compound of Comparative Example 14, holes moved faster leading to a decrease in the lifetime.

1. A heterocyclic compound represented by the following Chemical Formula 1: 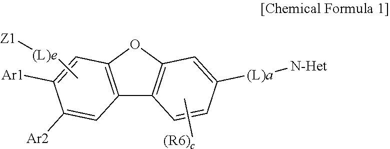
wherein, in Chemical Formula 1,
N-Het is a monocyclic or polycyclic C2 to C60 heterocyclic group substituted or unsubstituted and comprising one or more Ns;
L and L1 are the same as or different from each other, and each independently a direct bond; a substituted or unsubstituted C6 to C60 arylene group; or a substituted or unsubstituted C2 to C60 heteroarylene group;
Ar1 and Ar2 are the same as or different from each other, and each independently hydrogen; deuterium; —CN; or a substituted or unsubstituted C1 to C60 alkyl group; and
Z1 is a substituted or unsubstituted C6 to C60 aryl group; or represented by the following Chemical Formula A, 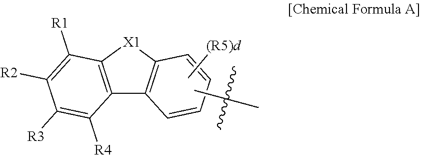
X1 is O; S; CR11R12; or NR13;
R1 to R4 are the same as or different from each other, and each independently hydrogen; a substituted or unsubstituted C1 to C60 alkyl group; a substituted or unsubstituted C6 to C60 aryl group; or a substituted or unsubstituted C2 to C60 heteroaryl group, or two or more groups adjacent to each other bond to each other to form a substituted or unsubstituted C6 to C60 aliphatic or aromatic hydrocarbon ring or a substituted or unsubstituted C2 to C60 heteroring;
R5 and R6 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)RR′; —SiRR′R″ and —NRR′;
R11 to R13, R, R′ and R″ are the same as or different from each other, and each independently a substituted or unsubstituted C1 to C60 alkyl group; a substituted or unsubstituted C6 to C60 aryl group; or a substituted or unsubstituted C2 to C60 heteroaryl group;
c and d are an integer of 0 to 3; and
a and e are an integer of 0 to 5.

2. The heterocyclic compound of claim 1, wherein Chemical Formula 1 is represented by the following Chemical Formula 1-A or 1-B: 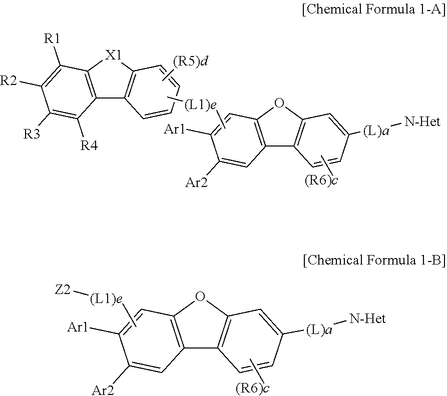
in Chemical Formulae 1-A and 1-B,
R1 to R6, N-Het, L, L1, X1, Ar1, Ar2, a, c, d and e have the same definitions as in Chemical Formula 1; and
Z2 is a substituted or unsubstituted C6 to C60 aryl group.

3. The heterocyclic compound of claim 1, wherein Chemical Formula A of Chemical Formula 1 is represented by any one of the following Chemical Formulae 1-1 to 1-6: 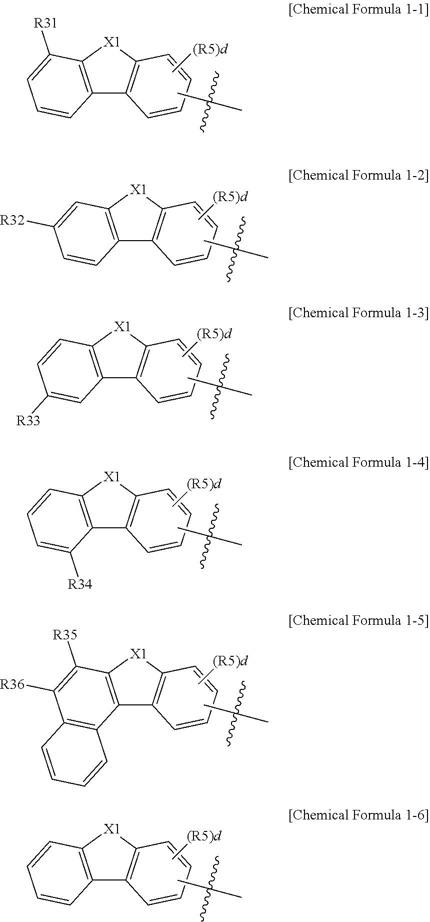
in Chemical Formulae 1-1 to 1-6,
X1, R5 and d have the same definition as in Chemical Formula 1; 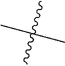
means a position linked to L1 of Chemical Formula 1;
R31 to R34 are the same as or different from each other, and each independently a substituted or unsubstituted C1 to C60 alkyl group; a substituted or unsubstituted C6 to C60 aryl group; or a substituted or unsubstituted C2 to C60 heteroaryl group; and
R35 and R36 are the same as or different from each other, and each independently hydrogen; a substituted or unsubstituted C1 to C60 alkyl group; a substituted or unsubstituted C6 to C60 aryl group; or a substituted or unsubstituted C2 to C60 heteroaryl group.

4. The heterocyclic compound of claim 1, wherein N-Het is any one selected from among the following structural formulae: 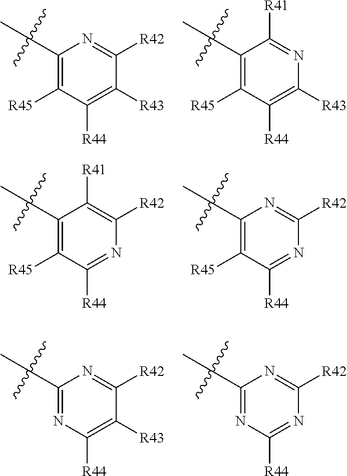
in the structural formulae, 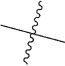
means a position linked to L of Chemical Formula 1; and
R41 to R45 are the same as or different from each other, and each independently hydrogen; a substituted or unsubstituted C1 to C60 alkyl group; a substituted or unsubstituted C6 to C60 aryl group; or a substituted or unsubstituted C2 to C60 heteroaryl group.

5. The heterocyclic compound of claim 1, wherein R5 and R6 are hydrogen.

6. The heterocyclic compound of claim 1, wherein Chemical Formula 1 is represented by any one of the following compounds:

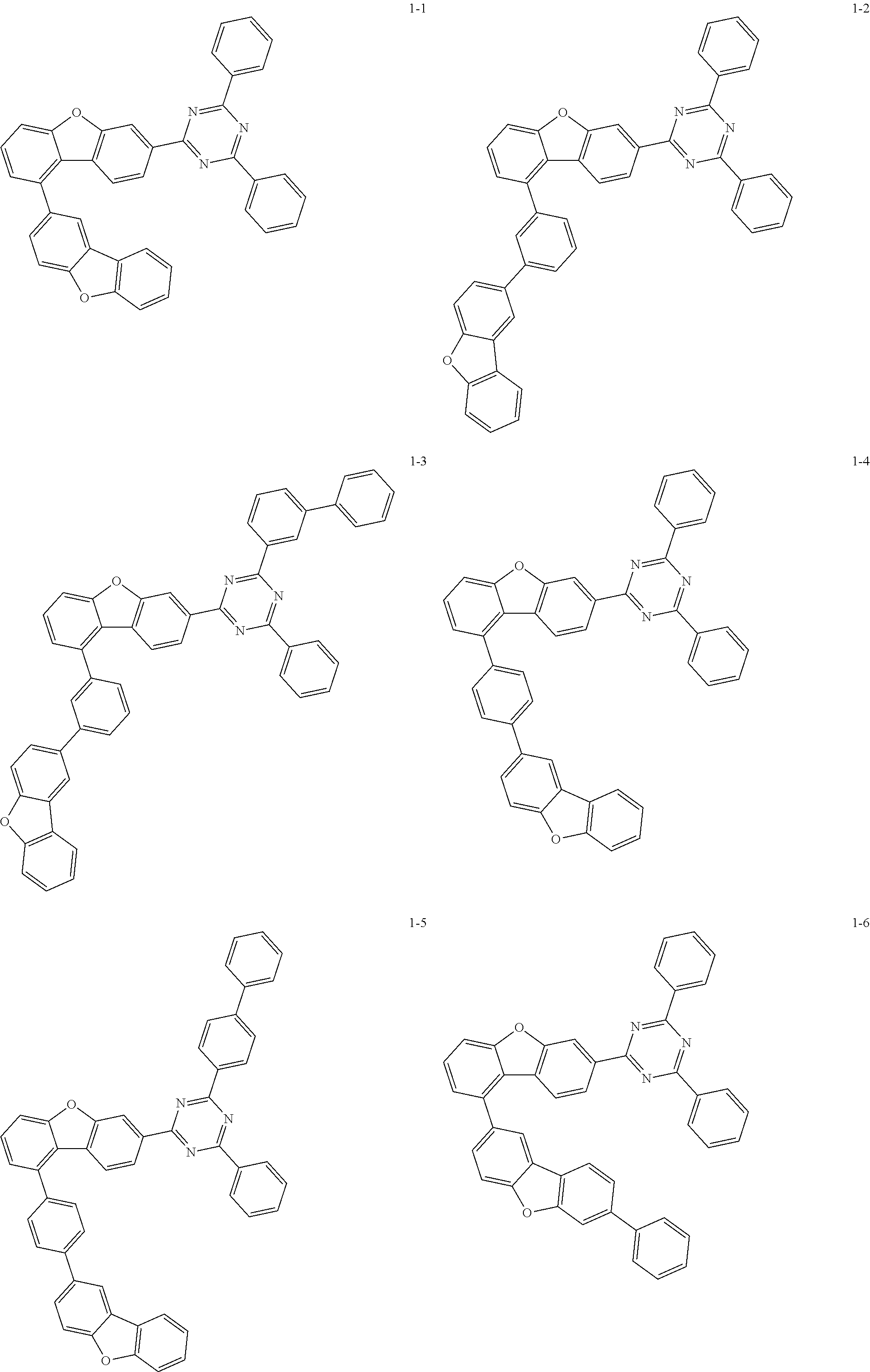

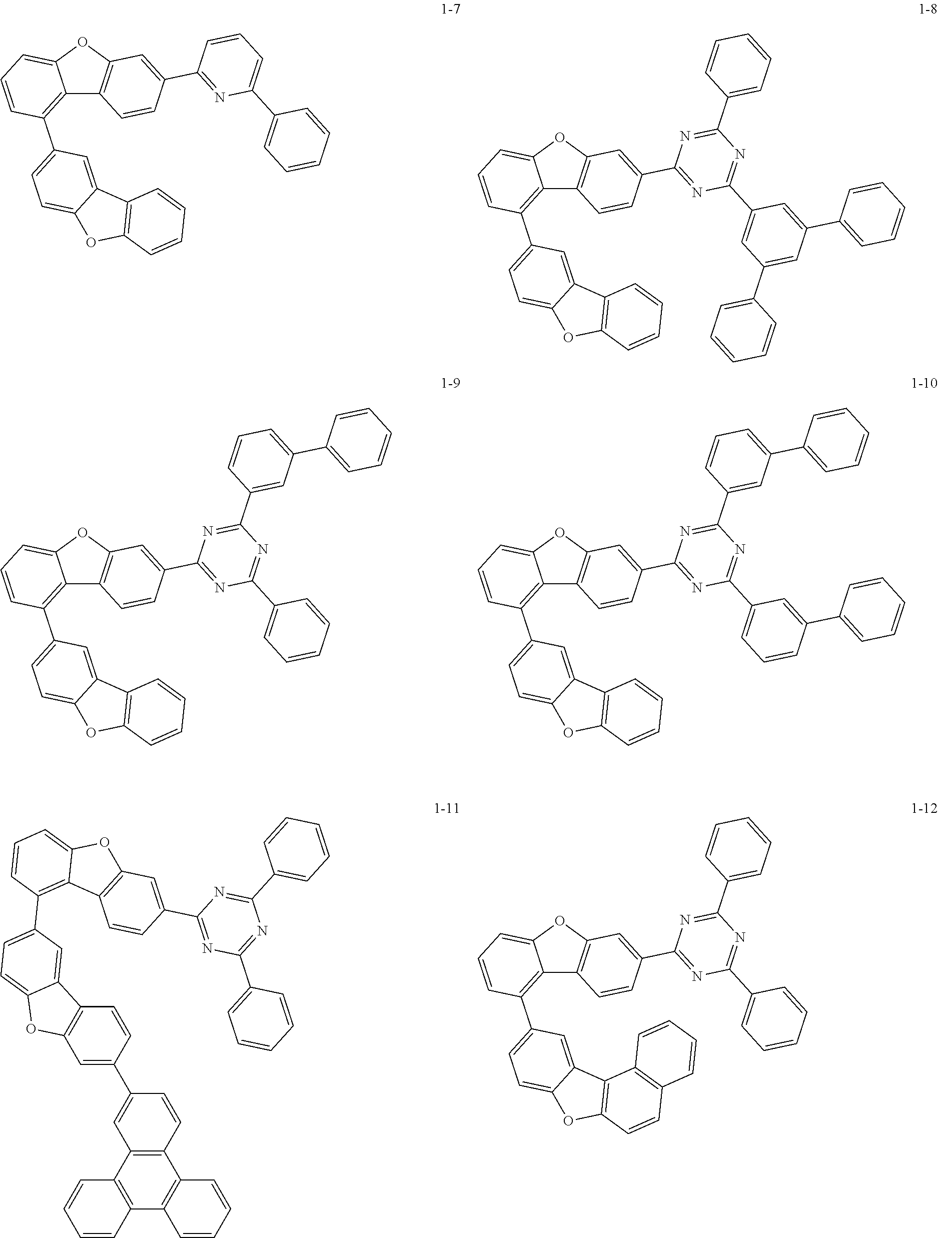

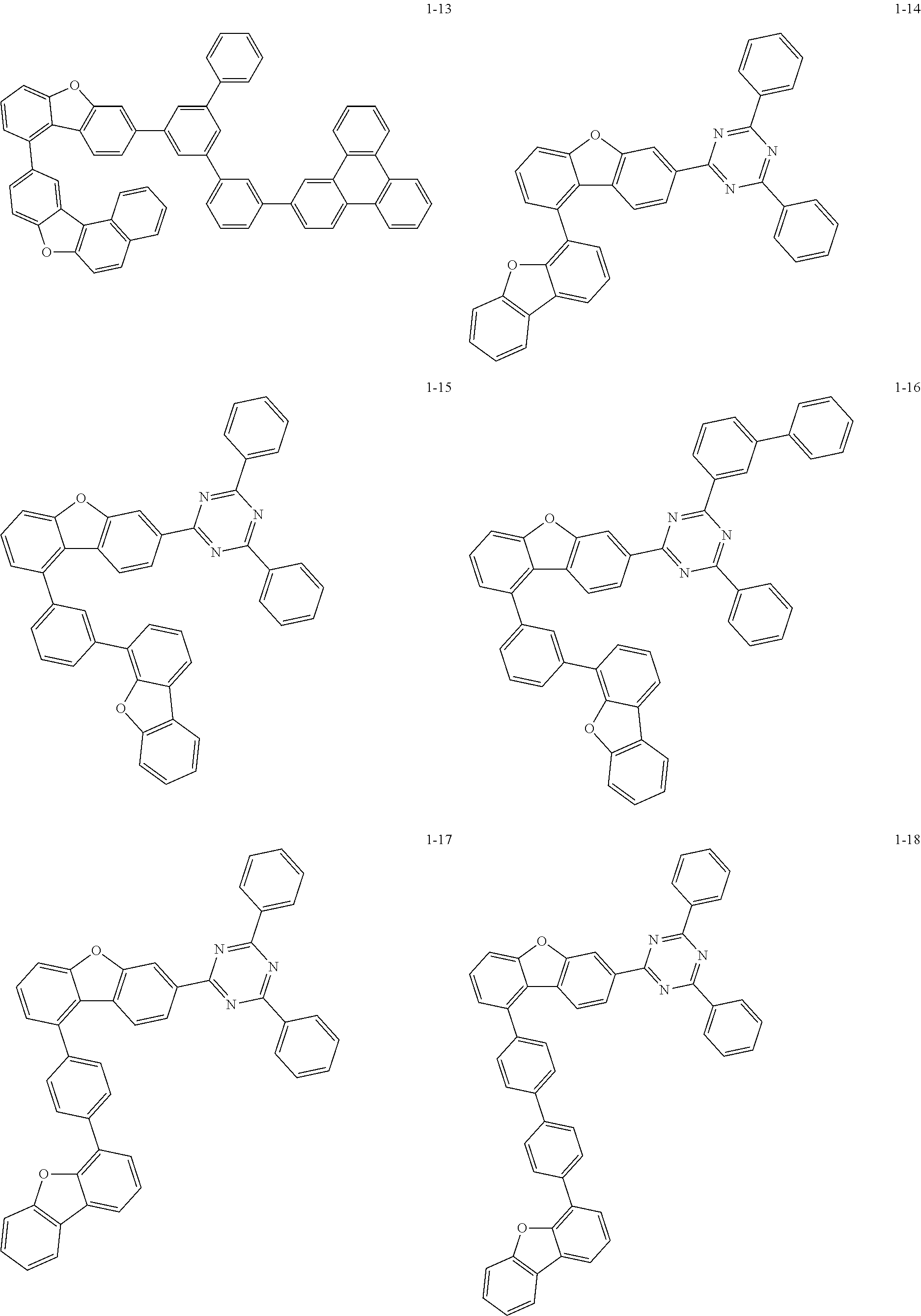

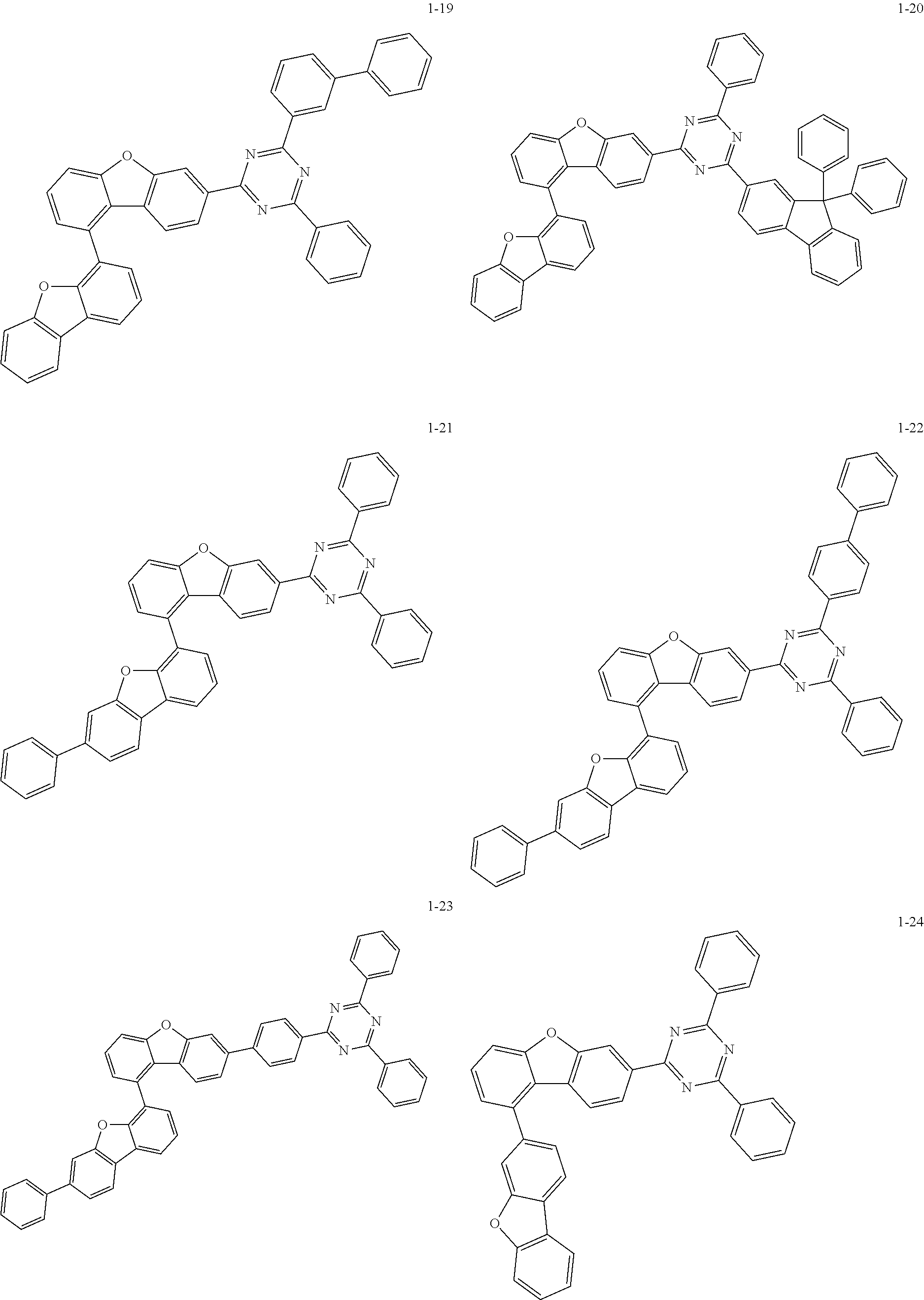

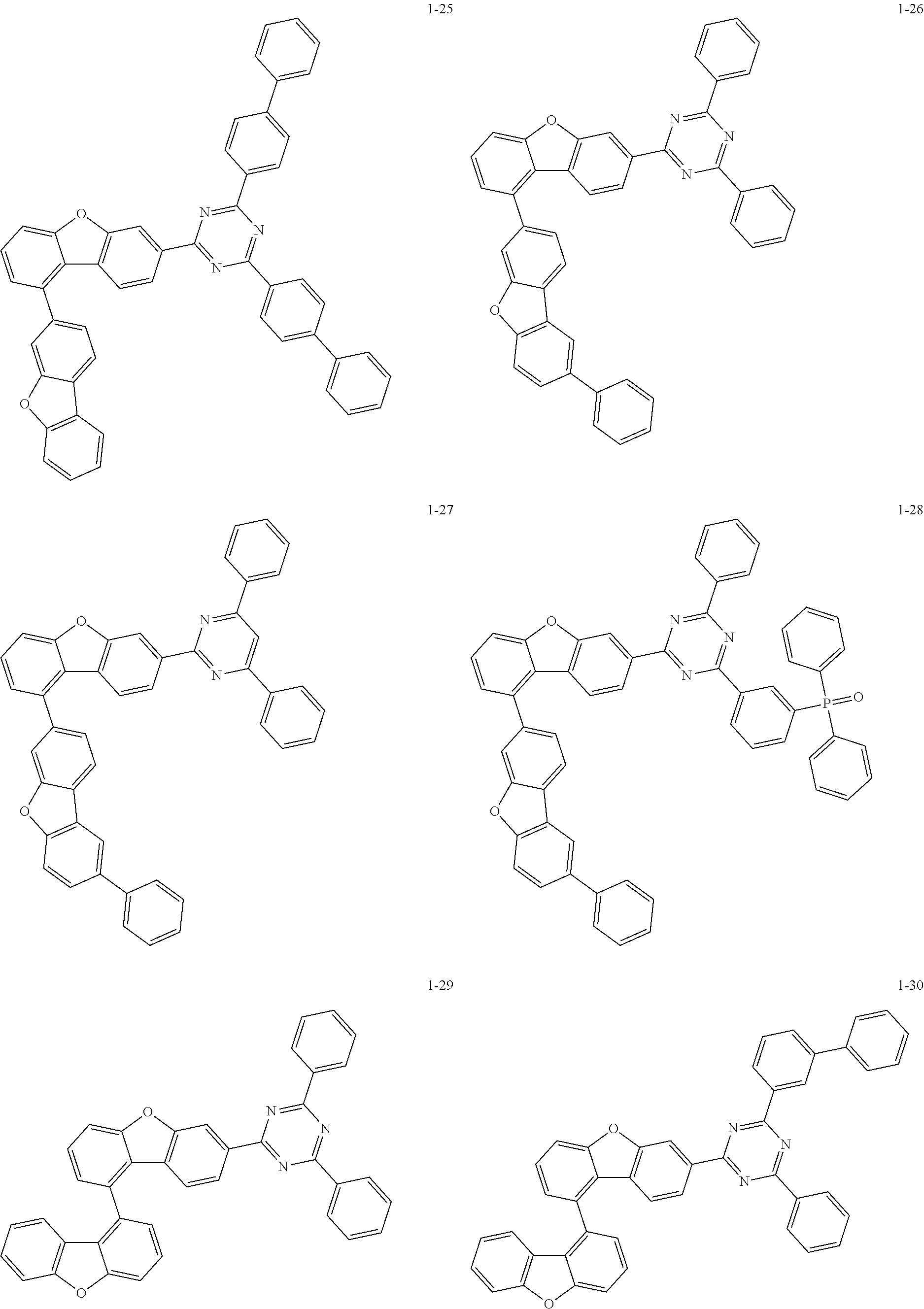

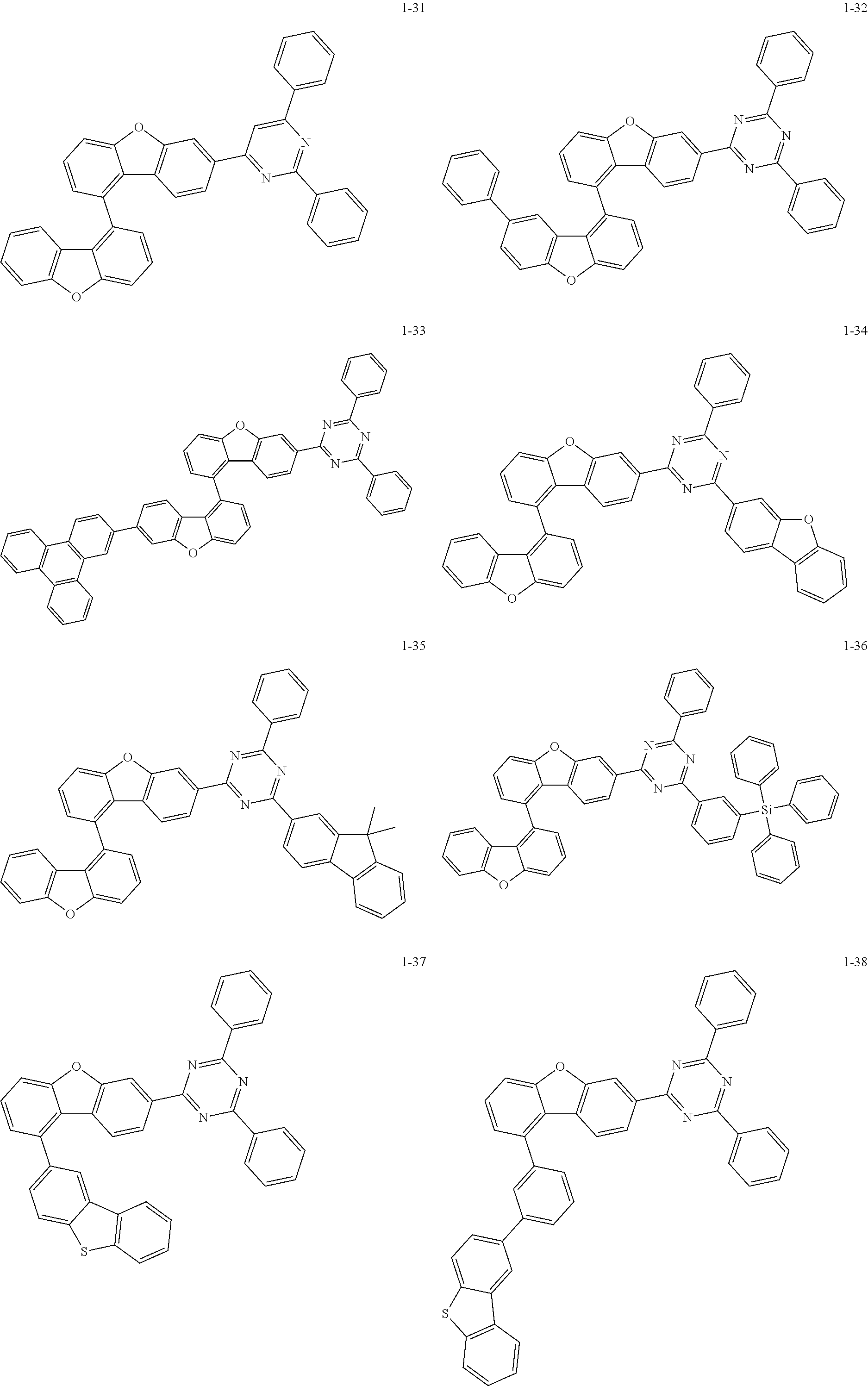

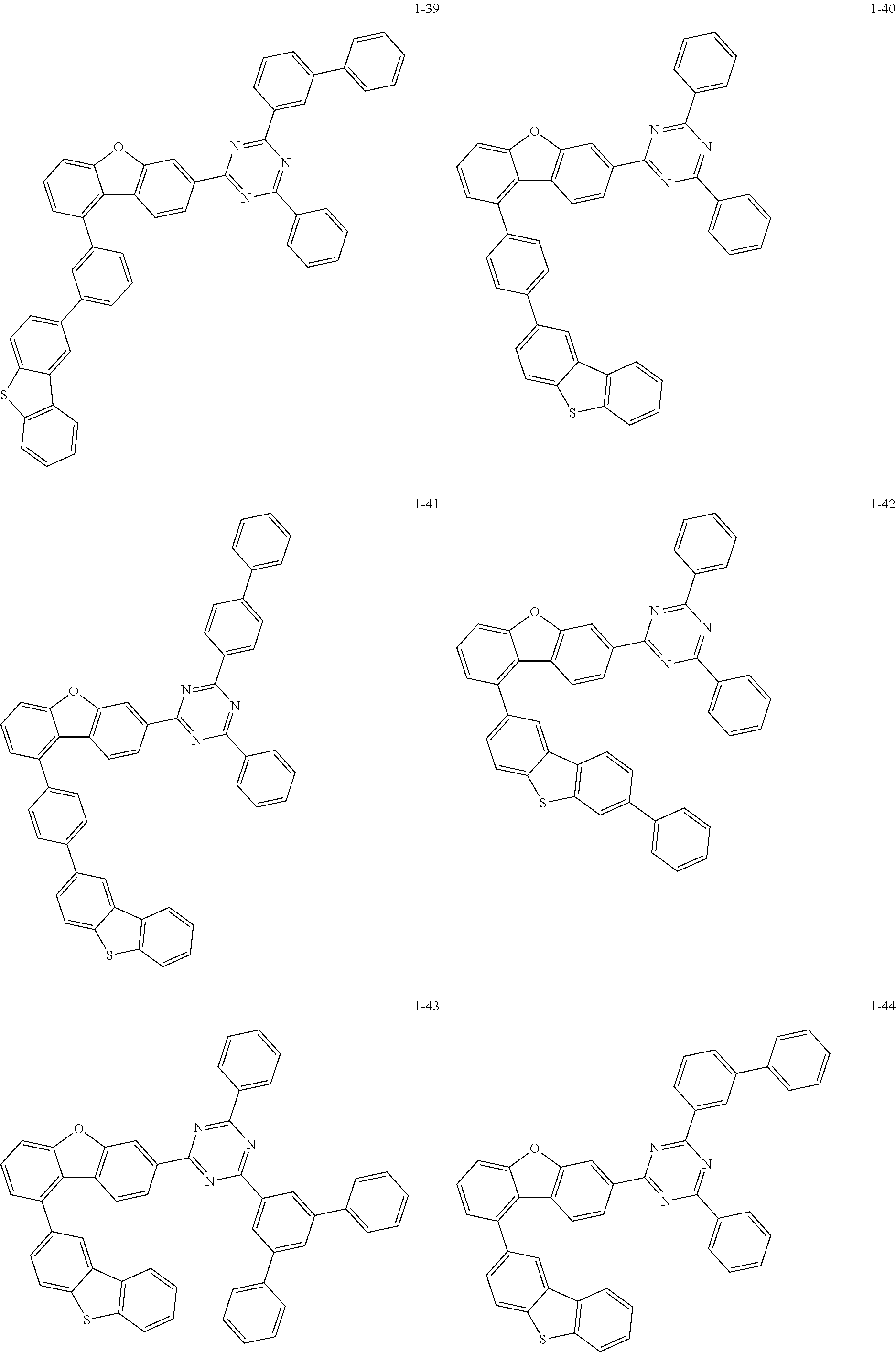

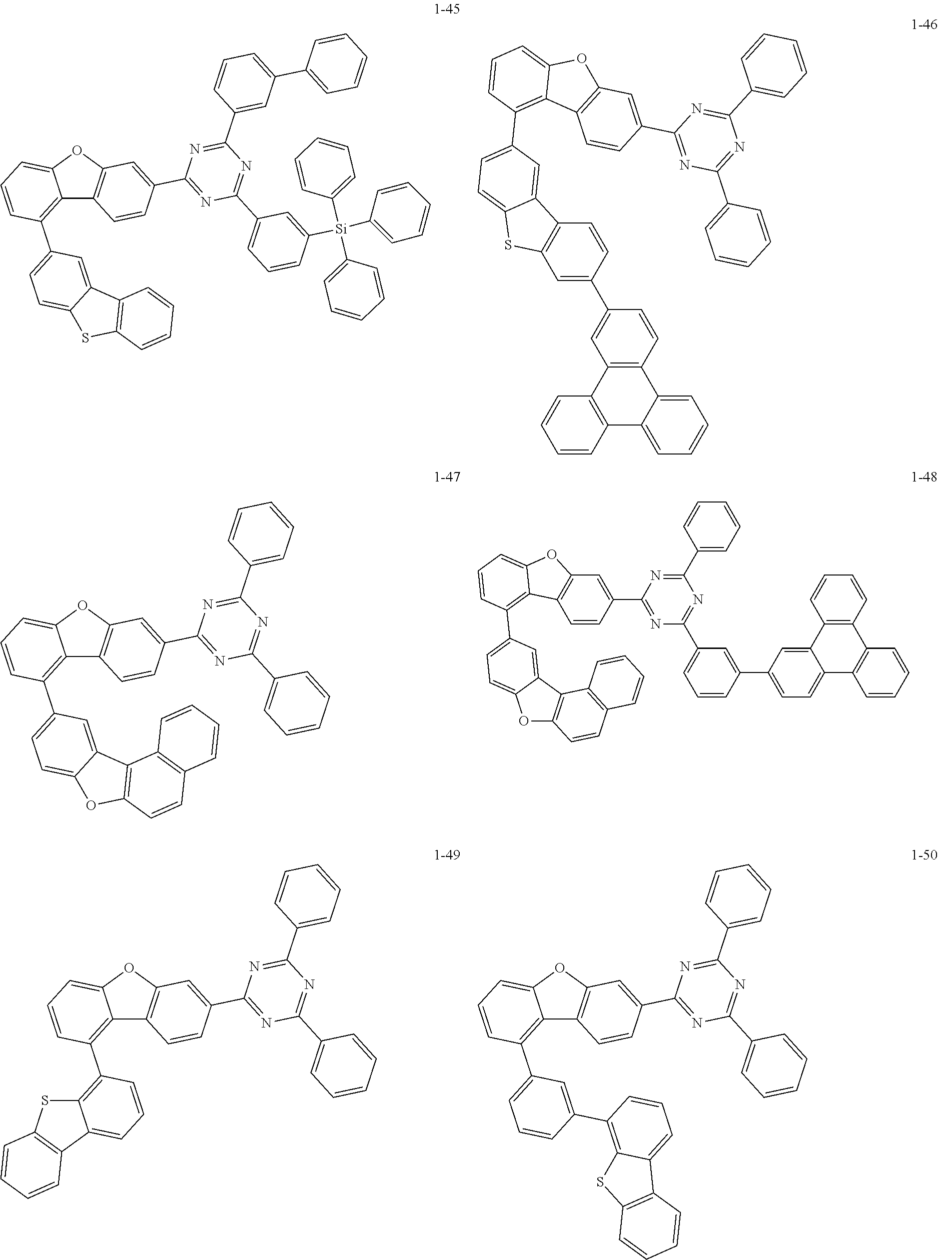

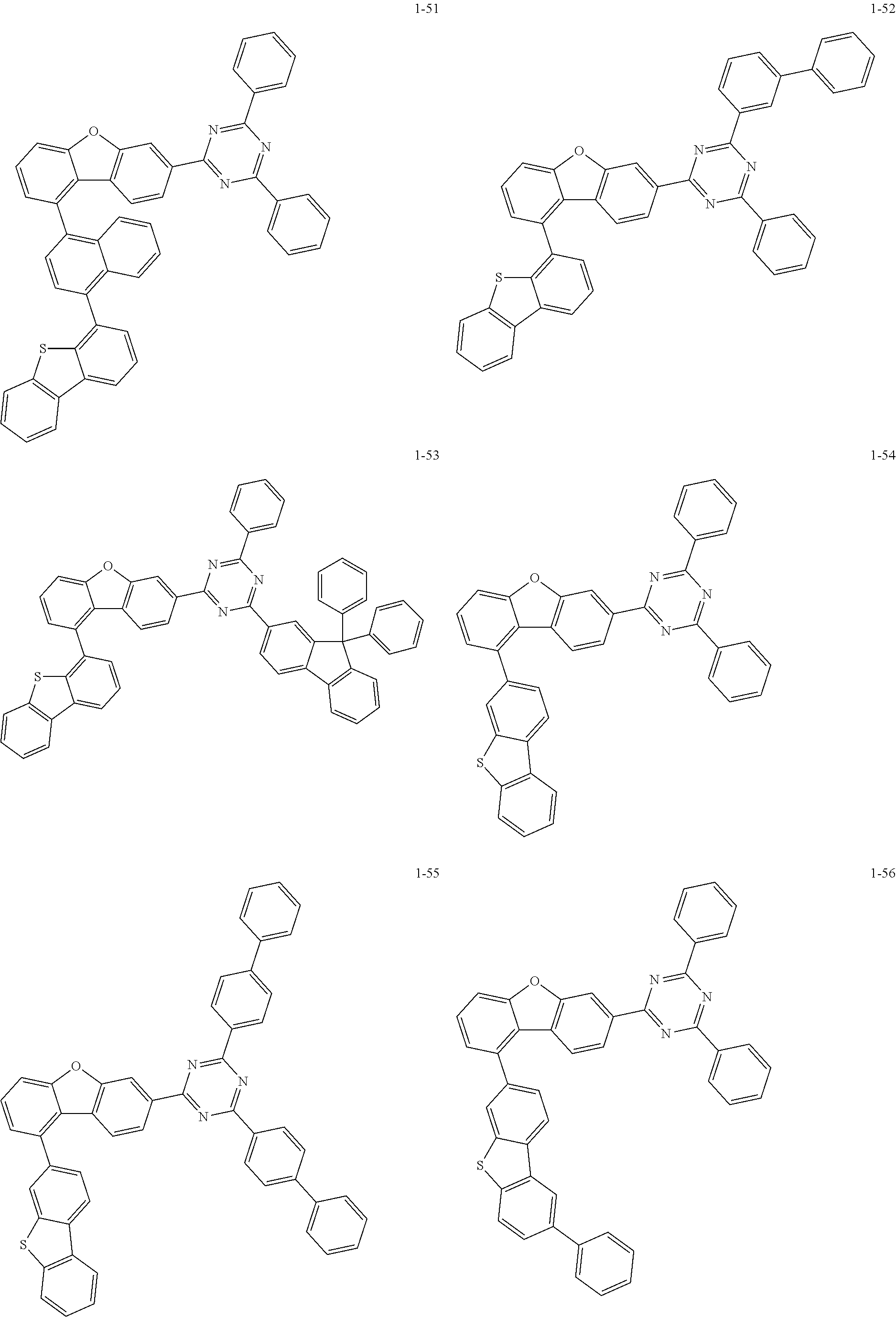

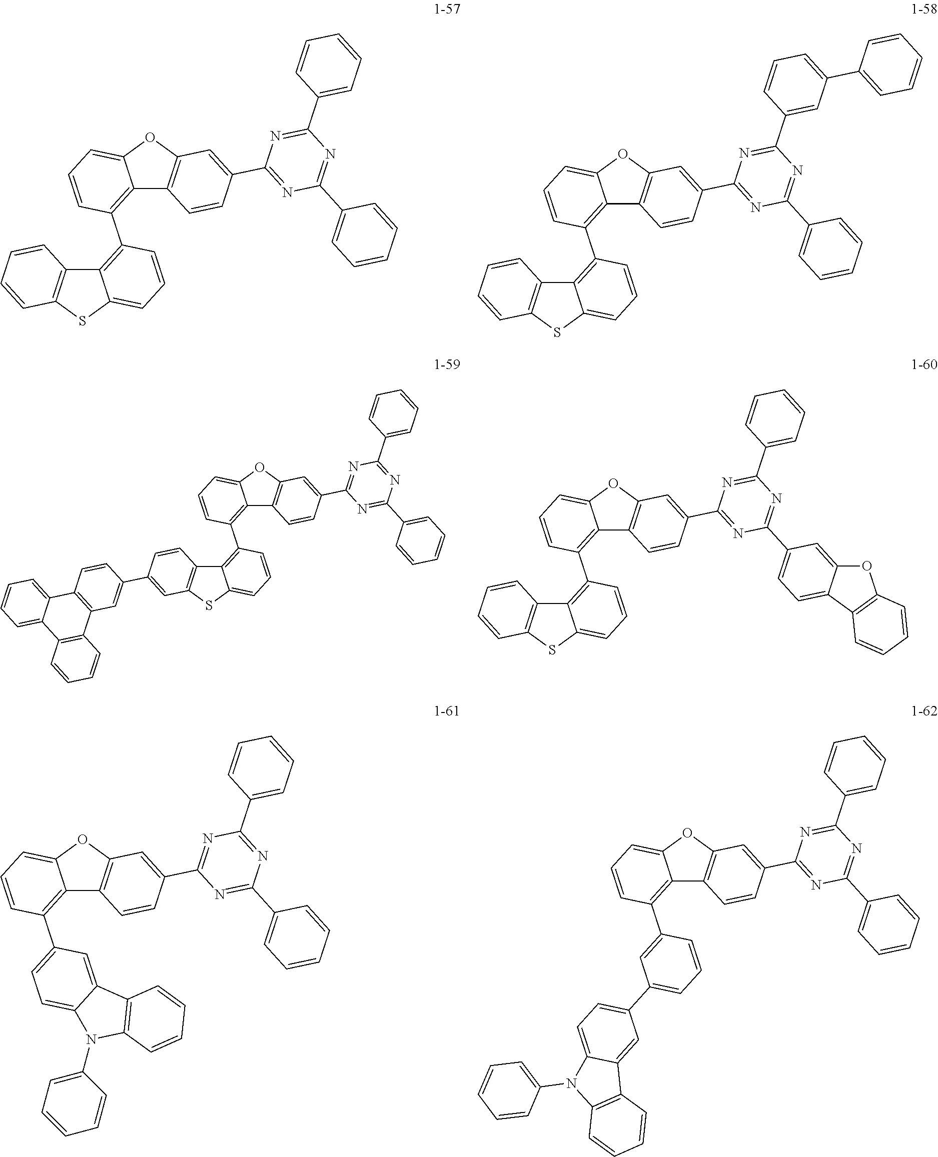

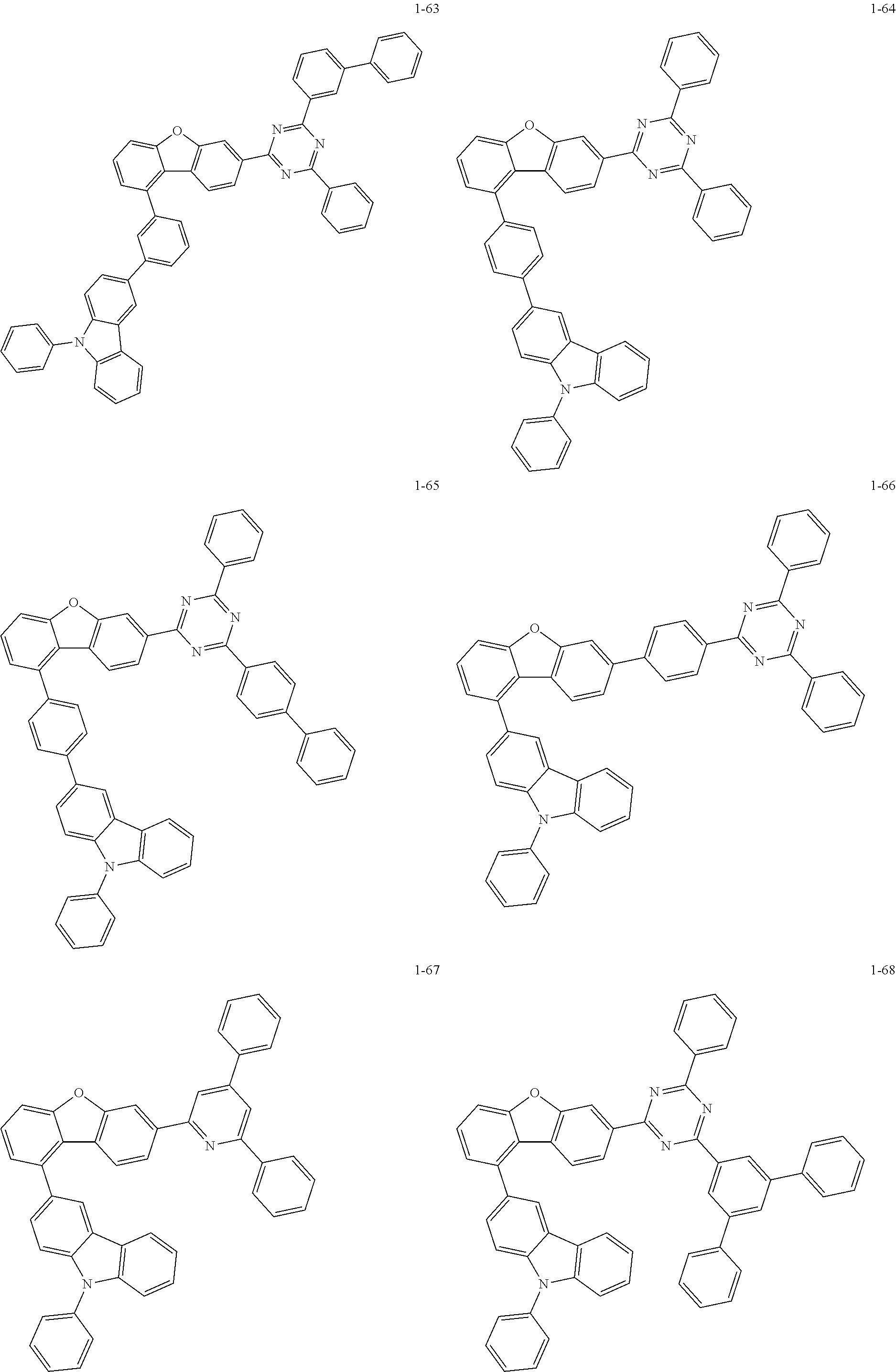

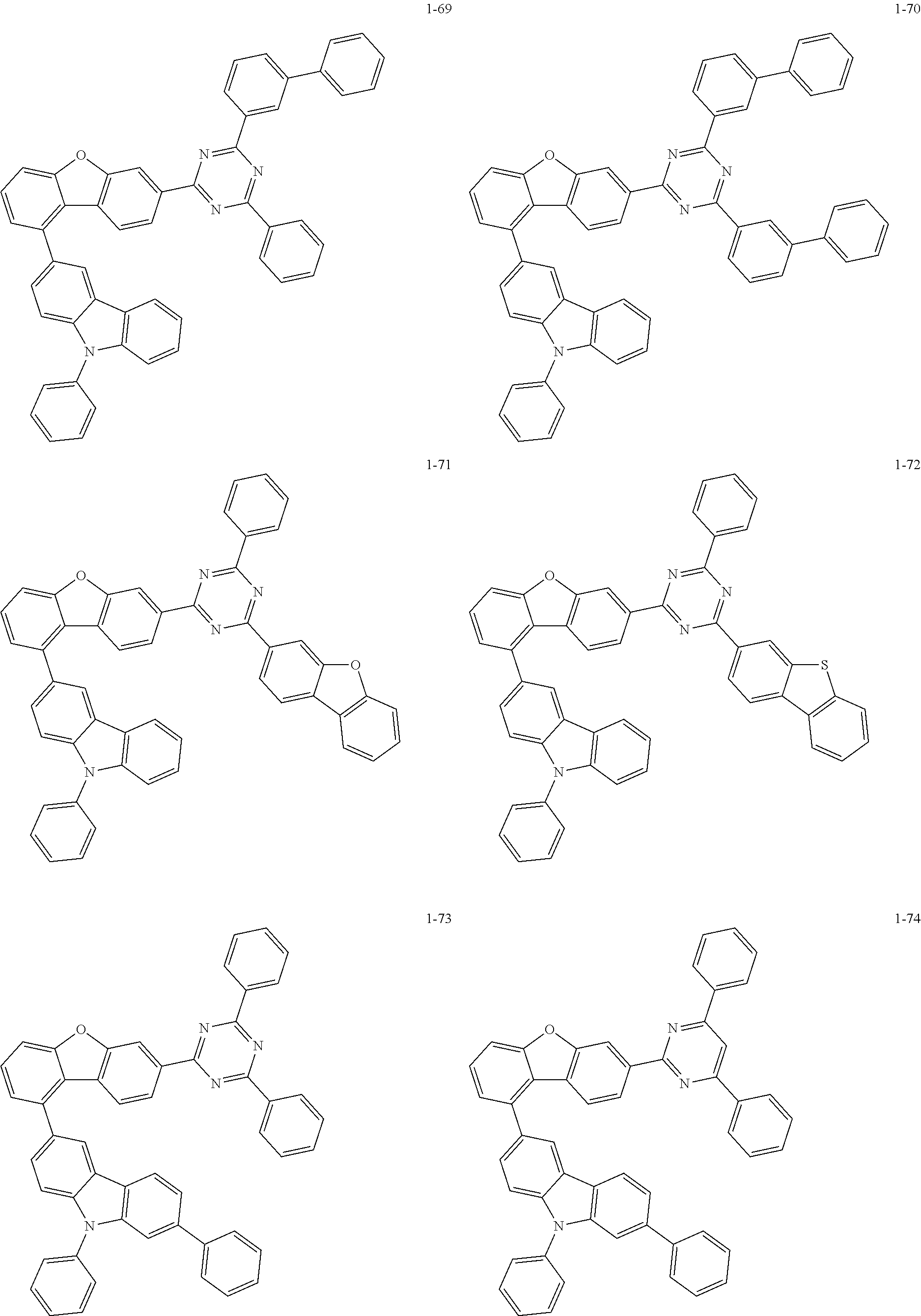

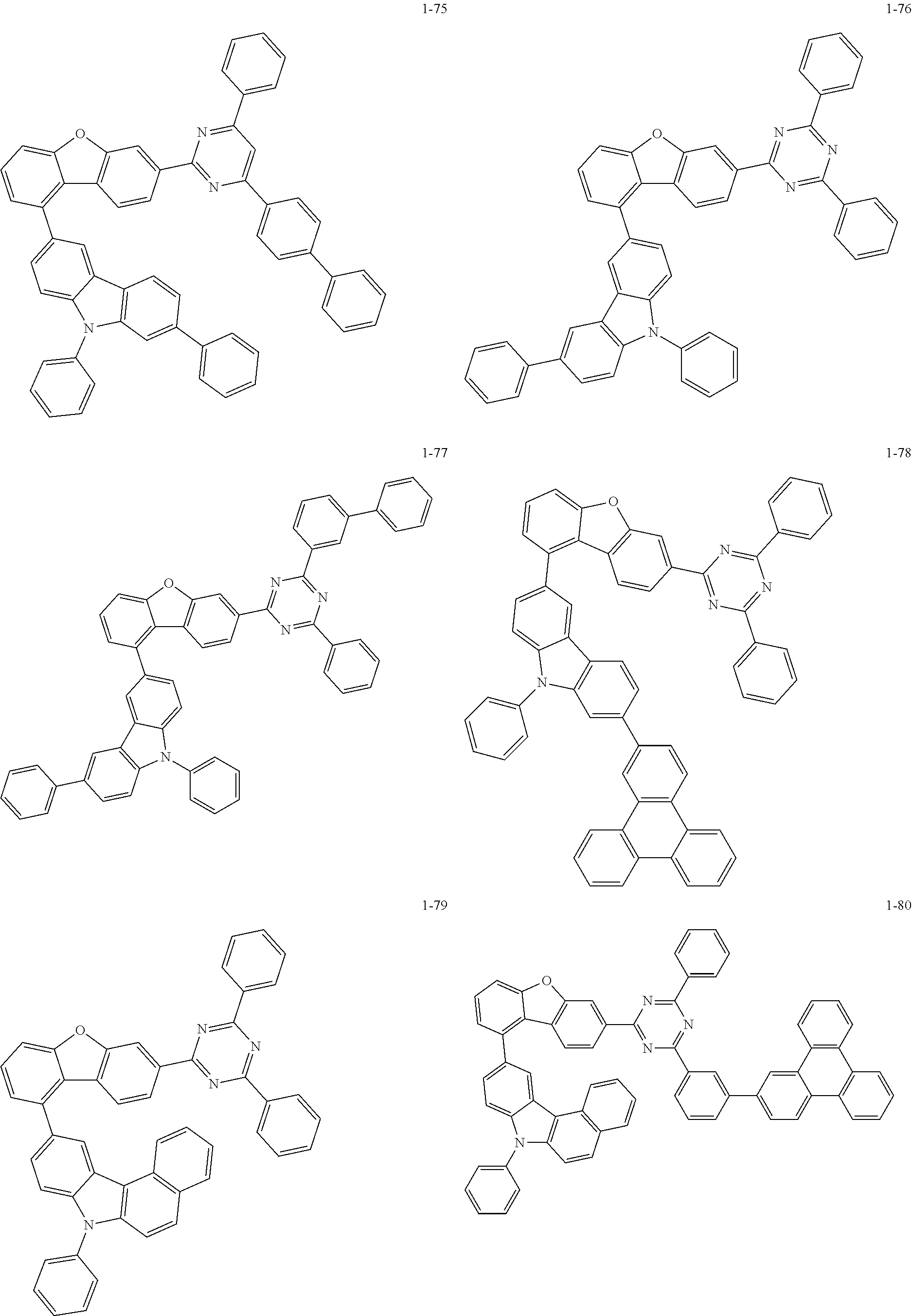

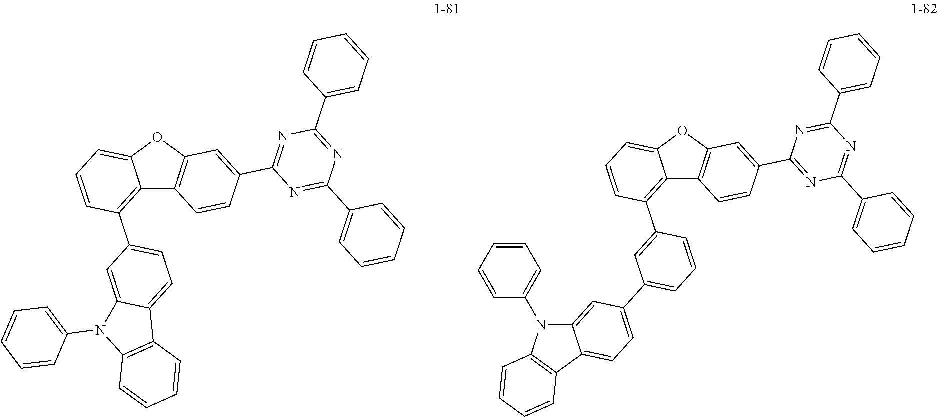 [SMILES: c1ccc(-c2nc(-c3ccccc3)nc(-c3ccc4c(c3)oc3cccc(-c5ccc6c7ccccc7n(-c7ccccc7)c6c5)c34)n2)cc1 ; c1ccc(-c2nc(-c3ccccc3)nc(-c3ccc4c(c3)oc3cccc(-c5cccc(-c6ccc7c8ccccc8n(-c8ccccc8)c7c6)c5)c34)n2)cc1]

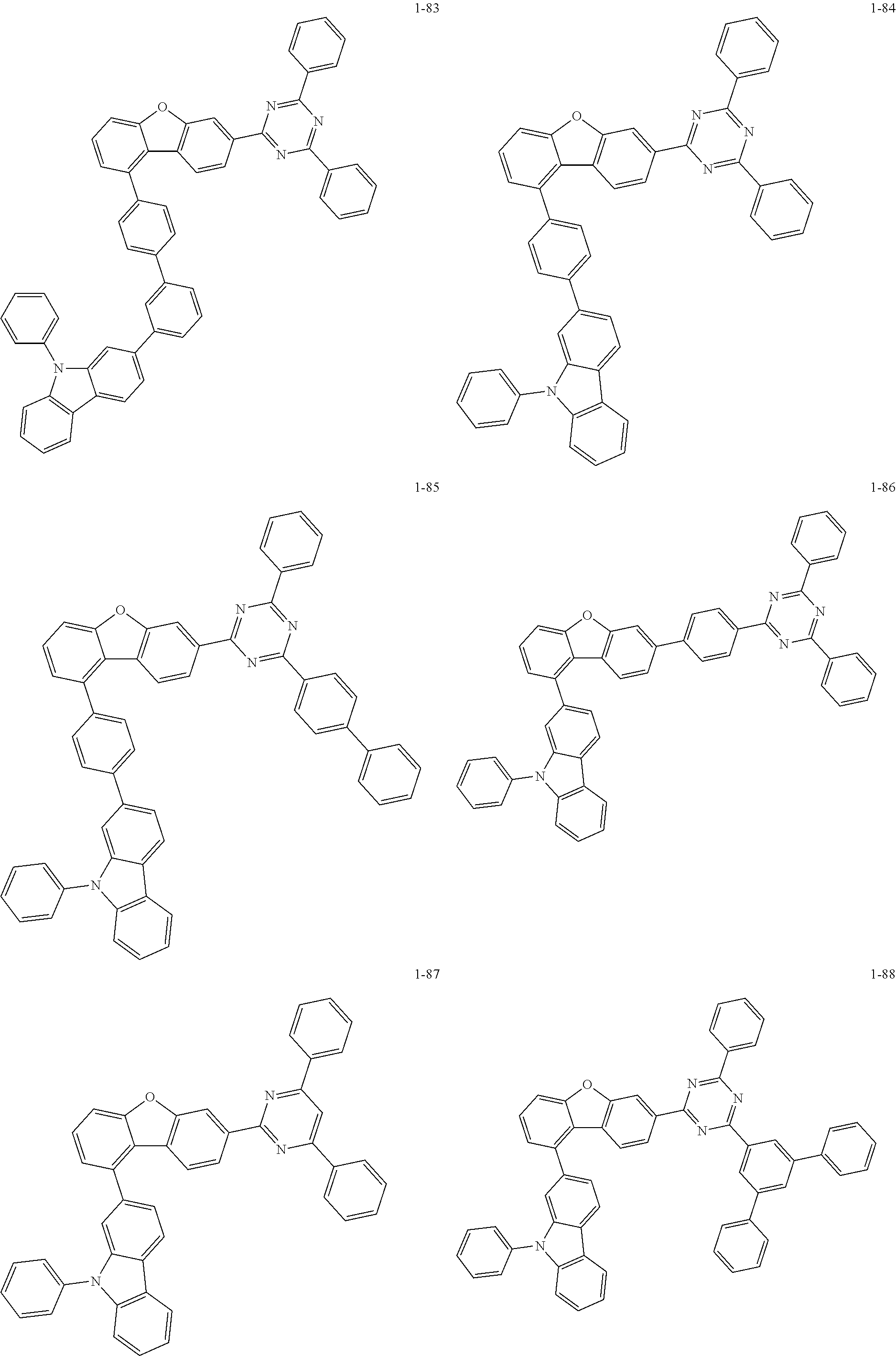

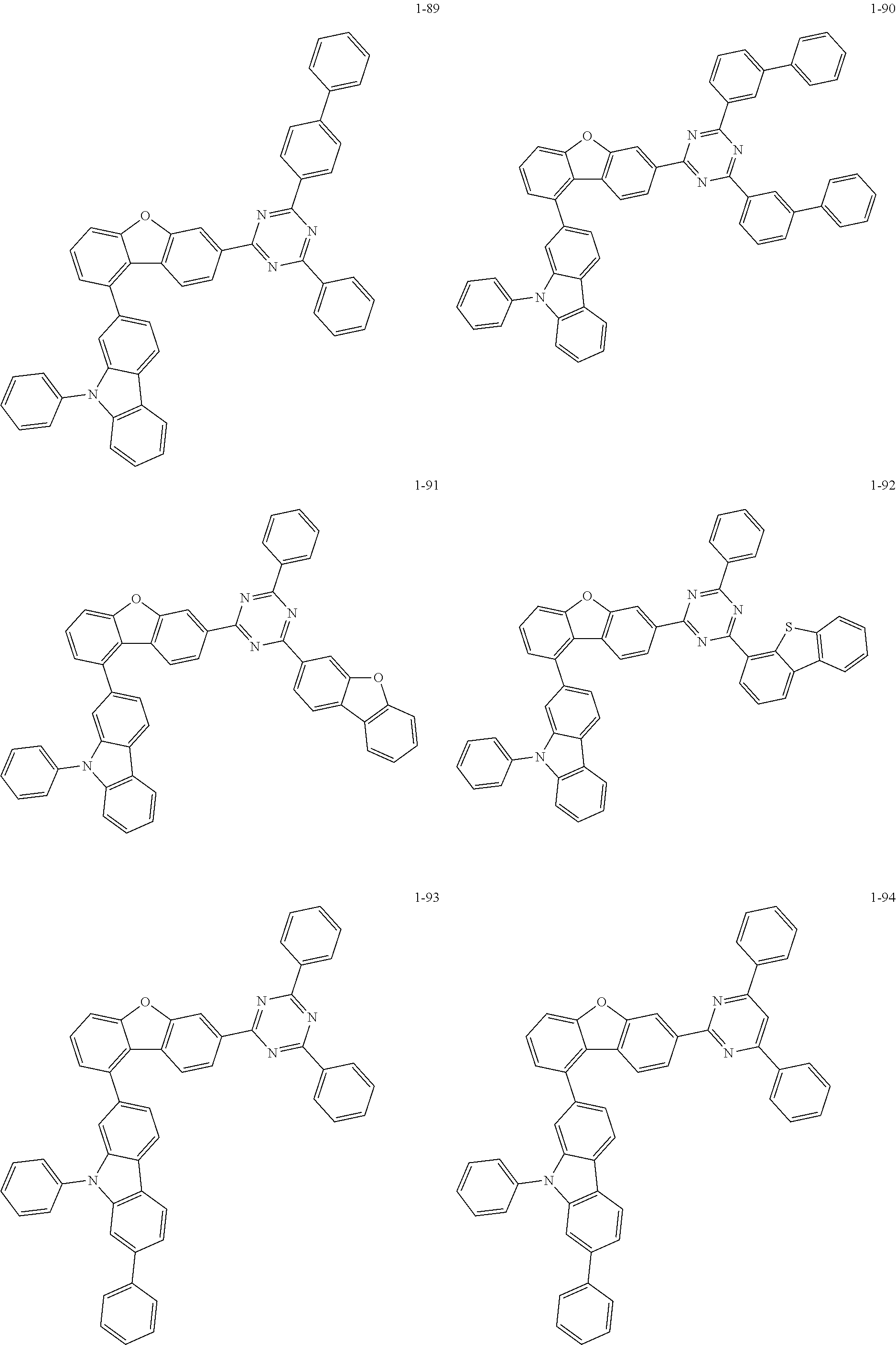

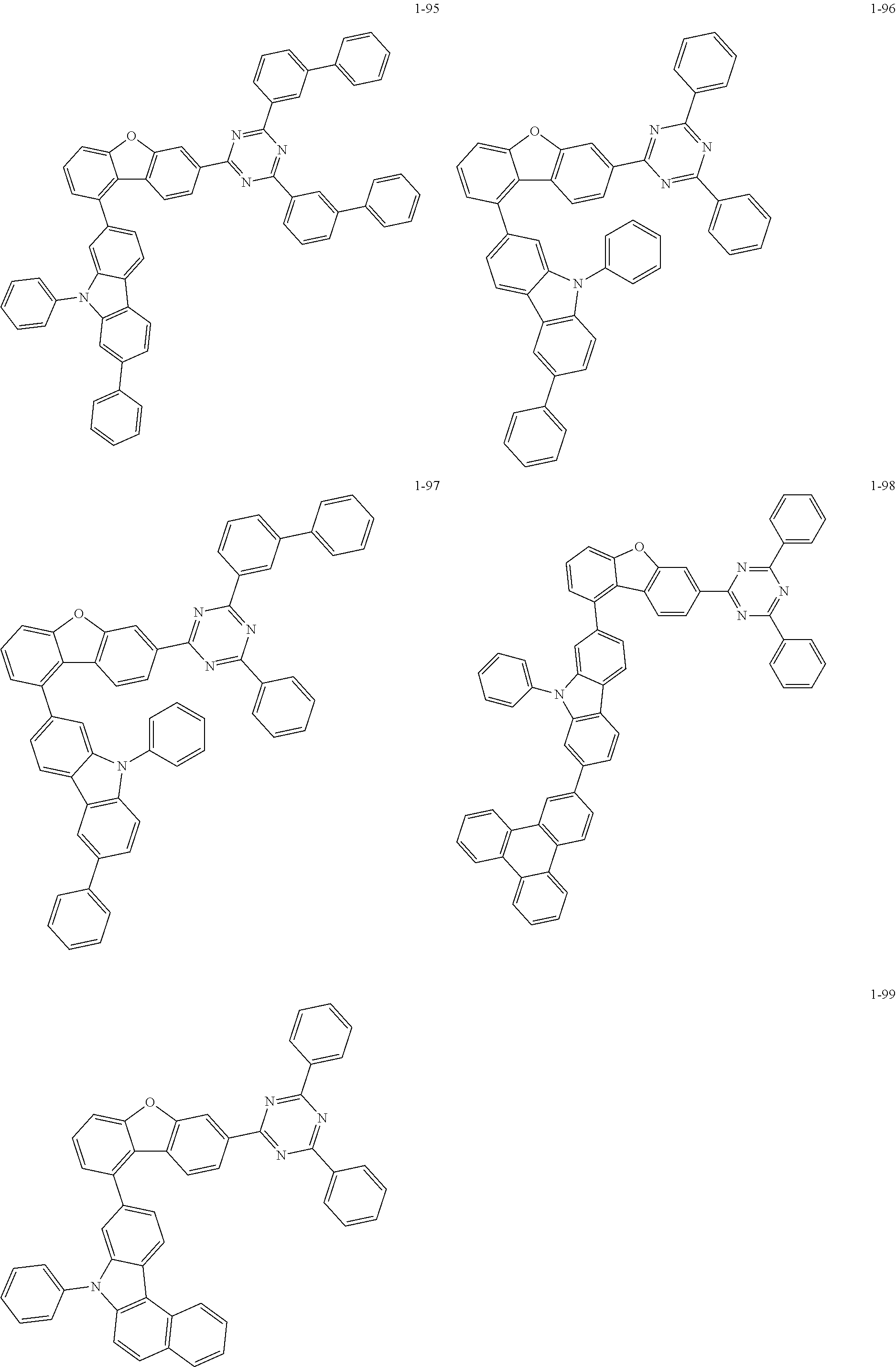

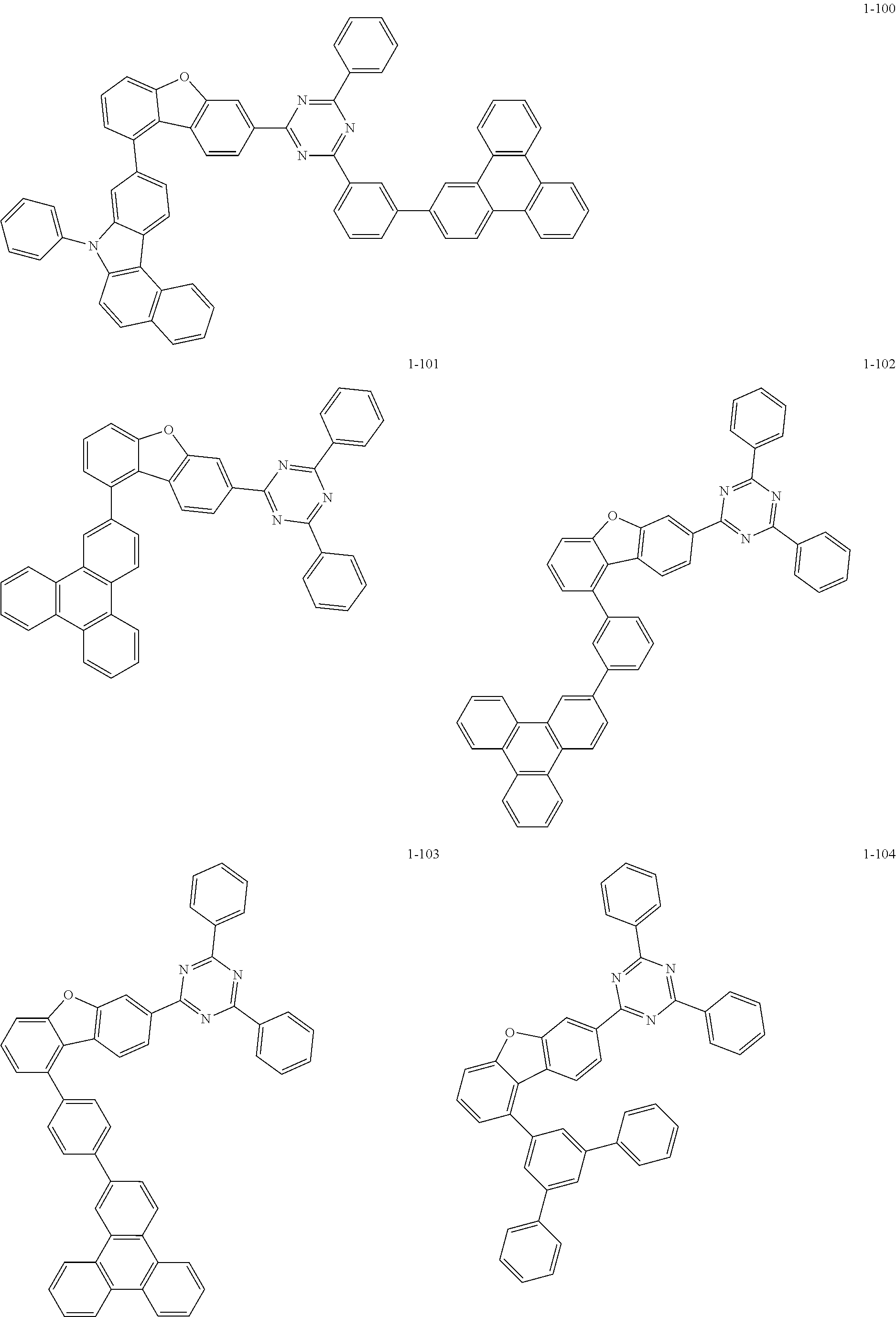 [SMILES: c1ccc(-c2nc(-c3cccc(-c4ccc5c6ccccc6c6ccccc6c5c4)c3)nc(-c3ccc4c(c3)oc3cccc(-c5ccc6c7c8ccccc8ccc7n(-c7ccccc7)c6c5)c34)n2)cc1 ; c1ccc(-c2nc(-c3ccccc3)nc(-c3ccc4c(c3)oc3cccc(-c5ccc6c7ccccc7c7ccccc7c6c5)c34)n2)cc1 ; c1ccc(-c2nc(-c3ccccc3)nc(-c3ccc4c(c3)oc3cccc(-c5cccc(-c6ccc7c8ccccc8c8ccccc8c7c6)c5)c34)n2)cc1 ; c1ccc(-c2nc(-c3ccccc3)nc(-c3ccc4c(c3)oc3cccc(-c5ccc(-c6ccc7c8ccccc8c8ccccc8c7c6)cc5)c34)n2)cc1 ; c1ccc(-c2cc(-c3ccccc3)cc(-c3cccc4oc5cc(-c6nc(-c7ccccc7)nc(-c7ccccc7)n6)ccc5c34)c2)cc1]

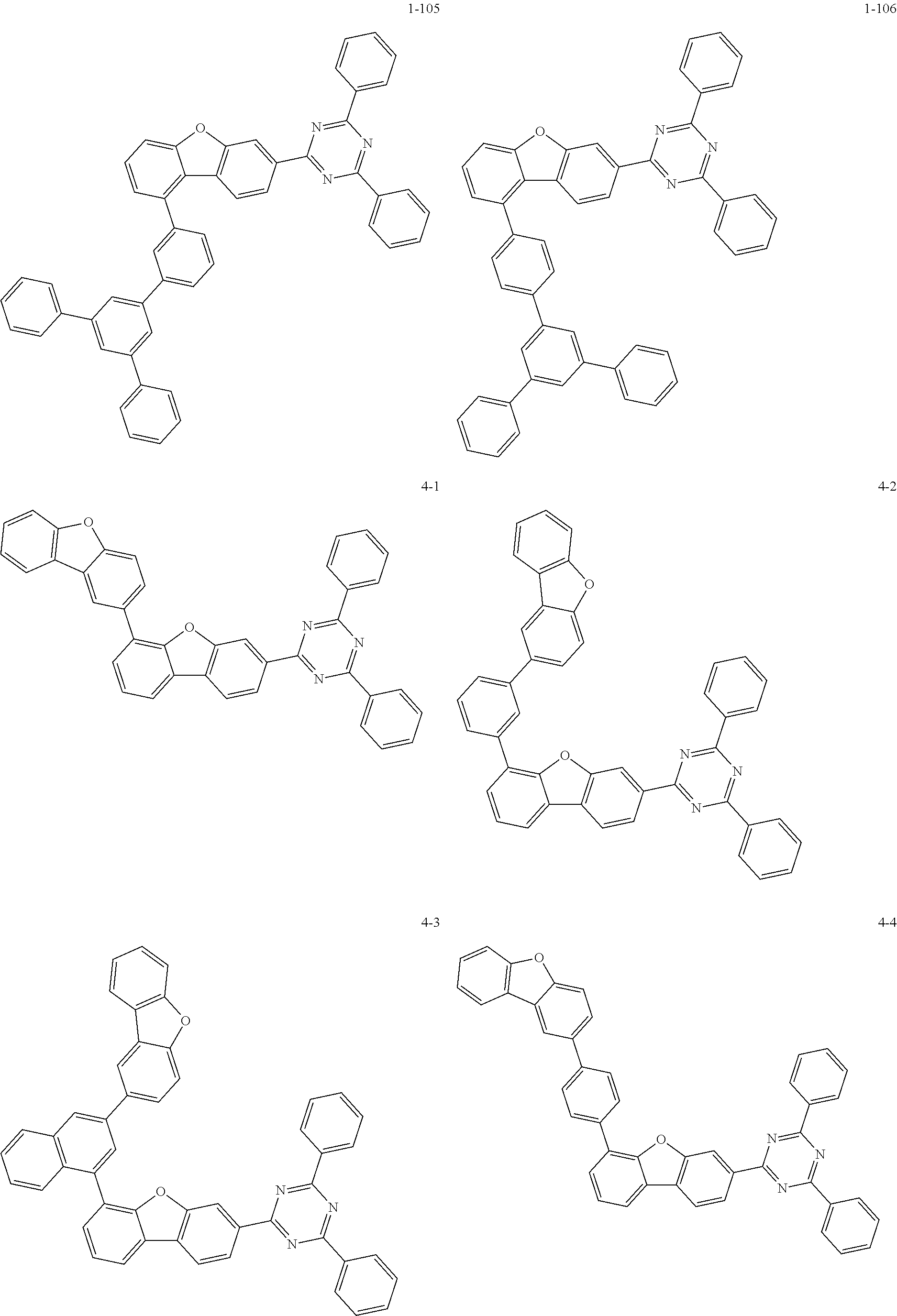

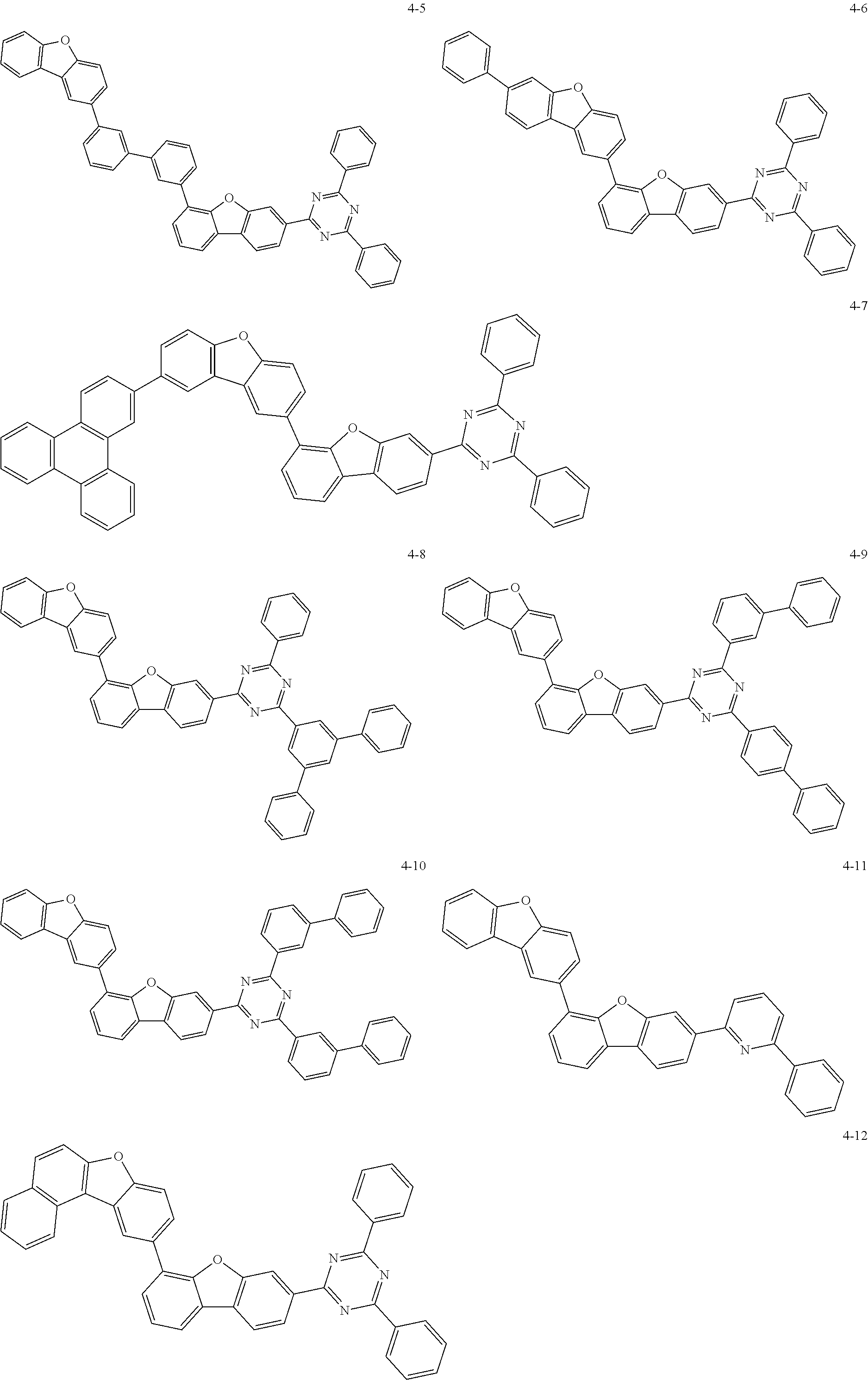

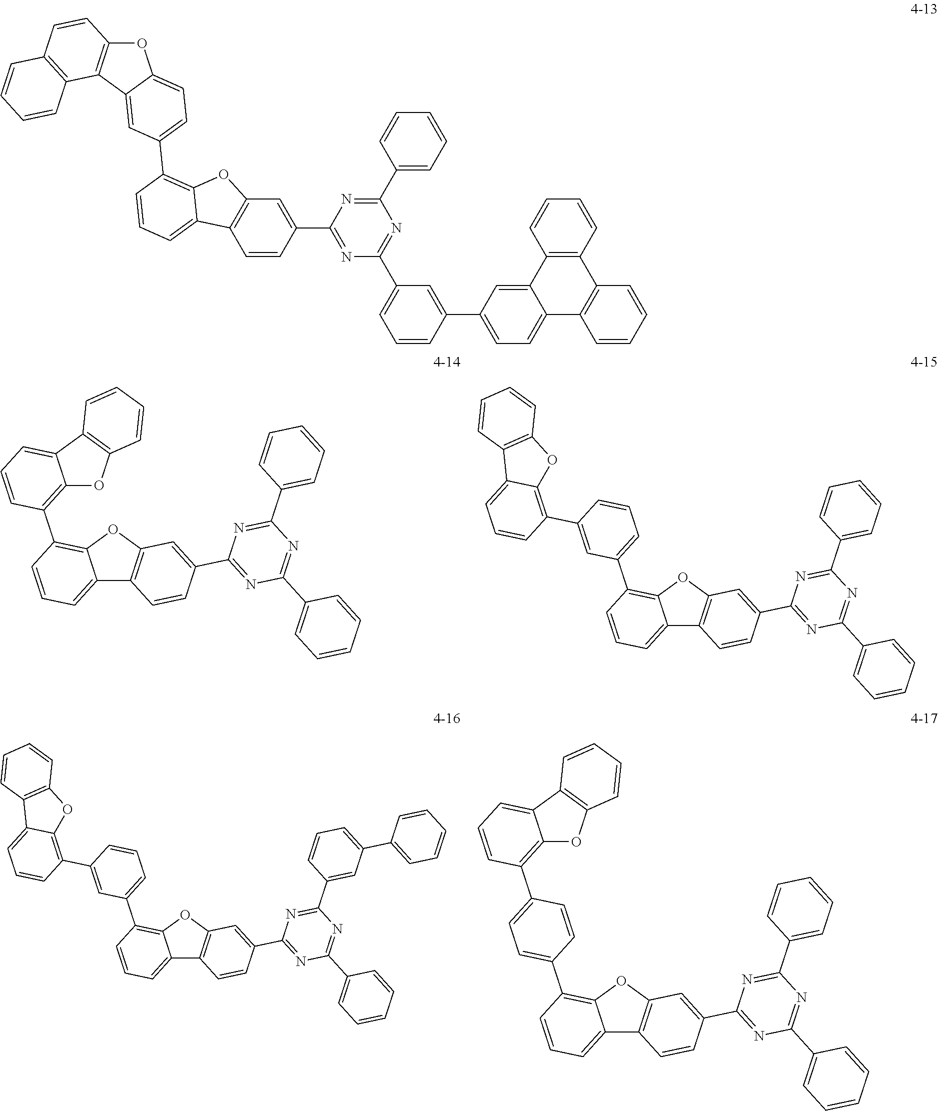

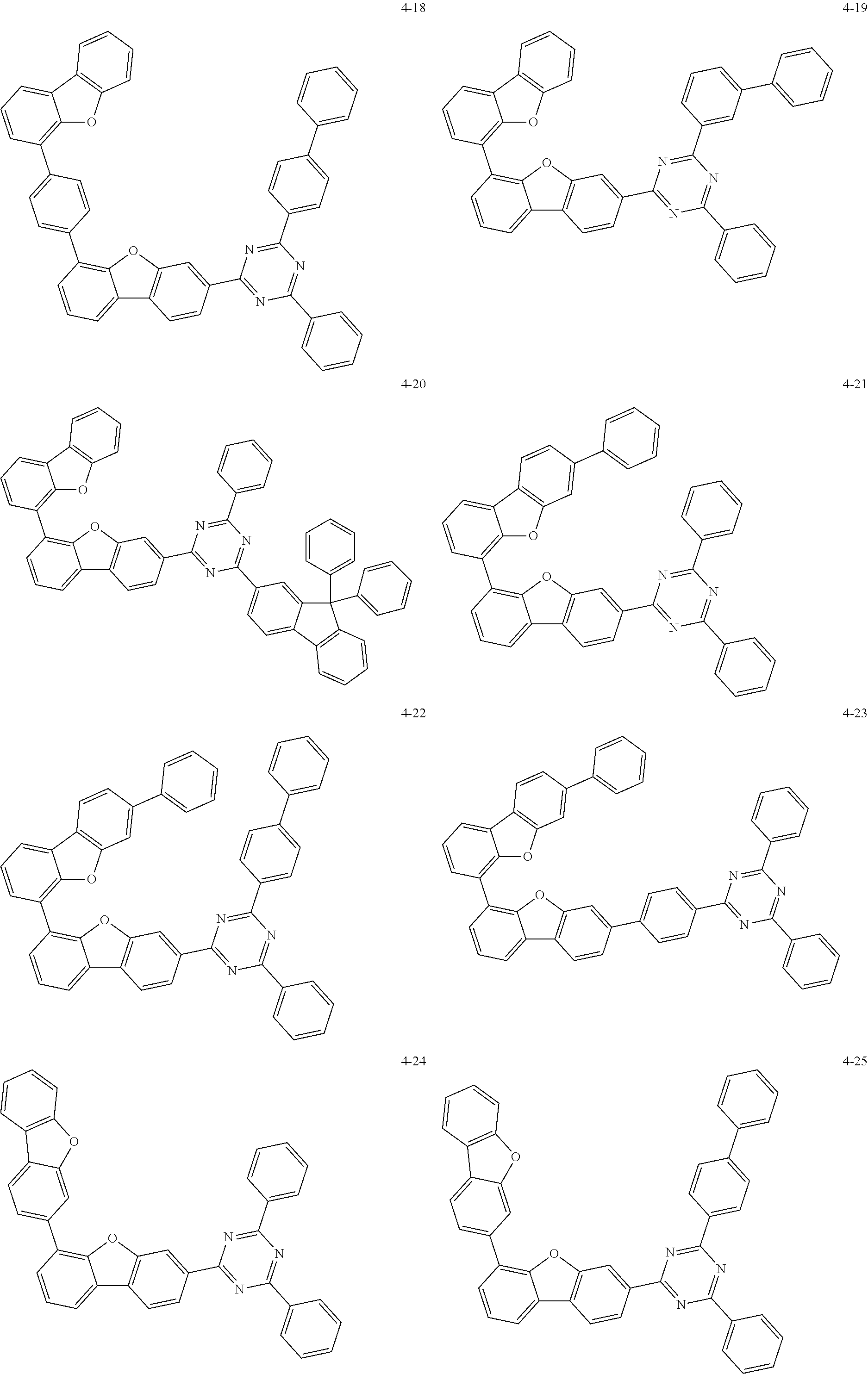

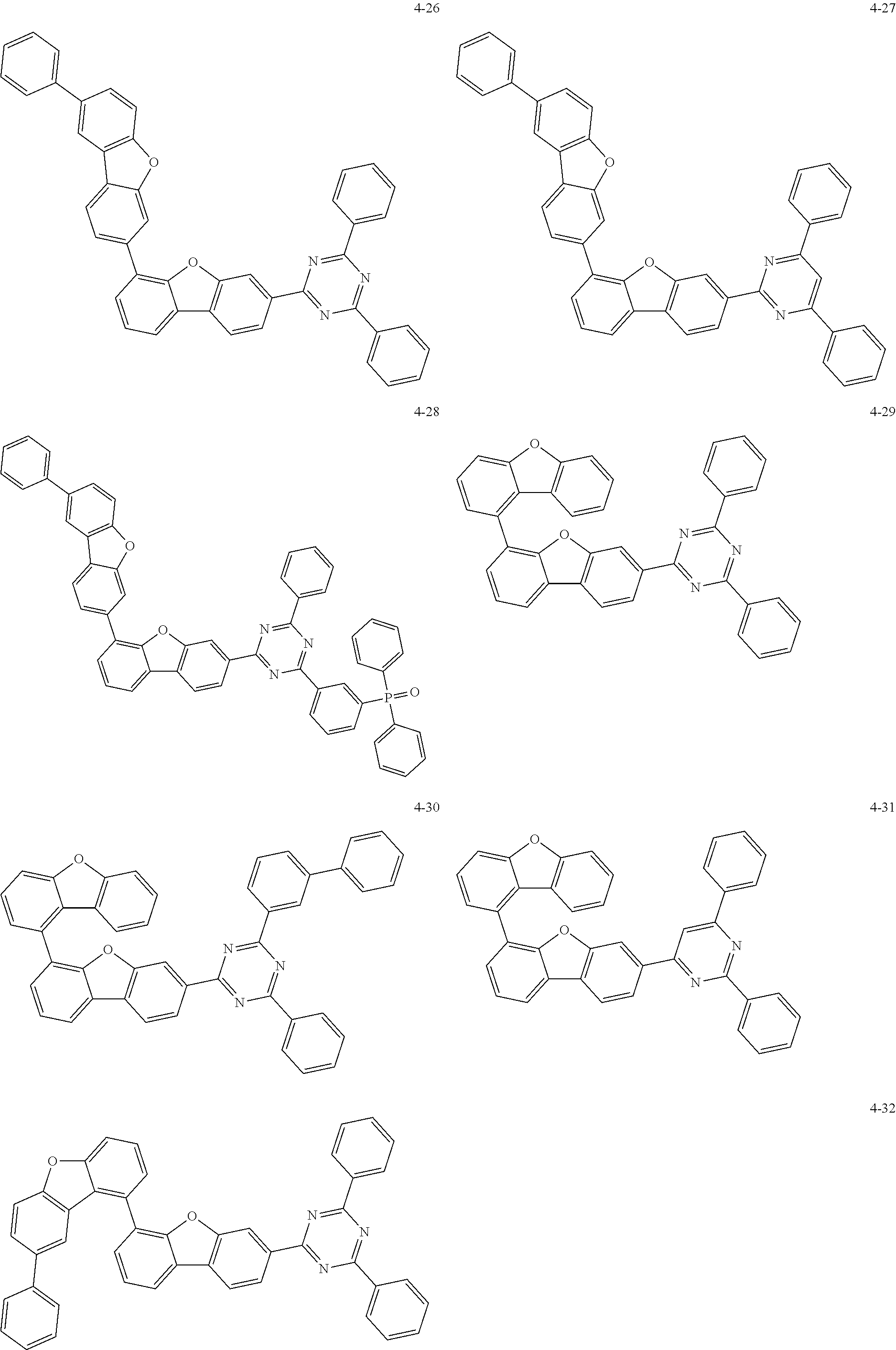

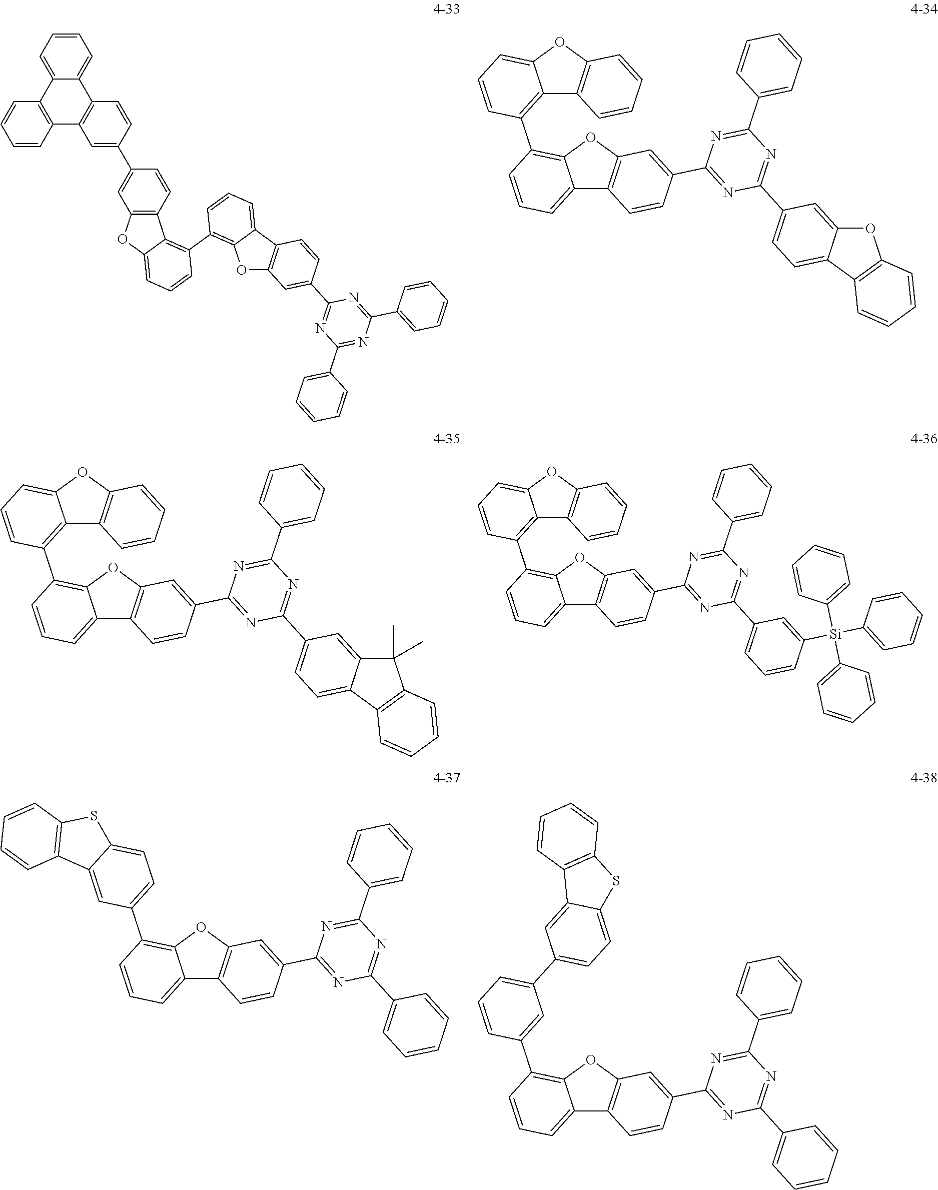

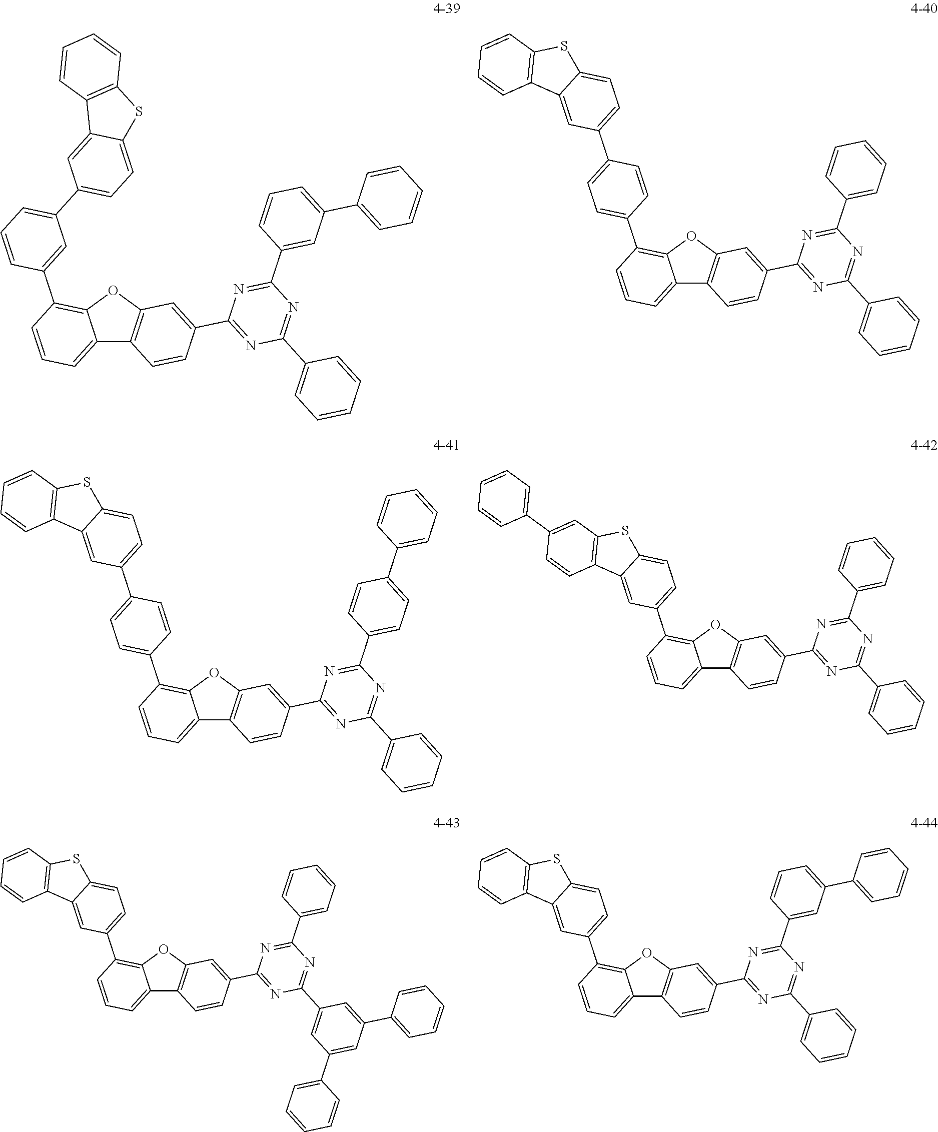

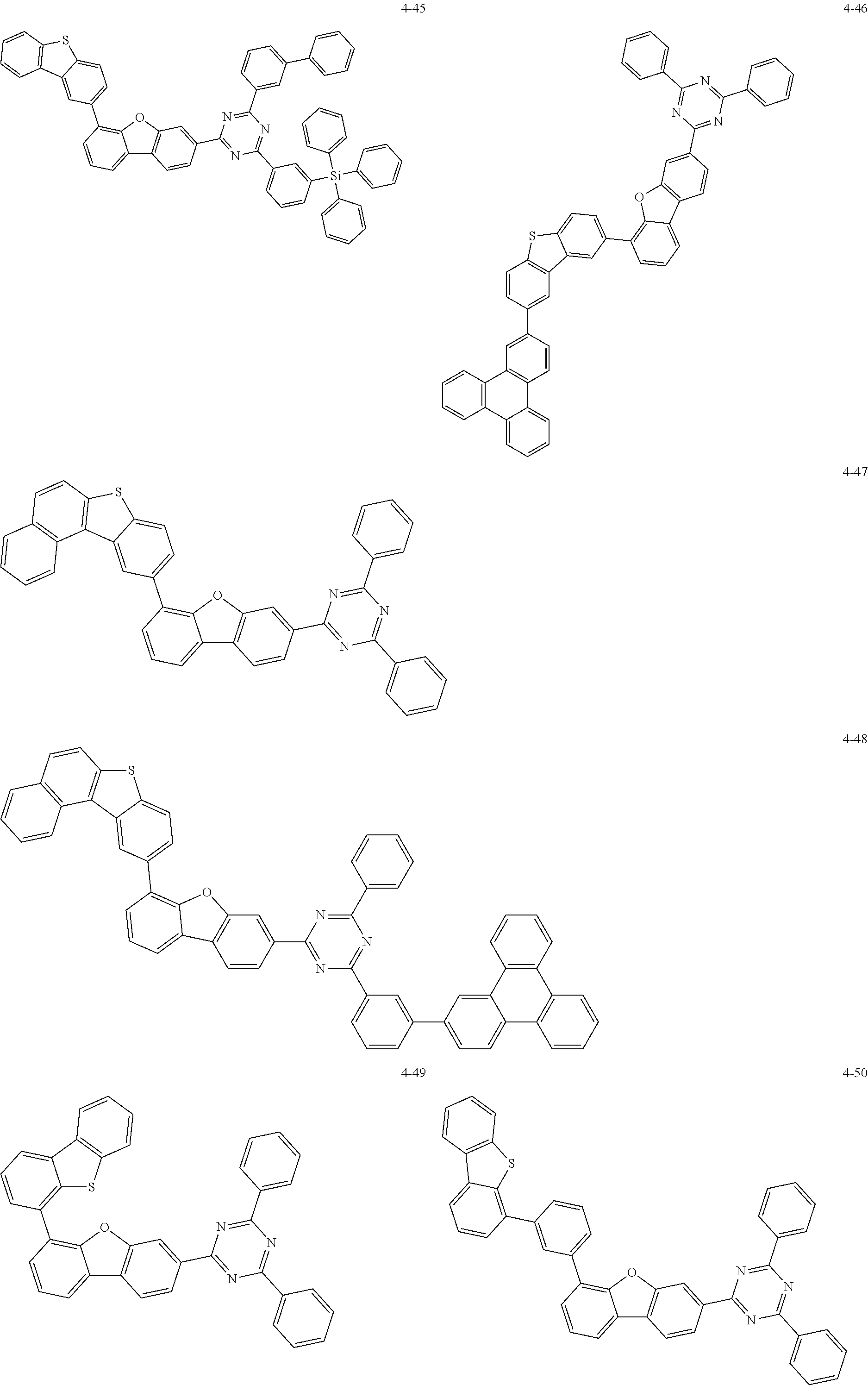

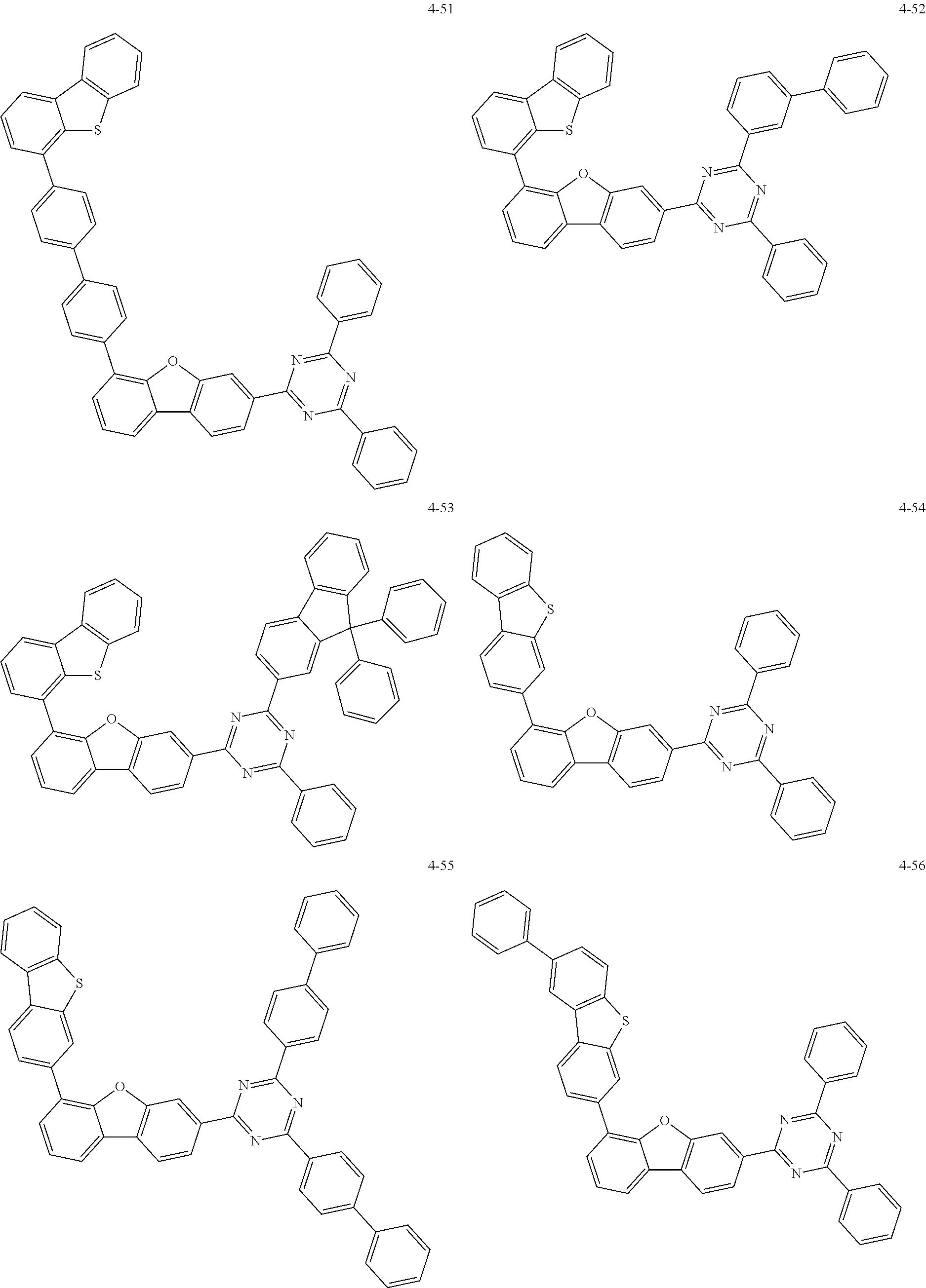

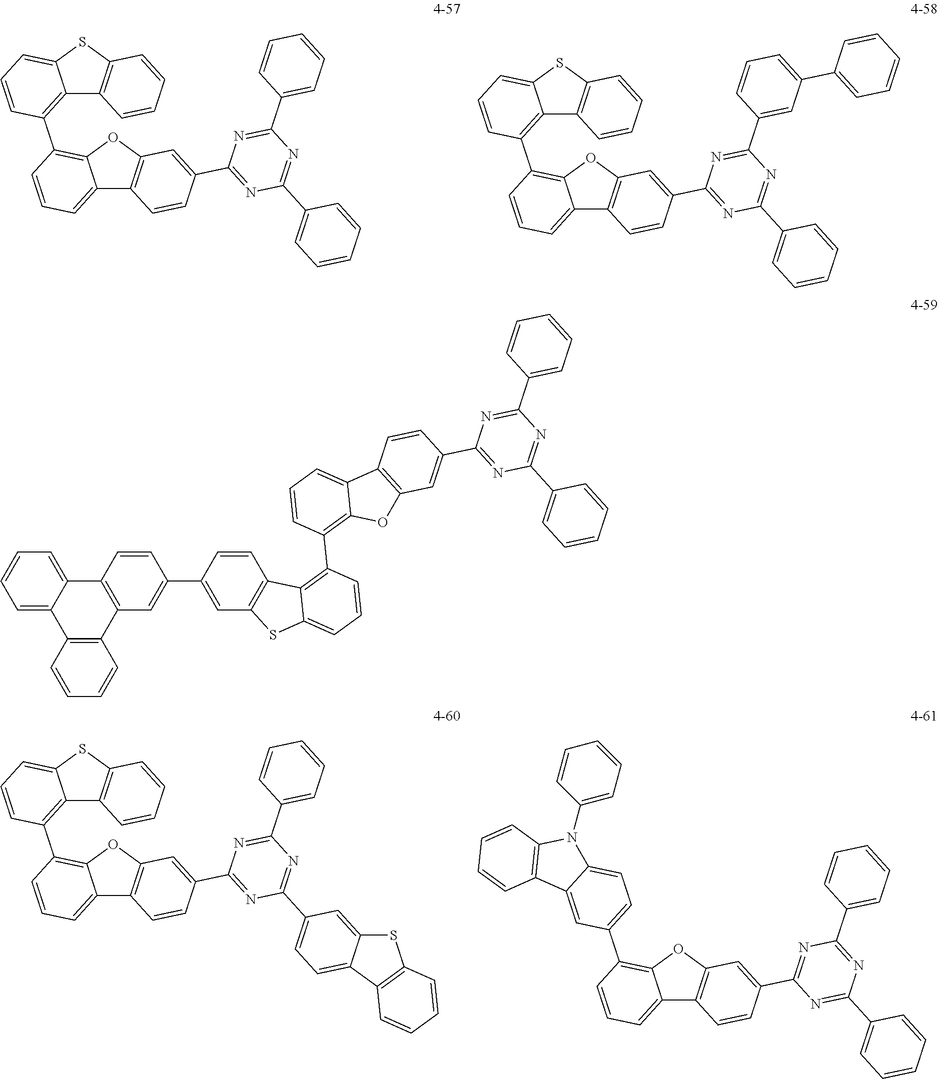 [SMILES: c1ccc(-c2nc(-c3ccccc3)nc(-c3ccc4c(c3)oc3c(-c5cccc6sc7ccccc7c56)cccc34)n2)cc1 ; c1ccc(-c2cccc(-c3nc(-c4ccccc4)nc(-c4ccc5c(c4)oc4c(-c6cccc7sc8ccccc8c67)cccc45)n3)c2)cc1 ; c1ccc(-c2nc(-c3ccccc3)nc(-c3ccc4c(c3)oc3c(-c5cccc6sc7cc(-c8ccc9c%10ccccc%10c%10ccccc%10c9c8)ccc7c56)cccc34)n2)cc1 ; c1ccc(-c2nc(-c3ccc4c(c3)oc3c(-c5cccc6sc7ccccc7c56)cccc34)nc(-c3ccc4c(c3)sc3ccccc34)n2)cc1 ; c1ccc(-c2nc(-c3ccccc3)nc(-c3ccc4c(c3)oc3c(-c5ccc6c(c5)c5ccccc5n6-c5ccccc5)cccc34)n2)cc1]

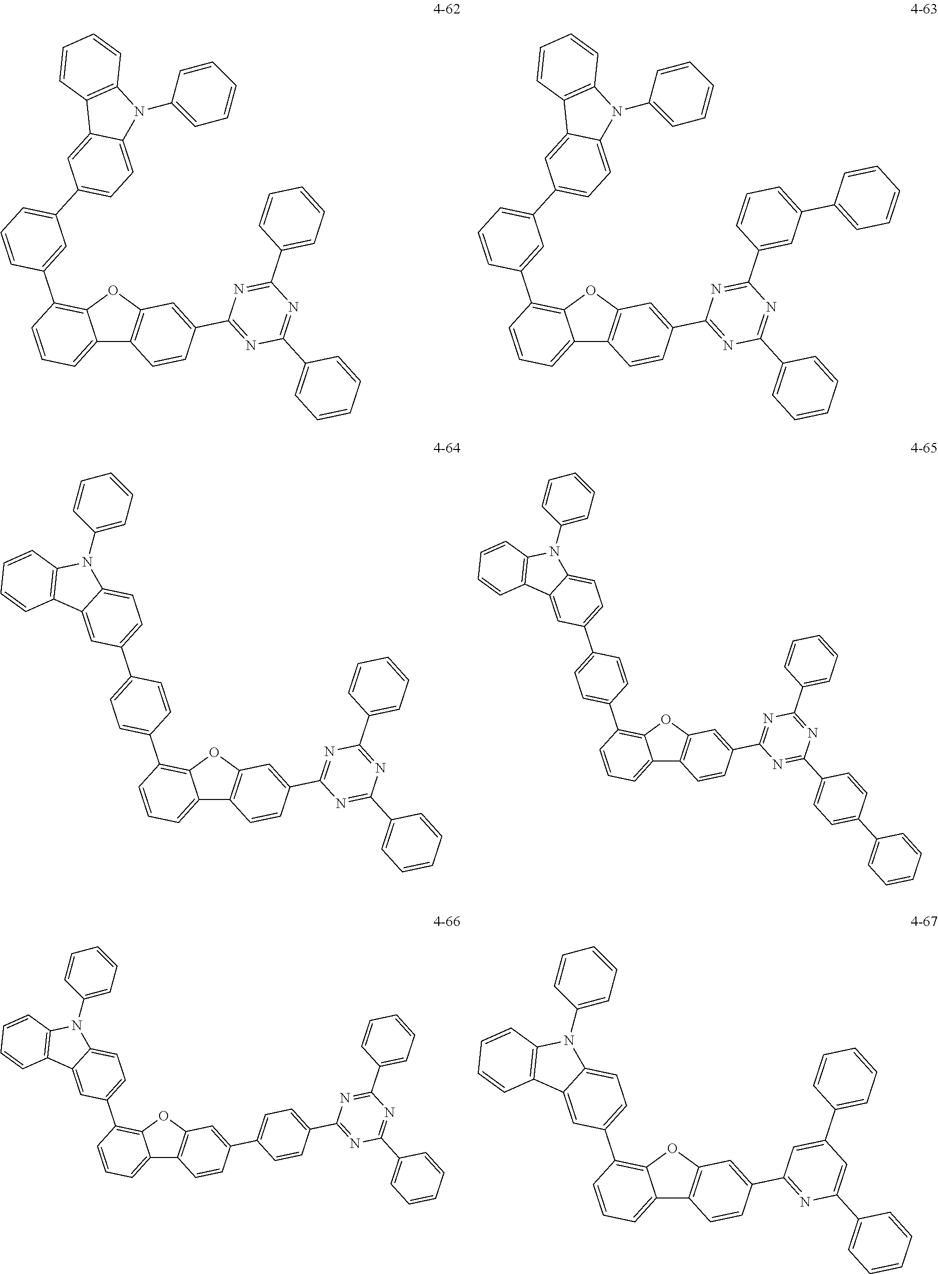

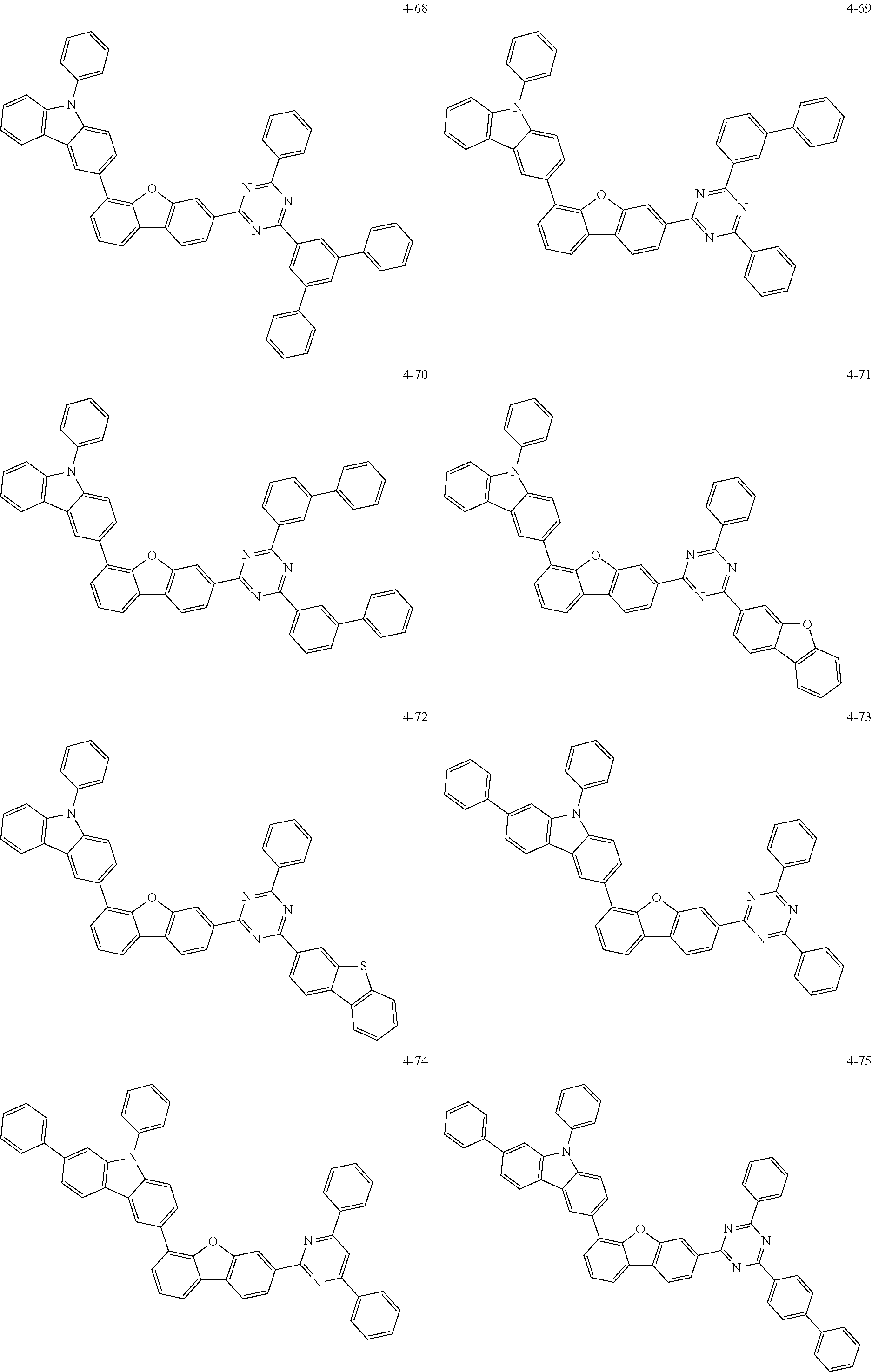

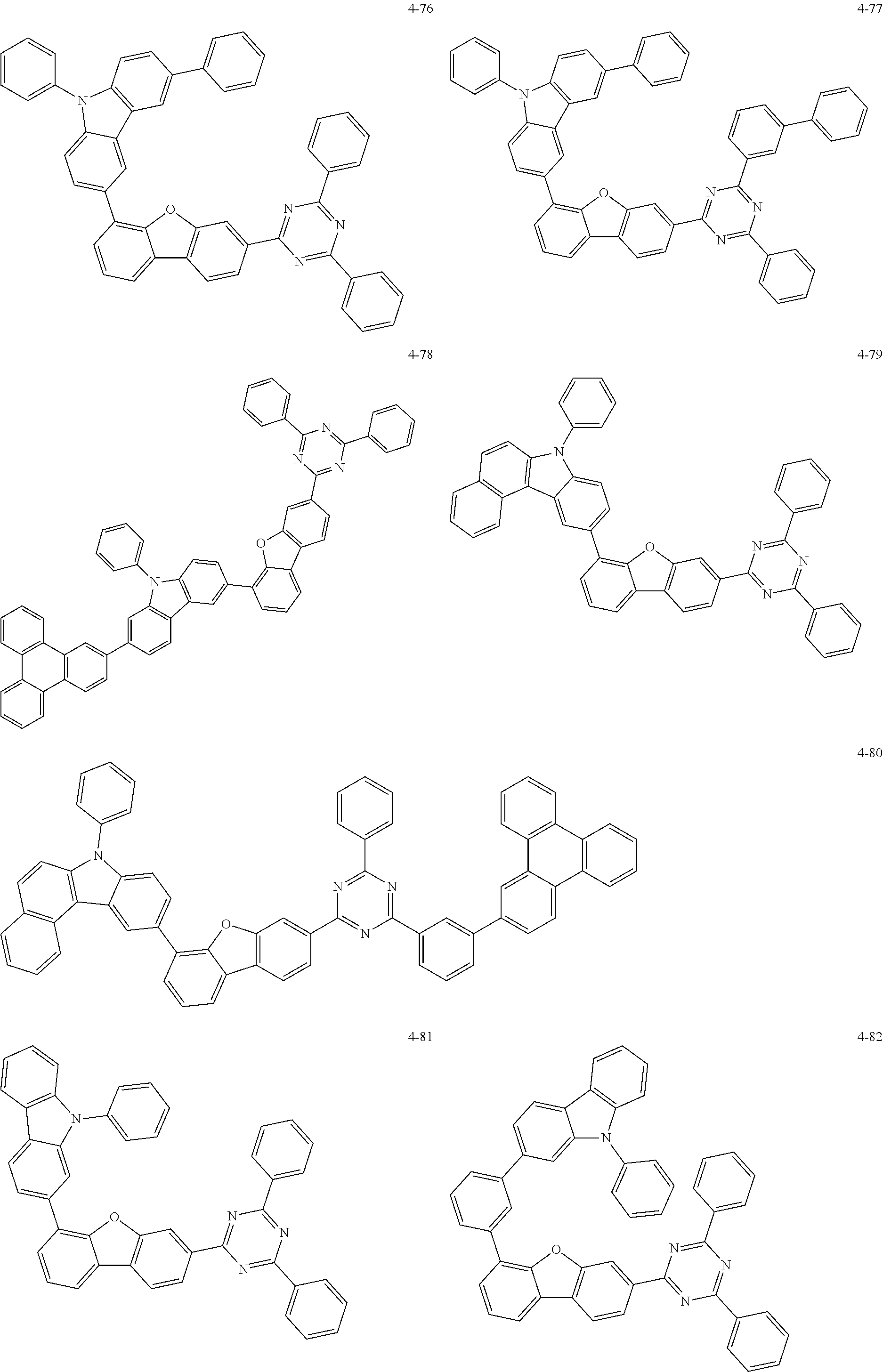

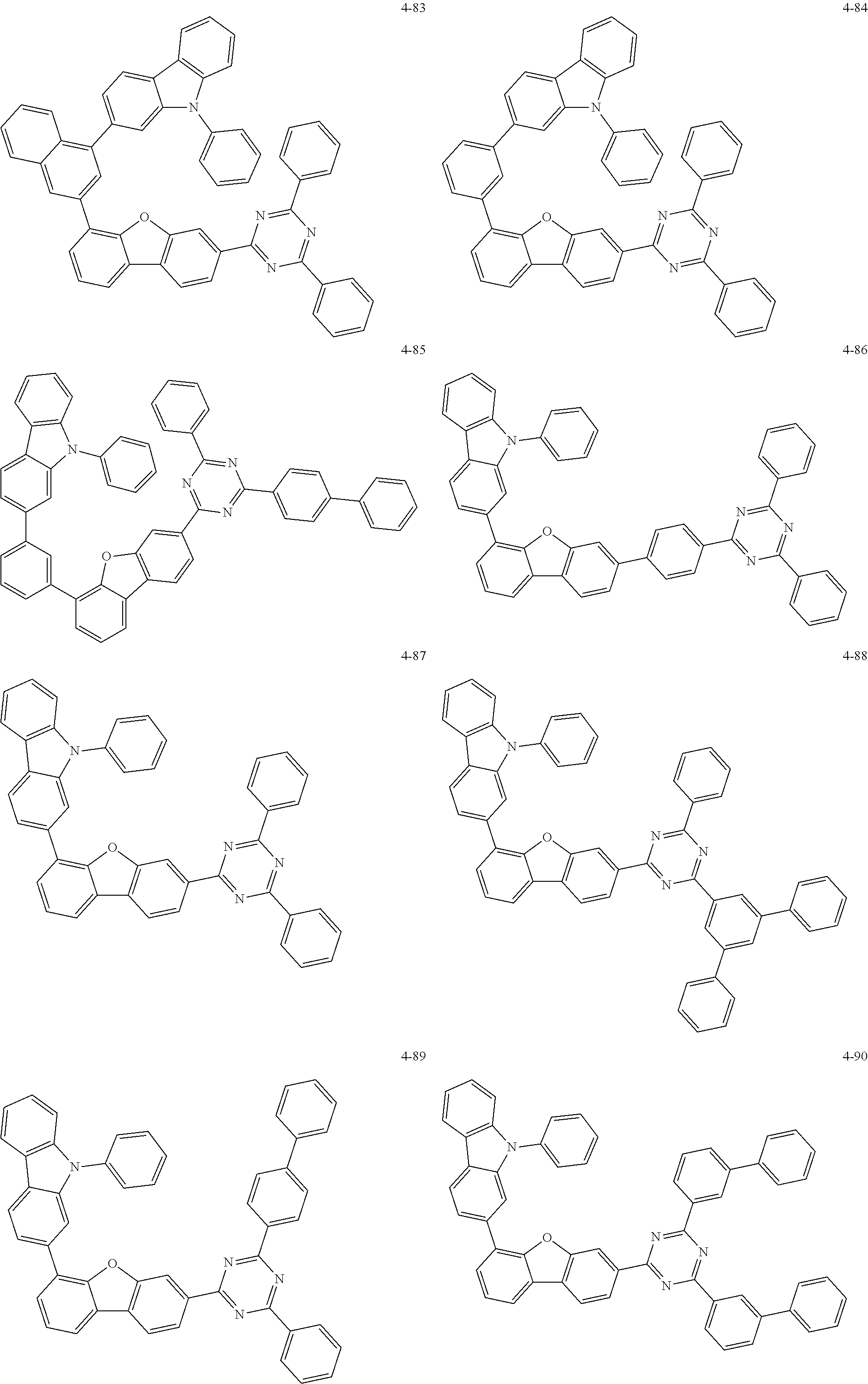

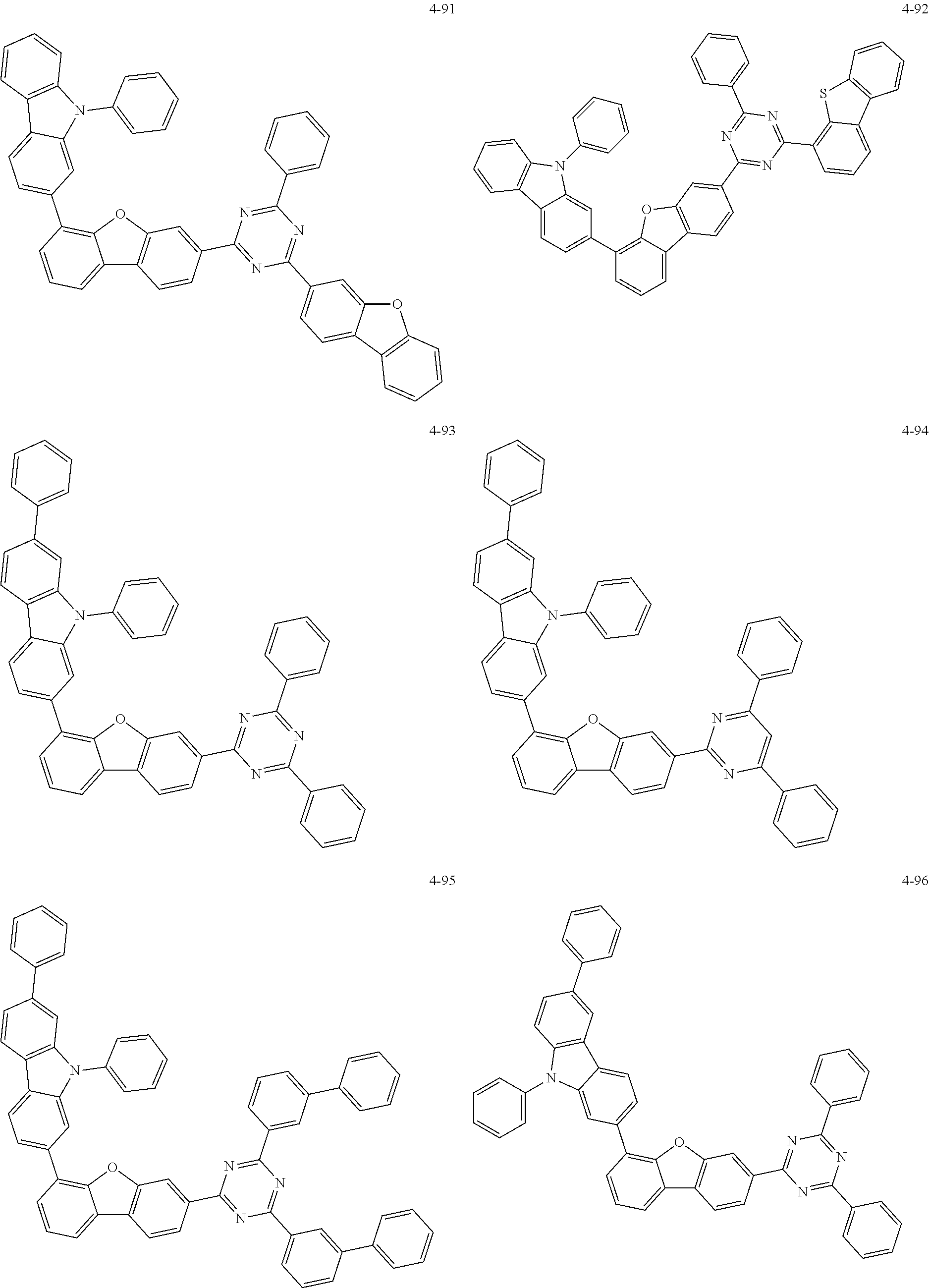

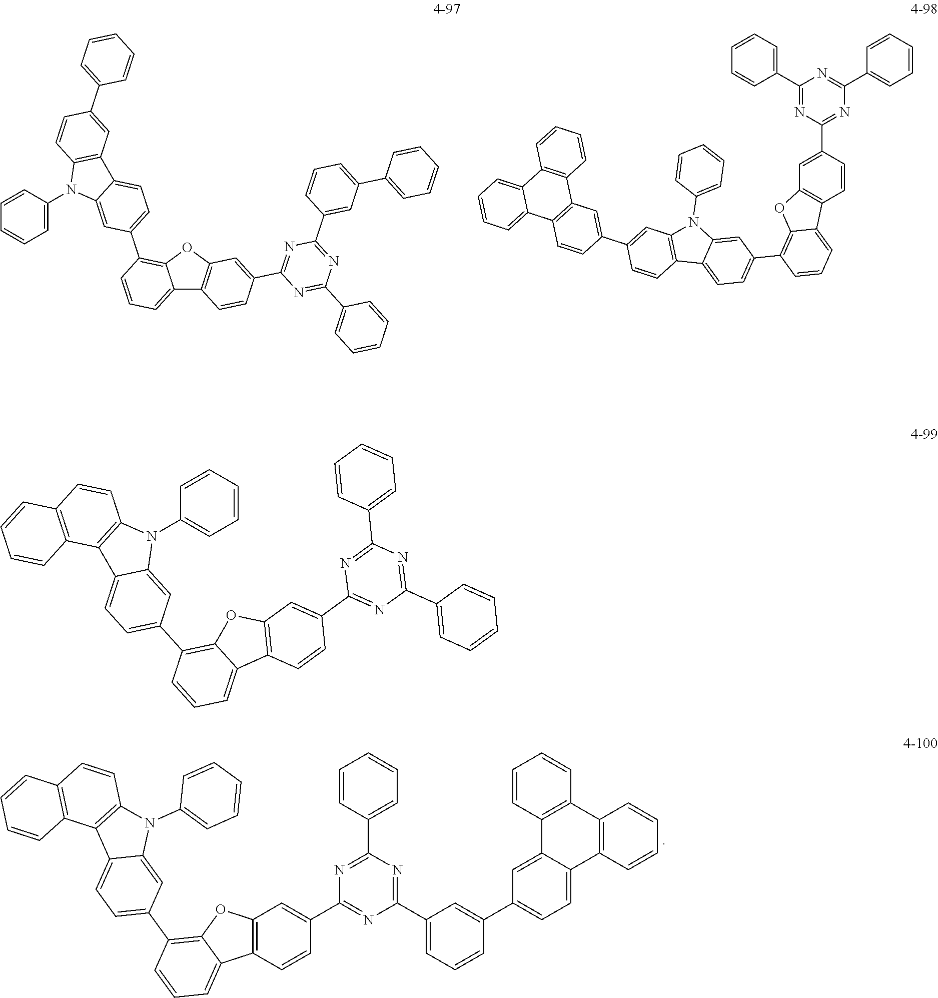 [SMILES: c1ccc(-c2cccc(-c3nc(-c4ccccc4)nc(-c4ccc5c(c4)oc4c(-c6ccc7c8cc(-c9ccccc9)ccc8n(-c8ccccc8)c7c6)cccc45)n3)c2)cc1 ; c1ccc(-c2nc(-c3ccccc3)nc(-c3ccc4c(c3)oc3c(-c5ccc6c7ccc(-c8ccc9c%10ccccc%10c%10ccccc%10c9c8)cc7n(-c7ccccc7)c6c5)cccc34)n2)cc1 ; c1ccc(-c2nc(-c3ccccc3)nc(-c3ccc4c(c3)oc3c(-c5ccc6c7c8ccccc8ccc7n(-c7ccccc7)c6c5)cccc34)n2)cc1 ; c1ccc(-c2nc(-c3cccc(-c4ccc5c6ccccc6c6ccccc6c5c4)c3)nc(-c3ccc4c(c3)oc3c(-c5ccc6c7c8ccccc8ccc7n(-c7ccccc7)c6c5)cccc34)n2)cc1]

7. An organic light emitting device comprising:
a first electrode;
a second electrode provided opposite to the first electrode; and
one or more organic material layers provided between the first electrode and the second electrode,
wherein one or more layers of the organic material layers comprise the heterocyclic compound of claim 1.

8. The organic light emitting device of claim 7, wherein the organic material layer comprises a light emitting layer, and the light emitting layer comprises the heterocyclic compound.

9. The organic light emitting device of claim 7, wherein the organic material layer comprises a light emitting layer, the light emitting layer comprises a host material, and the host material comprises the heterocyclic compound.

10. The organic light emitting device of claim 7, wherein the organic material layer comprises an electron injection layer or an electron transfer layer, and the electron transfer layer or the electron injection layer comprises the heterocyclic compound.

11. The organic light emitting device of claim 7, wherein the organic material layer comprises an electron blocking layer or a hole blocking layer, and the electron blocking layer or the hole blocking layer comprises the heterocyclic compound.

12. The organic light emitting device claim 7, further comprising one, two or more layers selected from the group consisting of a light emitting layer, a hole injection layer, a hole transfer layer, an electron injection layer, an electron transfer layer, an electron blocking layer and a hole blocking layer.

13. The organic light emitting device of claim 7, wherein the organic material layer further comprises a compound of the following Chemical Formula 2: 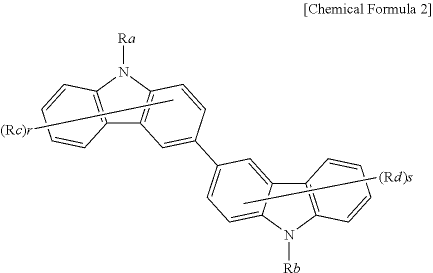
in Chemical Formula 2,
Ra and Rb are the same as or different from each other, and each independently a substituted or unsubstituted C6 to C60 aryl group; or a substituted or unsubstituted C2 to C60 heteroaryl group;
Rc and Rd are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; and a substituted or unsubstituted amine group; and
r and s are an integer of 0 to 7.

14. The organic light emitting device of claim 13, wherein Chemical Formula 2 is represented by any one of the following compounds:

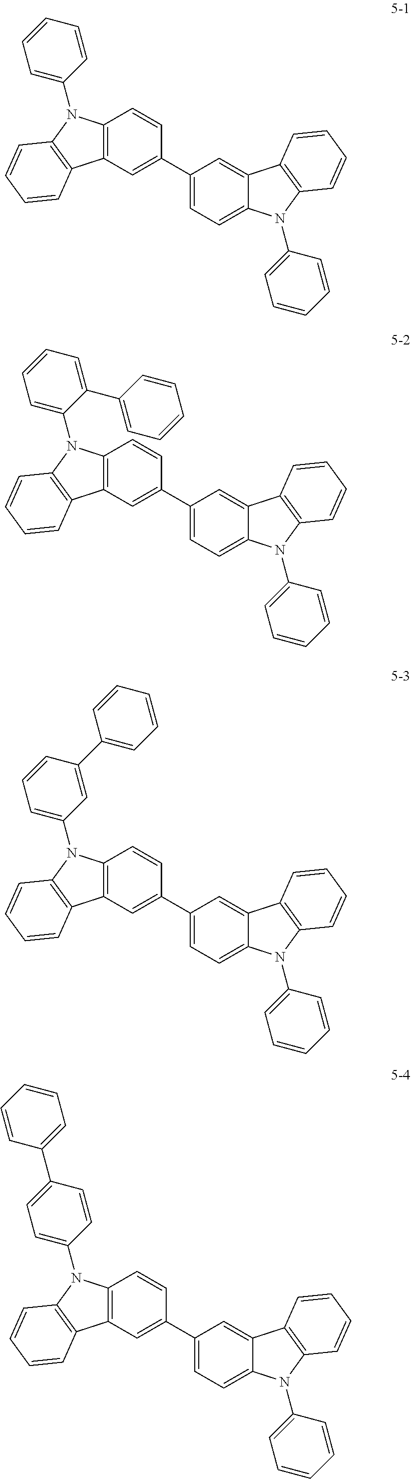 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1]

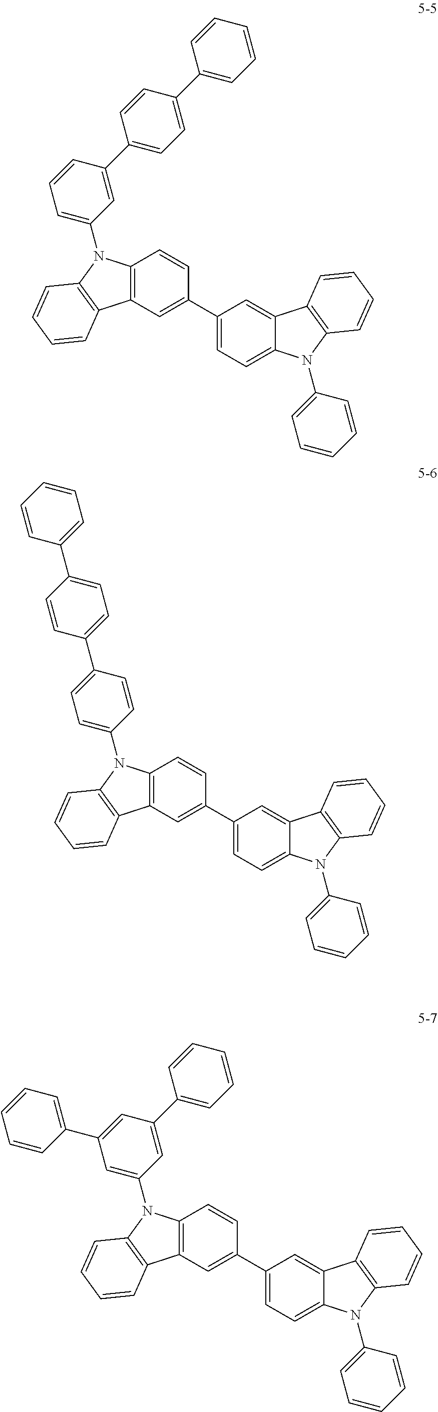 [SMILES: c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1 ; c1ccc(-c2cc(-c3ccccc3)cc(-n3c4ccccc4c4cc(-c5ccc6c(c5)c5ccccc5n6-c5ccccc5)ccc43)c2)cc1]

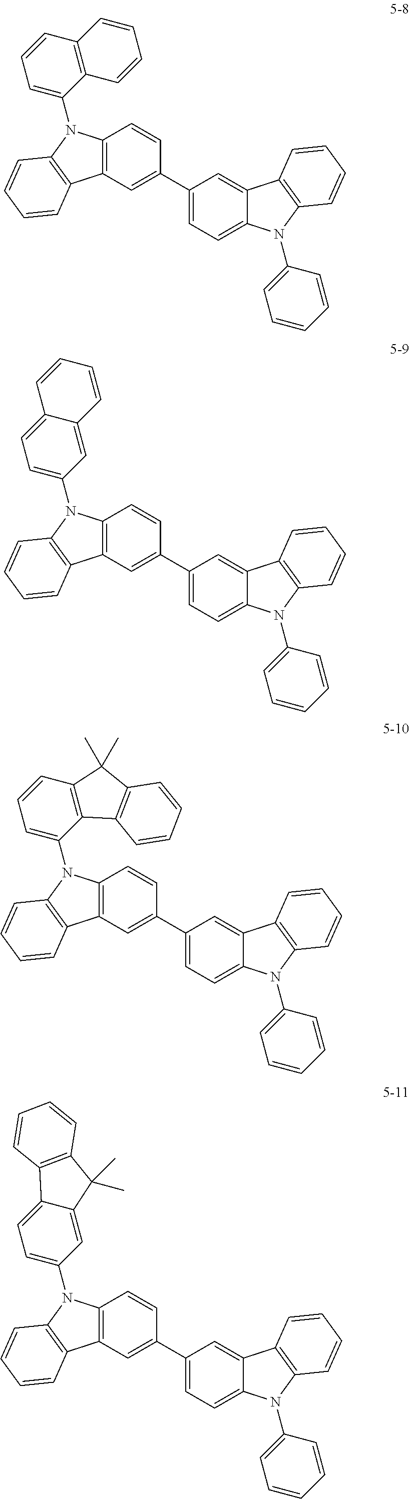 [SMILES: c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5ccccc5c4)ccc32)cc1 ; CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2ccc(-n3c4ccccc4c4cc(-c5ccc6c(c5)c5ccccc5n6-c5ccccc5)ccc43)cc21]

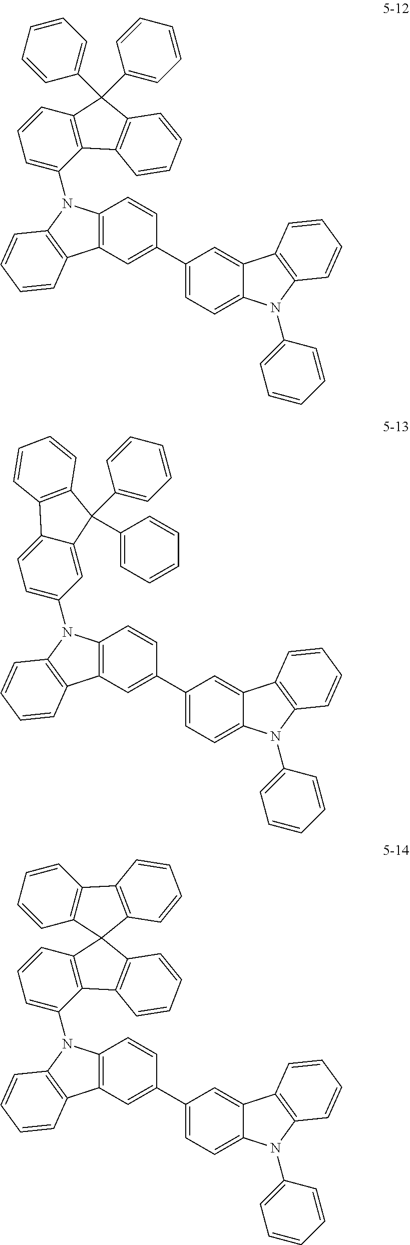 [SMILES: c1ccc(-n2c3ccccc3c3cc(-c4ccc5c(c4)c4ccccc4n5-c4cccc5c4-c4ccccc4C5(c4ccccc4)c4ccccc4)ccc32)cc1 ; c1ccc(-n2c3ccccc3c3cc(-c4ccc5c(c4)c4ccccc4n5-c4ccc5c(c4)C(c4ccccc4)(c4ccccc4)c4ccccc4-5)ccc32)cc1 ; c1ccc(-n2c3ccccc3c3cc(-c4ccc5c(c4)c4ccccc4n5-c4cccc5c4-c4ccccc4C54c5ccccc5-c5ccccc54)ccc32)cc1]

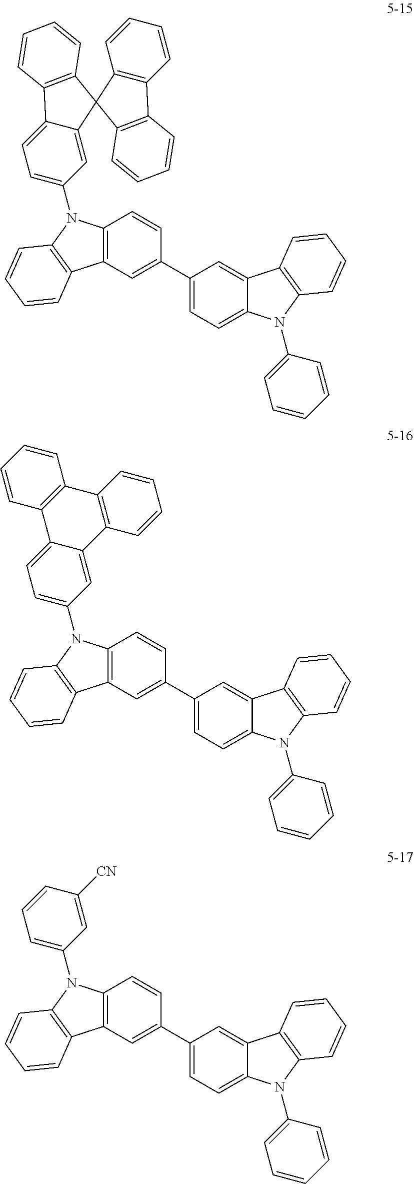 [SMILES: c1ccc(-n2c3ccccc3c3cc(-c4ccc5c(c4)c4ccccc4n5-c4ccc5c(c4)C4(c6ccccc6-c6ccccc64)c4ccccc4-5)ccc32)cc1 ; c1ccc(-n2c3ccccc3c3cc(-c4ccc5c(c4)c4ccccc4n5-c4ccc5c6ccccc6c6ccccc6c5c4)ccc32)cc1 ; N#Cc1cccc(-n2c3ccccc3c3cc(-c4ccc5c(c4)c4ccccc4n5-c4ccccc4)ccc32)c1]

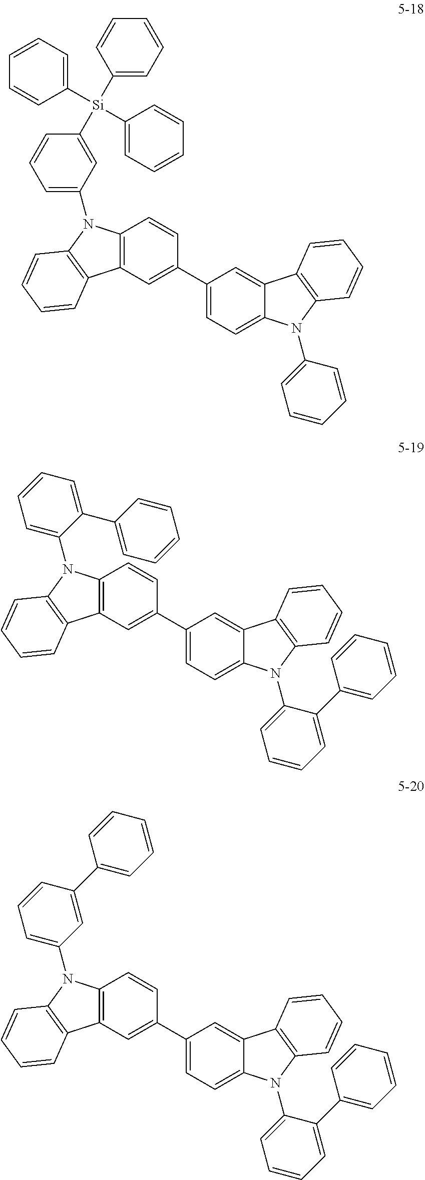 [SMILES: c1ccc(-n2c3ccccc3c3cc(-c4ccc5c(c4)c4ccccc4n5-c4cccc([Si](c5ccccc5)(c5ccccc5)c5ccccc5)c4)ccc32)cc1 ; c1ccc(-c2ccccc2-n2c3ccccc3c3cc(-c4ccc5c(c4)c4ccccc4n5-c4ccccc4-c4ccccc4)ccc32)cc1 ; c1ccc(-c2cccc(-n3c4ccccc4c4cc(-c5ccc6c(c5)c5ccccc5n6-c5ccccc5-c5ccccc5)ccc43)c2)cc1]

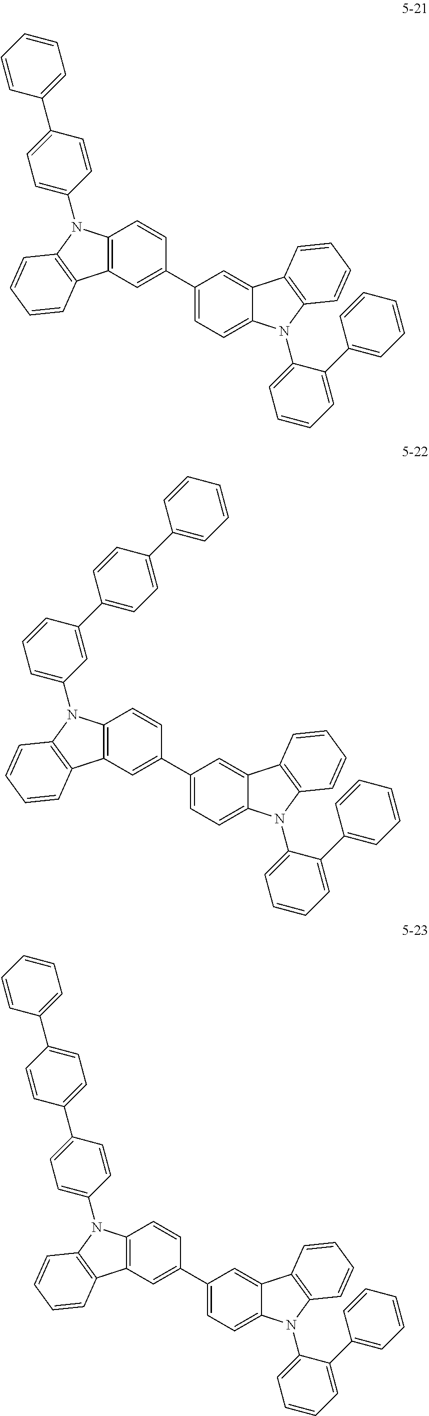 [SMILES: c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2ccc(-c3cccc(-n4c5ccccc5c5cc(-c6ccc7c(c6)c6ccccc6n7-c6ccccc6-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6-c6ccccc6)ccc54)cc3)cc2)cc1]

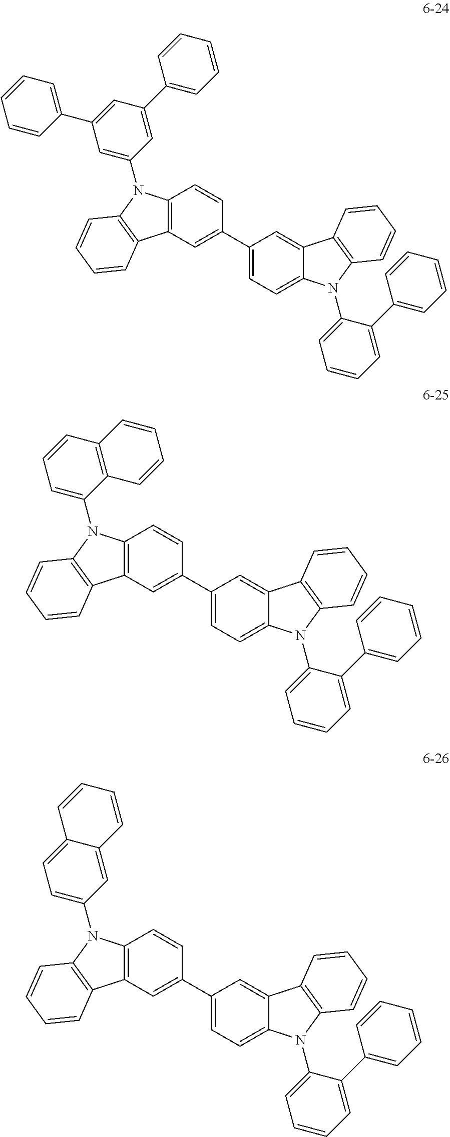 [SMILES: c1ccc(-c2cc(-c3ccccc3)cc(-n3c4ccccc4c4cc(-c5ccc6c(c5)c5ccccc5n6-c5ccccc5-c5ccccc5)ccc43)c2)cc1 ; c1ccc(-c2ccccc2-n2c3ccccc3c3cc(-c4ccc5c(c4)c4ccccc4n5-c4cccc5ccccc45)ccc32)cc1 ; c1ccc(-c2ccccc2-n2c3ccccc3c3cc(-c4ccc5c(c4)c4ccccc4n5-c4ccc5ccccc5c4)ccc32)cc1]

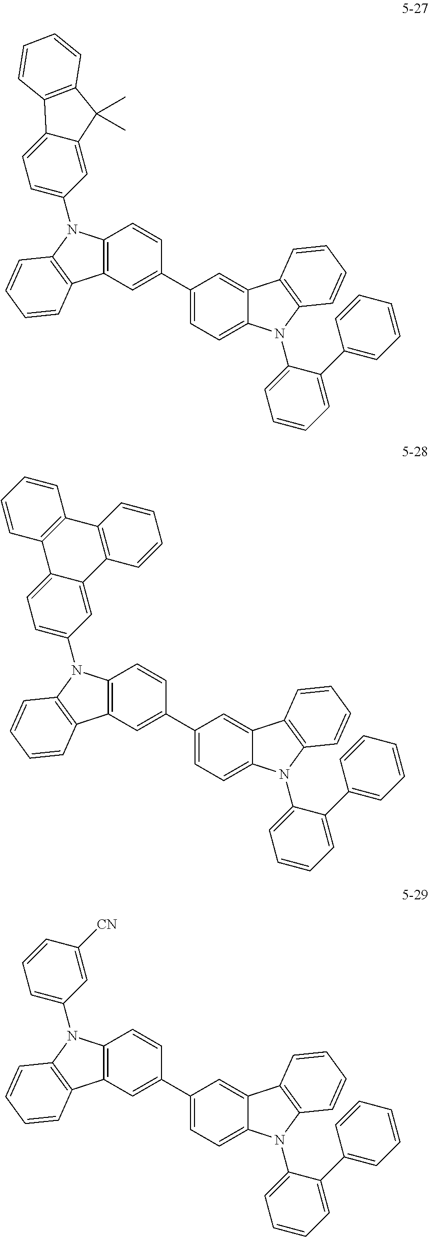 [SMILES: CC1(C)c2ccccc2-c2ccc(-n3c4ccccc4c4cc(-c5ccc6c(c5)c5ccccc5n6-c5ccccc5-c5ccccc5)ccc43)cc21 ; c1ccc(-c2ccccc2-n2c3ccccc3c3cc(-c4ccc5c(c4)c4ccccc4n5-c4ccc5c6ccccc6c6ccccc6c5c4)ccc32)cc1 ; N#Cc1cccc(-n2c3ccccc3c3cc(-c4ccc5c(c4)c4ccccc4n5-c4ccccc4-c4ccccc4)ccc32)c1]

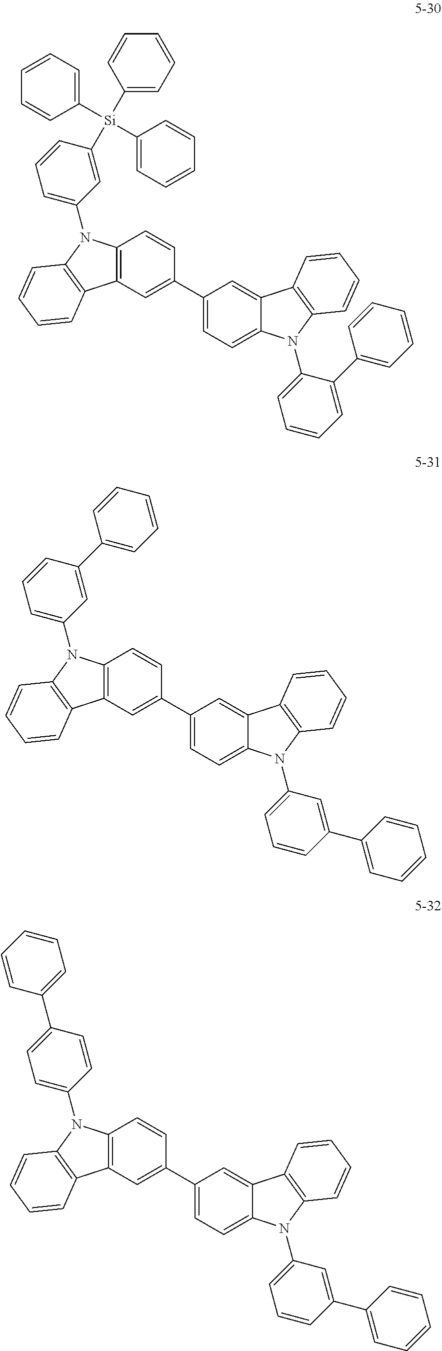 [SMILES: c1ccc(-c2ccccc2-n2c3ccccc3c3cc(-c4ccc5c(c4)c4ccccc4n5-c4cccc([Si](c5ccccc5)(c5ccccc5)c5ccccc5)c4)ccc32)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

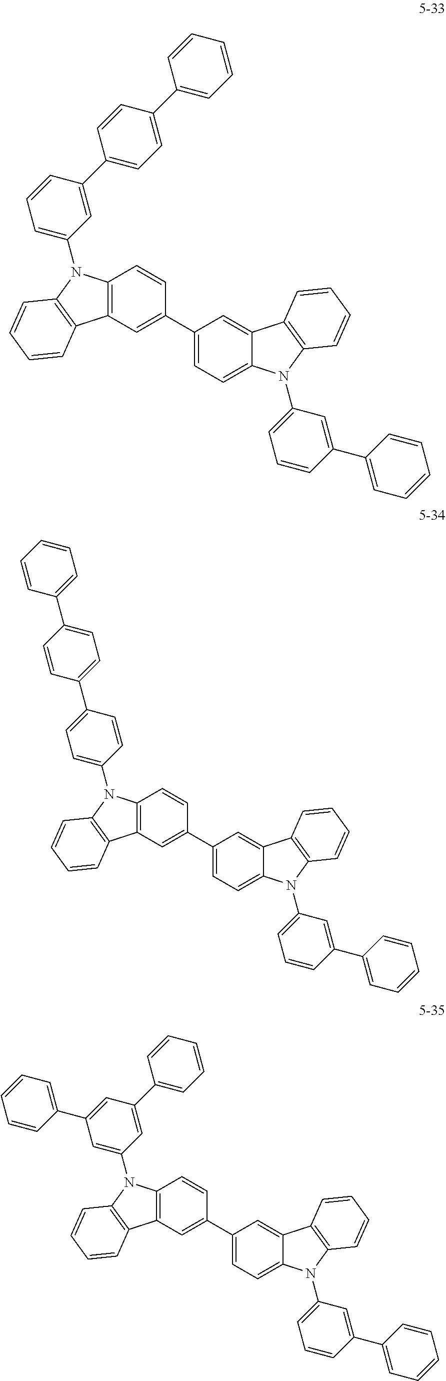 [SMILES: c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1 ; c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1]

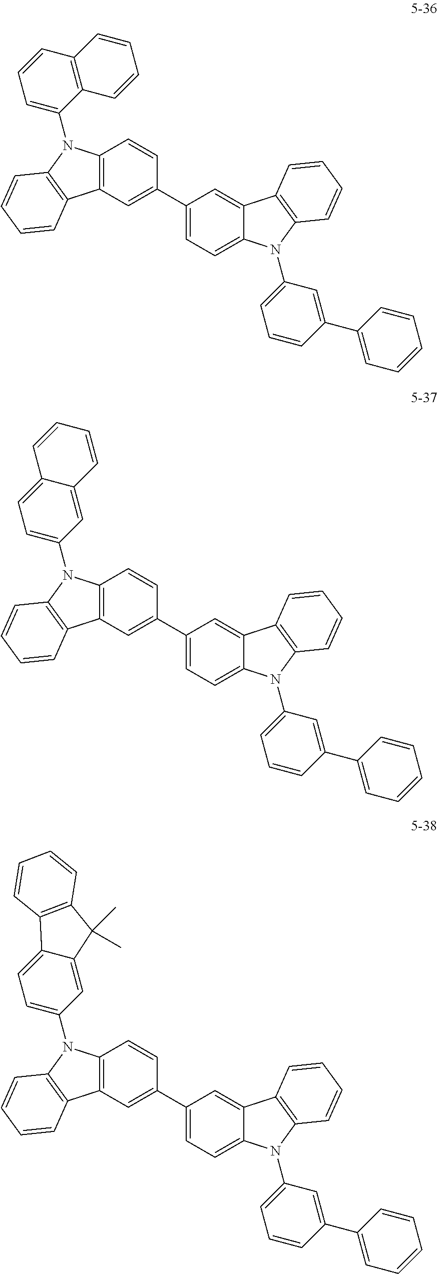 [SMILES: c1ccc(-c2cccc(-n3c4ccccc4c4cc(-c5ccc6c(c5)c5ccccc5n6-c5cccc6ccccc56)ccc43)c2)cc1 ; c1ccc(-c2cccc(-n3c4ccccc4c4cc(-c5ccc6c(c5)c5ccccc5n6-c5ccc6ccccc6c5)ccc43)c2)cc1 ; CC1(C)c2ccccc2-c2ccc(-n3c4ccccc4c4cc(-c5ccc6c(c5)c5ccccc5n6-c5cccc(-c6ccccc6)c5)ccc43)cc21]

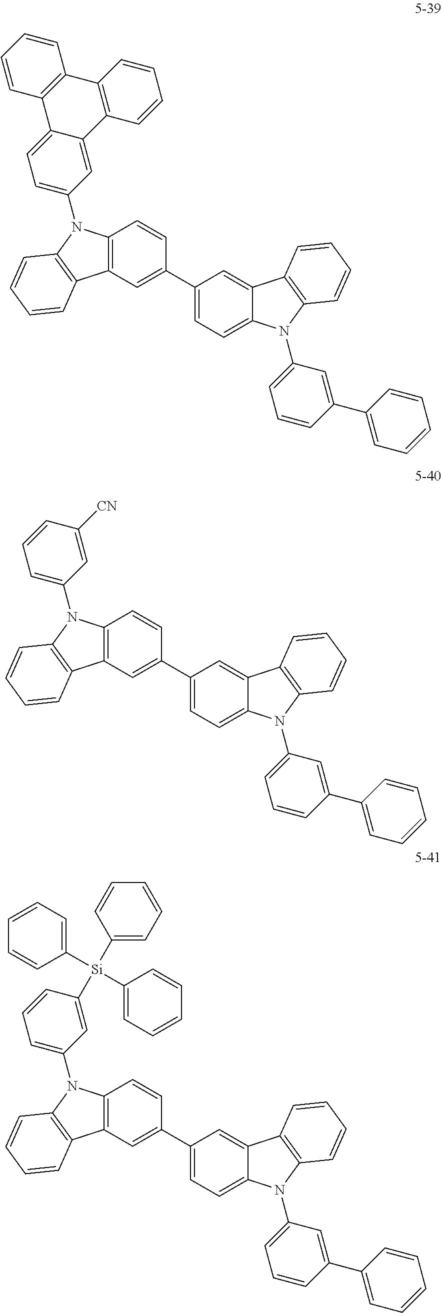 [SMILES: c1ccc(-c2cccc(-n3c4ccccc4c4cc(-c5ccc6c(c5)c5ccccc5n6-c5ccc6c7ccccc7c7ccccc7c6c5)ccc43)c2)cc1 ; N#Cc1cccc(-n2c3ccccc3c3cc(-c4ccc5c(c4)c4ccccc4n5-c4cccc(-c5ccccc5)c4)ccc32)c1 ; c1ccc(-c2cccc(-n3c4ccccc4c4cc(-c5ccc6c(c5)c5ccccc5n6-c5cccc([Si](c6ccccc6)(c6ccccc6)c6ccccc6)c5)ccc43)c2)cc1]

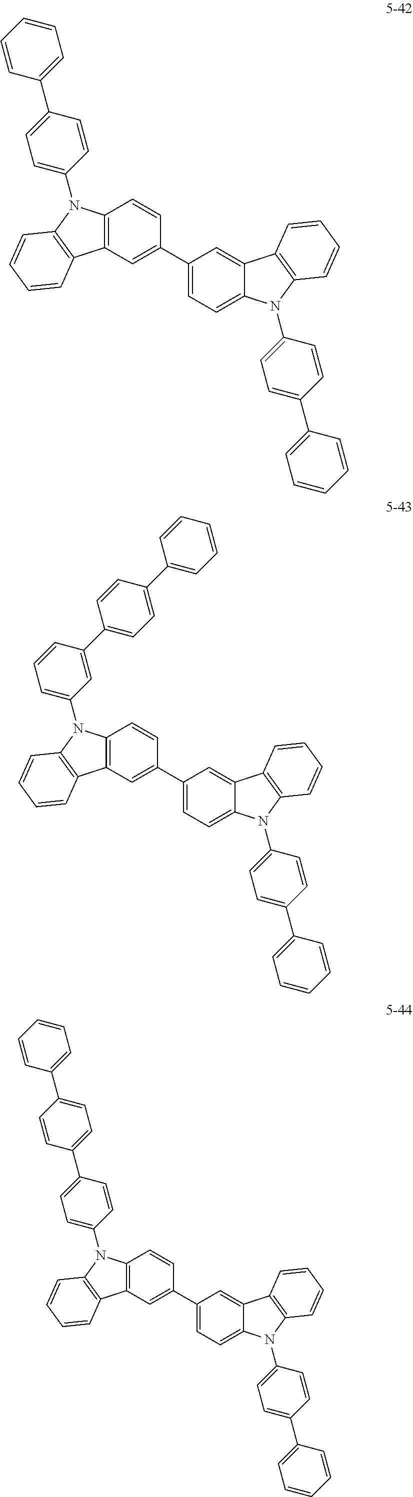 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1 ; c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1]

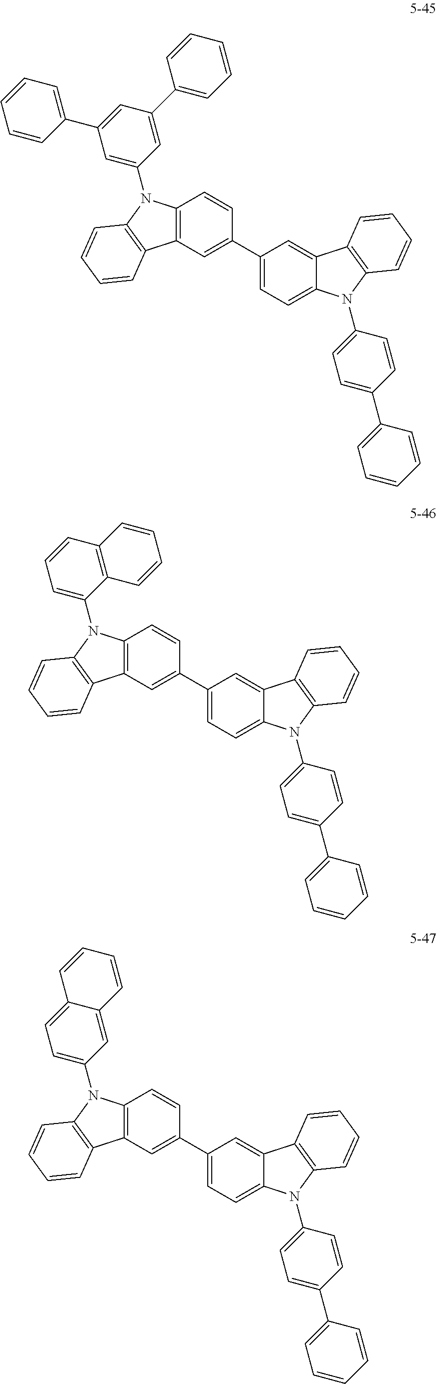 [SMILES: c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2ccc(-n3c4ccccc4c4cc(-c5ccc6c(c5)c5ccccc5n6-c5cccc6ccccc56)ccc43)cc2)cc1 ; c1ccc(-c2ccc(-n3c4ccccc4c4cc(-c5ccc6c(c5)c5ccccc5n6-c5ccc6ccccc6c5)ccc43)cc2)cc1]

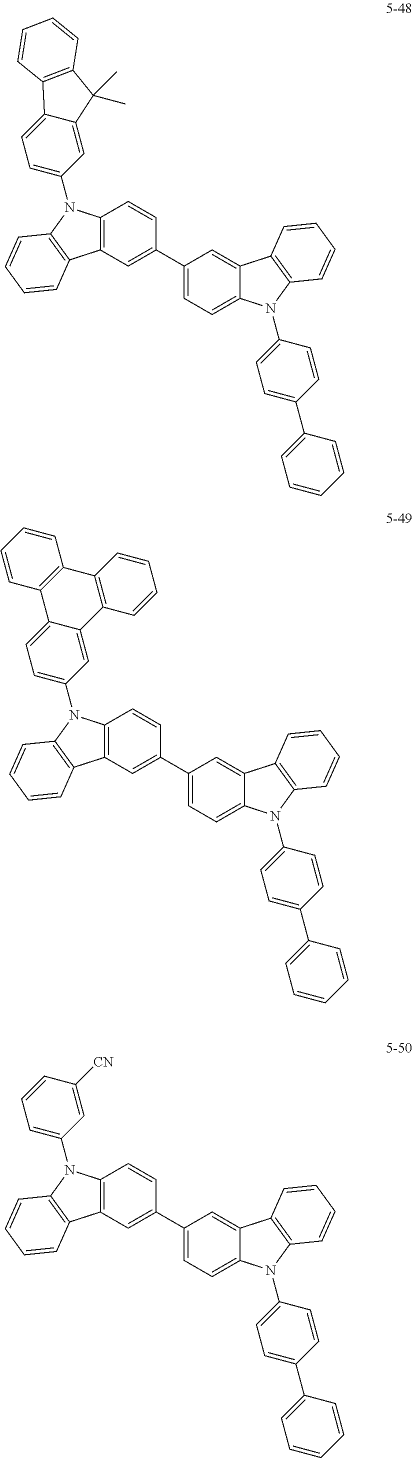 [SMILES: CC1(C)c2ccccc2-c2ccc(-n3c4ccccc4c4cc(-c5ccc6c(c5)c5ccccc5n6-c5ccc(-c6ccccc6)cc5)ccc43)cc21 ; c1ccc(-c2ccc(-n3c4ccccc4c4cc(-c5ccc6c(c5)c5ccccc5n6-c5ccc6c7ccccc7c7ccccc7c6c5)ccc43)cc2)cc1 ; N#Cc1cccc(-n2c3ccccc3c3cc(-c4ccc5c(c4)c4ccccc4n5-c4ccc(-c5ccccc5)cc4)ccc32)c1]

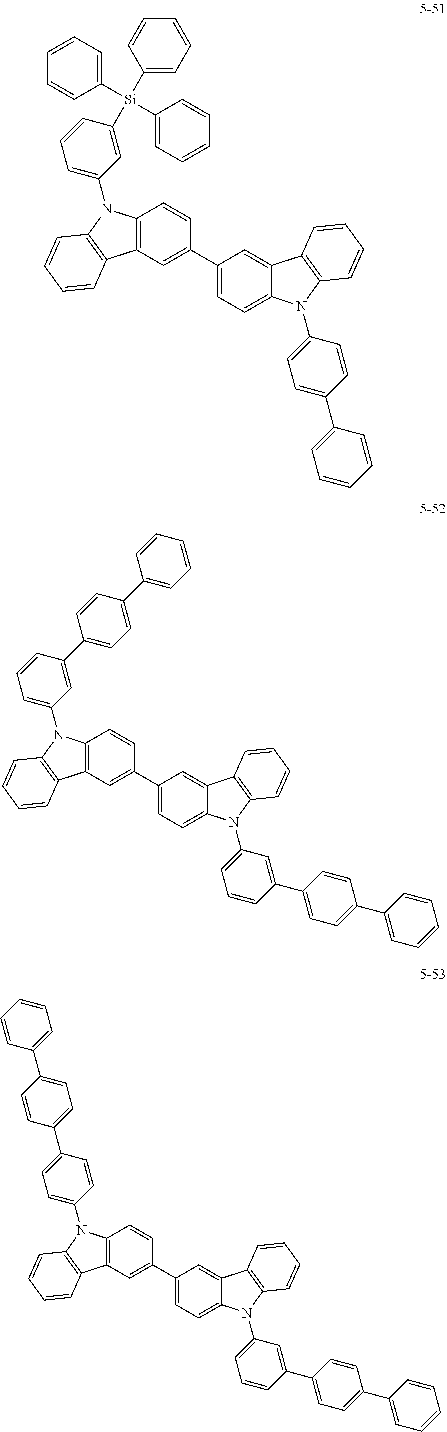 [SMILES: c1ccc(-c2ccc(-n3c4ccccc4c4cc(-c5ccc6c(c5)c5ccccc5n6-c5cccc([Si](c6ccccc6)(c6ccccc6)c6ccccc6)c5)ccc43)cc2)cc1 ; c1ccc(-c2ccc(-c3cccc(-n4c5ccccc5c5cc(-c6ccc7c(c6)c6ccccc6n7-c6cccc(-c7ccc(-c8ccccc8)cc7)c6)ccc54)c3)cc2)cc1 ; c1ccc(-c2ccc(-c3ccc(-n4c5ccccc5c5cc(-c6ccc7c(c6)c6ccccc6n7-c6cccc(-c7ccc(-c8ccccc8)cc7)c6)ccc54)cc3)cc2)cc1]

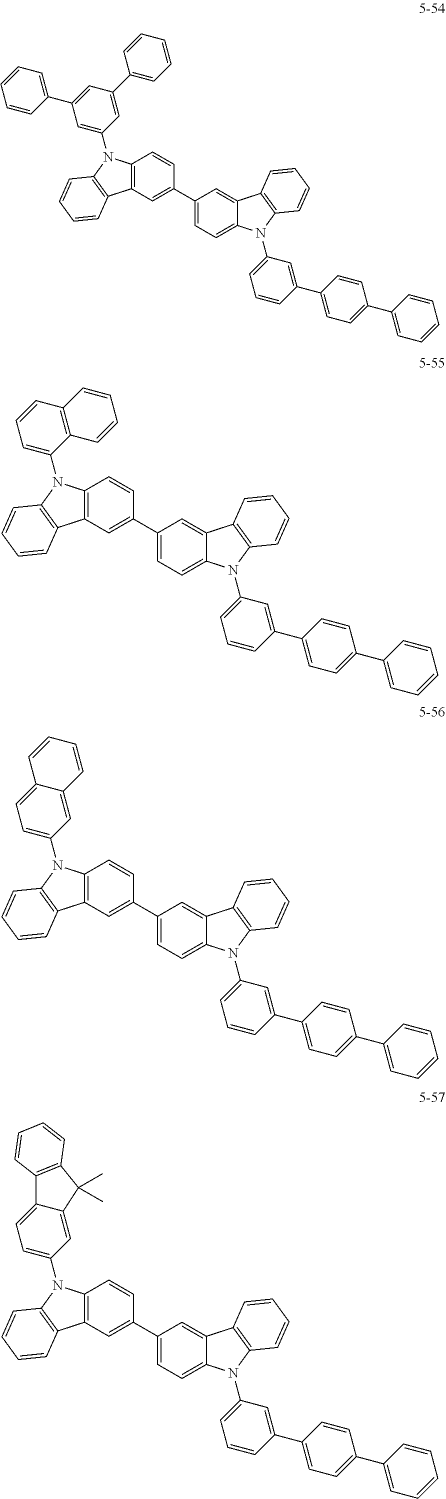 [SMILES: c1ccc(-c2ccc(-c3cccc(-n4c5ccccc5c5cc(-c6ccc7c(c6)c6ccccc6n7-c6cc(-c7ccccc7)cc(-c7ccccc7)c6)ccc54)c3)cc2)cc1 ; c1ccc(-c2ccc(-c3cccc(-n4c5ccccc5c5cc(-c6ccc7c(c6)c6ccccc6n7-c6cccc7ccccc67)ccc54)c3)cc2)cc1 ; c1ccc(-c2ccc(-c3cccc(-n4c5ccccc5c5cc(-c6ccc7c(c6)c6ccccc6n7-c6ccc7ccccc7c6)ccc54)c3)cc2)cc1 ; CC1(C)c2ccccc2-c2ccc(-n3c4ccccc4c4cc(-c5ccc6c(c5)c5ccccc5n6-c5cccc(-c6ccc(-c7ccccc7)cc6)c5)ccc43)cc21]

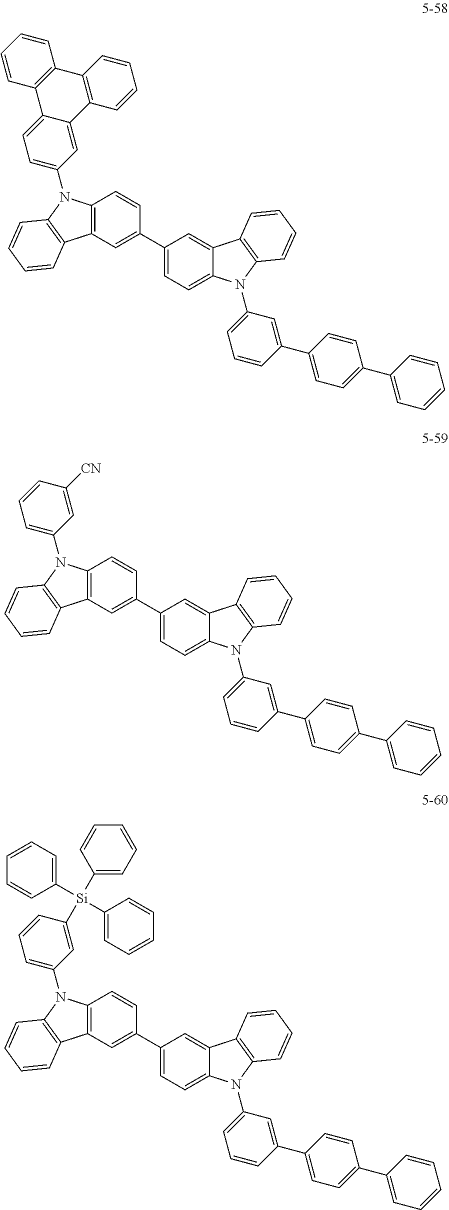 [SMILES: c1ccc(-c2ccc(-c3cccc(-n4c5ccccc5c5cc(-c6ccc7c(c6)c6ccccc6n7-c6ccc7c8ccccc8c8ccccc8c7c6)ccc54)c3)cc2)cc1 ; N#Cc1cccc(-n2c3ccccc3c3cc(-c4ccc5c(c4)c4ccccc4n5-c4cccc(-c5ccc(-c6ccccc6)cc5)c4)ccc32)c1 ; c1ccc(-c2ccc(-c3cccc(-n4c5ccccc5c5cc(-c6ccc7c(c6)c6ccccc6n7-c6cccc([Si](c7ccccc7)(c7ccccc7)c7ccccc7)c6)ccc54)c3)cc2)cc1]

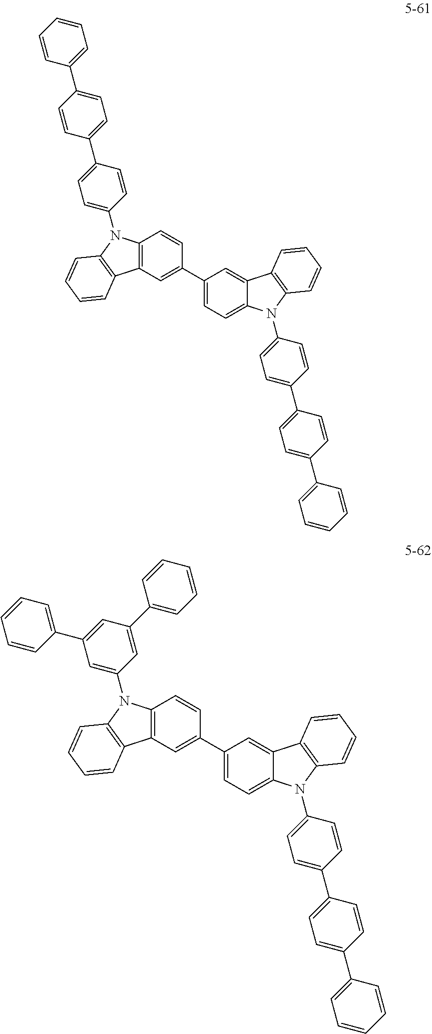 [SMILES: c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1 ; c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1]

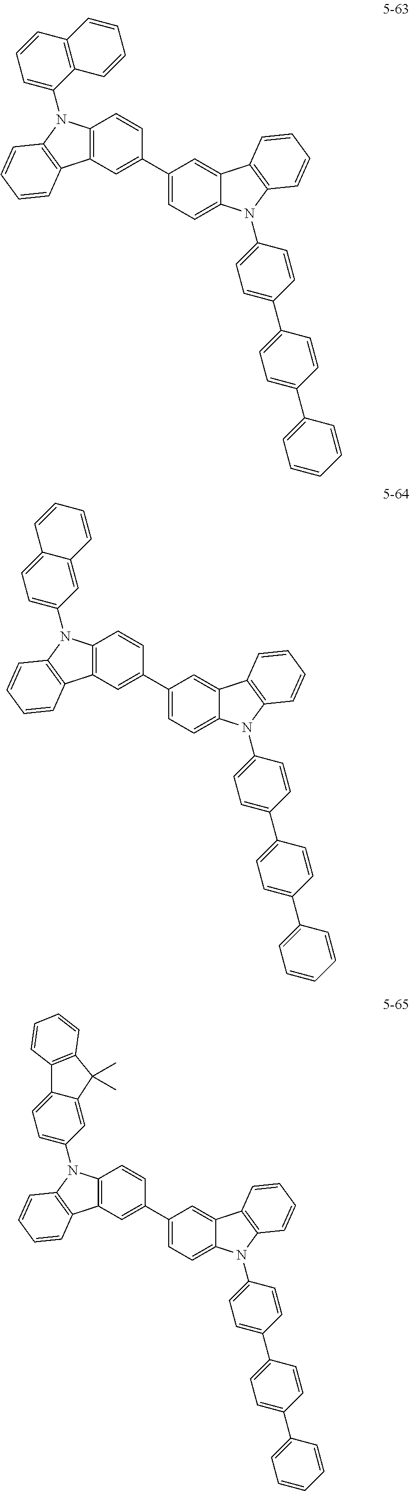 [SMILES: c1ccc(-c2ccc(-c3ccc(-n4c5ccccc5c5cc(-c6ccc7c(c6)c6ccccc6n7-c6cccc7ccccc67)ccc54)cc3)cc2)cc1 ; c1ccc(-c2ccc(-c3ccc(-n4c5ccccc5c5cc(-c6ccc7c(c6)c6ccccc6n7-c6ccc7ccccc7c6)ccc54)cc3)cc2)cc1 ; CC1(C)c2ccccc2-c2ccc(-n3c4ccccc4c4cc(-c5ccc6c(c5)c5ccccc5n6-c5ccc(-c6ccc(-c7ccccc7)cc6)cc5)ccc43)cc21]

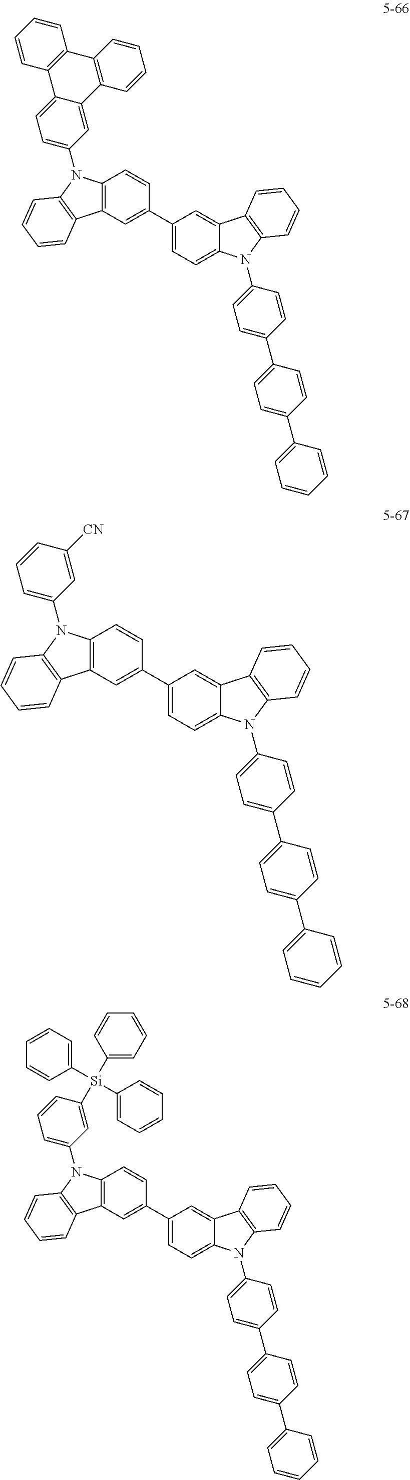 [SMILES: c1ccc(-c2ccc(-c3ccc(-n4c5ccccc5c5cc(-c6ccc7c(c6)c6ccccc6n7-c6ccc7c8ccccc8c8ccccc8c7c6)ccc54)cc3)cc2)cc1 ; N#Cc1cccc(-n2c3ccccc3c3cc(-c4ccc5c(c4)c4ccccc4n5-c4ccc(-c5ccc(-c6ccccc6)cc5)cc4)ccc32)c1 ; c1ccc(-c2ccc(-c3ccc(-n4c5ccccc5c5cc(-c6ccc7c(c6)c6ccccc6n7-c6cccc([Si](c7ccccc7)(c7ccccc7)c7ccccc7)c6)ccc54)cc3)cc2)cc1]

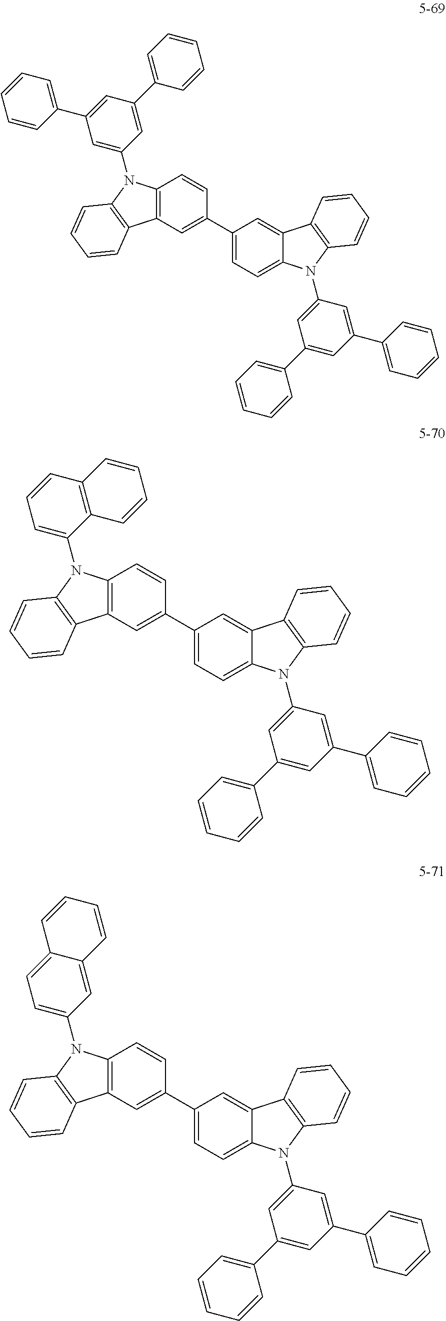 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cc(-c3ccccc3)cc(-n3c4ccccc4c4cc(-c5ccc6c(c5)c5ccccc5n6-c5cccc6ccccc56)ccc43)c2)cc1 ; c1ccc(-c2cc(-c3ccccc3)cc(-n3c4ccccc4c4cc(-c5ccc6c(c5)c5ccccc5n6-c5ccc6ccccc6c5)ccc43)c2)cc1]

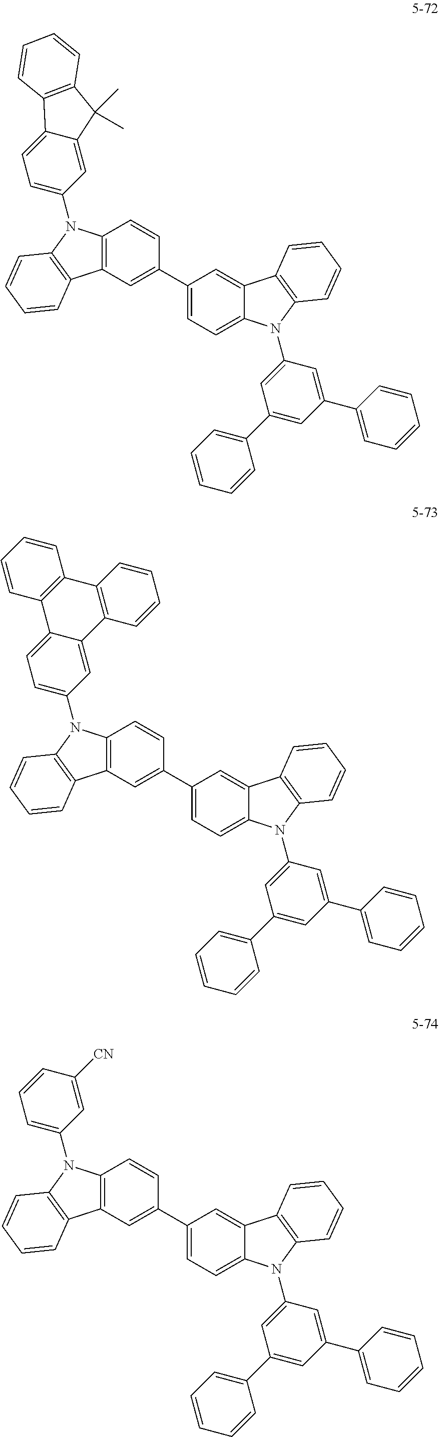 [SMILES: CC1(C)c2ccccc2-c2ccc(-n3c4ccccc4c4cc(-c5ccc6c(c5)c5ccccc5n6-c5cc(-c6ccccc6)cc(-c6ccccc6)c5)ccc43)cc21 ; c1ccc(-c2cc(-c3ccccc3)cc(-n3c4ccccc4c4cc(-c5ccc6c(c5)c5ccccc5n6-c5ccc6c7ccccc7c7ccccc7c6c5)ccc43)c2)cc1 ; N#Cc1cccc(-n2c3ccccc3c3cc(-c4ccc5c(c4)c4ccccc4n5-c4cc(-c5ccccc5)cc(-c5ccccc5)c4)ccc32)c1]

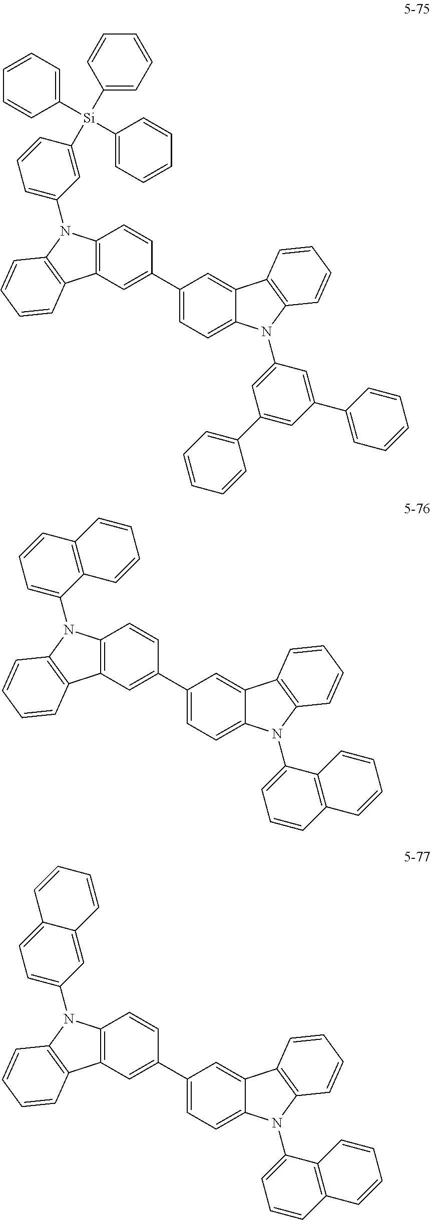 [SMILES: c1ccc(-c2cc(-c3ccccc3)cc(-n3c4ccccc4c4cc(-c5ccc6c(c5)c5ccccc5n6-c5cccc([Si](c6ccccc6)(c6ccccc6)c6ccccc6)c5)ccc43)c2)cc1 ; c1ccc2c(-n3c4ccccc4c4cc(-c5ccc6c(c5)c5ccccc5n6-c5cccc6ccccc56)ccc43)cccc2c1 ; c1ccc2cc(-n3c4ccccc4c4cc(-c5ccc6c(c5)c5ccccc5n6-c5cccc6ccccc56)ccc43)ccc2c1]

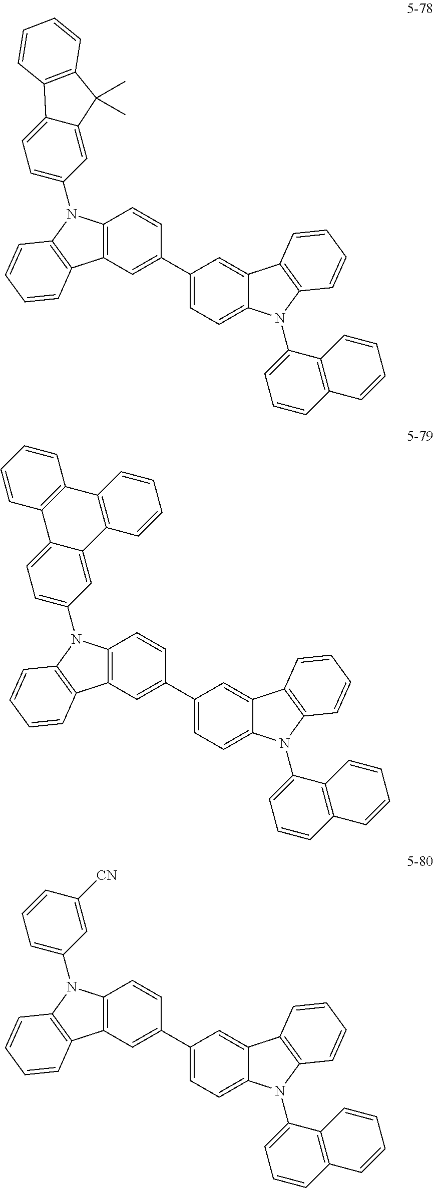 [SMILES: CC1(C)c2ccccc2-c2ccc(-n3c4ccccc4c4cc(-c5ccc6c(c5)c5ccccc5n6-c5cccc6ccccc56)ccc43)cc21 ; c1ccc2c(-n3c4ccccc4c4cc(-c5ccc6c(c5)c5ccccc5n6-c5ccc6c7ccccc7c7ccccc7c6c5)ccc43)cccc2c1 ; N#Cc1cccc(-n2c3ccccc3c3cc(-c4ccc5c(c4)c4ccccc4n5-c4cccc5ccccc45)ccc32)c1]

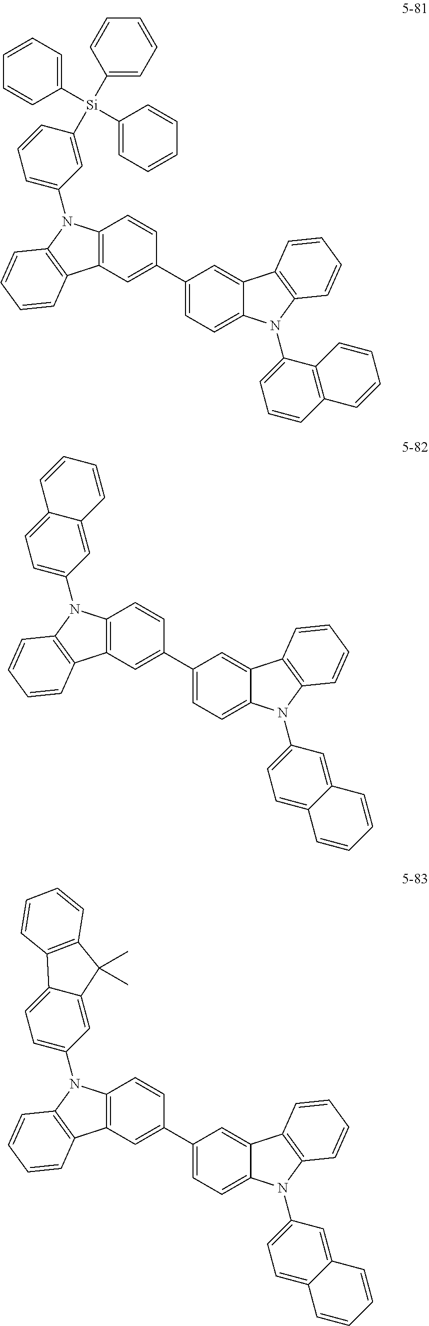 [SMILES: c1ccc([Si](c2ccccc2)(c2ccccc2)c2cccc(-n3c4ccccc4c4cc(-c5ccc6c(c5)c5ccccc5n6-c5cccc6ccccc56)ccc43)c2)cc1 ; c1ccc2cc(-n3c4ccccc4c4cc(-c5ccc6c(c5)c5ccccc5n6-c5ccc6ccccc6c5)ccc43)ccc2c1 ; CC1(C)c2ccccc2-c2ccc(-n3c4ccccc4c4cc(-c5ccc6c(c5)c5ccccc5n6-c5ccc6ccccc6c5)ccc43)cc21]

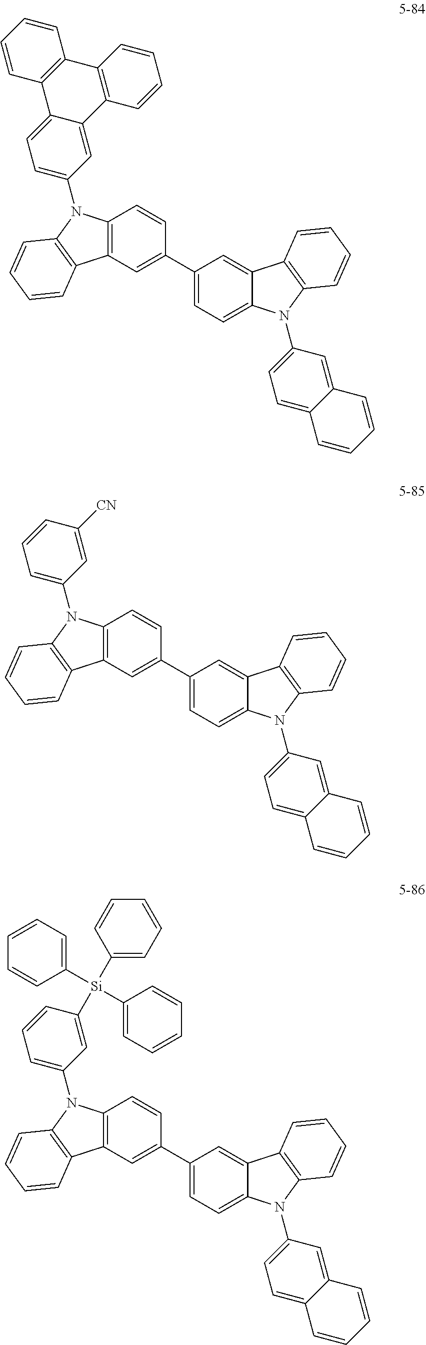 [SMILES: c1ccc2cc(-n3c4ccccc4c4cc(-c5ccc6c(c5)c5ccccc5n6-c5ccc6c7ccccc7c7ccccc7c6c5)ccc43)ccc2c1 ; N#Cc1cccc(-n2c3ccccc3c3cc(-c4ccc5c(c4)c4ccccc4n5-c4ccc5ccccc5c4)ccc32)c1 ; c1ccc([Si](c2ccccc2)(c2ccccc2)c2cccc(-n3c4ccccc4c4cc(-c5ccc6c(c5)c5ccccc5n6-c5ccc6ccccc6c5)ccc43)c2)cc1]

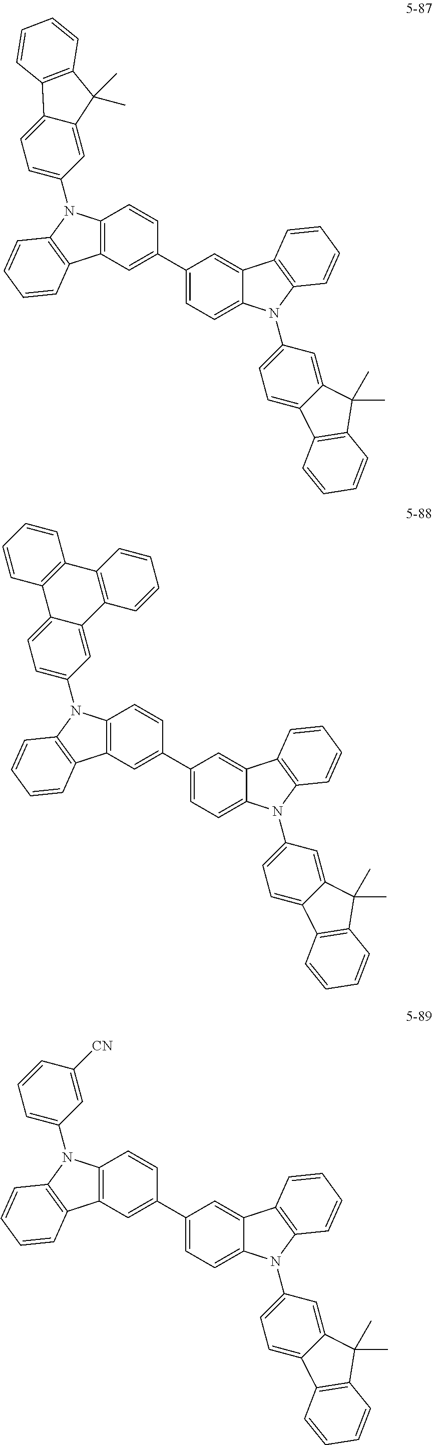 [SMILES: CC1(C)c2ccccc2-c2ccc(-n3c4ccccc4c4cc(-c5ccc6c(c5)c5ccccc5n6-c5ccc6c(c5)C(C)(C)c5ccccc5-6)ccc43)cc21 ; CC1(C)c2ccccc2-c2ccc(-n3c4ccccc4c4cc(-c5ccc6c(c5)c5ccccc5n6-c5ccc6c7ccccc7c7ccccc7c6c5)ccc43)cc21 ; CC1(C)c2ccccc2-c2ccc(-n3c4ccccc4c4cc(-c5ccc6c(c5)c5ccccc5n6-c5cccc(C#N)c5)ccc43)cc21]

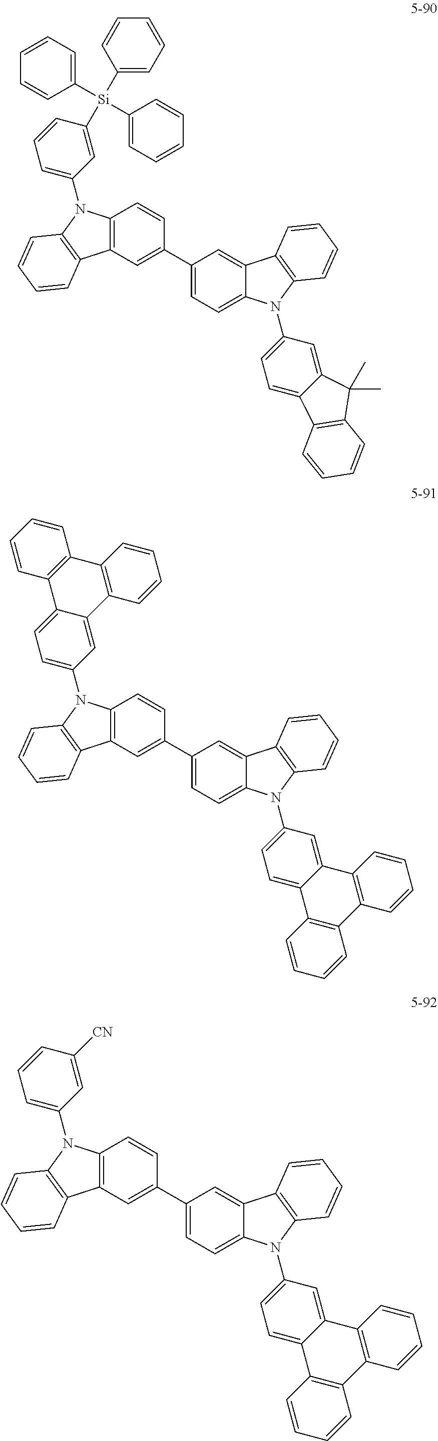 [SMILES: CC1(C)c2ccccc2-c2ccc(-n3c4ccccc4c4cc(-c5ccc6c(c5)c5ccccc5n6-c5cccc([Si](c6ccccc6)(c6ccccc6)c6ccccc6)c5)ccc43)cc21 ; c1ccc2c(c1)c1ccccc1c1cc(-n3c4ccccc4c4cc(-c5ccc6c(c5)c5ccccc5n6-c5ccc6c7ccccc7c7ccccc7c6c5)ccc43)ccc21 ; N#Cc1cccc(-n2c3ccccc3c3cc(-c4ccc5c(c4)c4ccccc4n5-c4ccc5c6ccccc6c6ccccc6c5c4)ccc32)c1]

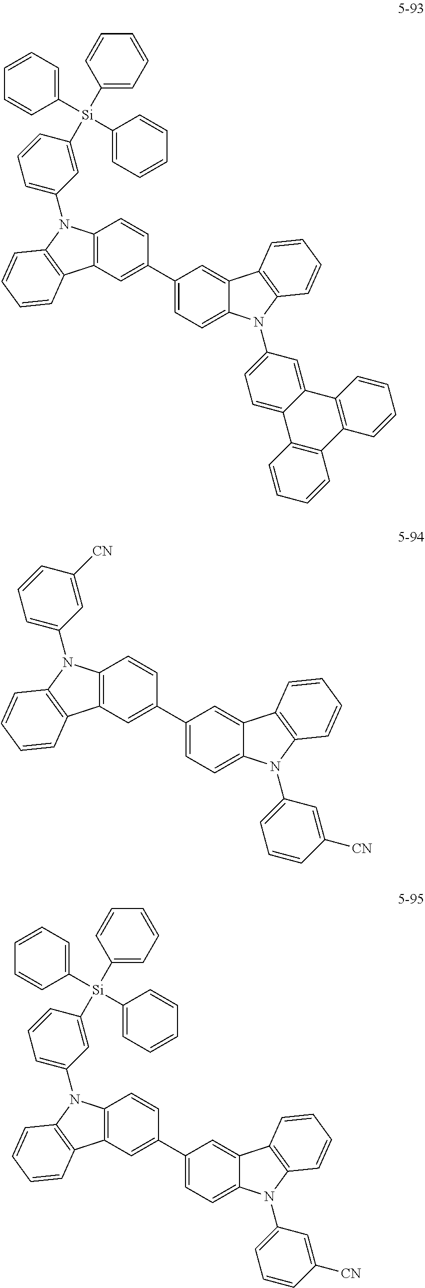 [SMILES: c1ccc([Si](c2ccccc2)(c2ccccc2)c2cccc(-n3c4ccccc4c4cc(-c5ccc6c(c5)c5ccccc5n6-c5ccc6c7ccccc7c7ccccc7c6c5)ccc43)c2)cc1 ; N#Cc1cccc(-n2c3ccccc3c3cc(-c4ccc5c(c4)c4ccccc4n5-c4cccc(C#N)c4)ccc32)c1 ; N#Cc1cccc(-n2c3ccccc3c3cc(-c4ccc5c(c4)c4ccccc4n5-c4cccc([Si](c5ccccc5)(c5ccccc5)c5ccccc5)c4)ccc32)c1]

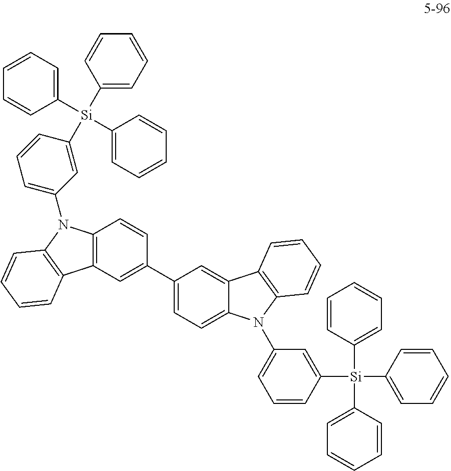 [SMILES: c1ccc([Si](c2ccccc2)(c2ccccc2)c2cccc(-n3c4ccccc4c4cc(-c5ccc6c(c5)c5ccccc5n6-c5cccc([Si](c6ccccc6)(c6ccccc6)c6ccccc6)c5)ccc43)c2)cc1]

15. A composition for an organic material layer of an organic light emitting device, the composition comprising:
the heterocyclic compound represented by Chemical Formula 1 of claim 1; and
a heterocyclic compound represented by the following Chemical Formula 2: 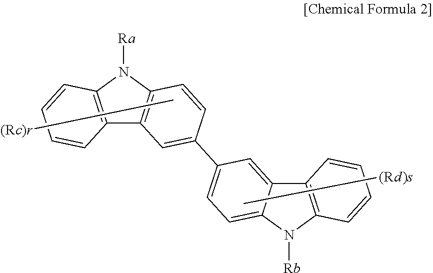
wherein, in Chemical Formula 2,
Ra and Rb are the same as or different from each other, and each independently a substituted or unsubstituted C6 to C60 aryl group; or a substituted or unsubstituted C2 to C60 heteroaryl group;
Rc and Rd are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; and a substituted or unsubstituted amine group; and
r and s are an integer of 0 to 7.

16. The composition for an organic material layer of an organic light emitting device of claim 15, wherein, in the composition, the heterocyclic compound represented by Chemical Formula 1 and the heterocyclic compound represented by Chemical Formula 2 have a weight ratio of 1:10 to 10:1.

17. A method for manufacturing an organic light emitting device, the method comprising:
preparing a substrate;
forming a fast electrode on the substrate;
forming one or more organic material layers on the first electrode; and
forming a second electrode on the organic material layer,
wherein the forming of organic material layers comprises forming one or more organic material layers using the composition for an organic material layer of claim 15.

18. The method for manufacturing an organic light emitting device of claim 17, wherein the forming of organic material layers is forming using a thermal vacuum deposition method after pre-mixing the heterocyclic compound of Chemical Formula 1 and the heterocyclic compound of Chemical Formula 2.
